(19) United States
(12) Patent Application Publication
(10) Pub. No.: US 2011/0178053 A1
(43) Pub. Date: Jul. 21, 2011

(54) AZACARBOLINE DERIVATIVES, PREPARATION METHOD THEREOF AND THERAPEUTIC USE OF SAME

(75) Inventors: Christopher Arendt, Bridgewater, NJ (US); Didier Babin, Paris (FR); Olivier Bedel, Paris (FR); Thierry Gouyon, Paris (FR); Mikhail Levit, Bridgewater, NJ (US); Ronghua Li, Bridgewater, NJ (US); Serge Mignani, Paris (FR); Neil Moorcroft, Bridgewater, NJ (US); David Papin, Paris (FR)

(73) Assignee: SANOFI-AVENTIS, Paris (FR)

(21) Appl. No.: 12/997,326

(22) PCT Filed: Jun. 11, 2009
(86) PCT No.: PCT/FR09/51100

§ 371 Date: Apr. 7, 2011

(30) Foreign Application Priority Data
Jun. 12, 2008 (FR) 08/03262

(51) Int. Cl. A61K 31/555; C07D 403/14; A61K 31/496; C07D 471/14; A61K 31/4375; C07D 413/14; A61K 31/5377; C07D 401/14; A61K 31/551; A61K 31/506; C07F 9/92; A61P 35/00

(52) U.S. Cl. 514/189; 544/361; 514/253.03; 546/82; 514/293; 544/126; 514/232.8; 540/575; 514/218; 544/316; 514/274; 546/4

(57) ABSTRACT

The invention relates to novel azacarbonlines having formula (I), wherein: R3, R4 represent independently H; hal; CF₃; substituted oxy, optionally substituted alkoxy; optionally substituted amino; substituted carbonyl; optionally substituted carboxyl; optionally substituted amide; sulphur, such as optionally substituted sulphones, sulphoxides or sulphides; linear, branched or cyclic C₁-C₁₀ alkyl optionally comprising an optionally substituted heteroatom; optionally substituted linear, branched or cyclic C₂-C₇ alkenyl; optionally substituted linear or branched C₂-C₆ alkynyl; optionally substituted aryl or heteroaryl; of which may be optionally substituted; in the form of a base or an acid addition salt. The invention also relates to the use of same in therapeutics for the treatment of cancer and to synthesis methods.

The present invention relates to α-aza-β-carboline derivatives, to their preparation and to their therapeutic use.

α-Aza-β-carbolines are defined by 1,7-diazacarbazole or 8-aza-β-carboline derivatives; in official nomenclature, the name of this tricyclic unit is 9H-pyrrolo[2,3-b:5,4-c′]dipyridine.

The present invention is directed towards compounds that act on kinase proteins, for instance: CHK1, CDK1, CDK2, dyrk2, Flt3, GSK3 beta, MNK2, PDGFR beta, PI3K, PIM1, PIM2, PIM3, PLK, TrkB, which are all involved in the development of cancers. More particularly, the present invention is directed towards compounds that act on a target known as Pim, which is involved in the development of cancers.

The Pim kinases, which include Pim-1, Pim-2 and Pim-3, form a distinct family of serine/threonine kinases, and play a functional role in cell growth, differentiation and apoptosis. One of the mechanisms via which the Pim kinases can increase the survival of cancer cells and promote the evolution of cancer proceeds via modulation of the activity of BAD, an apoptosis regulator. The Pim kinases are highly homologous with each other and show similar oncogenic behaviour.

Clinical reports highlight the importance of the role of the Pim kinases in the development of human cancers.

The Pim kinases, in particular Pim-1 and Pim-2, have been found to be abnormally expressed in a large number of malignant haematological diseases. Amson et al. report the overexpression of Pim-1 in acute myeloid leukaemia and acute lymphoid leukaemia, and that overexpression of Pim-1 appears to result from inappropriate activation in various leukaemias (Proc. Natl. Acad. Sci., Vol. 86., 8857-8861 (1989)). Studies have demonstrated the overexpression of Pim-1 in primitive and metastatic lymphoma of the CNS, which is an aggressive form of non-Hodgkin's lymphoma (Rubenstein et al., Blood, Vol. 107, No. 9, 3716-3723 (2006)). Hüttmann et al. have also discovered an overexpression of Pim-2 in B-cell chronic lymphocytic leukaemia and suggest that an up-regulation of Pim-2 may be associated with a more aggressive evolution of the disease (Leukemia, 20, 1774-1782 (2006)). Abnormal expression of Pim-1 and Pim-2 has been linked with multiple myeloma (Claudio et al., Blood, vol. 100, No. 6, 2175-2186 (2002)).

Hypermutations of Pim-1 have been identified in diffuse large-cell lymphomas (Pasqualucci et al., Nature, Vol. 412, 2001, p. 341-346 (2001)) and in standard and nodular Hodgkin's lymphoma with lymphocytic predominance (Liso et al., Blood, Vol. 108, No. 3, 1013-1020 (2006)).

Numerous studies have also linked abnormal expression of the Pim kinases to various non-haematological human cancers (of the prostate, pancreas, head and neck, etc.) and their presence is often associated with a more aggressive phenotype. For example, Pim-1 and Pim-2 have both been implicated in prostate cancer (Chen et al., Mol. Cancer. Res., 3(8) 443-451 (2005)). Valdman et al. have demonstrated an up-regulation of Pim-1 in the case of patients suffering from a prostate carcinoma and in high-grade prostate intraepithelial neoplasia (precancerous lesions) (The Prostate, (60) 367-371 (2004)), while Dai et al. Have suggested that overexpression of Pim-2 in prostate cancer is associated with more aggressive clinical characteristics (The Prostate, 65:276-286 (2005)). Xie et al. have discovered that the 44-kDa Pim-1 (Pim-1L) was significantly up-regulated in samples of human prostate tumour, and indicate that Pim-1L has an anti-apoptosis effect on human prostate cancer cells in response to chemotherapy drugs (Oncogene, 25, 70-78 (2006)).

Pim-2 is associated with perineural invasion (PNI), during which the cancer cells become wound around nerves, which is often found in certain cancers such as cancers of the prostate, of the pancreas, of the bile ducts and of the head and neck (Ayala et al., Cancer Research, 64, 6082-6090 (2004)). According to Li et al., Pim-3 is aberrantly expressed in human and murine hepatocarcinomas and human pancreatic cancer tissues (Cancer Res. 66 (13), 6741-6747 (2006)). An aberrant expression of Pim-3 has also been observed in gastric adenoma and the metastatic sites of gastric carcinoma (Zheng et al., J. Cancer Res. Clin. Oncol., 134:481-488 (2008)).

Together, these reports suggest that Pim kinase inhibitors are useful for treating cancer, especially leukaemias, lymphomas, myelomas and various solid tumours, especially cancers of the head and neck, bowel cancer, prostate cancer, pancreatic cancer, liver cancer and buccal cancer, for example. Insofar as cancer remains a disease for which the existing treatments are insufficient, it is manifestly necessary to identify novel Pim kinase inhibitors that are effective in treating cancer.

Among the patent applications claiming compounds of the azacarboline class, which is the subject of the present invention, mention may be made of the following documents:

Patent application WO 2007/044779 describes α-aza-β-carbolines having the following general formula, which is partially restricted, relative to the application as published:

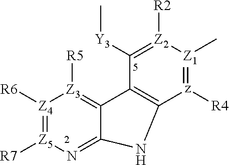

in which
Z5, Z4 and Z3 may represent C and
Z and Z2 may also represent C,
Z1 may, finally, represent C or N and
R2 may represent a carbon bond or an alkylene radical, each possibly being substituted with numerous possibilities including heteroaryloxy, heteroaryl(C1-C5)alkyl, heteroaryls and heterobicycloaryls.

The preparation process and all the examples of the said patent application are limited to the derivatives substituted in positions 2 and 8 and possibly in position 5.

Patent EP 1 209 158 claims compounds having the following formula:

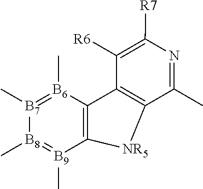

in which B6, B7, B8 and B9 may represent C or N, and R7 never represents a heteroaryl. The activity of the compounds of the said invention is particularly directed towards treating heart problems.

The present invention concerns compounds having the following general formula:

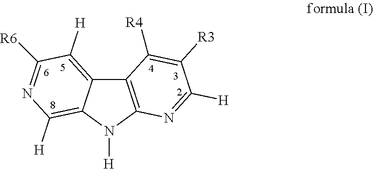

in which
R3 and R4 may be, independently of each other:
   1. H;
   2. halogen;
   3. CF₃;
   4. substituted oxy;
   5. optionally substituted alkoxy;
   6. optionally substituted amino;
   7. substituted carbonyl;
   8. optionally substituted carboxyl;
   9. optionally substituted amide;
   10. sulfur in different oxidation states (II or IV or VI) such as optionally substituted sulfides, sulfoxides or sulfones;
   11. linear, branched or cyclic C₁-C₁₀ alkyl optionally comprising an optionally substituted heteroatom;
   12. optionally substituted linear, branched or cyclic C₂-C₇ alkenyl;
   13. optionally substituted linear or branched C₂-C₆ alkynyl;
   14. optionally substituted aryl or heteroaryl;
   15. optionally substituted heterocycloalkyl;
R6 being a heteroaryl (5- or 6-membered with 1 to 4 heteroatoms chosen from N, S and O) bonded to the azacarboline unit either via a C or via an N belonging to R6, R6 being optionally substituted; R6 also possibly representing C(O)NR1aR1b or an optionally substituted heterocycloalkyl or —C(O) optionally substituted heterocycloalkyl, such that R1a and R1b may be, independently of each other:
   1. H;
   2. optionally monosubstituted or disubstituted linear or branched or cyclic (C₃-C₇) C₁-C₁₀ alkyl;
   3. optionally monosubstituted or disubstituted linear or branched C₂-C₆ alkenyl;
   4. optionally monosubstituted or disubstituted linear or branched C₂-C₆ alkynyl;
   5. optionally monosubstituted or disubstituted aryl;
   6. optionally monosubstituted or disubstituted heteroaryl;
   7. optionally monosubstituted or disubstituted benzyl;
   8. optionally monosubstituted or disubstituted COalkyl;
   9. optionally monosubstituted or disubstituted COaryl;
   10. optionally monosubstituted or disubstituted COheteroaryl;
   11. optionally monosubstituted or disubstituted CO2alkyl;
   12. optionally monosubstituted or disubstituted CO2aryl;
   13. optionally monosubstituted or disubstituted CO2heteroaryl;
   14. CONH2;
   15. optionally monosubstituted or disubstituted CONHalkyl;
   16. optionally monosubstituted or disubstituted CONHaryl;
   17. optionally monosubstituted or disubstituted CONHheteroaryl;
   18. optionally monosubstituted or disubstituted CON(alkyl)2;
   19. optionally monosubstituted or disubstituted CON(aryl)2;
   20. optionally monosubstituted or disubstituted CON(heteroaryl)2;
      the said products of formula (I) being in the form of the base or of an acid-addition salt.

The invention more specifically concerns compounds for which:
R3 and R4 may be, independently of each other:
   1. H;
   2. F;
   3. Cl;
   4. Br;
   5. I;
   6. CF₃;
   7. OR2a;
   8. NR1aR1b;
   9. COR2a;
   10. CO₂R2a;
   11. CO(NR1aR1b);
   12. SR2a;
   13. SOR2a;
   14. SO₂R2a;
   15. linear or branched or cyclic (C₃-C₇) C₁-C₁₀ alkyl optionally monosubstituted or disubstituted or trisubstituted with R2a, R2b, R2c;
   16. linear or branched or cyclic (C₃-C₇) C₂-C₆ alkenyl optionally monosubstituted or disubstituted or trisubstituted with R2a, R2b, R2c;
   17. linear or branched C₂-C₆ alkynyl optionally monosubstituted or disubstituted or trisubstituted with R2a, R2b, R2c;
   18. aryl or heteroaryl optionally monosubstituted or disubstituted or trisubstituted with R2a, R2b, R2c;
   19. heterocycloalkyl optionally monosubstituted or disubstituted or trisubstituted with R2a, R2b, R2c;
R6 being a heteroaryl (5- or 6-membered with 1 to 4 heteroatoms (N, S or O) bonded to the azacarboline unit either via a C or an N belonging to R6, R6 also possibly representing C(O)NR1aR1b or a heterocycloalkyl or —C(O)heterocycloalkyl, R6 being optionally monosubstituted or disubstituted or trisubstituted with R2a, R2b, R2c in which R2a, R2b and R2c are as described hereinabove or hereinbelow and especially in the examples.

It is pointed out that, in the products of formula (I) as defined hereinabove or hereinbelow, the groups R followed only by a figure (R3, R4 and R6) are substituents directly bonded to the tricyclic unit, whereas the groups R followed by a figure and a letter (for example R1a, R2b or R3a) correspond to higher degrees of substitution (for example substituent of R3, R4 or R6) and cannot be directly bonded to the tricyclic unit.

In the substituents mentioned hereinabove:
R1a and R1b may be, independently of each other:
   1. H;
   2. optionally monosubstituted or disubstituted linear or branched or cyclic (C₃-C₇) C₁-C₁₀ alkyl;
   3. optionally monosubstituted or disubstituted linear or branched C₂-C₆ alkenyl;
   4. optionally monosubstituted or disubstituted linear or branched C₂-C₆ alkynyl;
   5. optionally monosubstituted or disubstituted aryl;
   6. optionally monosubstituted or disubstituted heteroaryl;
   7. optionally monosubstituted or disubstituted benzyl;
   8. optionally monosubstituted or disubstituted COalkyl;
   9. optionally monosubstituted or disubstituted COaryl;
   10. optionally monosubstituted or disubstituted COheteroaryl;
   11. optionally monosubstituted or disubstituted CO₂alkyl;
   12. optionally monosubstituted or disubstituted CO₂aryl;
   13. optionally monosubstituted or disubstituted CO₂heteroaryl;
   14. CONH₂;
   15. optionally monosubstituted or disubstituted CONHalkyl;
   16. optionally monosubstituted or disubstituted CONHaryl;
   17. optionally monosubstituted or disubstituted CONHheteroaryl;
   18. optionally monosubstituted or disubstituted CON(alkyl)₂;
   19. optionally monosubstituted or disubstituted CON(aryl)₂;
   20. optionally monosubstituted or disubstituted CON(heteroaryl)₂.
The optional substituents R2a, R2b or R2c are chosen, independently of each other, from:
   1. F;
   2. Cl;
   3. Br;
   4. I;
   5. CF₃;
   6. optionally monosubstituted or polysubstituted linear or branched C₁-C₁₀ alkyl;
   7. optionally monosubstituted or polysubstituted C₃-C₇ cycloalkyl;
   8. optionally monosubstituted or polysubstituted C₂-C₆ alkenyl;
   9. optionally monosubstituted or polysubstituted C₂-C₆ alkynyl;
   10. OH;
   11. optionally monosubstituted or polysubstituted linear or branched O—(C₁-C₁₀)alkyl;
   12. optionally monosubstituted or polysubstituted O—(C₃-C₇)cycloalkyl;
   13. optionally monosubstituted or polysubstituted O-aryl;
   14. optionally monosubstituted or polysubstituted aryl;
   15. optionally monosubstituted or polysubstituted heteroaryl;
   16. optionally monosubstituted or polysubstituted heterocycloalkyl;
   17. NH₂;
   18. NH((C₁-C₁₀)alkyl or (C₃-C₇)cycloalkyl or heterocycloalkyl), each group being optionally monosubstituted or polysubstituted;
   19. N((C₁-C₁₀)alkyl or (C₃-C₇)cycloalkyl)₂, each group being optionally monosubstituted or polysubstituted;
   20. optionally monosubstituted or polysubstituted NH-(aryl or heteroaryl);
   21. N(aryl or heteroaryl)₂, each group being optionally monosubstituted or polysubstituted;
   22. N(aryl or heteroaryl)((C₁-C₁₀)alkyl or (C₃-C₇)cycloalkyl), each group being optionally monosubstituted or polysubstituted;
   23. NHC(O)R3a;
   24. N((C₁-C₁₀)alkyl)C(O)R3a;
   25. N(R3a)C(O)R3b;
   26. NHS(O)₂R3a;
   27. N((C₁-C₁₀)alkylS(O)₂R3a;
   28. N(R3a)S(O)₂R3b;
   29. CO₂R3a;
   30. SR3a;
   31. SOR3a;
   32. SO₂R3a; in which R3a is as defined in the examples.
The optional substituents on the groups R1a and R1b and on the groups R2a, R2b and R2c, also called groups R3a, R3b or R3c, are chosen from:
   1. halogen;
   2. CF₃;
   3. linear or branched C₁-C₁₀ alkyl;
   4. C₃-C₇ cycloalkyl;
   5. C₂-C₆ alkenyl;
   6. C₂-C₆ alkynyl;
   7. C₁-C₁₀ alkylhydroxy;
   8. C₁-C₁₀ alkoxy;
   9. C₁-C₁₀ alkylamino;
   10. OH;
   11. linear, branched or cyclic (C₃-C₇) O—(C₁-C₁₀)alkyl;
   12. O-aryl;
   13. aryl;
   14. heteroaryl;
   15. heterocycloalkyl;
   16. NH₂;
   17. NH—((C₁-C₁₀)alkyl or (C₃-C₇)cycloalkyl);
   18. N((C₁-C₁₀)alkyl or (C₃-C₇)cycloalkyl)₂;
   19. NH-(aryl or heteroaryl);
   20. N(aryl or heteroaryl)₂;
   21. N(aryl or heteroaryl)((C₁-C₁₀)alkyl or (C₃-C₇)cycloalkyl);
   22. NHC(O)—((C₁-C₁₀)alkyl or (C₃-C₇)cycloalkyl or heterocycloalkyl);
   23. NHC(O)-(aryl or heteroaryl);
   24. NHS(O)₂((C₁-C₁₀)alkyl or (C₃-C₇)cycloalkyl or heterocycloalkyl);
   25. NHS(O)₂-(aryl or heteroaryl);
   26. CO (linear or branched C₁-C₁₀ alkyl);
   27. CO(C₁-C₁₀ alkylamino);
   28. CO₂ (linear or branched C₁-C₁₀ alkyl);
   29. C(O)NH (linear or branched C₁-C₁₀ alkyl);
   30. C(O)N (linear or branched C₁-C₁₀ alkyl)₂;
   31. S (linear or branched C₁-C₁₀ alkyl);
   32. SO (linear or branched C₁-C₁₀ alkyl);
   33. SO₂ (linear or branched C₁-C₁₀ alkyl);
   34. C(O)(heterocycloalkyl).

The present invention relates to the set of compounds having the following general formula:

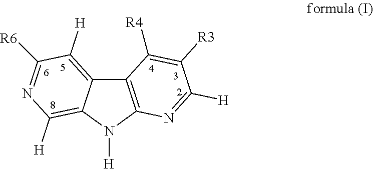

in which
R3 and R4 may be, independently of each other:
   1. H;
   2. F;
   3. Cl;
   4. Br;
   5. I;
   6. CF₃;
   7. OR2a;
   8. NR1aR1b;
   9. COR2a;
   10. CO₂R2a;
   11. CO(NR1aR1b);
   12. SR2a;
   13. SOR2a;
   14. SO₂R2a;
   15. linear or branched or cyclic (C₃-C₇) C₁-C₁₀ alkyl optionally monosubstituted or disubstituted or trisubstituted with R2a, R2b, R2c;
   16. linear or branched or cyclic (C₃-C₇) C₂-C₆ alkenyl optionally monosubstituted or disubstituted or trisubstituted with R2a, R2b, R2c;
   17. linear or branched C₂-C₆ alkynyl optionally monosubstituted or disubstituted or trisubstituted with R2a, R2b, R2c;
   18. aryl or heteroaryl optionally monosubstituted or disubstituted or trisubstituted with R2a, R2b, R2c;
   19. heterocycloalkyl optionally monosubstituted or disubstituted or trisubstituted with R2a, R2b, R2c;
R6 being a heteroaryl (5- or 6-membered with 1 to 4 heteroatoms N, S or O) bonded to the azacarboline unit either via a C or an N belonging to R6, R6 also possibly representing C(O)NR1aR1b or an optionally substituted heterocycloalkyl or —C(O) optionally substituted heterocycloalkyl; R6 being optionally monosubstituted or disubstituted or trisubstituted with R2a, R2b, R2c;
   in which:
R1a and R1b may be, independently of each other:
   1. H;
   2. linear or branched or cyclic (C₃-C₇) C₁-C₁₀ alkyl optionally monosubstituted or disubstituted with R2a R2b;
   3. linear or branched C₂-C₆ alkenyl optionally monosubstituted or disubstituted with R2a R2b;
   4. linear or branched C₂-C₆ alkynyl optionally monosubstituted or disubstituted with R2a R2b;
   5. aryl optionally monosubstituted or disubstituted with R2a R2b;
   6. heteroaryl optionally monosubstituted or disubstituted with R2a R2b;
   7. benzyl optionally monosubstituted or disubstituted with R2a R2b;
   8. COalkyl optionally monosubstituted or disubstituted with R2a R2b;
   9. COaryl optionally monosubstituted or disubstituted with R2a R2b;
   10. COheteroaryl optionally monosubstituted or disubstituted with R2a R2b;
   11. CO₂alkyl optionally monosubstituted or disubstituted with R2a R2b;
   12. CO₂aryl optionally monosubstituted or disubstituted with R2a R2b;
   13. CO₂heteroaryl optionally monosubstituted or disubstituted with R2a R2b;
   14. CONH₂;
   15. CONHalkyl optionally monosubstituted or disubstituted with R2a R2b;
   16. CONHaryl optionally monosubstituted or disubstituted with R2a R2b;
   17. CONHheteroaryl optionally monosubstituted or disubstituted with R2a R2b;
   18. CON(alkyl)₂ optionally monosubstituted or disubstituted with R2a R2b;
   19. CON(aryl)₂ optionally monosubstituted or disubstituted with R2a R2b;
   20. CON(heteroaryl)₂ optionally monosubstituted or disubstituted with R2a R2b;
in which R2a, R2b and R2c are chosen, independently of each other, from:
   1. F;
   2. Cl;
   3. Br;
   4. I;
   5. CF₃;
   6. linear or branched C₁-C₁₀ alkyl optionally monosubstituted or polysubstituted with different R3a;
   7. C₃-C₇ cycloalkyl optionally monosubstituted or polysubstituted with different R3a;
   8. C₂-C₆ alkenyl optionally monosubstituted or polysubstituted with different R3a;
   9. C₂-C₆ alkynyl optionally monosubstituted or polysubstituted with different R3a;
   10. OH;
   11. linear or branched O—(C₁-C₁₀)alkyl optionally monosubstituted or polysubstituted with different R3a;
   12. O—(C₃-C₇)cycloalkyl optionally monosubstituted or polysubstituted with different R3a;
   13. O-aryl optionally monosubstituted or polysubstituted with different R3a;
   14. aryl optionally monosubstituted or polysubstituted with different R3a;
   15. heteroaryl optionally monosubstituted or polysubstituted with different R3a;
   16. heterocycloalkyl optionally monosubstituted or polysubstituted with different R3a;
   17. NH₂;
   18. NH—((C₁-C₁₀)alkyl or (C₃-C₇)cycloalkyl or heterocycloalkyl), each group being optionally monosubstituted or polysubstituted with different R3a;
   19. N((C₁-C₁₀)alkyl or (C₃-C₇)cycloalkyl)₂, each group being optionally monosubstituted or polysubstituted with different R3a;
   20. NH-(aryl or heteroaryl) optionally monosubstituted or polysubstituted with different R3a;
   21. N(aryl or heteroaryl)₂, each group being optionally monosubstituted or polysubstituted with different R3a;
   22. N(aryl or heteroaryl)((C₁-C₁₀)alkyl or (C₃-C₇)cycloalkyl), each group being optionally monosubstituted or polysubstituted with different R3a;
   23. NHC(O)R3a;
   24. N((C₁-C₁₀)alkylC(O)R3a;
   25. N(R3a)C(O)R3b;
   26. NHS(O₂)R3a;
   27. N((C₁-C₁₀)alkylS(O₂)R3a;
   28. N(R3a)S(O)₂R3b;
   29. CO₂R3a;
   30. SR3a;
   31. SOR3a;
   32. SO₂R3a;
in which R3a and R3b are chosen from:
   1. halogen;
   2. CF₃;
   3. linear or branched C₁-C₁₀ alkyl;
   4. C₃-C₇ cycloalkyl;
   5. C₂-C₆ alkenyl;
   6. C₂-C₆ alkynyl;
   7. C₁-C₁₀ alkylhydroxy;
   8. C₁-C₁₀ alkoxy;
   9. C₁-C₁₀ alkylamino;
   10. OH;
   11. linear, branched or cyclic (C₃-C₇) O—(C₁-C₁₀)alkyl;
   12. O-aryl;
   13. aryl;
   14. heteroaryl;
   15. heterocycloalkyl;
   16. NH₂;
   17. NH—((C₁-C₁₀)alkyl or (C₃-C₇)cycloalkyl);
   18. N((C₁-C₁₀)alkyl or (C₃-C₇cycloalkyl)₂;
   19. NH-(aryl or heteroaryl);
   20. N(aryl or heteroaryl)₂;
   21. N(aryl or heteroaryl)((C₁-C₁₀)alkyl or (C₃-C₇)cycloalkyl);
   22. NHC(O)—(C₁-C₁₀)alkyl or (C₃-C₇)cycloalkyl or heterocycloalkyl);
   23. NHC(O)-(aryl or heteroaryl);
   24. NHS(O)₂—((C₁-C₁₀)alkyl or (C₃-C₇)cycloalkyl or heterocycloalkyl);
   25. NHS(O)₂-(aryl or heteroaryl);
   26. CO (linear or branched C₁-C₁₀ alkyl);
   27. CO(C₁-C₁₀ alkylamino);
   28. CO₂ (linear or branched C₁-C₁₀ alkyl);
   29. C(O)NH (linear or branched C₁-C₁₀ alkyl);
   30. C(O)N (linear or branched C₁-C₁₀ alkyl)₂;
   31. S (linear or branched C₁-C₁₀ alkyl);
   32. SO (linear or branched C₁-C₁₀ alkyl);
   33. SO₂ (linear or branched C₁-C₁₀ alkyl);
   34. C(O)(heterocycloalkyl);
      the said products of formula (I) being in the form of the base or of an acid-addition salt.

The present invention thus relates to compounds having the following general formula:

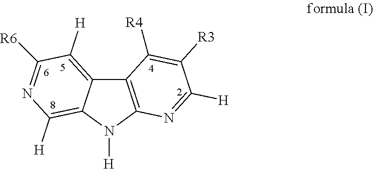

in which
R3 and R4 may be, independently of each other:
   1. H;
   2. halogen;
   3. CF₃;
   4. substituted oxy;
   5. optionally substituted alkoxy;
   6. optionally substituted amino;
   7. substituted carbonyl;
   8. optionally substituted carboxyl;
   9. optionally substituted amide;
   10. sulfur in different oxidation states (II or IV or VI) such as optionally substituted sulfide, sulfoxide or sulfone;
   11. C₁-C₁₀ linear, branched or cyclic alkyl optionally comprising an optionally substituted heteroatom;
   12. optionally substituted linear, branched or cyclic C₁-C₇ alkenyl;
   13. optionally substituted linear or branched C₂-C₆ alkynyl;
   14. optionally substituted aryl or heteroaryl;
R6 being a heteroaryl (5- or 6-membered with 1 to 4 heteroatoms chosen from N, S and O) bonded to the azacarboline unit either via a C or via an N belonging to R6, R6 being optionally substituted.

The invention more specifically relates to compounds for which:
R3 and R4 may be, independently of each other:
   1. H;
   2. F;
   3. Cl;
   4. Br;
   5. I;
   6. CF₃;
   7. OR2a;
   8. NR1aR1b;
   9. COR2a;
   10. CO₂R2a;
   11. CO(NR1aR1b);
   12. SR2a;
   13. SOR2a;
   14. SO₂R2a;
   15. linear or branched or cyclic (C₃-C₇) C₁-C₁₀ alkyl optionally monosubstituted or disubstituted or trisubstituted with R2a, R2b, R2c;
   16. linear or branched or cyclic (C₃-C₇) C₂-C₆ alkenyl optionally monosubstituted or disubstituted or trisubstituted with R2a, R2b, R2c;
   17. linear or branched C₂-C₆ alkynyl optionally monosubstituted or disubstituted or trisubstituted with R2a, R2b, R2c;
   18. aryl or heteroaryl optionally monosubstituted or disubstituted or trisubstituted with R2a, R2b, R2c;
   19. heterocycloalkyl optionally monosubstituted or disubstituted or trisubstituted with R2a, R2b, R2c;
R6 being a heteroaryl (5- or 6-membered with 1 to 4 heteroatoms (N, S or O) bonded to the azacarboline unit either via a C or an N belonging to R6, R6 being optionally monosubstituted or disubstituted or trisubstituted with R2a, R2b, R2c in which R2a, R2b and R2c are as described in the examples.

In the substituents mentioned above:
R1a and R1b may be, independently of each other:
   1. H;
   2. optionally monosubstituted or disubstituted linear or branched or cyclic (C₃-C₇) C₁-C₁₀ alkyl;
   3. optionally monosubstituted or disubstituted linear or branched C₂-C₆ alkenyl;
   4. optionally monosubstituted or disubstituted linear or branched C₂-C₆ alkynyl;
   5. optionally monosubstituted or disubstituted aryl;
   6. optionally monosubstituted or disubstituted heteroaryl;
   7. optionally monosubstituted or disubstituted benzyl;
   8. optionally monosubstituted or disubstituted COalkyl;
   9. optionally monosubstituted or disubstituted COaryl;
   10. optionally monosubstituted or disubstituted COheteroaryl;
   11. optionally monosubstituted or disubstituted CO₂alkyl;
   12. optionally monosubstituted or disubstituted CO₂aryl;
   13. optionally monosubstituted or disubstituted CO₂heteroaryl;
   14. CONH₂;
   15. optionally monosubstituted or disubstituted CONHalkyl;
   16. optionally monosubstituted or disubstituted CONHaryl;
   17. optionally monosubstituted or disubstituted CONHheteroaryl;
   18. optionally monosubstituted or disubstituted CON(alkyl)₂;
   19. optionally monosubstituted or disubstituted CON(aryl)₂;
   20. optionally monosubstituted or disubstituted CON(heteroaryl)₂.
The optional substituents R2a, R2b or R2c are chosen, independently of each other, from:
   1. F;
   2. Cl;
   3. Br;
   4. I;
   5. CF₃;
   6. optionally monosubstituted or polysubstituted linear or branched C₁-C₁₀ alkyl;
   7. optionally monosubstituted or polysubstituted C₃-C₇ cycloalkyl;
   8. optionally monosubstituted or polysubstituted C₂-C₆ alkenyl;
   9. optionally monosubstituted or polysubstituted C₂-C₆ alkynyl;
   10. OH;
   11. optionally monosubstituted or polysubstituted linear or branched O—(C₁-C₁₀)alkyl;
   12. optionally monosubstituted or polysubstituted O—(C₃-C₇)cycloalkyl;
   13. optionally monosubstituted or polysubstituted O-aryl;
   14. optionally monosubstituted or polysubstituted aryl;
   15. optionally monosubstituted or polysubstituted heteroaryl;
   16. optionally monosubstituted or polysubstituted heterocycloalkyl;
   17. NH₂;
   18. NH—((C₁-C₁₀)alkyl or (C₃-C₇)cycloalkyl or heterocycloalkyl), each group being optionally monosubstituted or polysubstituted;
   19. N((C₁-C₁₀)alkyl or (C₃-C₇)cycloalkyl)₂, each group being optionally monosubstituted or polysubstituted;
   20. optionally monosubstituted or polysubstituted NH-(aryl or heteroaryl);
   21. N(aryl or heteroaryl)₂, each group being optionally monosubstituted or polysubstituted;
   22. N(aryl or heteroaryl)((C₁-C₁₀)alkyl or (C₃-C₇)cycloalkyl), each group being optionally monosubstituted or polysubstituted;
   23. NHC(O)R3a;
   24. N((C₁-C₁₀)alkylC(O)R3a;
   25. NHC(O)—((C₁-C₁₀)alkyl or (C₃-C₇)cycloalkyl or heterocycloalkyl), each group being optionally monosubstituted or polysubstituted;
   26. NC(O)((C₁-C₁₀)alkyl or (C₃-C₇)cycloalkyl or heterocycloalkyl)₂, each group being optionally monosubstituted or polysubstituted;
   27. optionally monosubstituted or polysubstituted NHC(O)-(aryl or heteroaryl);
   28. NC(O)(aryl or heteroaryl)₂, each group being optionally monosubstituted or polysubstituted;
   29. NC(O)(aryl or heteroaryl)((C₁-C₁₀)alkyl or (C₃-C₇)cycloalkyl or heterocycloalkyl), each group being optionally monosubstituted or polysubstituted;
   30. NHS(O₂)R3a;
   31. N((C₁-C₁₀)alkylS(O₂)R3a;
   32. NHS(O₂)—((C₁-C₁₀)alkyl or (C₃-C₇)cycloalkyl or heterocycloalkyl), each group being optionally monosubstituted or polysubstituted;
   33. NS(O₂)((C₁-C₁₀)alkyl or (C₃-C₇)cycloalkyl or heterocycloalkyl)₂, each group being optionally monosubstituted or polysubstituted;
   34. optionally monosubstituted or polysubstituted NHS(O₂)-(aryl or heteroaryl);
   35. NS(O₂)(aryl or heteroaryl)₂, each group being optionally monosubstituted or polysubstituted;
   36. NS(O₂)(aryl or heteroaryl)((C₁-C₁₀)alkyl or (C₃-C₇)cycloalkyl or heterocycloalkyl), each group being optionally monosubstituted or polysubstituted;
   37. COR3a;
   38. CO₂R3a;
   39. SR3a;
   40. SOR3a;
   41. SO₂R3a; in which R3a is as defined in the examples.
The optional substituents on the groups R1a and R1b and on the groups R2a, R2b and R2c, also called groups R3a, R3b or R3c, are chosen from:
   1. halogen;
   2. CF₃;
   3. linear or branched C₁-C₁₀ alkyl;
   4. C₃-C₇ cycloalkyl;
   5. C₂-C₆ alkenyl;
   6. C₂-C₆ alkynyl;
   7. OH;
   8. linear, branched or cyclic (C₃-C₇) O—(C₁-C₁₀)alkyl;
   9. O-aryl;
   10. aryl;
   11. heteroaryl;
   12. heterocycloalkyl;
   13. NH₂;
   14. N11-((C₁-C₁₀)alkyl or (C₃-C₇)cycloalkyl);
   15. N((C₁-C₁₀)alkyl or (C₃-C₇)cycloalkyl)₂;
   16. NH-(aryl or heteroaryl);
   17. N(aryl or heteroaryl)₂;
   18. N(aryl or heteroaryl)((C₁-C₁₀)alkyl or (C₃-C₇)cycloalkyl);
   19. NHC(O)—((C₁-C₁₀)alkyl or (C₃-C₇)cycloalkyl or heterocycloalkyl);
   20. NC(O)((Q-C₁₀)alkyl or (C₃-C₇)cycloalkyl or heterocycloalkyl)₂;
   21. NHC(O)-(aryl or heteroaryl);
   22. NC(O)(aryl or heteroaryl)₂;
   23. NC(O)(aryl or heteroaryl)((C₁-C₁₀)alkyl or (C₃-C₇)cycloalkyl or heterocycloalkyl);
   24. NHS(O₂)—((C₁-C₁₀)alkyl or (C₃-C₇)cycloalkyl or heterocycloalkyl);
   25. NS(O₂)((C₁-C₁₀)alkyl or (C₃-C₇)cycloalkyl or heterocycloalkyl)₂;
   26. NHS(O₂)-(aryl or heteroaryl);
   27. NS(O₂)(aryl or heteroaryl)₂;
   28. NS(O₂)(aryl or heteroaryl)((C₁-C₁₀)alkyl or (C₃-C₇)cycloalkyl or heterocycloalkyl);
   29. CO (linear or branched C₁-C₁₀ alkyl);
   30. CO₂ (linear or branched C₁-C₁₀ alkyl);
   31. C(O)NH (linear or branched C₁-C₁₀ alkyl);
   32. C(O)N (linear or branched C₁-C₁₀ alkyl)₂;
   33. S (linear or branched C₁-C₁₀ alkyl);
   34. SO (linear or branched C₁-C₁₀ alkyl);
   35. SO₂ (linear or branched C₁-C₁₀ alkyl).

The group R6 is a 5- or 6-membered heteroaryl preferably chosen from pyridine, pyrazole, imidazole and triazole groups optionally substituted with R2a.

The present invention relates to the set of compounds having the following general formula:

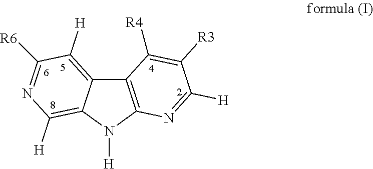

in which
R3 and R4 may be, independently of each other:
   1. H;
   2. F;
   3. Cl;
   4. Br;
   5. I;
   6. CF₃;
   7. OR2a;
   8. NR1aR1b;
   9. COR2a;
   10. CO₂R2a;
   11. CO(NR1aR1b);
   12. SR2a;
   13. SOR2a;
   14. SO₂R2a;
   15. linear or branched or cyclic (C₃-C₇) C₁-C₁₀ alkyl optionally monosubstituted or disubstituted or trisubstituted with R2a, R2b, R2c;
   16. linear or branched or cyclic (C₃-C₇) C₂-C₆ alkenyl optionally monosubstituted or disubstituted or trisubstituted with R2a, R2b, R2c;
   17. linear or branched C₂-C₆ alkynyl optionally monosubstituted or disubstituted or trisubstituted with R2a, R2b, R2c;
   18. aryl or heteroaryl optionally monosubstituted or disubstituted or trisubstituted with R2a, R2b, R2c;
   19. heterocycloalkyl optionally monosubstituted or disubstituted or trisubstituted with R2a, R2b, R2c;
R6 being a heteroaryl (5- or 6-membered with 1 to 4 heteroatoms N, S or O) bonded to the azacarboline unit either via a C or an N belonging to R6, R6 being optionally monosubstituted or disubstituted or trisubstituted with R2a, R2b, R2c;
in which R1a and R1b may be, independently of each other:
   1. H;
   2. linear or branched or cyclic (C₃-C₇) C₁-C₁₀ alkyl optionally monosubstituted or disubstituted with R2a R2b;
   3. linear or branched C₂-C₆ alkenyl optionally monosubstituted or disubstituted with R2a R2b;
   4. linear or branched C₂-C₆ alkynyl optionally monosubstituted or disubstituted with R2a R2b;
   5. aryl optionally monosubstituted or disubstituted with R2a R2b;
   6. heteroaryl optionally monosubstituted or disubstituted with R2a R2b;
   7. benzyl optionally monosubstituted or disubstituted with R2a R2b;
   8. COalkyl optionally monosubstituted or disubstituted with R2a R2b;
   9. COaryl optionally monosubstituted or disubstituted with R2a R2b;
   10. COheteroaryl optionally monosubstituted or disubstituted with R2a R2b;
   11. CO₂alkyl optionally monosubstituted or disubstituted with R2a R2b;
   12. CO₂aryl optionally monosubstituted or disubstituted with R2a R2b;
   13. CO₂heteroaryl optionally monosubstituted or disubstituted with R2a R2b;
   14. CONH₂;
   15. CONHalkyl optionally monosubstituted or disubstituted with R2a R2b;
   16. CONHaryl optionally monosubstituted or disubstituted with R2a R2b;
   17. CONHheteroaryl optionally monosubstituted or disubstituted with R2a R2b;
   18. CON(alkyl)₂ optionally monosubstituted or disubstituted with R2a R2b;
   19. CON(aryl)₂ optionally monosubstituted or disubstituted with R2a R2b;
   20. CON(heteroaryl)₂ optionally monosubstituted or disubstituted with R2a R2b;
in which R2a, R2b and R2c are chosen, independently of each other, from:
   1. F;
   2. Cl;
   3. Br;
   4. I;
   5. CF₃,
   6. linear or branched C₁-C₁₀ alkyl optionally monosubstituted or polysubstituted with different R3a;
   7. C₃-C₇cycloalkyl optionally monosubstituted or polysubstituted with different R3a;
   8. C₂-C₆ alkenyl optionally monosubstituted or polysubstituted with different R3a;
   9. C₂-C₆ alkynyl optionally monosubstituted or polysubstituted with different R3a;
   10. OH;
   11. linear or branched O—(C₁-C₁₀)alkyl optionally monosubstituted or polysubstituted with different R3a;
   12. O—(C₃-C₇)cycloalkyl optionally monosubstituted or polysubstituted with different R3a;
   13. O-aryl optionally monosubstituted or polysubstituted with different R3a;
   14. aryl optionally monosubstituted or polysubstituted with different R3a;
   15. heteroaryl optionally monosubstituted or polysubstituted with different R3a;
   16. heterocycloalkyl optionally monosubstituted or polysubstituted with different R3a;
   17. NH₂;
   18. NH—((C₁-C₁₀)alkyl or (C₃-C₇)cycloalkyl or heterocycloalkyl), each group being optionally monosubstituted or polysubstituted with different R3a;
   19. N((C₁-C₁₀)alkyl or (C₃-C₇)cycloalkyl)₂, each group being optionally monosubstituted or polysubstituted with different R3a;
   20. NH-(aryl or heteroaryl) optionally monosubstituted or polysubstituted with different R3a;
   21. N(aryl or heteroaryl)₂, each group being optionally monosubstituted or polysubstituted with different R3a;
   22. N(aryl or heteroaryl)((C₁-C₁₀)alkyl or (C₃-C₇)cycloalkyl), each group being optionally monosubstituted or polysubstituted with different R3a;
   23. NHC(O)R3a;
   24. N((C₁-C₁₀)alkylC(O)R3a;
   25. NHC(O)—((C₁-C₁₀)alkyl or (C₃-C₇)cycloalkyl or heterocycloalkyl), each group being optionally monosubstituted or polysubstituted with different R3a;
   26. NC(O)((C₁-C₁₀)alkyl or (C₃-C₇)cycloalkyl or heterocycloalkyl)₂, each group being optionally monosubstituted or polysubstituted with different R3a;
   27. NHC(O)-(aryl or heteroaryl) optionally monosubstituted or polysubstituted with different R3a;
   28. NC(O)(aryl or heteroaryl)₂, each group being optionally monosubstituted or polysubstituted with different R3a;
   29. NC(O)(aryl or heteroaryl)((C₁-C₁₀)alkyl or (C₃-C₇)cycloalkyl or heterocycloalkyl), each group being optionally monosubstituted or polysubstituted with different R3a;
   30. NHS(O₂)R3a;
   31. N((C₁-C₁₀)alkylS(O₂)R3a;
   32. NHS(O₂)—((C₁-C₁₀)alkyl or (C₃-C₇)cycloalkyl or heterocycloalkyl), each group being optionally monosubstituted or polysubstituted with different R3a;
   33. NS(O₂)((C₁-C₁₀)alkyl or (C₃-C₇)cycloalkyl or heterocycloalkyl)₂, each group being optionally monosubstituted or polysubstituted with different R3a;
   34. NHS(O₂)-(aryl or heteroaryl) optionally monosubstituted or polysubstituted with different R3a;
   35. NS(O₂)(aryl or heteroaryl)₂, each group being optionally monosubstituted or polysubstituted with different R3a;
   36. NS(O₂)(aryl or heteroaryl)((C₁-C₁₀)alkyl or (C₃-C₇)cycloalkyl or heterocycloalkyl), each group being optionally monosubstituted or polysubstituted with different R3a; COR3a;
   37. CO₂R3a;
   38. SR3a;
   39. SOR3a;
   40. SO₂R3a;
in which R3a is chosen from:
   1. halogen;
   2. CF₃;
   3. linear or branched C₁-C₁₀ alkyl;
   4. C₃-C₇ cycloalkyl;
   5. C₂-C₆ alkenyl;
   6. C₂-C₆ alkynyl;
   7. OH;
   8. linear, branched or cyclic (C₃-C₇) O—(C₁-C₁₀)alkyl;
   9. O-aryl;
   10. aryl;
   11. heteroaryl;
   12. heterocycloalkyl;
   13. NH₂;
   14. NH—((C₁-C₁₀)alkyl or (C₃-C₇)cycloalkyl);
   15. N((C₁-C₁₀)alkyl or (C₃-C₇)cycloalkyl)₂;
   16. NH-(aryl or heteroaryl);
   17. N(aryl or heteroaryl)₂;
   18. N(aryl or heteroaryl)((C₁-C₁₀)alkyl or (C₃-C₇)cycloalkyl);
   19. NHC(O)—((C₁-C₁₀)alkyl or (C₃-C₇)cycloalkyl or heterocycloalkyl);
   20. NC(O)((C₁-C₁₀)alkyl or (C₃-C₇)cycloalkyl or heterocycloalkyl)₂;
   21. NHC(O)-(aryl or heteroaryl);
   22. NC(O)(aryl or heteroaryl)₂;
   23. NC(O)(aryl or heteroaryl)((C₁-C₁₀)alkyl or (C₃-C₇)cycloalkyl or heterocycloalkyl);
   24. NHS(O₂)—((C₁-C₁₀)alkyl or (C₃-C₇)cycloalkyl or heterocycloalkyl);
   25. NS(O₂)((C₁-C₁₀)alkyl or (C₃-C₇)cycloalkyl or heterocycloalkyl)₂;
   26. NHS(O₂)-(aryl or heteroaryl);
   27. NS(O₂)-(aryl or heteroaryl)₂;
   28. NS(O₂)-(aryl or heteroaryl)((C₁-C₁₀)alkyl or (C₃-C₇)cycloalkyl or heterocycloalkyl);
   29. CO (linear or branched C₁-C₁₀ alkyl);
   30. CO₂ (linear or branched C₁-C₁₀ alkyl);
   31. C(O)NH (linear or branched C₁-C₁₀ alkyl);
   32. C(O)N (linear or branched C₁-C₁₀ alkyl)₂;
   33. S (linear or branched C₁-C₁₀ alkyl);
   34. SO (linear or branched C₁-C₁₀ alkyl);
   35. SO₂ (linear or branched C₁-C₁₀ alkyl).

In the context of the present invention, positions 2 and 8 should not be substituted, in contrast with the documents of the prior art.

(C₁-C₁₀)Alkyl or C₁-C₁₀ alkyl means any saturated, linear or branched carbon chain of 1 to 10 carbon atoms.

Aryl means phenyl or naphthyl.

(C₃-C₇)Cycloalkyl means any non-aromatic ring formed solely of carbon atoms, especially cyclopropane, cyclobutane, cyclopentane, cyclohexane or cycloheptane; but also possibly bearing an unsaturation, for example cyclopentene, cyclohexene, cycloheptene, etc.

C₁-C₁₀ alkylhydroxy means any saturated, linear or branched carbon chain of 1 to 10 carbons bearing at least one hydroxyl group (OH).

C₁-C₁₀ alkoxy means any saturated, linear or branched carbon chain of 1 to 10 carbons bearing at least one ether function (C—O—C).

C₁-C₁₀ alkylamino means any saturated, linear or branched carbon chain of 1 to 10 carbons bearing at least one amine (primary, secondary or tertiary) function.

Heteroaryl means any 5-, 6- or 7-membered aromatic monocycle containing at least one heteroatom (N, O or S), especially: pyridine, pyrimidine, imidazole, pyrazole, triazole, thiophene, furan, thiazole, oxazole, etc., and also bicyclic aromatic systems containing at least one heteroatom (N, O or S), especially indole, benzimidazole, azaindole, benzofuran, benzothiophene, quinoline, etc.

Heterocycloalkyl means any non-aromatic monocycle or bicycle (spiro or non-spiro) containing at least one heteroatom (N, O or S) with or without unsaturation, especially: morpholine, piperazine, 4-methylpiperazine, 4-methylsulfonylpiperazine, piperidine, pyrrolidine, oxetane, epoxide, dioxane, imidazolone, imidazolinedione, etc.

The compounds of formula (I) may comprise one or more asymmetric carbon atoms. They may thus exist in the form of enantiomers or diastereoisomers. These enantiomers and diastereoisomers, and also mixtures thereof, including racemic mixtures, form part of the invention.

The compounds of formula (I) may exist in the form of bases or of acid-addition salts. Such addition salts form part of the invention.

These salts may be prepared with pharmaceutically acceptable acids, but the salts of other acids that are useful, for example, for purifying or isolating the compounds of formula (I) also form part of the invention.

In the products of formula (I) as defined hereinabove or hereinbelow, the group R6 is a 5- or 6-membered heteroaryl preferably chosen from pyridine, pyrazole, imidazole, thiophene, quinoline, thiazole or triazole groups optionally substituted with R2a. R6 may also represent C(O)NR1aR1b or alternatively an optionally substituted heterocycloalkyl or optionally substituted C(O)heterocycloalkyl as indicated hereinabove or hereinbelow.

Among the compounds of formula (I) that are subjects of the invention, a first group of compounds is formed by the compounds for which:
R3 represents
   1. hydrogen;
   2. F;
   3. Cl;
   4. Br;
   5. (C₁-C₁₀) alkyl;
   6. OR2a;
   7. NR1aR1b;
   8. CO2R1a;
   9. CONR1aR1b;
   10. aryl optionally monosubstituted or disubstituted with R2a R2b;
   11. heteroaryl optionally monosubstituted or disubstituted with R2a R2b; and/or R6 represents a heteroaryl group, especially a pyridine, pyrazole, imidazole, thiophene, quinoline, thiazole or triazole group.

Among the compounds of formula (I) that are subjects of the invention, a second group of compounds is formed by the compounds for which
R4 represents
   1. hydrogen;
   2. Cl;
   3. OR1a;
   4. (C₁-C₁₀)alkyl;
   5. (C₂-C₆) alkenyl;
   6. (C₂-C₆) alkynyl;
   7. (C₃-C₇)cycloalkyl;
   8. COR1a;
   9. CO₂R1a;
   10. NR1aR1b;
   11. CO(NR1aR1b);
   12. heterocycloalkyl;
   13. aryl;
   14. heteroaryl;
      each being optionally substituted with R2a, R2b and R2c
      and/or R6 represents a heteroaryl group, especially a pyridine, pyrazole, imidazole or triazole group.

Among the compounds of formula (I) that are subjects of the invention, a third group of compounds is formed by the compounds for which
R2a, R2b and R2c are chosen from:
   1. F;
   2. Cl:
   3. (C₁-C₁₀) alkyl;
   4. OH;
   5. O-alkyl;
   6. NH2;
   7. NHSO2alkyl;
   8. NHSO2cycloalkyl;
   9. NHSO2aryl;
   10. NHC(O)alkyl;
   11. NHC(O)cycloalkyl;
   12. CF3;
   13. CO2alkyl;
   14. C(O)Nhalkyl;
   15. heterocycloalkyl;
each being optionally substituted with R3a, R3b and R3c, chosen from:
   1. F;
   2. Cl;
   3. (C₁-C₁₀) alkyl;
   4. OH;
   5. O-alkyl;
   6. NH2;
   7. NH—((C₁-C₁₀)alkyl or (C₃-C₇)cycloalkyl);
   8. N((C₁-C₁₀) alkyl or (C₃-C₇)cycloalkyl)₂;
   9. heterocycloalkyl.

Among the compounds of formula (I) that are subjects of the invention, a first group of compounds is formed by the compounds for which:
R3 represents
   1. hydrogen;
   2. F;
   3. Cl;
   4. Br;
   5. (C₁-C₁₀) alkyl;
   6. OR2a;
   7. NR1aR1b;
   8. CO2R1a;
   9. CONR1aR1b;.
      and/or R6 represents a heteroaryl group, especially a pyridine, pyrazole, imidazole or triazole group.

Among the compounds of formula (I) that are subjects of the invention, a second group of compounds is formed by the compounds for which:
R4 represents
   1. hydrogen;
   2. Cl;
   3. OR1a;
   4. (C₁-C₁₀)alkyl;
   5. (C₂-C₆) alkenyl;
   6. (C₂-C₆) alkynyl;
   7. (C₃-C₇)cycloalkyl;
   8. COR1a;
   9. CO₂R1a;
   10. NR1aR1b;
   11. CO(NR1aR1b);
   12. heterocycloalkyl;
   13. aryl;
   14. heteroaryl;
      each being optionally substituted with R2a, R2b and R2c,
      and/or R6 represents a heteroaryl group, especially a pyridine, pyrazole, imidazole or triazole group.

Among the compounds of formula (I) that are subjects of the invention, a third group of compounds is formed by the compounds for which:
R2a. R2b and R2c are chosen from
   1. F;
   2. Cl;
   3. (C₁-C₁₀) alkyl;
   4. OH;
   5. O-alkyl;
   6. NH2;
   7. NHSO2alkyl;
   8. NHSO2cycloalkyl;
   9. NHSO2aryl;
   10. NHC(O)alkyl;
   11. NHC(O)cycloalkyl;
   12. CF3;
   13. CO2alkyl;
   14. C(O)Nhalkyl;
   15. heterocycloalkyl.

Among the compounds of formula (I), mention may be made, independently of each other, of the following compounds:
N-{-4-[3-fluoro-6-(pyrid-3-yl)-9H-pyrrolo[2,3-b:5,4-c′]dipyrid-4-yl]phenyl}methanesulfonamide;
N-{-4-[3-methoxy-6-(pyrid-3-yl)-9H-pyrrolo[2,3-b:5,4-c′]dipyrid-4-yl]phenyl}methanesulfonamide;
4-(3,5-dimethoxyphenyl)-3-fluoro-6-(pyrid-3-yl)-9H-pyrrolo[2,3-b:5,4-c′]dipyridine;
4-cyclopropyl-3-fluoro-6-(pyrid-3-yl)-9H-pyrrolo[2,3-b:5,4-c′]dipyridine;
3-methoxy-6-(pyrid-3-yl)-9H-pyrrolo[2,3-b:5,4-c′]dipyridine;
N-cyclopropyl-4-[3-fluoro-6-(pyrid-3-yl)-9H-pyrrolo[2,3-b:5,4-c′]dipyrid-4-yl]benzenesulfonamide;
3-hydroxy-2,2-dimethylpropyl 6-(pyrid-3-yl)-9H-pyrrolo[2,3-b:5,4-c′]dipyridine-3-carboxylate;
4-methoxy-6-(pyrid-3-yl)-9H-pyrrolo[2,3-b:5,4-c′]dipyridine;
3-[3-fluoro-6-(pyrid-3-yl)-9H-pyrrolo[2,3-b:5,4-c′]dipyrid-4-yl]phenol;
4-[(E)-2-cyclopropylethenyl]-3-fluoro-6-(pyrid-3-yl)-9H-pyrrolo[2,3-b:5,4-c′]dipyridine;
4-(3,5-difluorophenyl)-3-fluoro-6-(pyrid-3-yl)-9H-pyrrolo[2,3-b:5,4-c′]dipyridine;
2-methylpropan-2-yl 6-(pyrid-3-yl)-9H-pyrrolo[2,3-b:5,4-c′]dipyridine-3-carboxylate;
3-fluoro-4-iodo-6-(pyrid-3-yl)-9H-pyrrolo[2,3-b:5,4-c′]dipyridine;
4-[3-fluoro-6-(pyrid-3-yl)-9H-pyrrolo[2,3-b:5,4-c′]dipyrid-4-yl]butane-1,2-diol;
[3-fluoro-6-(pyrid-3-yl)-9H-pyrrolo[2,3-b:5,4-c′]dipyrid-4-yl](phenyl)methanone;
3-[3-fluoro-6-(pyrid-3-yl)-9H-pyrrolo[2,3-b:5,4-c′]dipyrid-4-yl]benzenesulfonamide;
3-(morpholin-4-yl)-6-(pyrid-3-yl)-9H-pyrrolo[2,3-b:5,4-c′]dipyridine;
6-(1-methyl-1H-pyrazol-4-yl)-9H-pyrrolo[2,3-b:5,4-c′]dipyridine;
3-fluoro-4-(morpholin-4-yl)-6-(pyrid-3-yl)-9H-pyrrolo[2,3-b:5,4-c′]dipyridine;
2-methylpropyl 6-(pyrid-3-yl)-9H-pyrrolo[2,3-b:5,4-c′]dipyridine-3-carboxylate;
N-methyl-N-propyl-6-(pyrid-3-yl)-9H-pyrrolo[2,3-b:5,4-c′]dipyrid-3-amine;
ethyl 6-(pyrid-3-yl)-9H-pyrrolo[2,3-b:5,4-c′]dipyridine-3-carboxylate;
6-(pyrid-3-yl)-9H-pyrrolo[2,3-b:5,4-c′]dipyridine;
3-fluoro-4-methyl-6-(pyrid-3-yl)-9H-pyrrolo[2,3-b:5,4-c′]dipyridine;
3-fluoro-6-(pyrid-3-yl)-9H-pyrrolo[2,3-b:5,4-c′]dipyridine;
4-chloro-3-fluoro-6-(pyrid-3-yl)-9H-pyrrolo[2,3-b:5,4-c′]dipyridine;
3-fluoro-4-[(E)-2-phenylethenyl]-6-(pyrid-3-yl)-9H-pyrrolo[2,3-b:5,4-c′]dipyridine;
3-chloro-6-(pyrid-3-yl)-9H-pyrrolo[2,3-b:5,4-c′]dipyridine;
3-bromo-6-(pyrid-3-yl)-9H-pyrrolo[2,3-b:5,4-c′]dipyridine;
ethyl (2E)-3-[3-fluoro-6-(pyrid-3-yl)-9H-pyrrolo[2,3-b:5,4-c′]dipyrid-4-yl]prop-2-enoate;
3-fluoro-4-[3-(morpholin-4-yl)phenyl]-6-(pyrid-3-yl)-9H-pyrrolo[2,3-b:5,4-c′]dipyridine;
6-(pyrid-3-yl)-9H-pyrrolo[2,3-b:5,4-c′]dipyridine-3-carboxylic acid;
[6-(pyrid-3-yl)-9H-pyrrolo[2,3-b:5,4-c′]dipyrid-3-yl]methanol;
methyl 6-(pyrid-3-yl)-9H-pyrrolo[2,3-b:5,4-c′]dipyridine-3-carboxylate;
N-methyl-N-propyl-6-(pyrid-3-yl)-9H-pyrrolo[2,3-b:5,4-c′]dipyridine-3-carboxamide;
3-fluoro-N-methyl-N-phenyl-6-(pyrid-3-yl)-9H-pyrrolo[2,3-b:5,4-c′]dipyridine-4-carboxamide;
4-{methyl[6-(pyrid-3-yl)-9H-pyrrolo[2,3-b:5,4-c′]dipyrid-3-yl]amino}-1-(pyrrolidin-1-yl)butan-1-one;
6-(furan-3-yl)-9H-pyrrolo[2,3-b:5,4-c′]dipyridine;
[3-fluoro-6-(pyrid-3-yl)-9H-pyrrolo[2,3-b:5,4-c′]dipyrid-4-yl](morpholin-4-yl)methanone;
6-(5-fluoropyrid-3-yl)-9H-pyrrolo[2,3-b:5,4-c′]dipyridine;
2-[6-(pyrid-3-yl)-9H-pyrrolo[2,3-b:5,4-c′]dipyrid-3-yl]propan-2-ol;
6-(6-fluoropyrid-3-yl)-9H-pyrrolo[2,3-b:5,4-c′]dipyridine;
N,N-diethyl-6-(pyrid-3-yl)-9H-pyrrolo[2,3-b:5,4-c′]dipyrid-3-amine;
3-(pyrid-3-yl)-9H-pyrrolo[2,3-b:5,4-c′]dipyridine;
3-methoxy-6-(pyrid-3-yl)-9H-pyrrolo[2,3-c:5,4-c′]dipyridine;
1-chloro-N-{4-[3-fluoro-6-(pyrid-3-yl)-9H-pyrrolo[2,3-b:5,4-c′]dipyrid-4-yl]phenyl}methanesulfonamide;
3-(4-methylpiperazin-1-yl)-6-(pyrid-3-yl)-9H-pyrrolo[2,3-b:5,4-c′]dipyridine;
N-{-4-[3-fluoro-6-(pyrid-3-yl)-9H-pyrrolo[2,3-b:5,4-c′]dipyrid-4-yl]phenyl}cyclopropanesulfonamide;
N-{4-[3-fluoro-6-(pyrid-3-yl)-9H-pyrrolo[2,3-b:5,4-c′]dipyrid-4-yl]-2-methoxyphenyl}methanesulfonamide;
N-{4-[3-fluoro-6-(1-methyl-1H-pyrazol-4-yl)-9H-pyrrolo[2,3-b:5,4-c′]dipyrid-4-yl]phenyl}methanesulfonamide;
3-fluoro-6-(5-methoxypyrid-3-yl)-9H-pyrrolo[2,3-b:5,4-c′]dipyridine;
3-fluoro-6-(4-methoxypyrid-3-yl)-9H-pyrrolo[2,3-b:5,4-c′]dipyridine;
6-(1-benzyl-1H-pyrazol-4-yl)-3-fluoro-9H-pyrrolo[2,3-b:5,4-c′]dipyridine;
3-fluoro-6-(1-methyl-1H-pyrazol-4-yl)-9H-pyrrolo[2,3-b:5,4-c′]dipyridine;
3-fluoro-6-[1-(2-methylpropyl)-1H-pyrazol-4-yl]-9H-pyrrolo[2,3-b:5,4-c′]dipyridine;
3-fluoro-6-[5-(methylsulfanyl)pyrid-3-yl]-9H-pyrrolo[2,3-b:5,4-c′]dipyridine;
4-[3-fluoro-6-(pyrid-3-yl)-9H-pyrrolo[2,3-b:5,4-c′]dipyrid-4-yl]-2-methylbut-3-yn-2-ol;
4-[3-fluoro-6-(pyrid-3-yl)-9H-pyrrolo[2,3-b:5,4-c′]dipyrid-4-yl]-2-methylbut-3-yn-2-amine;
N-{-4-[3-fluoro-6-(pyrid-3-yl)-9H-pyrrolo[2,3-b:5,4-c′]dipyrid-4-yl]-2-methylbut-3-yn-2-yl}-methanesulfonamide;
3-fluoro-4-[3-methyl-3-(piperazin-1-yl)but-1-yn-1-yl]-6-(pyrid-3-yl)-9H-pyrrolo[2,3-b:5,4-c′]-dipyridine;
4-[3-methoxy-6-(pyrid-3-yl)-9H-pyrrolo[2,3-b:5,4-c′]dipyrid-4-yl]-2-methylbut-3-yn-2-ol;
4-[3-methoxy-6-(pyrid-3-yl)-9H-pyrrolo[2,3-b:5,4-c′]dipyrid-4-yl]-2-methylbut-3-yn-2-amine;
N-{4-[3-methoxy-6-(pyrid-3-yl)-9H-pyrrolo[2,3-b:5,4-c′]dipyrid-4-yl]-2-methylbut-3-yn-2-yl}-methanesulfonamide;
3-methoxy-4-[3-methyl-3-(piperazin-1-yl)but-1-yn-1-yl]-6-(pyrid-3-yl)-9H-pyrrolo[2,3-b:5,4-c′]-dipyridine;
3-fluoro-4-[4-(4-methylpiperazin-1-yl)piperid-1-yl]-6-(pyrid-3-yl)-9H-pyrrolo[2,3-b:5,4-c′]-dipyridine;
2-(4-{1-[3-fluoro-6-(pyrid-3-yl)-9H-pyrrolo[2,3-b:5,4-c′]dipyrid-4-yl]piperid-4-yl}piperazin-1-yl)ethanol;
3-fluoro-4-[4-(morpholin-4-yl)piperid-1-yl]-6-(pyrid-3-yl)-9H-pyrrolo[2,3-b:5,4-c′]dipyridine;
3-fluoro-4-[4-(propan-2-yl)piperazin-1-yl]-6-(pyrid-3-yl)-9H-pyrrolo[2,3-b:5,4-c′]dipyridine;
4-(4-cyclopropylpiperazin-1-yl)-3-fluoro-6-(pyrid-3-yl)-9H-pyrrolo[2,3-b:5,4-c′]dipyridine;
4-(4-ethylpiperazin-1-yl)-3-fluoro-6-(pyrid-3-yl)-9H-pyrrolo[2,3-b:5,4-c′]dipyridine;
3-fluoro-4-[4-(1-methylpiperid-4-yl)piperazin-1-yl]-6-(pyrid-3-yl)-9H-pyrrolo[2,3-b:5,4-c′]-dipyridine;
3-methoxy-4-[4-(4-methylpiperazin-1-yl)piperid-1-yl]-6-(pyrid-3-yl)-9H-pyrrolo[2,3-b:5,4-c′]-dipyridine;
2-(4-{1-[3-methoxy-6-(pyrid-3-yl)-9H-pyrrolo[2,3-b:5,4-c′]dipyrid-4-yl]piperid-4-yl}piperazin-1-yl)ethanol;
3-methoxy-4-[4-(morpholin-4-yl)piperid-1-yl]-6-(pyrid-3-yl)-9H-pyrrolo[2,3-b:5,4-c′]dipyridine;
3-methoxy-4-[4-(1-methylpiperid-4-yl)piperazin-1-yl]-6-(pyrid-3-yl)-9H-pyrrolo[2,3-b:5,4-c′]-dipyridine;
3-methoxy-4-[4-(propan-2-yl)piperazin-1-yl]-6-(pyrid-3-yl)-9H-pyrrolo[2,3-b:5,4-c′]dipyridine;
4-(4-cyclopropylpiperazin-1-yl)-3-methoxy-6-(pyrid-3-yl)-9H-pyrrolo[2,3-b:5,4-c′]dipyridine;
4-(4-ethylpiperazin-1-yl)-3-methoxy-6-(pyrid-3-yl)-9H-pyrrolo[2,3-b:5,4-c′]dipyridine;
3-methoxy-4-[4-(methylsulfonyl)piperazin-1-yl]-6-(pyrid-3-yl)-9H-pyrrolo[2,3-b:5,4-c′]dipyridine;
3-fluoro-4-[4-(methyl sulfonyl)piperazin-1-yl]-6-(pyrid-3-yl)-9H-pyrrolo[2,3-b:5,4-c′]dipyridine;
3-{-4-[3-fluoro-6-(pyrid-3-yl)-9H-pyrrolo[2,3-b:5,4-c′]dipyrid-4-yl]phenyl}propanoic acid;
3-fluoro-4-(6-methoxypyrid-3-yl)-6-(pyrid-3-yl)-9H-pyrrolo[2,3-b:5,4-c′]dipyridine;
N-{3-[3-fluoro-6-(pyrid-3-yl)-9H-pyrrolo[2,3-b:5,4-c′]dipyrid-4-yl]phenyl}methanesulfonamide;
3-fluoro-4-(4-methylthiophen-2-yl)-6-(pyrid-3-yl)-9H-pyrrolo[2,3-b:5,4-c′]dipyridine;
3-fluoro-4-(1H-indol-6-yl)-6-(pyrid-3-yl)-9H-pyrrolo[2,3-b:5,4-c′]dipyridine;
{2-[3-fluoro-6-(pyrid-3-yl)-9H-pyrrolo[2,3-b:5,4-c′]dipyrid-4-yl]phenyl}methanol;
3-fluoro-4-(4-methylthiophen-3-yl)-6-(pyrid-3-yl)-9H-pyrrolo[2,3-b:5,4-c′]dipyridine;
3-[3-fluoro-6-(pyrid-3-yl)-9H-pyrrolo[2,3-b:5,4-c′]dipyrid-4-yl]-N,N-dimethylaniline;
3-fluoro-4-(5-methylfuran-2-yl)-6-(pyrid-3-yl)-9H-pyrrolo[2,3-b:5,4-c′]dipyridine;
3-fluoro-4-(1-methyl-1H-indol-5-yl)-6-(pyrid-3-yl)-9H-pyrrolo[2,3-b:5,4-c′]dipyridine;
3-fluoro-4-(1-methyl-1H-pyrazol-4-yl)-6-(pyrid-3-yl)-9H-pyrrolo[2,3-b:5,4-c′]dipyridine;
N-{4-[3-fluoro-6-(pyrid-3-yl)-9H-pyrrolo[2,3-b:5,4-c′]dipyrid-4-yl]benzyl}acetamide;
N-{3-[3-fluoro-6-(pyrid-3-yl)-9H-pyrrolo[2,3-b:5,4-c′]dipyrid-4-yl]benzyl}methanesulfonamide;
3-fluoro-4-(2-methoxyphenyl)-6-(pyrid-3-yl)-9H-pyrrolo[2,3-b:5,4-c′]dipyridine;
4-(2-ethoxypyrid-3-yl)-3-fluoro-6-(pyrid-3-yl)-9H-pyrrolo[2,3-b:5,4-c′]dipyridine;
4-({3-[3-fluoro-6-(pyrid-3-yl)-9H-pyrrolo[2,3-b:5,4-c′]dipyrid-4-yl]phenyl}amino)-4-oxobutanoic acid;
N-{4-[3-fluoro-6-(pyrid-3-yl)-9H-pyrrolo[2,3-b:5,4-c′]dipyrid-4-yl]benzyl}methanesulfonamide;
{-4-[3-fluoro-6-(pyrid-3-yl)-9H-pyrrolo[2,3-b:5,4-c′]dipyrid-4-yl]phenyl}(morpholin-4-yl)methanone;
3-fluoro-4-(1-methyl-1H-pyrazol-5-yl)-6-(pyrid-3-yl)-9H-pyrrolo[2,3-b:5,4-c′]dipyridine;
1-{2-[3-fluoro-6-(pyrid-3-yl)-9H-pyrrolo[2,3-b:5,4-c′]dipyrid-4-yl]phenyl}-N,N-dimethylmethanamine;
2-[3-fluoro-6-(pyrid-3-yl)-9H-pyrrolo[2,3-b:5,4-c′]dipyrid-4-yl]benzonitrile.
1-chloro-N-{-4-[3-fluoro-6-(pyrid-3-yl)-9H-pyrrolo[2,3-b:5,4-c′]dipyrid-4-yl]phenyl}methanesulfonamide;
3-(4-methylpiperazin-1-yl)-6-(pyrid-3-yl)-9H-pyrrolo[2,3-b:5,4-c′]dipyridine;
N-{-4-[3-fluoro-6-(pyrid-3-yl)-9H-pyrrolo[2,3-b:5,4-c′]dipyrid-4-yl]phenyl}cyclopropanesulfonamide;
N-{-4-[3-fluoro-6-(pyrid-3-yl)-9H-pyrrolo[2,3-b:5,4-c′]dipyrid-4-yl]-2-methoxyphenyl}methanesulfonamide;
N-{4-[3-fluoro-6-(1-methyl-1,1-pyrazol-4-yl)-9H-pyrrolo[2,3-b:5,4-c′]dipyrid-4-yl]phenyl}methane-sulfonamide;
3-fluoro-6-(5-methoxypyrid-3-yl)-9H-pyrrolo[2,3-b:5,4-c′]dipyridine;
3-fluoro-6-(4-methoxypyrid-3-yl)-9H-pyrrolo[2,3-b:5,4-c′]dipyridine;
6-(1-benzyl-1H-pyrazol-4-yl)-3-fluoro-9H-pyrrolo[2,3-b:5,4-c′]dipyridine;
3-fluoro-6-(4-methyl-1H-pyrazol-4-yl)-9H-pyrrolo[2,3-b:5,4-c′]dipyridine;
3-fluoro-6-[1-(2-methylpropyl)-1H-pyrazol-4-yl]-9H-pyrrolo[2,3-b:5,4-c′]dipyridine;
3-fluoro-6-[5-(methyl sulfanyl)pyrid-3-yl]-9H-pyrrolo[2,3-b:5,4-c′]dipyridine;
3-fluoro-6-{1-[2-(morpholin-4-yl)ethyl]-1H-pyrazol-4-yl}-9H-pyrrolo[2,3-b:5,4-c′]dipyridine;
3-fluoro-4-[4-(propan-2-yl)piperazin-1-yl]-6-(pyrid-3-yl)-9H-pyrrolo[2,3-b:5,4-c′]dipyridine;
3-fluoro-4-(piperid-1-yl)-6-(pyrid-3-yl)-9H-pyrrolo[2,3-b:5,4-c′]dipyridine;
4-[3-fluoro-6-(pyrid-3-yl)-9H-pyrrolo[2,3-b:5,4-c′]dipyrid-4-yl]-2-methylbut-3-yn-2-amine;
4-[3-fluoro-6-(pyrid-3-yl)-9H-pyrrolo[2,3-b:5,4-c′]dipyrid-4-yl]-2-methylbut-3-yn-2-ol;
4-[3-(4-ethylpiperazin-1-yl)-3-methylbut-1-yn-1-yl]-3-fluoro-6-(pyrid-3-yl)-9H-pyrrolo[2,3-b:5,4-c′]dipyridine;
3-{-4-[3-fluoro-6-(pyrid-3-yl)-9H-pyrrolo[2,3-b:5,4-c′]dipyrid-4-yl]phenyl}propanoic acid;
3-fluoro-4-(6-methoxypyrid-3-yl)-6-(pyrid-3-yl)-9H-pyrrolo[2,3-b:5,4-c′]dipyridine;
N-{3-[3-fluoro-6-(pyrid-3-yl)-9H-pyrrolo[2,3-b:5,4-c′]dipyrid-4-yl]phenyl}methanesulfonamide;
3-fluoro-4-(4-methylthiophen-2-yl)-6-(pyrid-3-yl)-9H-pyrrolo[2,3-b:5,4-c′]dipyridine;
3-fluoro-4-(1H-indol-6-yl)-6-(pyrid-3-yl)-9H-pyrrolo[2,3-b:5,4-c′]dipyridine;
{2-[3-fluoro-6-(pyrid-3-yl)-9H-pyrrolo[2,3-b:5,4-c′]dipyrid-4-yl]phenyl}methanol;
3-fluoro-4-(4-methylthiophen-3-yl)-6-(pyrid-3-yl)-9H-pyrrolo[2,3-b:5,4-c′]dipyridine;
3-[3-fluoro-6-(pyrid-3-yl)-9H-pyrrolo[2,3-b:5,4-c′]dipyrid-4-yl]-N,N-dimethylaniline;
3-fluoro-4-(1-methyl-1H-indol-5-yl)-6-(pyrid-3-yl)-9H-pyrrolo[2,3-b:5,4-c′]dipyridine;
3-fluoro-4-(1-methyl-1H-pyrazol-4-yl)-6-(pyrid-3-yl)-9H-pyrrolo[2,3-b:5,4-c′]dipyridine;
N-{4-[3-fluoro-6-(pyrid-3-yl)-9H-pyrrolo[2,3-b:5,4-c′]dipyrid-4-yl]benzyl}acetamide;
N-{3-[3-fluoro-6-(pyrid-3-yl)-9H-pyrrolo[2,3-b:5,4-c′]dipyrid-4-yl]benzyl}methanesulfonamide;
3-fluoro-4-(2-methoxyphenyl)-6-(pyrid-3-yl)-9H-pyrrolo[2,3-b:5,4-c′]dipyridine;
4-(2-ethoxypyrid-3-yl)-3-fluoro-6-(pyrid-3-yl)-9H-pyrrolo[2,3-b:5,4-c′]dipyridine;
4-({3-[3-fluoro-6-(pyrid-3-yl)-9H-pyrrolo[2,3-b:5,4-c′]dipyrid-4-yl]phenyl}amino)-4-oxobutanoic acid;
N-{-4-[3-fluoro-6-(pyrid-3-yl)-9H-pyrrolo[2,3-b:5,4-c′]dipyrid-4-yl]benzyl}methanesulfonamide;
3-fluoro-4-(1-methyl-1H-pyrazol-5-yl)-6-(pyrid-3-yl)-9H-pyrrolo[2,3-b:5,4-c′]dipyridine;
N-{-4-[3-fluoro-6-(pyrid-3-yl)-9H-pyrrolo[2,3-b:5,4-c′]dipyrid-4-yl]phenyl}-2-methylpropanamide;
3-fluoro-4,6-di(pyrid-3-yl)-9H-pyrrolo[2,3-b:5,4-c′]dipyridine;
N-{2-[3-fluoro-6-(pyrid-3-yl)-9H-pyrrolo[2,3-b:5,4-c′]dipyrid-4-yl]phenyl}methanesulfonamide;
3-fluoro-4-(1 H-pyrazol-4-yl)-6-(pyrid-3-yl)-9H-pyrrolo[2,3-b:5,4-c′]dipyridine;
3-fluoro-4-[3-(methylsulfonyl)phenyl]-6-(pyrid-3-yl)-9H-pyrrolo[2,3-b:5,4-c′]dipyridine;
3-fluoro-4-(2-methoxypyrimidin-5-yl)-6-(pyrid-3-yl)-9H-pyrrolo[2,3-b:5,4-c′]dipyridine;
5-[3-fluoro-6-(pyrid-3-yl)-9H-pyrrolo[2,3-b:5,4-c′]dipyrid-4-yl]pyrid-2-amine;
3-fluoro-4-[4-(1-methylpiperid-4-yl)piperazin-1-yl]-6-(pyrid-3-yl)-9H-pyrrolo[2,3-b:5,4-c′]-dipyridine;
3-fluoro-4-[4-(morpholin-4-yl)piperid-1-yl]-6-(pyrid-3-yl)-9H-pyrrolo[2,3-b:5,4-c′]dipyridine;
N,N-diethyl-2-{-4-[3-fluoro-6-(pyrid-3-yl)-9H-pyrrolo[2,3-b:5,4-c′]dipyrid-4-yl]piperazin-1-yl}-ethanamine;
3-fluoro-4-(4-methyl-1,4-diazepan-1-yl)-6-(pyrid-3-yl)-9H-pyrrolo[2,3-b:5,4-c′]dipyridine;
2-{-4-[3-fluoro-6-(pyrid-3-yl)-9H-pyrrolo[2,3-b:5,4-c′]dipyrid-4-yl]piperazin-1-yl}ethanol;
3-fluoro-4-[4-(4-methylpiperazin-1-yl)piperid-1-yl]-6-(pyrid-3-yl)-9H-pyrrolo[2,3-b:5,4-c′]-dipyridine;
N-{-4-[3-fluoro-6-(pyrid-3-yl)-9H-pyrrolo[2,3-b:5,4-c′]dipyrid-4-yl]phenyl}-N-methylmethanesulfonamide;
3-(piperazin-1-yl)-6-(pyrid-3-yl)-9H-pyrrolo[2,3-b:5,4-c′]dipyridine;
6-(pyrid-3-yl)-9H-pyrrolo[2,3-b:5,4-c′]dipyrid-3-amine;
4-(1.4′-bipiperid-1′-yl)-3-fluoro-6-(pyrid-3-yl)-9H-pyrrolo[2,3-b:5,4-c′]dipyridine;
1-[3-fluoro-6-(pyrid-3-yl)-9H-pyrrolo[2,3-b:5,4-c′]dipyrid-4-yl]-N,N-dimethylpiperid-4-amine;
3-fluoro-6-(pyrid-3-yl)-4-[4-(pyrrolidin-1-yl)piperid-1-yl]-9H-pyrrolo[2,3-b:5,4-c′]dipyridine;
3-fluoro-4-{4-[3-(piperid-1-yl)propyl]piperazin-1-yl}-6-(pyrid-3-yl)-9H-pyrrolo[2,3-b:5,4-c′]-dipyridine;
3-fluoro-4-{4-[3-(morpholin-4-yl)propyl]piperazin-1-yl}-6-(pyrid-3-yl)-9H-pyrrolo[2,3-b:5,4-c′]-dipyridine;
3-{4-[3-fluoro-6-(pyrid-3-yl)-9H-pyrrolo[2,3-b:5,4-c′]dipyrid-4-yl]piperazin-1-yl}-N,N-dipropyl-propan-1-amine;
3-ethoxy-6-(pyrid-3-yl)-9H-pyrrolo[2,3-b:5,4-c′]dipyridine;
3-iodo-6-(pyrid-3-yl)-9H-pyrrolo[2,3-b:5,4-c′]dipyridine;
3-{1-[2-(morpholin-4-yl)ethyl]-1H-pyrazol-4-yl}-6-(pyrid-3-yl)-9H-pyrrolo[2,3-b:5,4-c′]dipyridine;
3-(1-methyl-1H-pyrazol-3-yl)-6-(pyrid-3-yl)-9H-pyrrolo[2,3-b:5,4-c′]dipyridine;
N,N-diethyl-3-{-4-[3-fluoro-6-(pyrid-3-yl)-9H-pyrrolo[2,3-b:5,4-c′]dipyrid-4-yl]piperazin-1-yl}-propan-1-amine;
N,N-diethyl-2-{4-[6-(pyrid-3-yl)-9H-pyrrolo[2,3-b:5,4-c′]dipyrid-3-yl]-1H-pyrazol-1-yl}ethan-amine;
3-fluoro-4-methoxy-6-(pyrid-3-yl)-9H-pyrrolo[2,3-b:5,4-c′]dipyridine;
3-[1-(2-methylpropyl)-1 H-pyrazol-4-yl]-6-(pyrid-3-yl)-9H-pyrrolo[2,3-b:5,4-c′]dipyridine;
3-[4-(morpholin-4-yl)phenyl]-6-(pyrid-3-yl)-9H-pyrrolo[2,3-b:5,4-c′]dipyridine;
N-propyl-6-(pyrid-3-yl)-9H-pyrrolo[2,3-b:5,4-c′]dipyrid-3-amine;
3-{4-[4-(propan-2-yl)piperazin-1-yl]phenyl}-6-(pyrid-3-yl)-9H-pyrrolo[2,3-b:5,4-c′]dipyridine;
6-(pyrid-3-yl)-3-(2,2,2-trifluoroethoxy)-9H-pyrrolo[2,3-b:5,4-c′]dipyridine;
3-fluoro-9H-pyrrolo[2,3-b:5,4-c′]dipyridine-6-carbonitrile;
3-(2-methoxyethoxy)-6-(pyrid-3-yl)-9H-pyrrolo[2,3-b:5,4-c′]dipyridine;
3-{1-[3-(4-methylpiperazin-1-yl)propyl]-1H-pyrazol-4-yl}-6-(pyrid-3-yl)-9H-pyrrolo[2,3-b:5,4-c′]-dipyridine;
{3-[6-(pyrid-3-yl)-9H-pyrrolo[2,3-b:5,4-c′]dipyrid-3-yl]phenyl}methanol;
N,N-diethyl-3-[6-(pyrid-3-yl)-9H-pyrrolo[2,3-b:5,4-c′]dipyrid-3-yl]benzamide;
3-(3,5-dimethyl-1H-pyrazol-4-yl)-6-(pyrid-3-yl)-9H-pyrrolo[2,3-b:5,4-c′]dipyridine;
2-{3,5-dimethyl-4-[6-(pyrid-3-yl)-9H-pyrrolo[2,3-b:5,4-c′]dipyrid-3-yl]-1H-pyrazol-1-yl}-N,N-diethylethanamine;
3-methoxy-6-(1-methyl-1H-pyrazol-4-yl)-9H-pyrrolo[2,3-b:5,4-c′]dipyridine;
methyl 4-{6-[1-(prop-2-en-1-yl)-1H-pyrazol-4-yl]-9H-pyrrolo[2,3-b:5,4-c′]dipyrid-4-yl}benzoate;
N,N-diethyl-2-[4-(3-methoxy-9H-pyrrolo[2,3-b:5,4-c′]dipyrid-6-yl)-3,5-dimethyl-1H-pyrazol-1-yl]ethanamine;
N-[2-(dimethylamino)ethyl]-2-[4-(3-methoxy-9H-pyrrolo[2,3-b:5,4-c′]dipyrid-6-yl)-1H-pyrazol-1-yl]acetamide;
3-(1H-pyrazol-4-yl)-6-(pyrid-3-yl)-9H-pyrrolo[2,3-b:5,4-c′]dipyridine;
N,N-diethyl-3-{4-[6-(pyrid-3-yl)-9H-pyrrolo[2,3-b:5,4-c′]dipyrid-3-yl]-1H-pyrazol-1-yl}propan-1-amine;
N,N-diethyl-3-[4-(3-methoxy-9H-pyrrolo[2,3-b:5,4-c′]dipyrid-6-yl)-1H-pyrazol-1-yl]propan-1-amine;
9H-pyrrolo[2,3-b:5,4-c′]dipyridine-6-carboxylic acid;
N-[3-(dimethylamino)propyl]-N-{-4-[3-fluoro-6-(pyrid-3-yl)-9H-pyrrolo[2,3-b:5,4-c′]dipyrid-4-yl]-phenyl}methanesulfonamide;
(4-methylpiperazin-1-yl)(9H-pyrrolo[2,3-b:5,4-c′]dipyrid-6-yl)methanone;
5-[4-(3-methoxy-9H-pyrrolo[2,3-b:5,4-c′]dipyrid-6-yl)-1H-pyrazol-1-yl]pentan-1-amine;
2-methyl-2-propyl {5-[4-(3-methoxy-9H-pyrrolo[2,3-b:5,4-c′]dipyrid-6-yl)-1H-pyrazol-1-yl]-pentyl}carbamate;
3-methoxy-6-{1-[2-(1-methylpiperid-2-yl)ethyl]-1H-pyrazol-4-yl}-9H-pyrrolo[2,3-b:5,4-c′]-dipyridine;
3-{-4-[3-fluoro-6-(pyrid-3-yl)-9H-pyrrolo[2,3-b:5,4-c′]dipyrid-4-yl]phenoxy}-N,N-dimethylpropan-1-amine;
4-[3-fluoro-6-(pyrid-3-yl)-9H-pyrrolo[2,3-b:5,4-c′]dipyrid-4-yl]phenol;
2-{-4-[3-fluoro-6-(pyrid-3-yl)-9H-pyrrolo[2,3-b:5,4-c′]dipyrid-4-yl]phenoxy}-N,N-dimethylethanamine;
3-{1-[(1-ethylpyrrolidin-2-yl)methyl]-1H-pyrazol-4-yl}-6-(pyrid-3-yl)-9H-pyrrolo[2,3-b:5,4-c′]-dipyridine;
3-fluoro-6-(pyrid-3-yl)-4-{4-[2-(pyrrolidin-1-yl)ethoxy]phenyl}-9H-pyrrolo[2,3-b:5,4-c′]dipyridine;
3-fluoro-6-(thiophen-3-yl)-9H-pyrrolo[2,3-b:5,4-c′]dipyridine;
2-methyl-2-propyl 4-{4-[6-(pyrid-3-yl)-9H-pyrrolo[2,3-b:5,4-c′]dipyrid-3-yl]phenyl}piperazine-1-carboxylate;
3-{-4-[3-fluoro-6-(pyrid-3-yl)-9H-pyrrolo[2,3-b:5,4-c′]dipyrid-4-yl]phenoxy}-N,N,2-trimethyl-propan-1-amine;
3-fluoro-4-{4-[2-(morpholin-4-yl)ethoxy]phenyl}-6-(pyrid-3-yl)-9H-pyrrolo[2,3-b:5,4-c′]dipyridine;
N,N-diethyl-2-{-4-[3-fluoro-6-(pyrid-3-yl)-9H-pyrrolo[2,3-b:5,4-c′]dipyrid-4-yl]phenoxy}ethan-amine;
N-[2-(dimethylamino)ethyl]-5-[3-fluoro-6-(pyrid-3-yl)-9H-pyrrolo[2,3-b:5,4-c′]dipyrid-4-yl]-pyridine-2-carboxamide;
1-{-4-[3-fluoro-6-(pyrid-3-yl)-9H-pyrrolo[2,3-b:5,4-c′]dipyrid-4-yl]phenoxy}-3-(morpholin-4
N-ethyl-3-{4-[3-fluoro-6-(pyrid-3-yl)-9H-pyrrolo[2,3-b:5,4-c′]dipyrid-4-yl]phenoxy}propan-1-amine;
4-[6-(pyrid-3-yl)-9H-pyrrolo[2,3-b:5,4-c′]dipyrid-3-yl]phenol;
3-[4-(piperazin-1-yl)phenyl]-6-(pyrid-3-yl)-9H-pyrrolo[2,3-b:5,4-c′]dipyridine;
3-fluoro-6-(isoquinolin-4-yl)-9H-pyrrolo[2,3-b:5,4-c′]dipyridine;
N,N-dimethyl-3-{4-[6-(pyrid-3-yl)-9H-pyrrolo[2,3-b:5,4-c′]dipyrid-3-yl]phenoxy}propan-1-amine;
3-{4-[3-(piperid-1-yl)propoxy]phenyl}-6-(pyrid-3-yl)-9H-pyrrolo[2,3-b:5,4-c′]dipyridine;
3-{4-[2-(morpholin-4-yl)ethoxy]phenyl}-6-(pyrid-3-yl)-9H-pyrrolo[2,3-b:5,4-c′]dipyridine;
3-{4-[3-(morpholin-4-yl)propoxy]phenyl}-6-(pyrid-3-yl)-9H-pyrrolo[2,3-b:5,4-c′]dipyridine;
3-{4-[2-(1H-imidazol-1-yl)ethoxy]phenyl}-6-(pyrid-3-yl)-9H-pyrrolo[2,3-b:5,4-c′]dipyridine;
3-(4-{3-[4-(methylsulfonyl)piperazin-1-yl]propoxy}phenyl)-6-(pyrid-3-yl)-9H-pyrrolo[2,3-b:5,4-c′]-dipyridine;
N,N-diethyl-2-{3-[6-(pyrid-3-yl)-9H-pyrrolo[2,3-b:5,4-c′]dipyrid-3-yl]phenoxy}ethanamine;
2-methyl-2-propyl 4-{3-[6-(pyrid-3-yl)-9H-pyrrolo[2,3-b:5,4-c′]dipyrid-3-yl]phenyl}piperazine-1-carboxylate;
N,N,4-triethyl-5-[6-(pyrid-3-yl)-9H-pyrrolo[2,3-b:5,4-c′]dipyrid-3-yl]pyrid-2-amine;
3-[3-(piperazin-1-yl)phenyl]-6-(pyrid-3-yl)-9H-pyrrolo[2,3-b:5,4-c′]dipyridine hydrochloride;
N,N-diethyl-2-({-4-[3-fluoro-6-(pyrid-3-yl)-9H-pyrrolo[2,3-b:5,4-c′]dipyrid-4-yl]-2-methylbut-3-yn-2-yl}oxy)ethanamine;
4-[3-fluoro-6-(pyrid-3-yl)-9H-pyrrolo[2,3-b:5,4-c′]dipyrid-4-yl]-N-(prop-2-en-1-yl)aniline;
N-(2-methylpropan-2-yl)-5-(9H-pyrrolo[2,3-b:5,4-c′]dipyrid-6-yl)pyridine-3-carboxamide;
5-(3-fluoro-9H-pyrrolo[2,3-b:5,4-c′]dipyrid-6-yl)-N-(2-methylpropan-2-yl)pyridine-3-carboxamide;
3-fluoro-6-(1H-pyrazol-4-yl)-9H-pyrrolo[2,3-b:5,4-c′]dipyridine;
(2E)-N-[4-(dimethylamino)butyl]-3-[3-fluoro-6-(pyrid-3-yl)-9H-pyrrolo[2,3-b:5,4-c′]dipyrid-4-yl]prop-2-enamide;
6-chloro-3-fluoro-9H-pyrrolo[2,3-b:5,4-c′]dipyridine;
3-{4-[6-(pyrid-3-yl)-9H-pyrrolo[2,3-b:5,4-c′]dipyrid-3-yl]phenoxy}propan-1-amine;

Among the compounds of formula (I) that may also be mentioned, independently of each other, are the following compounds:
3-{1-[3-(4-methylpiperazin-1-yl)propyl]-1H-pyrazol-4-yl}-6-(1-methyl-1H-pyrazol-4-yl)-9H-pyrrolo[2,3-b:5,4-c′]dipyridine;
3-[3-(4-methylpiperazin-1-yl)phenyl]-6-(1-methyl-1H-pyrazol-4-yl)-9H-pyrrolo[2,3-b:5,4-c′]-dipyridine;
N,N-diethyl-2-{-4-[6-(1-methyl-1H-pyrazol-4-yl)-9H-pyrrolo[2,3-b:5,4-c′]dipyrid-3-yl]-1H-pyrazol-1-yl}ethanamine;
6-(1-methyl-1H-pyrazol-4-yl)-3-{4-[3-(morpholin-4-yl)propoxy]phenyl}-9H-pyrrolo[2,3-b:5,4-c′]-dipyridine;
N,N-diethyl-2-{3-[6-(1-methyl-1H-pyrazol-4-yl)-9H-pyrrolo[2,3-b:5,4-c′]dipyrid-3-yl]phenoxy}-ethanamine;
3-fluoro-6-(1-methyl-1H-pyrazol-4-yl)-4-{4-[3-(piperid-1-yl)propyl]piperazin-1-yl}-9H-pyrrolo[2,3-b:5,4-c′]dipyridine;
4-[3-(4-ethylpiperazin-1-yl)-3-methylbut-1-yn-1-yl]-3-fluoro-6-(1-methyl-1 H-pyrazol-4-yl)-9H-pyrrolo[2,3-b:5,4-c′]dipyridine;
N-[3-(dimethylamino)propyl]-N-{-4-[3-fluoro-6-(1-methyl-1 H-pyrazol-4-yl)-9H-pyrrolo[2,3-b:5,4-c′]dipyrid-4-yl]phenyl}methanesulfonamide;
N-ethyl-3-{-4-[3-fluoro-6-(4-methyl-1H-pyrazol-4-yl)-9H-pyrrolo[2,3-b:5,4-c′]dipyrid-4-yl]-phenoxy}propan-1-amine;
N,N-diethyl-2-{-4-[3-fluoro-6-(1-methyl-1H-pyrazol-4-yl)-9H-pyrrolo[2,3-b:5,4-c′]dipyrid-4-yl]-phenoxy}ethanamine;
3-{4-[3-fluoro-6-(1-methyl-1H-pyrazol-4-yl)-9H-pyrrolo[2,3-b:5,4-c′]dipyrid-4-yl]phenoxy}-N,N,2-trimethylpropan-1-amine;
1-{-4-[3-fluoro-6-(1-methyl-1H-pyrazol-4-yl)-9H-pyrrolo[2,3-b:5,4-c′]dipyrid-4-yl]phenoxy}-3-(piperid-1-yl)propan-2-ol;
1-{4-[3-(2-methoxyethoxy)-6-(1-methyl-1H-pyrazol-4-yl)-9H-pyrrolo[2,3-b:5,4-c′]dipyrid-4-yl]-phenoxy}-3-(piperid-1-yl)propan-2-ol;
3-(2-methoxyethoxy)-6-(1-methyl-1H-pyrazol-4-yl)-4-{4-[3-(piperid-1-yl)propyl]piperazin-1-yl}-9H-pyrrolo[2,3-b:5,4-c′]dipyridine;
4-[3-(4-ethylpiperazin-1-yl)-3-methylbut-1-yn-1-yl]-3-(2-methoxyethoxy)-6-(1-methyl-1H-pyrazol-4-yl)-9H-pyrrolo[2,3-b:5,4-c′]dipyridine;
N-[3-(dimethylamino)propyl]-N-{4-[3-(2-methoxyethoxy)-6-(1-methyl-1H-pyrazol-4-yl)-9H-pyrrolo[2,3-b:5,4-c′]dipyrid-4-yl]phenyl}methanesulfonamide;
N-ethyl-3-{4-[3-(2-methoxyethoxy)-6-(1-methyl-1H-pyrazol-4-yl)-9H-pyrrolo[2,3-b:5,4-c′]dipyrid-4-yl]phenoxy}propan-1-amine;
3-{4-[3-(2-methoxyethoxy)-6-(1-methyl-1H-pyrazol-4-yl)-9H-pyrrolo[2,3-b:5,4-c′]dipyrid-4-yl]-phenoxy}-N,N,2-trimethylpropan-1-amine;
N,N-diethyl-2-{4-[3-(2-methoxyethoxy)-6-(1-methyl-1H-pyrazol-4-yl)-9H-pyrrolo[2,3-b:5,4-c′]-dipyrid-4-yl]phenoxy}ethanamine;
1-{4-[3-(2-methoxyethoxy)-6-(1-methyl-1H-pyrazol-4-yl)-9H-pyrrolo[2,3-b:5,4-c′]dipyrid-4-yl]-phenoxy}-3-(piperid-1-yl)propan-2-ol;
3-amino-1-{-4-[3-fluoro-6-(pyrid-3-yl)-9H-pyrrolo[2,3-b:5,4-c′]dipyrid-4-yl]phenyl}pyrrolidine-2,5-dione;
4-({[3-fluoro-6-(pyrid-3-yl)-9H-pyrrolo[2,3-b:5,4-c′]dipyrid-4-yl]oxy}methyl)-N,N-dimethylaniline;
3-fluoro-6-(pyrid-3-yl)-9H-pyrrolo[2,3-b:5,4-c′]dipyrid-4-yl[4-(dimethylamino)phenyl]carbamate;
3-fluoro-6-(pyrid-3-yl)-9H-pyrrolo[2,3-b:5,4-c′]dipyrid-4-yl[3-(dimethylamino)propyl]carbamate;
3-[(3-fluoro-9H-pyrrolo[2,3-b:5,4-c′]dipyrid-6-yl)carbonyl]-1.5.5-trimethylimidazolidine-2.4-dione;
3-[(3-fluoro-9H-pyrrolo[2,3-b:5,4-c′]dipyrid-6-yl)carbonyl]-1-methylimidazolidine-2.4-dione;
3-[(3-fluoro-9H-pyrrolo[2,3-b:5,4-c′]dipyrid-6-yl)carbonyl]-5.5-dimethyl-1-(propan-2-yl)imidazolidine-2,4-dione;
1-[(3-fluoro-9H-pyrrolo[2,3-b:5,4-c′]dipyrid-6-yl)carbonyl]-4.4-dimethyl-3-(propan-2-yl)imidazolidin-2-one;
1-[(3-fluoro-9H-pyrrolo[2,3-b:5,4-c′]dipyrid-6-yl)carbonyl]-3.4.4-trimethylimidazolidin-2-one;
1-[(3-fluoro-9H-pyrrolo[2,3-b:5,4-c′]dipyrid-6-yl)carbonyl]-3-methylimidazolidin-2-one;
3-fluoro-6-(1-methyl-1 H-imidazol-5-yl)-9H-pyrrolo[2,3-b:5,4-c′]dipyridine;
3-fluoro-6-{1-methyl-5-[3-methyl-3-(4-methylpiperazin-1-yl)but-1-yn-1-yl]-1H-pyrazol-4-yl}-9H-pyrrolo[2,3-b:5,4-c′]dipyridine;
6-(5-chloro-1-methyl-1 H-pyrazol-4-yl)-3-fluoro-9H-pyrrolo[2,3-b:5,4-c′]dipyridine;
6-(5-bromo-1-methyl-1H-pyrazol-4-yl)-3-fluoro-9H-pyrrolo[2,3-b:5,4-c′]dipyridine;
N-{4-[3-(dimethylamino)propoxy]benzyl}-6-(pyrid-3-yl)-9H-pyrrolo[2,3-b:5,4-c′]dipyrid-3-amine;
N-{4-[2-(dimethylamino)ethoxy]benzyl}-6-(pyrid-3-yl)-9H-pyrrolo[2,3-b:5,4-c′]dipyrid-3-amine;
6-(pyrid-3-yl)-N-{[2-(pyrid-4-yl)cyclopropyl]methyl}-9H-pyrrolo[2,3-b:5,4-c′]dipyrid-3-amine;
N-[3-fluoro-4-(piperazin-1-yl)benzyl]-6-(pyrid-3-yl)-9H-pyrrolo[2,3-b:5,4-c′]dipyrid-3-amine;
6-(pyrid-3-yl)-N-{[1-(pyrid-3-ylmethyl)-1H-pyrrol-2-yl]methyl}-9H-pyrrolo[2,3-b:5,4-c′]dipyrid-3-amine;
N-{4-[dimethylamino)methyl]benzyl}-6-(pyrid-3-yl)-9H-pyrrolo[2,3-b:5,4-c′]dipyrid-3-amine;
4-methyl-N1-[6-(pyrid-3-yl)-9H-pyrrolo[2,3-b:5,4-c′]dipyrid-3-yl]pentane-1,4-diamine;
N-(4-methyl-4-nitropentyl)-6-(pyrid-3-yl)-9H-pyrrolo[2,3-b:5,4-c′]dipyrid-3-amine;
N,N-dimethyl-N′-[6-(pyrid-3-yl)-9H-pyrrolo[2,3-b:5,4-c′]dipyrid-3-yl]butane-1,4-diamine;
piperazin-1-yl[4-({[6-(pyrid-3-yl)-9H-pyrrolo[2,3-b:5,4-c′]dipyrid-3-yl]amino}methyl)phenyl]methanone;
N-[4-(aminomethyl)benzyl]-6-(pyrid-3-yl)-9H-pyrrolo[2,3-b:5,4-c′]dipyrid-3-amine;
2-methyl-2-propyl[4-({[6-(pyrid-3-yl)-9H-pyrrolo[2,3-b:5,4-c′]dipyrid-3-yl]amino}methyl)benzyl]-carbamate;
2-methyl-2-propyl 4-{[4-({[6-(pyrid-3-yl)-9H-pyrrolo[2,3-b:5,4-c′]dipyrid-3-yl]amino}methyl)-phenyl]carbonyl}piperazine-1-carboxylate;
N-[4-(dimethylamino)benzyl]-6-(pyrid-3-yl)-9H-pyrrolo[2,3-b:5,4-c′]dipyrid-3-amine;
N-{4-[(4-methyl-1,4-diazepan-1-yl)methyl]benzyl}-6-(pyrid-3-yl)-9H-pyrrolo[2,3-b:5,4-c′]dipyrid-3-amine;
4-(4-methyl-1,4-diazepan-1-yl)-N-[6-(pyrid-3-yl)-9H-pyrrolo[2,3-b:5,4-c′]dipyrid-3-yl]benzamide;
N-[4-(4-methyl-1,4-diazepan-1-yl)benzyl]-6-(pyrid-3-yl)-9H-pyrrolo[2,3-b:5,4-c′]dipyrid-3-amine;
3-(4-methyl-1,4-diazepan-1-yl)-N-[6-(pyrid-3-yl)-9H-pyrrolo[2,3-b:5,4-c′]dipyrid-3-yl]propanamide;
3-[(4-methyl-1,4-diazepan-1-yl)methyl]-N-[6-(pyrid-3-yl)-9H-pyrrolo[2,3-b:5,4-c′]dipyrid-3-yl]-benzamide;
N-{3-[(4-methyl-1,4-diazepan-1-yl)methyl]benzyl-}6-(pyrid-3-yl)-9H-pyrrolo[2,3-b:5,4-c′]dipyrid-3-amine;
N-[2-(4-methyl-1,4-diazepan-1-yl)ethyl]-6-(pyrid-3-yl)-9H-pyrrolo[2,3-b:5,4-c′]dipyrid-3-amine;
6-(pyrid-3-yl)-9H-pyrrolo[2,3-b:5,4-c′]dipyridine-3-carbonitrile;
6-(3,5-dimethyl-1H-pyrazol-4-yl)-3-(pyrid-3-yl)-9H-β-carboline;
2-{3-[6-(pyrid-3-yl)-9H-pyrrolo[2,3-b:5,4-c′]dipyrid-3-yl]phenoxy}ethanamine;
3-(4-{[6-(pyrid-3-yl)-9H-pyrrolo[2,3-b:5,4-c′]dipyrid-3-yl]oxy}phenyl)propan-1-ol;
N,N-dimethyl-2-(4-{[6-(pyrid-3-yl)-9H-pyrrolo[2,3-b:5,4-c′]dipyrid-3-yl]oxy}phenyl)ethanamine;
2-(4-{[6-(pyrid-3-yl)-9H-pyrrolo[2,3-b:5,4-c′]dipyrid-3-yl]oxy}phenyl)acetamide;
N-methyl-2-(4-{[6-(pyrid-3-yl)-9H-pyrrolo[2,3-b:5,4-c′]dipyrid-3-yl]oxy}phenyl)acetamide;
N-cyclopropyl-2-(4-{[6-(pyrid-3-yl)-9H-pyrrolo[2,3-b:5,4-c′]dipyrid-3-yl]oxy}phenyl)acetamide;
N-(propan-2-yl)-1-(4-{[6-(pyrid-3-yl)-9H-pyrrolo[2,3-b:5,4-c′]dipyrid-3-yl]oxy}phenyl)propan-2-amine;
6-(pyrid-3-yl)-3-{4-[2-(pyrrolidin-1-yl)propyl]phenoxy}-9H-pyrrolo[2,3-b:5,4-c′]dipyridine;
N,N-diethyl-3-(4-{[6-(pyrid-3-yl)-9H-pyrrolo[2,3-b:5,4-c′]dipyrid-3-yl]oxy}phenyl)propan-1-amine;
N,N-diethyl-2-{[6-(pyrid-3-yl)-9H-pyrrolo[2,3-b:5,4-c′]dipyrid-3-yl]oxy}ethanamine.

A subject of the present invention is also processes for preparing the products of formula (I) as defined above and described especially in Schemes 1 to 11 below.

A subject of the present invention is especially the process for preparing the products of formula (I) as defined above and described in Scheme 1 below, in which the substituents R3 and R4 have the meanings given hereinabove or hereinbelow, and R represents either the values of R6 as defined above or the following values: OH, OCH₃, OS(O)₂CF₃, Cl, SCH₃, CN.

The strategy for synthesizing the tricyclic nucleus is based on two coupling reactions: a carbon-carbon bond is first created between two suitably selected pyridines, and formation of an intramolecular carbon-nitrogen bond then leads to the 9H-pyrrolo[2,3-b:5,4-c′]dipyridine unit (see Scheme 1 below).

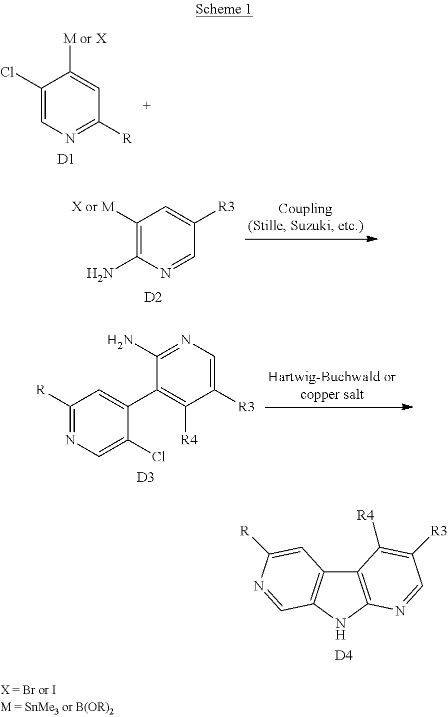

The starting materials D1 and D2 of Scheme 1 may be commercially available or may be prepared according to the usual methods known to those skilled in the art.

A subject of the present invention is also processes for preparing D1 and/or D2 as defined especially in Schemes 2 and 7 below.

A subject of the present invention is thus also, as novel industrial products, certain compounds D1 and/or D2 as defined hereinabove or hereinbelow.

A subject of the present invention is also, as novel industrial products, the synthetic intermediates D3 in which the substituents R3, R4 and R have the meanings given hereinabove or hereinbelow. A subject of the present invention is also, as novel industrial products, the synthetic intermediates D3 in which the substituent R3 represents a fluorine atom or a methoxy radical, and the substituent R4 represents a hydrogen atom, R being chosen from the values defined above.

Compounds D4 represent products of formula (I) as defined above when R represents the values of R6 as defined above, R3 and R4 having any of the meanings given above.

A subject of the present invention is also, as novel industrial products, the synthetic intermediates D4 in which R represents the following values: OH, OCH₃, OS(O)₂CF₃, Cl, SCH₃, CN, R3 and R4 having any of the meanings given above.

The process for preparing the compounds according to the invention consists, in a first step, in reacting the following products:

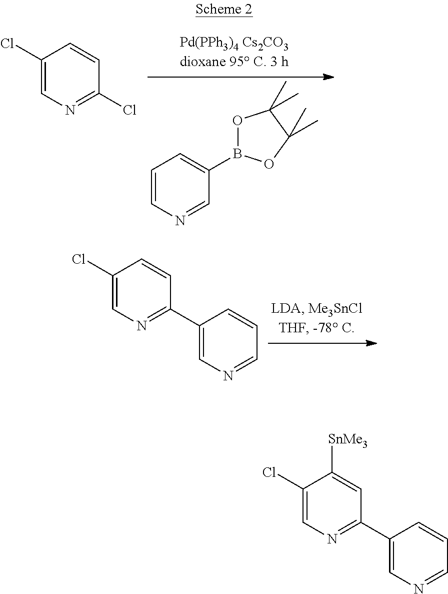

In a second step, the following step is performed:

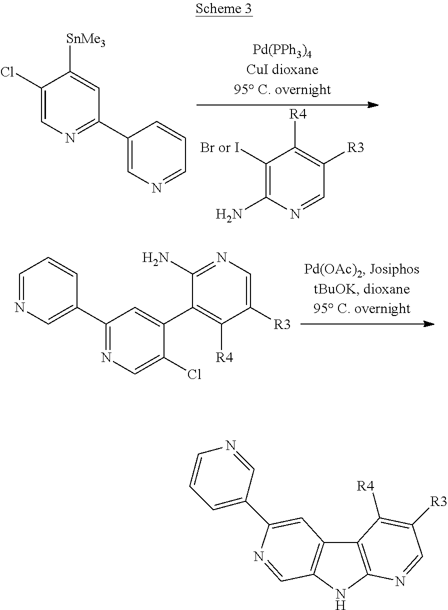

in which Josiphos is a compound having the following formula:

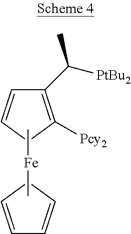

When the groups R3 and R4 are not present before the coupling-cyclization sequence, the following strategies have been developed. In the case of position 4, before protection of position 9, a metallation is performed via the action of a strong base in the presence of a suitable ortho-directing group R3. When the anion is trapped, for example via the action of diiodine, an intermediate iodinated in position 4 is then obtained, this compound allowing the preparation of numerous compounds functionalized at 4 via coupling reactions catalysed with organometallic complexes (Suzuki reaction, introduction of amine via a reaction of Hartwig-Buchwald type, introduction of alkyne via a Sonogashira reaction).

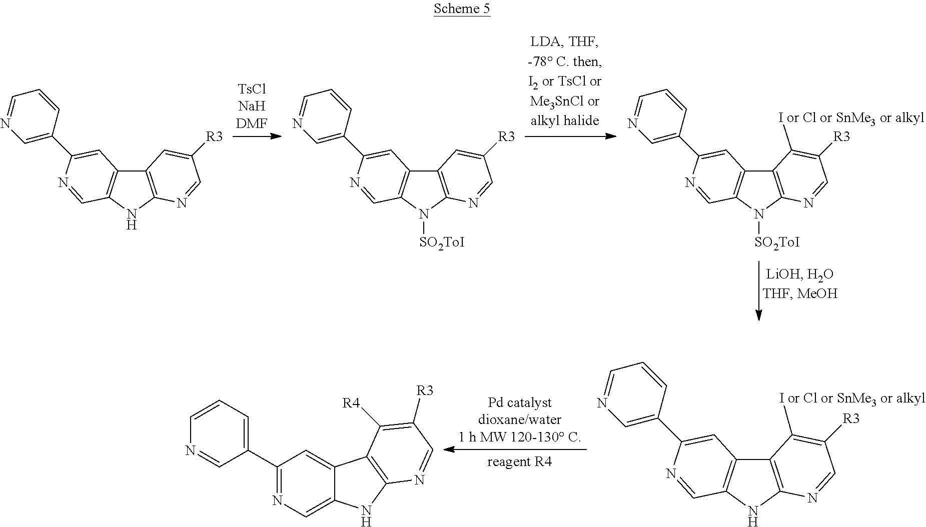

The variations of structure at position 3 are produced from the derivative 3-bromo-6-(pyrid-3-yl)-9H-pyrrolo[2,3-b:5,4-c′]dipyridine obtained via the action of dibromine in acetic acid on 6-(pyrid-3-yl)-9H-pyrrolo[2,3-b:5,4-c′]dipyridine. Once again, coupling reactions catalysed with palladium complexes (introduction of aryl or heteroaryl via a Suzuki reaction, introduction of amine via a reaction of Hartwig-Buchwald type) or copper complexes (introduction of an alkoxy group) allow the production of the various compounds functionalized in position 3.

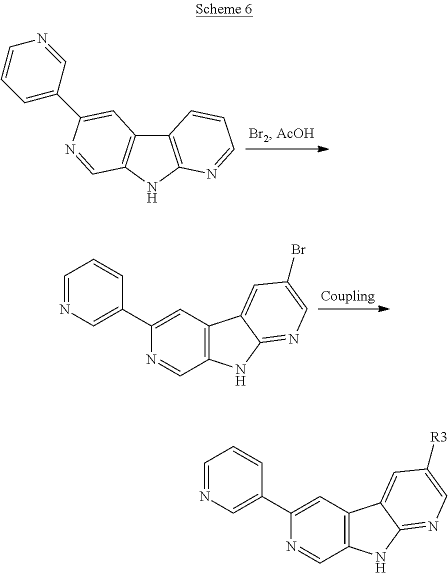

The first step of the process for preparing compounds containing a unit other than the (3-pyridyl) group in position 6 according to the invention consists of one of the following two reactions:

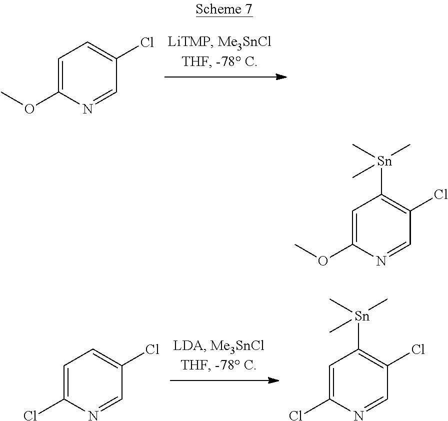

In a second step, Stille coupling is performed with a 2-amino-3-(bromo or iodo)pyridine derivative optionally substituted in position 4 or 5, followed by a reaction of intramolecular aryl amination type, catalysed either with a palladium complex or with copper (I) iodide:

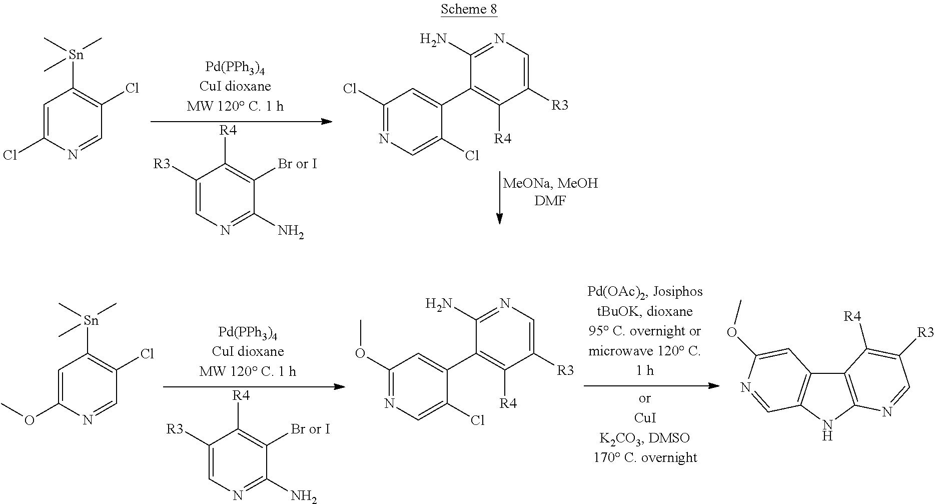

Installation of the 1′-methyl-1′ H-pyrazol-4′-yl unit (or any other aryl or heteroaryl that may be introduced via a coupling reaction catalysed with a palladium complex) is performed via a sequence of three steps comprising: a demethylation reaction, the formation of a triflate derivative, and a coupling reaction of Suzuki type. The synthesis of a carboxamide group in position 6 is also possible from triflate: a nitrile function is first introduced by reacting zinc cyanide in the presence of a palladium complex, and in the next step the nitrile is hydrolysed in acidic medium to give the corresponding carboxylic acid. The final step is a formation of amide via the acyl chloride obtained via the action of thionyl chloride.

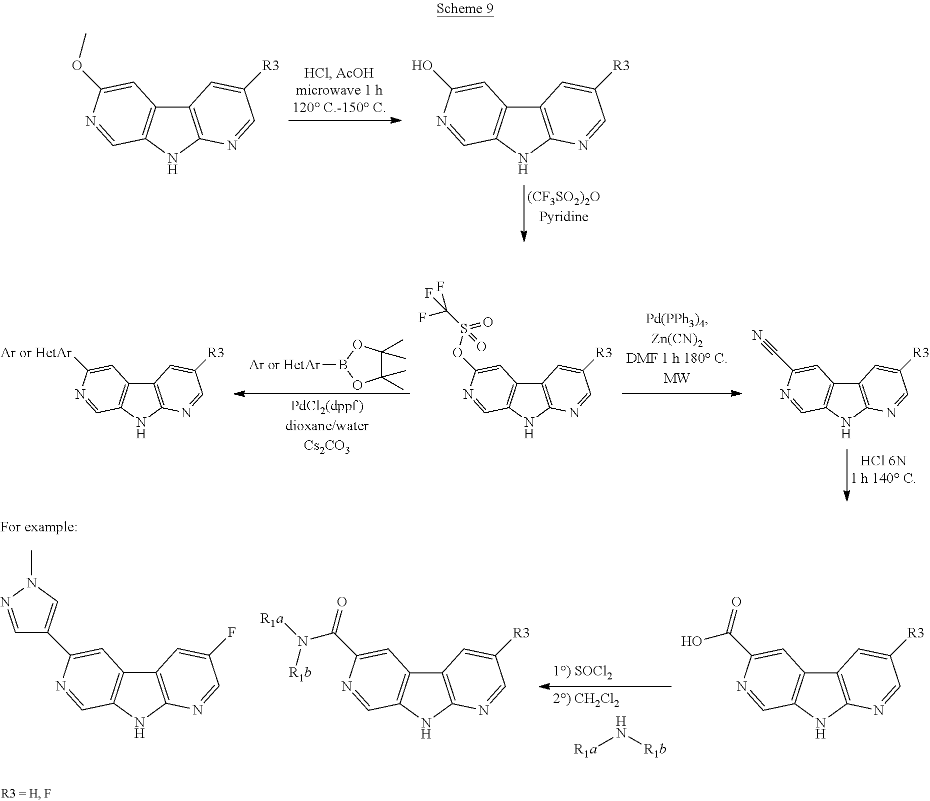

The derivative 3-fluoro-6-methoxy-9H-pyrrolo[2,3-b:5,4-c′]dipyridine may also be employed in a metallation-iodination reaction already described hereinabove. After a Suzuki reaction, the compound obtained can be subjected to the same sequence as previously (demethylation, formation of the triflate and then introduction of the heteroaryl via a Suzuki coupling).

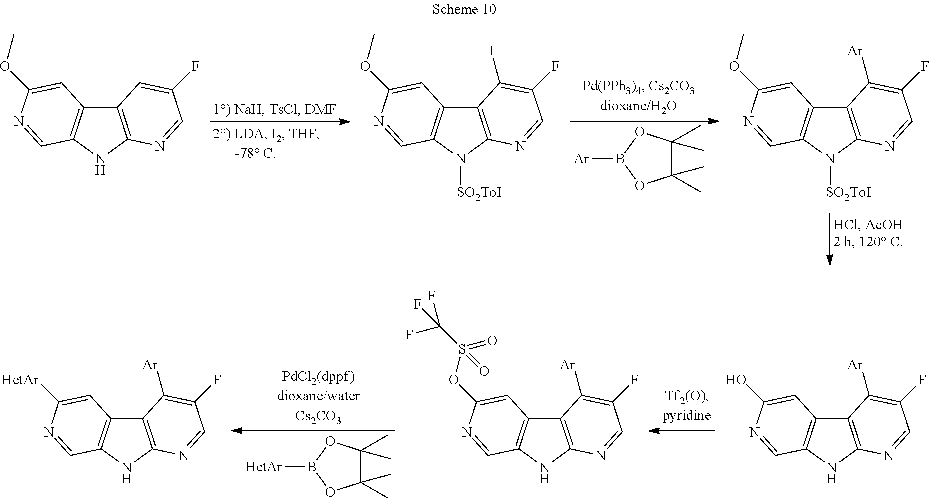

In certain cases, the variations in position 4 may be produced via a triflate group obtained from the corresponding methoxy. In this case, the coupling-cyclization sequence is performed with the stannyl derivative described hereinabove and 2-amino-3-iodo-4-methoxypyridine. The dimethoxy tricyclic compound is then converted into the corresponding ditriflate in two steps. This ditriflate preferentially reacts in position 4 during a Suzuki coupling, which makes it possible to selectively and sequentially introduce an aryl group into position 4 and a heteroaryl group into position 6.

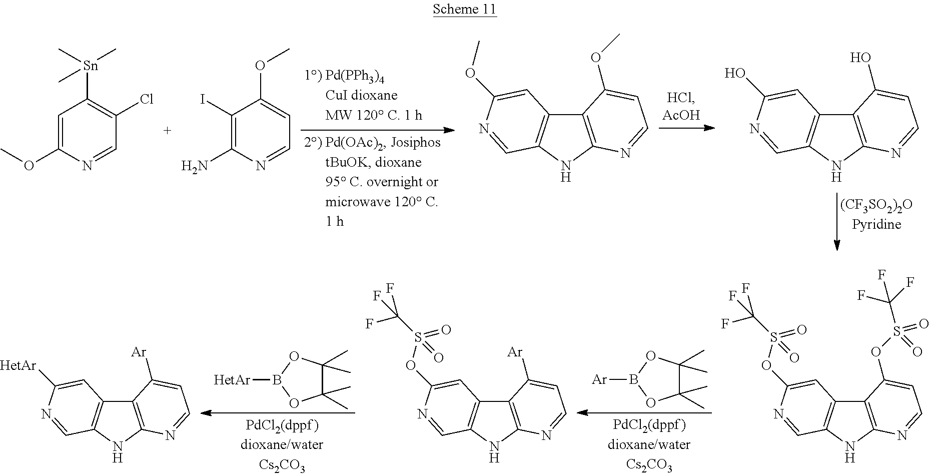

According to another of its aspects, the present invention relates to pharmaceutical compositions comprising, as active principle, a compound according to the invention. These pharmaceutical compositions contain an effective dose of at least one compound according to the invention, or a pharmaceutically acceptable salt of the said compound, and also at least one pharmaceutically acceptable excipient.

The said excipients are chosen, according to the pharmaceutical form and the desired mode of administration, from the usual excipients known to those skilled in the art.

In the pharmaceutical compositions of the present invention for oral, sublingual, subcutaneous, intramuscular, intravenous, topical, local, intratracheal, intranasal, transdermal or rectal administration, the active principle of formula (I) above, or the salt thereof, may be administered in a unit administration form, as a mixture with standard pharmaceutical excipients, to man and animals for the treatment of the above disorders or diseases.

The appropriate unit administration forms include oral forms such as tablets, soft or hard gel capsules, powders, granules and oral solutions or suspensions, sublingual, buccal, intratracheal, intraocular, intranasal and inhalation administration forms, topical, transdermal, cutaneous, intramuscular or intravenous administration forms, rectal administration forms and implants. For topical application, the compounds according to the invention can be used in creams, gels, ointments or lotions.

These medicaments find their therapeutic use especially in the treatment of cancers that are sensitive to Pim kinase deregulation.

The Pim kinase inhibitors that are the subjects of the present invention are useful for treating cancer, especially leukaemias, lymphomas and myelomas. They may also be used for treating various solid tumours, especially cancers of the head and neck, bowel cancer, prostate cancer, pancreatic cancer, liver cancer and buccal cancer, for example. Insofar as cancer remains a disease for which the existing treatments are insufficient, it is manifestly necessary to identify novel Pim kinase inhibitors that are effective in treating cancer.

One subject of the present invention is thus a medicament, characterized in that it comprises a compound of formula (I) as defined above, or an addition salt of this compound with a pharmaceutically acceptable acid.

One subject of the present invention is thus pharmaceutical compositions containing, as active principle, a compound of formula (I) as defined above and also at least one pharmaceutically acceptable excipient.

One subject of the present invention is thus these pharmaceutical compositions used for treating cancer.

One subject of the present invention is thus the use of a compound of formula (I) as defined above for the preparation of a medicament for treating diseases that are sensitive to Pim kinase deregulation.

One subject of the present invention is thus the use of a compound of formula (I) as defined above for the preparation of a medicament for treating cancer.

One subject of the present invention is thus the use of the products of formula (I) as defined above for the preparation of medicaments intended for cancer chemotherapy.

One subject of the present invention is thus the compounds of formula (I) as defined above, as kinase inhibitors.

One subject of the present invention is thus the compounds of formula (I) as defined above, as Pim kinase inhibitors.

According to another of its aspects, the present invention also relates to a method for treating the pathologies indicated above, which comprises the administration, to a patient, of an effective dose of a compound according to the invention, or a pharmaceutically acceptable salt thereof.

The examples that follow describe the preparation of certain compounds in accordance with the invention. These examples are not limiting, and serve merely to illustrate the present invention. The numbers of the illustrated compounds refer to those given in the table hereinbelow, which illustrates the chemical structures and the physical properties of a number of compounds according to the invention.

### EXPERIMENTAL SECTION

### General Conditions

All the reactions are performed with anhydrous solvents of the Acros Organics AcroSeal range. The solvents used for the extractions and the chromatographies are obtained from SDS. The purifications on silica gel are performed using silica cartridges (15-40 μm silica gel 60). The preparative HPLC purifications are performed on Macherey-Nagel columns (Nucleodur C18 phase) or on other phases (Chiralcel OD-I or OJ-H or AS-H, Chiralpak, Krornasil C₁₈) with suitable eluents.

LC-MS-DAD-ELSD analysis: 2 possible experimental conditions:

1 LC-MS-DAD-ELSD analysis (or LC-MS (7 min)): MS=Waters ZQ; electrospray mode +/−; mass range m/z=100-1200; LC=Agilent HP 1100; LC column=Waters X Bridge 18 C, 3.0×50 mm-2.5 μm; LC oven=60° C.; flow rate=1.1 ml/minute.

Eluents: A=water+0.1% formic acid, B=acetonitrile with the following gradient:

| Time | A % | B % |
|---|---|---|
| 0.0 | 95 | 5.0 |
| 5.0 | 5.0 | 100 |
| 5.5 | 5.0 | 100 |
| 6.5 | 95.0 | 5.0 |
| 7.0 | 95.0 | 5.0 |

2 LC-MS-DAD-ELSD analysis (or LC-MS (7 min)): MS=Platform II Waters Micromass; electrospray +/−; mass range m/z=100-1100; Waters LC Alliance 2695; Waters X Terra 18C column; 4.6 mm×75 mm 2.5 μm; LC oven=60° C.; flow rate=1.0 ml/minute.

Eluents: A=water+0.1% formic acid, B=acetonitrile with the following gradient:

| Time | A % | B % |
|---|---|---|
| 0 | 95 | 5 |
| 6.0 | 5 | 95 |
| 8.0 | 5 | 95 |
| 9.0 | 95 | 5 |
| 13.0 | 95 | 5 |

UPLC-MS-DAD-ELSD analysis: 2 possible experimental conditions:

1 UPLC-MS-DAD-ELSD analysis: MS=Waters Quattro Premier XE; electrospray +/−; mass range m/z=100-1100; Waters UPLC; Acquity UPLC BeH C18 1.7 μm 3 mm×50 mm column; UPLC oven=70° C., flow rate=0.7 ml/minute.

Eluents: A=water+0.1% formic acid, B=acetonitrile+0.1% formic acid with the gradient:

| Time | A % | B % |
|---|---|---|
| 0 | 95 | 5 |
| 5 | 0 | 100 |
| 5.5 | 95 | 5 |
| 6.0 | 95 | 5 |

2 UPLC-MS-DAD-ELSD analysis: MS=Waters SQD; electrospray +/−; mass range m/z=100-1100; Waters UPLC; Acquity UPLC Beh C18 1.7 μm 3 mm×50 mm column; UPLC oven=70° C., flow rate=1 ml/minute.

Eluents: A=water+0.1% formic acid, B=acetonitrile+0.1% formic acid with the ingredient:

| Time | A % | B % |
|---|---|---|
| 0 | 95 | 5 |
| 0.8 | 50 | 50 |
| 1.2 | 0 | 100 |
| 1.85 | 0 | 100 |
| 1.95 | 95 | 5 |
| 2.00 | 95 | 5 |

For the detection:
DAD wavelength considered λ=210-400 nm
ELSD: Sedere SEDEX 85; nebulization temperature=35° C.; nebulization pressure=3.7 bar
N.B: As a function of the analysed structures, the dilution solvents are: dimethyl sulfoxide; methanol; acetonitrile; dichloromethane.

### Synthetic Processes

### Synthesis of 5-chloro-4-trimethylstannyl-2-(3′-pyridyl)pyridine 2

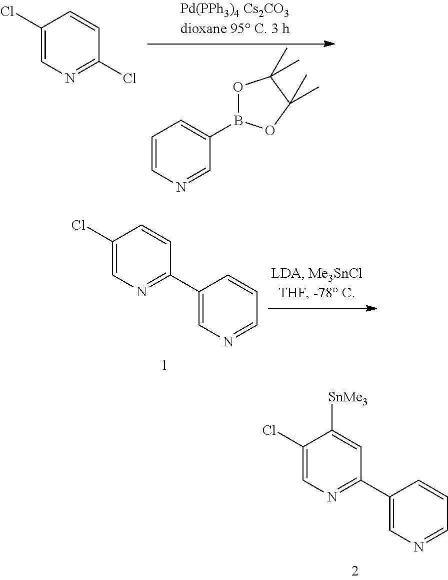

### Step 1:

1.4 g of 2,5-dichloropyridine, 2.04 g of 3-(4,4,5,5-tetramethyl-1,3,2-dioxaborolan-2-yl)pyridine, 0.76 g of tetrakis(triphenylphosphine)palladium(0) and 7.7 g of caesium carbonate are introduced into a 20 mL microwave tube under argon, followed by addition of 15.5 mL of 1,4-dioxane and 0.7 mL of water. The mixture is heated by microwave for 1 hour at 125° C. The reaction may also be performed by standard heating (duration of 6 hours in the refluxing solvent). After cooling, the reaction mixture is poured into 75 mL of a 10% sodium bicarbonate solution and 25 mL of water, extracted twice with 100 mL of ethyl acetate, dried over sodium sulfate, filtered and concentrated to dryness under reduced pressure. 2.8 g of a crude product are obtained, and are purified by chromatography on silica gel, eluting with a mixture of heptane and ethyl acetate (70/30 by volume), thus giving 1.2 g (67%) of 5-chloro-2-(3′-pyridyl)pyridine 1.

LC-MS-DAD-ELSD: 191(+)=(M+H)(+) Rt (min)=2.28

### Step 2:

1.4 mL of diisopropylamine and 5 mL of tetrahydrofuran are introduced into a dry round-bottomed flask under an argon atmosphere, equipped with a magnetic stirrer. The solution is cooled to −78° C. and 3.95 mL of n-butyllithium (2.5 M in hexane) are then added. After stirring for 15 minutes, 1.45 g of 1 predissolved in 20 mL of tetrahydrofuran are added. After stirring for 2 hours, 10 mL of a 1 M solution of trimethyltin chloride in hexane are added. The mixture is thus stirred for 1 hour at −78° C. The reaction medium is hydrolysed with 120 mL of 10% ammonium chloride solution and 30 mL of water. The resulting mixture is extracted twice with 50 mL of ethyl acetate, and then dried over sodium sulfate, filtered and concentrated to dryness under reduced pressure. 3.2 g of a crude product are obtained, and are purified by chromatography on silica gel with a gradient of heptane and ethyl acetate eluent (from 100/0 to this 70/30 by volume), thus giving 1.7 g (63%) g of 5-chloro-4-trimethylstannyl-2-(3′-pyridyl)pyridine 2.

LC-MS-DAD-ELSD: 354(+)=(M+H)(+) (isotope profile corresponding to a tin derivative) Rt (min)=4.36

### Examples 1 to 8 (5a-5h)

### General Procedure for the Sequence: Stille Coupling/Amination Cyclization Under the Hartwig-Buchwald Conditions

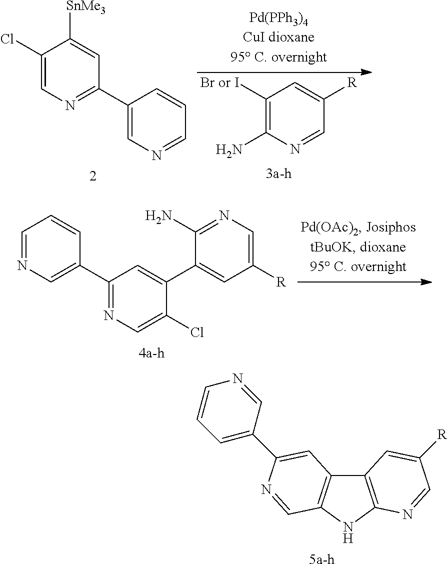

10 mmol of 2-amino-3-halo (bromo or iodo)pyridine 3a-h (see Table 1), 10.5 mmol of 5-chloro-4-trimethylstannyl-2-(3′-pyridyl)pyridine 2, 1 mmol of tetrakis(triphenylphosphine)palladium(0) and 2 to 3 mmol of copper iodide in 30 mL of 1,4-dioxane are introduced into a 100 mL round-bottomed flask. The reaction mixture is heated at 100° C. overnight. After cooling, the reaction mixture is poured into 200 mL of a 10% sodium bicarbonate solution and 25 mL of water, extracted twice with 200 mL of ethyl acetate, dried over sodium sulfate, filtered and concentrated to dryness under reduced pressure. The crude product is purified by chromatography on silica gel with a gradient of ethyl acetate and methanol or of dichloromethane and methanol eluent (from 100/0 to 90/10 by volume). The coupled products 4a-h are obtained in yields of between 40% and 75%. 5 mmol of the coupled product 4 are dissolved in 30 mL of 1,4-dioxane, under an argon atmosphere, in a dry 150 mL round-bottomed flask. 0.35 mmol of palladium (II) acetate and 0.75 mmol of (R)-(−)-1-[(S)-2-(dicyclohexylphosphino)ferrocenyl]ethyldi-tert-butylphosphine are placed in a dry tube under argon, 6 mL of 1,4-dioxane are added and the mixture is stirred for 10 minutes under argon. This catalyst solution is added to the solution of 3 along with 7 to 12 mmol of potassium tert-butoxide. The resulting mixture is heated overnight at 100° C. After cooling, 10 mL of methanol and 150 mL of ethyl acetate are added. The organic phase is washed with aqueous sodium bicarbonate solution, dried and evaporated. The crude product is purified by chromatography on silica gel with a gradient of ethyl acetate and methanol or of dichloromethane and methanol eluents (from 100/0 to 90/10 by volume). The cyclized products 5a-h are detailed in Table 1 (yield of between 35% and 80% depending on the substrate).

The cyclization may also be performed using another catalytic system: in this case, product 4 (1 mmol) is placed in a 5 mL microwave tube with 0.05 mmol of tris(dibenzylidene-acetone)dipalladium(0), 0.11 mmol of 2-dicyclohexylphosphino-2-(N,N-dimethylamino)biphenyl and 1.5 mmol of potassium tert-butoxide. The tube is sealed and placed under an argon atmosphere, and 4 mL of 1,4-dioxane are then added. The mixture is heated by microwave for 1 hour at 150° C. The work-up and purification of compound 5 are performed in the manner described above. The yields are generally lower than those obtained with the Pd(OAc)₂/Josiphos system.

All the steps of this sequence may be performed either by heating with microwaves (between 110 and 150° C.) or by standard heating (reflux).

TABLE 1

| Reagent 3 | Structure obtained 5 | Name | Analysis |
|---|---|---|---|
| 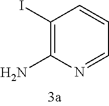 | 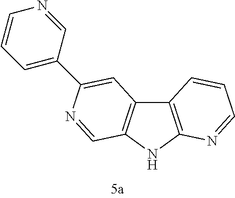 | 6-pyrid-3-yl-9H- pyrrolo[2,3-b:5,4- c′]dipyridine | 1H NMR (400 MHz, DMSO-d₆) δ ppm: 7.35 (dd, J = 8.0, 4.5 Hz, 1 H) 7.53 (dd, J = 8.5, 4.5 Hz, 1 H) 8.51 (dt, J = 8.5, 1.5 Hz, 1 H) 8.59 (dd, J = 4.5, 1.5 Hz, 1 H) 8.62 (dd, J = 4.5, 1.5 Hz, 1 H) 8.72 (dd, J = 8.0, 1.5 Hz, 1 H) 8.91 (d, J = 1.0 Hz, 1 H) 9.03 (d, J = 1.0 Hz, 1H) 9.37 (d, J = 1.5 Hz, 1 H) 12.3 (broad m, 1H) LC-MS-DAD-ELSD: 247(+) = (M + H)(+) |
| 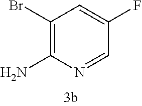 | 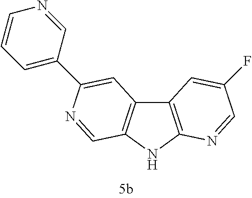 | 3-fluoro-6-(pyrid-3- yl)-9H-pyrrolo[2,3- b:5,4-c′] dipyridine | 1H NMR (400 MHz, DMSO-d₆) δ ppm: 7.54 (dd, J = 8.1, 4.5 Hz, 1 H) 8.48 (dt, J = 8.1, 2.0 Hz, 1 H) 8.60 (dd, J = 4.5, 2.0 Hz, 1 H) 8.62-8.69 (m, 2 H) 8.91 (d, J = 1.0 Hz, 1 H) 9.05 (d, J = 1.0 Hz, 1 H) 9.34 (d, J = 2.0 Hz, 1 H) 12.35 (broad m, 1 H) LC-MS-DAD-ELSD: 263(−) = (M − H)(−); 265(+) = (M + H)(+) |
| 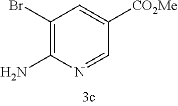 | 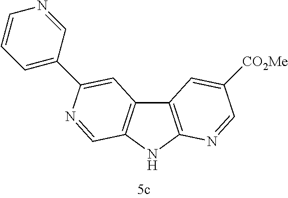 | methyl 6-(pyrid-3- yl)-9H-pyrrolo[2,3- b:5,4-c′]dipyridine- 3-carboxylate | 1H NMR (400 MHz, DMSO-d₆) δ ppm: 3.95 (s, 3 H) 7.54 (dd, J = 8.0, 5.0 Hz, 1 H) 8.53 (dt, J = 8.0, 2.0 Hz, 1 H) 8.59 (dd, J = 5.0, 2.0 Hz, 1 H) 9.07 (d, J = 1.0 Hz, 1 H) 9.10 (broad s, 1 H) 9.15 (d, J = 2.0 Hz, 1 H) 9.32 (d, J = 2.0 Hz, 1 H) 9.38 (d, J = 2.0 Hz, 1 H) 12.5 (very broad m, 1H ) LC-MS-DAD-ELSD: 303(−) = (M − H)(−); 305(+) = (M + H)(+) |
| 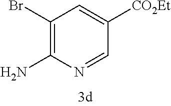 | 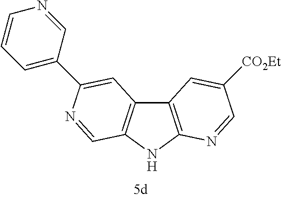 | ethyl 6-(pyrid-3-yl)- 9H-pyrrolo [2,3- b:5,4-c′] dipyridine- 3-carboxylate | 1H NMR (400 MHz, DMSO-d₆) δ ppm: 1.41 (t, J = 7.2 Hz, 3 H) 4.42 (q, J = 7.2 Hz, 2 H) 7.55 (dd, J = 8.0, 4.9 Hz, 1 H) 8.53 (dt, J = 8.0, 2.0 Hz, 1 H) 8.60 (dd, J = 4.9, 2.0 Hz, 1 H) 9.08 (broad s, 1 H) 9.13 (d, J = 1.0 Hz, 1 H) 9.17 (d, J = 2.0 Hz, 1 H) 9.33 (d, J = 2.0 Hz, 1 H) 9.39 (d, J = 2.0 Hz, 1 H) 12.7 (broad m, 1 H) LC-MS-DAD-ELSD: 317(−) = (M − H)(−); 319(+) = (M + H)(+) |
| 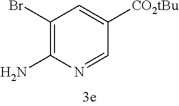 | 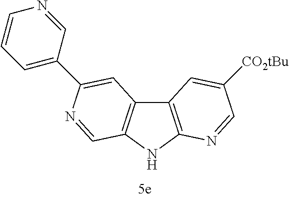 | 2-methylpropan-2-yl 6-(pyrid-3-yl)-9H- pyrrolo[2,3-b:5,4- c′]dipyridine-3- carboxylate | 1H NMR (400 MHz, DMSO-d₆) δ ppm: 1.63 (s, 9 H) 7.55 (dd, J = 8.0, 4.5 Hz, 1 H) 8.53 (dt, J = 8.0, 2.0 Hz, 1 H) 8.60 (dd, J = 4.5, 2.0 Hz, 1 H) 9.07 (s, 1 H) 9.10 (s, 1 H) 9.11 (d, J = 2.5 Hz, 1 H) 9.24 (d, J = 2.5 Hz, 1 H) 9.39 (d, J = 2.0 Hz, 1 H) 12.66 (broad m, 1 H) LC-MS-DAD-ELSD: 345(−) = (M − H)(−); 347(+) = (M + H)(+) |
| 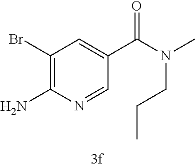 | 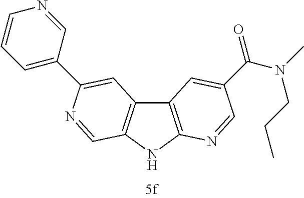 | N-methyl-N-propyl- 6-(pyrid-3-yl)-9H- pyrrolo[2,3-b:5,4- c′]dipyridine-3- carboxamide | 1H NMR (400 MHz, DMSO-d6) δ ppm: 0.59-1.09 (broad m, 3H) 1.51-1.73 (broad m, 2 H) 3.05 (s, 3H) 3.25-3.55 (partially masked m, 2 H) 7.54 (dd, J = 8.0, 4.9 Hz, 1 H) 8.50 (dt, J = 8.0, 2.0 Hz, 1 H) 8.59 (dd, J = 4.9, 2.0 Hz, 1 H) 8.66 (broad s, 1 H) 8.83 (broad s, 1 H) 8.99 (broad s, 1 H) 9.05 (d, J = 1.0 Hz, 1H) 9.36 (d, J = 2.0 Hz, 1 H) 12.5 (broad m, 1 H) LC-MS-DAD-ELSD: 344(−) = (M − H)(−); 346(+) = (M + H)(+) |
| 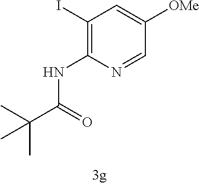 | 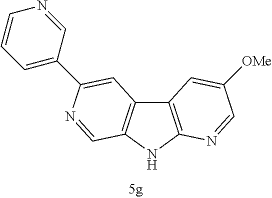 | 3-methoxy-6-(pyrid- 3-yl)-9H-pyrrolo- [2,3-b:5,4-c′]- dipyridine | 1H MMR (400 MHz, DMSO-d6) δ ppm: 3.94 (s, 3 H) 7.53 (dd, J = 7.9, 4.8 Hz, 1H) 8.39 (s, 2 H) 8.49 (dt, J = 8.0, 2.0 Hz, 1 H) 8.58 (dd, J = 4.8, 2.0 Hz, 1 H) 8.90 (broad s, 1 H) 8.99 (d, J = 1.0 Hz, 1 H) 9.35 (d, J = 2.0 Hz, 1 H) 12.08 (s, 1 H) UPLC-SQD: Rt (min) = 0.41; MH+ = 277 +; Purity: 98% |
| 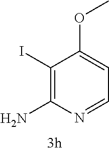 | 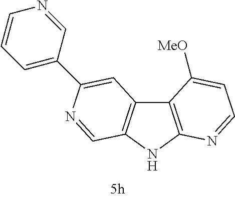 | 4-methoxy-6-(pyrid- 3-yl)-9H-pyrrolo- [2,3-b:5,4-c′]- dipyridine | 1H NMR (400 MHz, DMSO-d6) δ ppm 4.16 (s, 3 H) 6.97 (d, J = 5.6 Hz, 1 H) 7.52 (ddd, J = 8.0, 4.8, 0.8 Hz, 1 H) 8.47 (ddd, J = 8.0, 2.2, 1.7 Hz, 1 H) 8.50 (d, J = 5.6 Hz, 1 H) 8.53 (d, J = 1.2 Hz, 1 H) 8.59 (dd, J = 4.8, 1.7 Hz, 1 H) 8.98 (d, J = 1.2 Hz, 1 H) 9.31 (dd, J = 2.2, 0.8 Hz, 1 H) 12.22 (broad m, 1 H) UPLC-SQD: Rt (min) = 0.35; MH+ = 277 +; Purity: 98% |

### Synthesis of 3e and 3f

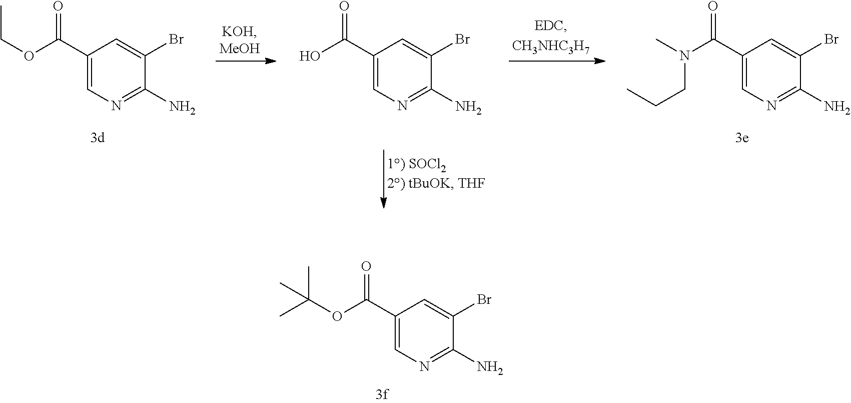

2.41 g of ethyl 2-amino-3-bromo-5-pyridinecarboxylate 3d in 120 mL of methanol and 2.8 g of potassium hydroxide in 40 mL of water are placed in a round-bottomed flask. The mixture is stirred with heating at 60° C. for 3 hours. The methanol is evaporated off. After cooling, 10 mL of 5N hydrochloric acid are added. The precipitate is filtered off to give 2.07 g of 6-amino-5-bromonicotinic acid.

LC-MS-DAD-ELSD: 217(+) and 219(+)=(M+H)(+) Rt (min)=1.71

### For 3e:

543 mg of 6-amino-5-bromonicotinic acid and 385 mL of methylpropylamine in 20 mL of dichloromethane are placed in a round-bottomed flask. After stirring, 1.05 g of 1-(3-dimethylaminopropyl)-3-ethylcarbodiimide hydrochloride are added. After stirring overnight, the reaction mixture is poured into 50 mL of a 10% sodium bicarbonate solution and 10 mL of water, extracted twice with 25 mL of dichloromethane, dried over sodium sulfate, filtered and concentrated to dryness under reduced pressure. 859 mg of a crude product are obtained, and are purified by chromatography on silica gel with a gradient of heptane and ethyl acetate eluent (from 95/5 to 50/50 by volume), thus giving 485 mg of 6-amino-5-bromo-N-methyl-N-propylnicotinamide 3e.

LC-MS-DAD-ELSD: 272(+)=(M+H)(+) Rt (min)=2.59

### For 3f:

1.5 g of 6-amino-5-bromonicotinic acid and 10 mL of thionyl chloride are placed in a round-bottomed flask. The mixture is refluxed for 3 hours. The excess thionyl chloride is evaporated off and the mixture is then taken up in 25 mL of tetrahydrofuran and 3.103 g of potassium tert-butoxide predissolved in 25 mL, of tetrahydrofuran are added. After stirring for one hour, the mixture is concentrated to dryness under reduced pressure and the residue is purified by chromatography on silica gel with a gradient of heptane and ethyl acetate eluents (from 100/0 to 80/20 by volume), thus giving 230 mg of tert-butyl 6-amino-5-bromonicotinate.

LC-MS-DAD-ELSD: 217(+) and 219(+)=(M+H—C₄H₉)(+) 273 and 275=(M+H)(+) Rt (min)=1.71

### Synthesis of 3g

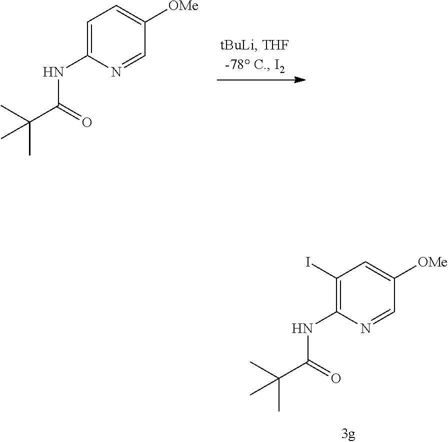

A solution of 15 mmol of N-(5-methoxypyrid-2-yl)-2,2-dimethylpropionamide in dry tetrahydrofuran (70 mL) is introduced by syringe into a dry one-necked flask under argon. The solution is cooled to −78° C., and 37.5 mmol of tert-butyllithium (1.5 M in pentane) are then added over 15 minutes. The temperature is allowed to rise to 0° C. and the mixture is left stirring for 2 hours. The reaction mixture is again cooled to −78° C. and a solution of 37.5 mmol of diiodine in 5 mL of dry tetrahydrofuran is then added. The reaction mixture is then poured into aqueous ammonium chloride solution, which is extracted with ethyl acetate. The organic phase is washed with aqueous sodium thiosulfate solution and then dried over magnesium sulfate and concentrated under reduced pressure. The crude product is purified by chromatography on silica gel (gradient: dichloromethane to 95/5 dichloromethane/methanol). 1.7 g (34%) of compound 3g are obtained.

UPLC-MS-DAD-ELSD: 334(+)=(M+H)(+) Rt (min)=0.88

### Example 9

### Synthesis of 3-bromo-6-(pyrid-3-yl)-9H-pyrrolo[2,3-b:5,4-c′]dipyridine 6

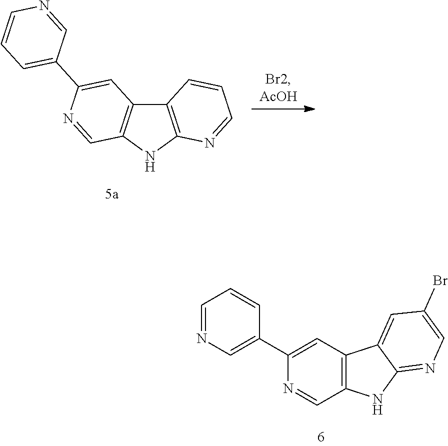

360 mg of 5a, 15 mL of acetic acid and 10 mL of dimethylformamide are placed in a round-bottomed flask. After stirring, 0.3 mL of bromine is added dropwise. After stirring for 3 hours at room temperature, the precipitate is filtered off and then suction-filtered with aqueous sodium thiosulfate solution and water. After drying, 463 mg (97%) of 3-bromo-6-(pyrid-3-yl)-9H-pyrrolo[2,3-b:5,4-c′]-dipyridine 6 are obtained.

1H NMR (400 MHz, DMSO-d6) δ ppm: 7.54 (dd, J=8.0, 4.9 Hz, 1 H) 8.47 (dt, J=8.0, 2.0 Hz, 1 H) 8.60 (dd, J=4.9, 2.0 Hz, 1 H) 8.69 (d, J=2.4 Hz, 1 H) 8.93 (s, 1 H) 9.00 (d, J=2.4 Hz, 1 H) 9.05 (s, 1 H) 9.34 (d, J=2.0 Hz, 1 H) 12.55 (broad m, 1 H)

LC-MS-DAD-ELSD: 323(−)/ . . . =(M−H)(−)/ . . . ; 325(+)/ . . . =(M+H)(+)/ . . . (1 Br present)

### Examples 10 to 12 (9a-9c)

### General Procedure for the Amination Reaction Under the Buchwald Conditions

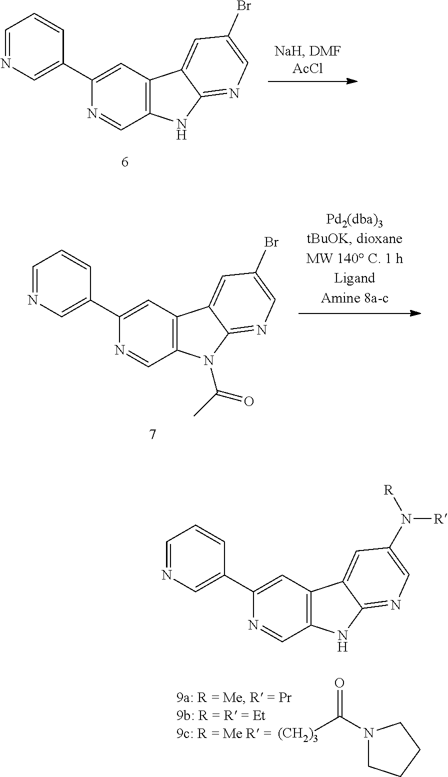

325 mg of 6 in 30 mL of dimethylformamide are placed in a 50 mL round-bottomed flask. 80 mg of sodium hydride are added under argon. After stirring for two hours, 0.194 mL of acetyl chloride in 2 mL of dimethylformamide is added. After stirring for two hours, the reaction mixture is poured into 50 mL of a 10% sodium bicarbonate solution and 20 mL of water, extracted twice with 50 mL of ethyl acetate, dried over magnesium sulfate, filtered and concentrated to dryness under reduced pressure. 355 mg of 1-(3-bromo-6-pyrid-3-yldipyrido[2,3-b:4′,3′-d]pyrrol-9-yl)ethanone 7 are obtained, and are used in the following step without purification.

0.25 mmol of 7, 17.5 μmol of tris(dibenzylideneacetone)dipalladium(0), 37.5 μmol of ligand (see Table 2) and 0.625 mmol of potassium tert-butoxide in 2 mL of 1,4-dioxane are placed in a 5 mL microwave tube. Next, between 0.8 and 2.5 mmol of amine 8a-c (see Table 2) are added. The mixture is heated by microwave for 1 hour at 140° C. After cooling, the reaction mixture is poured into 50 mL of a 10% sodium bicarbonate solution and 20 mL of water, extracted twice with 60 mL of ethyl acetate, dried over sodium sulfate, filtered and concentrated to dryness under reduced pressure. The products 9a-c are purified by preparative HPLC and obtained in yields of between 7% and 30%.

TABLE 2

| Amine 8 | Ligand | Structure 9 | Name | Analysis |
|---|---|---|---|---|
| 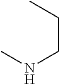 | 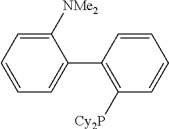 | 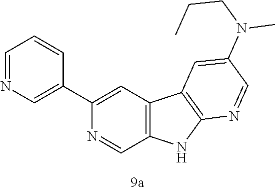 | N-methyl-N-propyl- 6-(pyrid-3-yl)-9H- pyrrolo[2,3-b:5,4- c′]dipyrid-3-amine | 1H NMR (400 MHz, DMSO-d6) δ ppm: 0.94 (t, J = 1.6 Hz, 3 H) 1.60 (m, 2 H) 2.99 (s, 3 H) 3.37 (t, J = 7.6 Hz, 2 H) 7.51 (dd, J = 8.5, 4.5 Hz, 1 H) 8.08 (d, J = 2.9 Hz, 1 H) 8.29 (d, J = 2.9 Hz, 1 H) 8.49 (dt, J = 8.5, 2.0 Hz, 1 H) 8.56 (dd, J = 4.5, 2.0 Hz, 1 H) 8.88 (d, J = 1.0 Hz, 1 H) 8.92 (d, J = 1.0 Hz, 1 H) 9.36 (d, J = 2.0 Hz, 1 H) 11.80 (broad s, 1 H) LC-MS-DAD-ELSD 317(+) = M(+) 288(+) = 317(+) − Et |
| 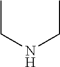 | 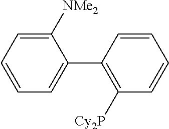 | 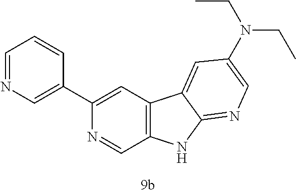 | N,N-diethyl-6- (pyrid-3-yl)-9H- pyrrolo[2,3-b:5,4- c′]dipyrid-3-amine | 1H NMR (400 MHz, DMSO-d6) δ ppm: 1.14 (t, J = 7.2 Hz, 6 H) 3.42 (q, J = 7.2 Hz, 4H) 7.51 (dd, J = 8.0, 4.6 Hz, 1 H) 8.10 (d, J = 2.9 Hz, 1 H) 8.27 (d, J = 2.9 Hz, 1 H) 8.42 (s, 1 H) 8.49 (dt, J = 8.0, 2.0 Hz, 1 H) 8.56 (dd, J = 4.6, 2.0 Hz, 1 H) 8.89 (d, J = 1.0 Hz, 1 H) 8.92 (d, J = 1.0 Hz, 1 H) 9.36 (d, 7 = 2.0 Hz, 1 H) 11.8 (very broad m, 1 H) UPLC-MS-DAD- ELSD: 318(+) = (M + H)(+) |
| 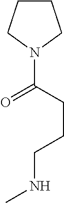 | 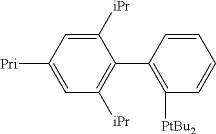 | 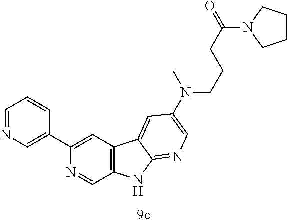 | 4-{methyl[6-(pyrid- 3-yl)-9H-pyrrolo- [2,3-b:5,4-c′]dipy- rid-3-yl] amino}-1- (pyrrolidin-1-yl)- butan-1-one | 1H NMR (400 MHz, DMSO-d6) δ ppm: 1.72 (m, 2 H) 1.81 (m, 4 H) 2.31 (t, J = 6.8 Hz, 2 H) 2.99 (s, 3 H) 3.20-3.38 (partially masked m, 4 H) 3.45 (m, 2 H) 7.51 (dd, J = 7.8, 4.9 Hz, 1 H) 8.10 (d, J = 2.9 Hz, 1H) 8.34 (d, J = 2.9 Hz, 1 H) 8.49 (dt, J = 7.8, 2.0 Hz, 1 H) 8.56 (dd, J = 4.9, 2.0 Hz, 1 H) 8.85 (s, 1 H) 8.92 (s, 1 H) 9.35 (d, J = 2.0 Hz, 1H) 11.8 (s, 1H) LC-MS-DAD-ELSD 415(+) = (M + H)(+) |

### Synthesis of the Amine 8c

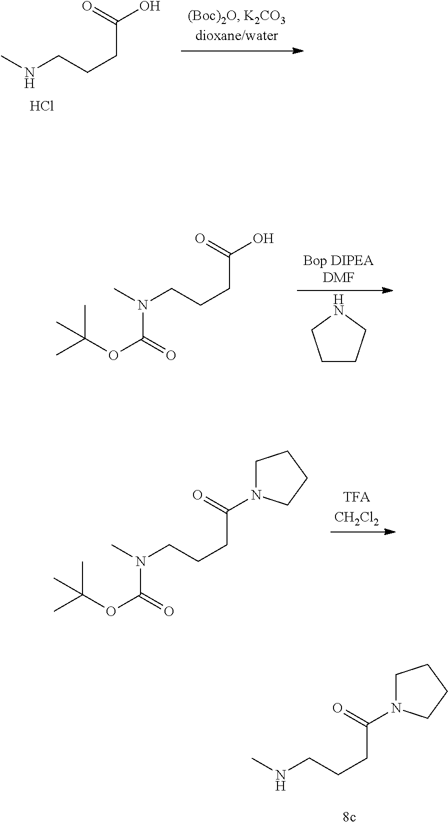

### Step 1:

3 g of 4-(methylamino)butyric acid hydrochloride, 7 g of potassium carbonate in 40 mL of 1,4-dioxane and 20 mL of water are placed in a round-bottomed flask. Next, 4.86 g of di-tert-butyl dicarbonate are added. After stirring for 6 hours, the dioxane is evaporated off and 30 mL of water are then added. Aqueous 1 M potassium bisulfate solution is added until pH 2 is obtained. The resulting mixture is extracted twice with 10 mL of ethyl acetate, dried over sodium sulfate, filtered and concentrated to dryness under reduced pressure. 4.37 g of 4-(tert-butoxycarbonylmethylamino)butyric acid are obtained.

### Step 2:

1 g of 4-(tert-butoxycarbonylmethylamino)butyric acid in 10 mL of dimethylformamide, 1.925 g of [dimethylamino-(1,2,3-triazolo[4,5-b]pyrid-3-yloxy)methylene]dimethylammonium hexafluorophosphate, 713 mg of N,N-diisopropylethylamine and 360 mg of pyrrolidine are introduced into a 100 mL round-bottomed flask. The solution is stirred overnight at room temperature. The reaction mixture is poured into 50 mL of water and extracted three times with 200 mL of ethyl acetate. The product is taken up in dichloromethane and filtered through 2 cm of silica. The filtrate is evaporated to dryness under vacuum. 806 mg of tert-butyl methyl(4-oxo-4-pyrrolidin-1-ylbutyl)carbamate are thus obtained.

### Step 3:

400 mg of tert-butyl methyl(4-oxo-4-pyrrolidin-1-ylbutyl)carbamate and 5 mL of trifluoroacetic acid in 25 mL of dichloromethane are placed in a round-bottomed flask. The mixture is stirred for 3 hours 30 minutes at room temperature. The reaction mixture is concentrated under reduced pressure. The product is purified by chromatography (SCX), eluting with a mixture of methanol and 2N aqueous ammonia. 125 mg of 4-methylamino-1-pyrrolidin-1-ylbutan-1-one 8c are obtained.

### Example 13

### Synthesis of 3-chloro-6-(pyrid-3-yl)-9H-pyrrolo[2,3-b:5,4-c′]dipyridine 10

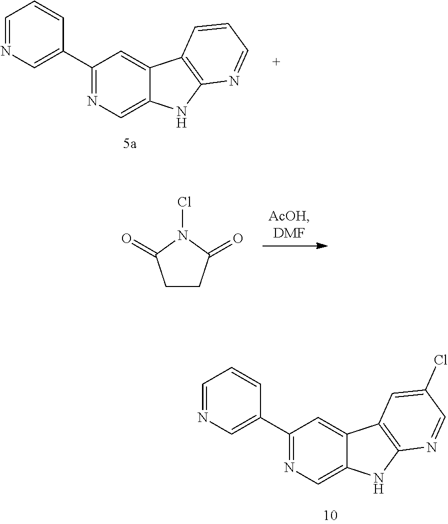

To a solution of 493 mg of 5a in 20 ml of acetic acid and 5 mL of dimethylformamide are added dropwise 802 mg of N-chlorosuccinimide dissolved in 5 mL of dimethylformamide. The reaction mixture is then stirred at 25° C. overnight. The suspension obtained is concentrated under reduced pressure, and the dry extract is taken up in a methanol/dichloromethane mixture with 1 g of silica and then concentrated under reduced pressure in order to produce a solid deposit on the column. The crude product is purified by chromatography on silica gel (gradient: 100% ethyl acetate to 90/10 ethyl acetate/methanol). The fractions containing the expected product are combined and concentrated under reduced pressure to give 380 mg of 3-chloro-6-(pyrid-3-yl)-9H-pyrrolo[2,3-b:5,4-c′]dipyridine in an NMR purity of 80%. The product obtained is taken up in 10 mL of DMSO, the insoluble matter is separated out by filtration and the filtrate is purified by preparative HPLC to give 60 mg of 3-chloro-6-(pyrid-3-yl)-9H-pyrrolo[2,3-b:5,4-c′]dipyridine 10, the characteristics of which are as follows:

1 H NMR (400 MHz, DMSO-d6) δ ppm: 7.55 (dd, J=8.1, 4.8 Hz, 1 H) 8.48 (dt, J=8., 2.0 Hz, 1 H) 8.60 (dd, J=4.8, 2.0 Hz, 1 H) 8.65 (d, J=2.4 Hz, 1 H) 8.89 (d, J=2.4 Hz, 1 H) 8.94 (d, J=1.0 Hz, 1 H) 9.06 (d, J=1.0 Hz, 1 H) 9.34 (d, J=2.0 Hz, 1 H) 12.09 (broad m, 1 H)

UPLC-MS-DAD-ELSD: 279(−)/ . . . =(M−H)(−)/ . . . ; 281(+)/ . . . =(M+H)(+)/ . . . (1 C1 present) (Rt32 0.52 min)

### Example 14

### Synthesis of 3-(morpholin-4-yl)-6-(pyrid-3-yl)-9H-pyrrolo[2,3-b:5,4-c′]dipyridine 11

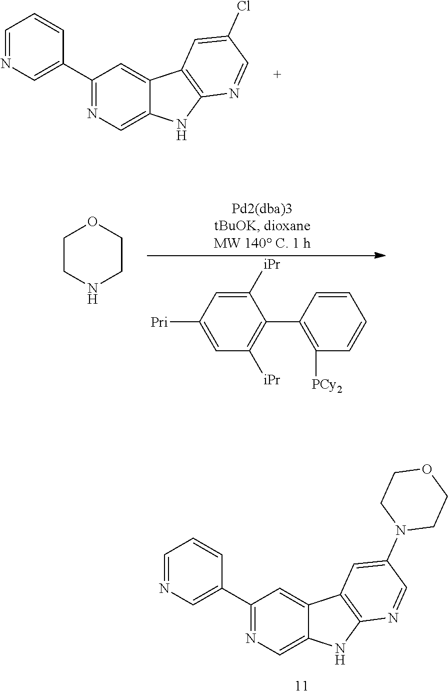

50 mg of 10, 11.4 mg of tris(dibenzylideneacetone)dipalladium(0), 11.3 mg of 2-di-t-butylphosphino-2′,4′,6′-tri-i-propyl-1,1′-biphenyl, 49.9 mg of potassium tert-butoxide, 77.5 mg of morpholine and 2.5 ml, of dioxane are introduced into a microwave reactor of suitable size. The mixture is heated for 1 hour at 140° C. After adding 2 mL of methanol, the reaction medium is poured into 10 mL of ethyl acetate. 500 mg of silica are then added, and the mixture obtained is concentrated under reduced pressure in order to produce a solid deposit on a column. The product is purified by chromatography on silica gel (25 g of silica) using a gradient of 3% to 10% methanol in dichloromethane to give 52 mg of crude product, which is then purified by preparative HPLC, to give 14.6 mg (23%) of 3-(morpholin-4-yl)-6-(pyrid-3-yl)-9H-pyrrolo[2,3-b:5,4-c′]dipyridine 11.

1H NMR (400 MHz, DMSO-d6) δ ppm: 3.20 (m, 4 H) 3.84 (m, 4 H) 7.53 (dd, J=8.3, 4.9 Hz, 1 H) 8.34 (d, J=2.9 Hz, 1 H) 8.47 (partially masked m, 1 H) 8.49 (d, J=2.9 Hz, 1 H) 8.57 (dd, J=4.9, 2.0 Hz, 1H) 8.88 (d, J=1.1 Hz, 1 H) 8.96 (d, J=1.1 Hz, 1 H) 9.34 (d, J=2.0 Hz, 1 H) 11.99 (broad m, 1 H)

UPLC-MS-DAD-ELSD: 330(−)=(M−H)(−); 332(+)=(M+H)(+) (Rt32 0.41 min)

### Example 15

### Synthesis of 3-hydroxy-2,2-dimethylpropyl 6-(pyrid-3-yl)-9H-pyrrolo[2,3-b:5,4-c′]-dipyridine-3-carboxylate 12

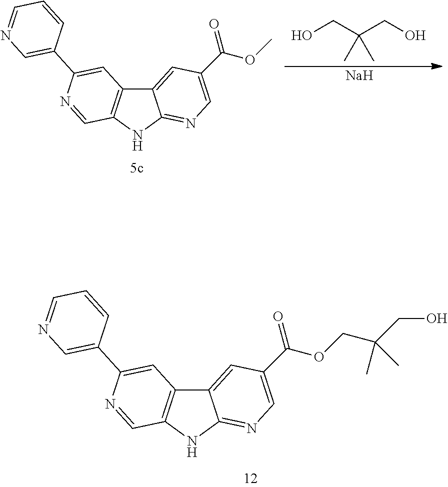

100 mg of 5c, 2.5 g of 2,2-dimethyl-1,3-propanediol and 13 mg of sodium hydride are introduced into a microwave reactor of suitable size. The mixture is heated by microwave for 30 minutes at 160° C. The solid is then dissolved in 80 ml of a 1/1 water/EtOAc mixture. The organic phase is extracted, dried over magnesium sulfate, filtered and evaporated. The crude residue is then purified by chromatography on silica gel (15 g of silica) using a gradient of 2% to 5% of methanol in dichloromethane. The fractions containing the expected product are combined and concentrated under reduced pressure to give 31 mg (25%) of 3-hydroxy-2,2-dimethylpropyl 6-(pyrid-3-yl)-9H-pyrrolo-[2,3-b:5,4-c′]dipyridine-3-carboxylate 12.

1H NMR (400 MHz, DMSO-d6) δ ppm 1.00 (s, 6 H) 3.36 (d, J=5.4 Hz, 2 H) 4.15 (s, 2 H) 4.71 (t, J=5.4 Hz, 1 H) 7.55 (dd, J=8.1, 4.7 Hz, 1 H) 8.54 (dt, J=8.1, 2.0 Hz, 1 H) 8.60 (dd, J=4.7, 2.0 Hz, 1 H) 9.08 (s, 1 H) 9.13 (s, 1 H) 9.18 (d, J=2.4 Hz, 1 H) 9.30 (d, J=2.4 Hz, 1 H) 9.40 (broad d, J=2.0 Hz, 1 H) 12.78 (broad s, 1 H)

UPLC-MS-DAD-ELSD: 375(−)=(M−)(−); 377(+)=(M+H)(+) (Rt32 0.54 min)

### Example 16

### Synthesis of 2-[6-(pyrid-3-yl)-9H-pyrrolo[2,3-b:5,4-c′]dipyrid-3-yl]propan-2-ol 13

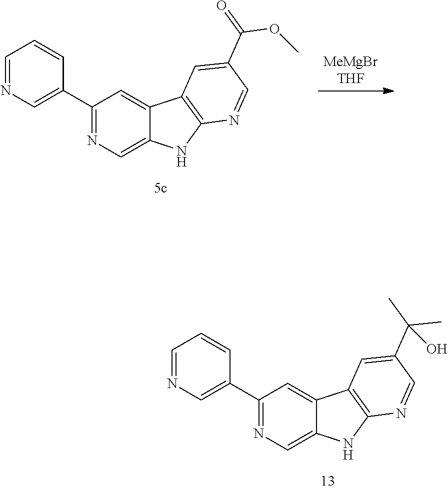

130 mg of 5c and 7 mL of THF are placed in a dry one-necked flask under argon. The mixture is cooled to −20° C., and 0.710 ml of a 3M solution of methylmagnesium bromide in ethyl ether is added over 10 minutes. After stirring for 3 hours, 0.5 mL of methanol is added, and the reaction medium is then hydrolysed with 25 mL of aqueous 10% ammonium chloride solution and 25 mL of water. The aqueous phase is extracted twice with 40 mL of ethyl acetate, dried over magnesium sulfate, filtered and concentrated under reduced pressure. The dry residue is then purified by chromatography on silica gel (25 g of silica) using a gradient of 3% to 10% methanol in dichloromethane. The fractions containing the expected product are combined and concentrated under reduced pressure to give 94 mg (72%) of 2-[6-(pyrid-3-yl)-9H-pyrrolo[2,3-b:5,4-c′]dipyrid-3-yl]propan-2-ol 13, the characteristics of which are as follows:

1H NMR (400 MHz, DMSO-d6) δ ppm: 1.59 (s, 6 H) 5.29 (s, 1 H) 7.52 (dd, J=7.8, 4.9 Hz, 1 H) 8.52 (dt, J=7.8, 2.0 Hz, 1 H) 8.58 (dd, J=4.9, 2.0 Hz, 1 H) 8.76 (d, J=2.4 Hz, 1 H) 8.82 (d, J=2.4 Hz, 1 H) 8.96 (d, J=0.8 Hz, 1 H) 8.99 (d, J=0.8 Hz, 1 H) 9.38 (d, J=2.0 Hz, 1 H) 12.15 (broad s, 1 H)

LC-MS-DAD-ELSD: 303(−)=(M−H); 305(+)=(M+H)(+) (Rt32 2.15 min)

### Example 17

### Synthesis of [6-(pyrid-3-yl)-9H-pyrrolo[2,3-b:5,4-c′]dipyrid-3-yl]methanol 14

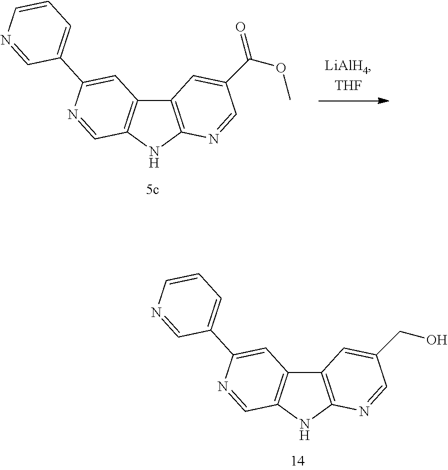

To a mixture of 120 mg of methyl 6-pyrid-3-yl-9H-pyrrolo[2,3-b:5,4-c′]dipyridine-3-carboxylate (product described in step 2 of Example 30) in 3.5 mL, of THF, under argon, is added over 5 minutes 0.6 ml of 1M lithium aluminium hydride in THF. After stirring for 2 hours, 0.2 mL of methanol is added to the reaction medium, and the medium is then poured into a mixture of 100 mL of ethyl acetate and 100 mL of aqueous 1M potassium sodium tartrate solution, and stirred vigorously for 1 hour. The organic phase is extracted, dried over magnesium sulfate, filtered and concentrated under reduced pressure. The crude residue is purified by chromatography on silica gel (15 g of silica) using a gradient of 0 to 10% methanol in ethyl acetate. The fractions containing the expected product are combined and concentrated under reduced pressure. The crude product obtained is then purified by preparative HPLC to give 31.9 mg (30%) of [6-(pyrid-3-yl)-9H-pyrrolo[2,3-b:5,4-c′]dipyrid-3-yl]-methanol 14.

1H NMR (400 MHz, DMSO-d6) δ ppm: 4.71 (d, J=5.6 Hz, 2 H) 5.36 (t, J=5.6 Hz, 1 H) 7.53 (broad dd, J=8.0, 4.8 Hz, 1 H) 8.52 (dt, J=8.0, 2.0 Hz, 1 H) 8.56-8.60 (m, 2 H) 8.68 (d, J=2.0 Hz, 1 H) 8.93 (d, J=1.0 Hz, 1 H) 9.01 (d, J=1.0 Hz, 1 H) 9.37 (broad d, =2.0 Hz, 1 H) 12.20 (broad s, 1 H)

UPLC-MS-DAD-ELSD: 275(−)=(M−H)(−); 277(+)=(M+H)(+) (Rt32 0.30 min)

### Example 18

### Synthesis of 2-methylpropyl 6-(pyrid-3-yl)-9H-pyrrolo[2,3-b:5,4-c′]dipyridine-3-carboxylate 15

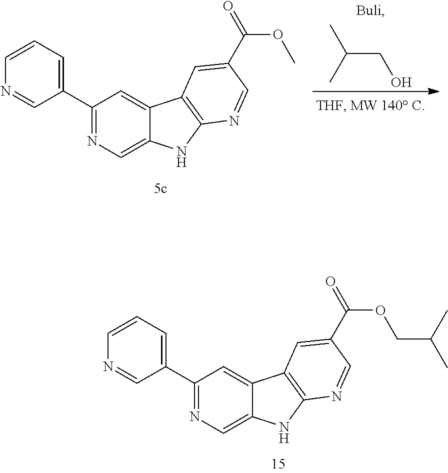

2.5 mmol of 2-methyl-1-propanol and 1 mL of THF are placed in a microwave tube, the tube is cooled using an ice bath, and 2.5 mmol of n-butyllithium are then added 0.5 mmol of the ethyl ester 5c are added and the tube is sealed. The reaction medium is irradiated by microwave for 30 minutes at 140° C., and 2 mL of ethyl acetate are then added. After stirring for 5 minutes at 25° C., 2 ml of saturated aqueous potassium dihydrogen phosphate solution are added and the precipitate obtained is filtered off by suction, washed with tetrahydrofuran and dried to give 124 mg (72%) of 2-methylpropyl 6-(pyrid-3-yl)-9H-pyrrolo[2,3-b:5,4-c′]dipyridine-3-carboxylate 15.

1H NMR (400 MHz, DMSO-d6) δ ppm 1.05 (d, J=6.6 Hz, 6 H) 2.11 (m, 1 H) 4.17 (d, J=6.6 Hz, 2 H) 7.54 (dd, J=7.8, 4.8 Hz, 1 H) 8.54 (dt, J=7.8, 1.7 Hz, 1 H) 8.60 (dd, J=4.8, 1.7 Hz, 1 H) 9.07 (s, 1 H) 9.12 (s, 1 H) 9.17 (d, J=2.1 Hz, 1 H) 9.31 (d, J=2.1 Hz, 1 H) 9.40 (d, J=1.7 Hz, 1 H) 12.75 (s, 1 H)

### Example 19

### Synthesis of 6-(pyrid-3-yl)-9H-pyrrolo[2,3-b:5,4-c′]dipyridine-3-carboxylic acid 16

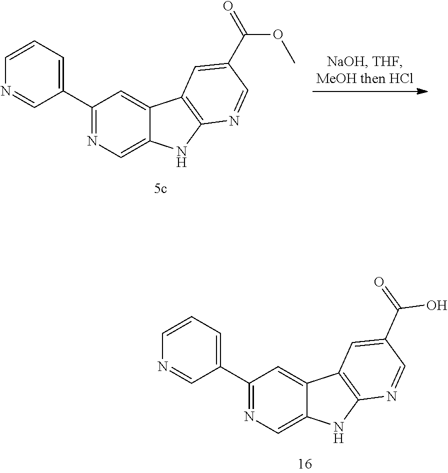

0.66 mmol of the methyl ester 5c in 1 mL of methanol and 1 mL of tetrahydrofuran are placed in a microwave tube, 2 mL of aqueous 1N sodium hydroxide solution are added and the tube is sealed. The reaction medium is irradiated by microwave for 30 minutes at 140° C., followed by addition of 2 ml of aqueous 1N hydrochloric acid solution. The precipitate obtained is filtered off and dried to give 6-(pyrid-3-yl)-9H-pyrrolo[2,3-b:5,4-c′]dipyridine-3-carb oxylic acid 16 quantitatively.

1H NMR (400 MHz, DMSO-d6) δ ppm: 7.53 (dd, J=7.9, 4.9 Hz, 1 H) 8.53 (dt, J=7.9, 2.0 Hz, 1 H) 8.60 (dd, J=4.9, 2.0 Hz, 1 H) 9.05 (d, J=1.0 Hz, 1 H) 9.08 (d, J=1.0 Hz, 1 H) 9.14 (d, J=2.0 Hz, 1 H) 9.27 (d, J=2.0 Hz, 1 H) 9.39 (d, J=2.0 Hz, 1 H) 11.5 (very broad m, 1 H)

LC-MS-DAD-ELSD: 289(−)=(M−H)(−); 291(+)=(M+H)(+) (Rt32 1.91 min)

### Example 20

### Synthesis of 3-fluoro-4-iodo-6-(pyrid-3-yl)-9H-pyrrolo[2,3-b:5,4-c′]dipyridine 19

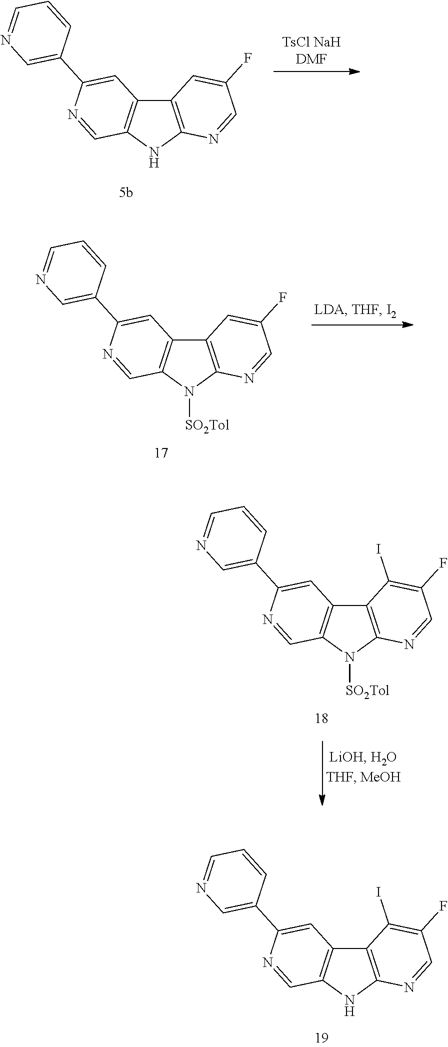

### Step 1:

3.2 g of 5b and 90 mL of dimethylformamide are placed in a 250 mL three-necked flask. The mixture is stirred, followed by addition, under argon, of 847 mg of sodium hydride. After two hours, 4.61 g of tosyl chloride in 10 mL of dimethylformamide are added. After stirring for 2 hours at room temperature, 250 mL of a 10% sodium bicarbonate solution and 250 mL of water are added and the mixture is extracted twice with 300 mL of ethyl acetate, dried over magnesium sulfate and filtered. The filtrate is purified by chromatography on silica gel (gradient: 100/0 to 95/5 dichloromethane/methanol). 4.75 g (94%) of the intermediate 3-fluoro-6-pyrid-3-yl-9-(toluene-4-sulfonyl)-9H-pyrrolo[2,3-b:5,4-c′]dipyridine 17 are obtained.

UPLC-MS-DAD-ELSD: 419(+)=(M+H)(+) (Rt32 1.19 min)

### Step 2:

0.73 mL of diisopropylamine is placed in 20 mL of THF in a dry round-bottomed flask under an argon atmosphere. The solution is cooled to −78° C., followed by addition of 1.94 mL of n-butyllithium (2.5 M in hexane). The resulting mixture is stirred for 15 minutes, followed by dropwise addition of 1.35 g of 17 predissolved in 80 mL of tetrahydrofuran. After stirring for 2 hours at −78° C., 1.31 g of diiodine predissolved in 5 mL of tetrahydrofuran are added dropwise. The mixture is stirred for 10 minutes. The reaction medium is poured into 250 mL of ammonium chloride solution and the resulting mixture is extracted with 500 mL of ethyl acetate. The organic phase is washed with 200 mL of aqueous sodium thiosulfate solution and then dried over magnesium sulfate, filtered and concentrated to dryness under reduced pressure. 1.65 g (91%) of the expected compound 3-fluoro-4-iodo-6-pyrid-3-yl-9-(toluene-4-sulfonyl)-9H-pyrrolo[2,3-b:5,4-c′]dipyridine 18 are thus obtained, and the product is used in the subsequent steps without further purification.

UPLC-MS-DAD-ELSD: 545(+)=(M+H)(+) (Rt32 1.32 min)

### Step 3:

700 mg of 18, 15 mL of methanol and 35 mL of tetrahydrofuran are placed in a round-bottomed flask. Aqueous lithium hydroxide solution (420 mg of LiOH.H₂O dissolved in 25 mL of water) is added. The mixture is stirred for 2 hours. 50 mL of water are added and the reaction medium is then neutralized with 5 mL of aqueous 2M hydrochloric acid solution. The precipitate is filtered off and then dried. 380 mg of 3-fluoro-4-iodo-6-(pyrid-3-yl)-9H-pyrrolo[2,3-b:5,4-c′]dipyridine 19 are thus obtained.

1H NMR (400 MHz, DMSO-d6) δ ppm 7.56 (dd, J=8.0, 4.5 Hz, 1 H) 8.43 (dt, J=8.0, 2.0 Hz, 1 H) 8.56 (s, 1 H) 8.62 (dd, J=4.5, 2.0 Hz, 1 H) 9.10 (d, J=8.0 Hz, 2 H) 9.27 (d, J=2.0 Hz, 1 H) 12.6 (broad m, 1 H)

LC-MS-DAD-ELSD: 391(+)=(M+H)(+)

### Examples 21 to 31 (21a-21k)

### General Procedure for the Suzuki Coupling in Position 4

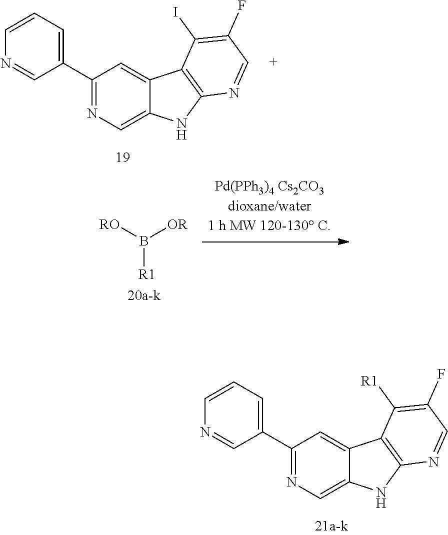

100 mg (0.25 mmol) of 3-fluoro-4-iodo-6-(pyrid-3-yl)-9H-pyrrolo[2,3-b:5,4-c′]dipyridine 19, 0.75 mmol of a boronate 20a-k, 26.6 mg of tetrakis(triphenylphosphine)palladium(0), 125 mg of caesium carbonate, 2 mL of dioxane and 0.5 mL of water are introduced into a microwave reactor of suitable size. The mixture is irradiated for 1 hour between 120 and 130° C. The suspension obtained is taken up in water and ethyl acetate, and the solid is suction-filtered through a sinter funnel (0.45 μm), washed with ethyl acetate and dried to obtain the expected compound 21a-k.

When the purity of the solid is less than 90%, or in the cases where crystallization does not take place, the crude reaction product is purified by preparative HPLC (VP240/50 mm Nucleodur 100-10 C18ec column) using a gradient of acetonitrile in water (Milli-Q+0.07% TFA). The fractions containing the expected product are combined and concentrated under reduced pressure to give the expected compound 21a-k. The results of these experiments are collated in Table 3.

| Reagent 20 | Structure 21 | Name | Yield | Analysis |
|---|---|---|---|---|
| 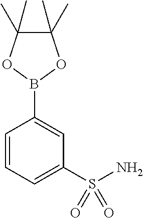 | 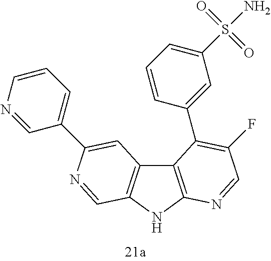 | 3-[3-Fluoro-6-(pyrid-3- yl)-9H-pyrrolo[2,3-b:5,4- c′]dipyrid-4-yl]- benzenesulfonamide | 99% | 1H NMR (400 MHz, DMSO-d6) δ ppm 7.44 (dd, J = 8.1, 4.9 Hz, 1 H) 7.56 (broad m, 2 H) 7.68 (d, J = 1.2 Hz, 1 H) 7.95 (t, J = 7.8 Hz, 1 H) 8.03 (dd, J = 7.8, 1.5 Hz, 1 H) 8.14 (dt, J = 7.8, 1.5 Hz, 1 H) 8.19 (dt, J = 8.1, 1.8 Hz, 1 H) 8.24 (t, J = 1.5 Hz, 1 H) 8.53 (dd, J = 4.9, 1.8 Hz, 1 H) 8.80 (d, J = 2.4 Hz, 1 H) 9.02 (d, J = 1.8 Hz, 1 H) 9.10 (d, J = 1.2 Hz, 1 H) 12.67 (broad m, 1 H) |
| 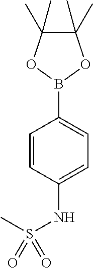 | 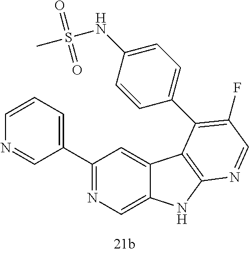 | N-[4-(3-Fluoro-6-(pyrid- 3-yl)-9H-pyrrolo[2,3- b:5,4-c′]dipyrid-4-yl)- phenyl]methanesulfon- amide | 50% | 1H NMR (400 MHz, DMSO-d6) δ ppm: 3.11 (s, 3 H) 7.46 (dd, J = 8.0, 4.8 Hz, 1 H) 7.49 (d, J = 8.6 Hz, 2H) 7.71 (d, J = 8.6 Hz, 2 H) 7.81 (s, 1 H) 8.16 (dt, J = 8.0, 2.0 Hz, 1 H) 8.55 (dd, J = 4.8, 2.0 Hz, 1 H) 8.72 (d, J = 2.7 Hz, 1 H) 8.99 (d, J = 2.0 Hz, 1 H) 9.06 (s, 1 H) 10.24 (very broad m, 1 H) 12.55 (broad m, 1 H) |
| 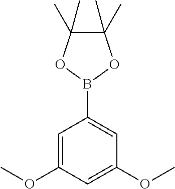 | 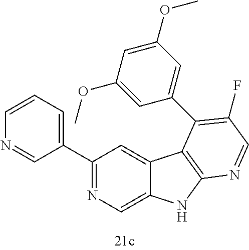 | 4-(3,5-dimethoxy- phenyl)-3-fluoro-6- (pyrid-3-yl)-9H-pyrrolo- [2,3-b:5,4-c′]dipyridine | 82% | 1H NMR (400 MHz, DMSO-d6) δ ppm: 3.83 (s, 6 H) 6.83 (t, J = 2.0 Hz, 1 H) 6.89 (d, J = 2.0 Hz, 2 H) 7.48 (dd, J = 8.1, 4.8 Hz, 1 H) 7.83 (d, J = 0.9 Hz, 1 H) 8.15 (dt, J = 8.1, 1.9 Hz, 1 H) 8.54 (dd, J = 4.8, 1.9 Hz, 1 H) 8.72 (d, J = 2.4 Hz, 1 H) 8.94 (d, J = 1.9 Hz, 1 H) 9.06 (d, J = 0.9 Hz, 1 H) 12.53 (broad m, 1 H) |
| 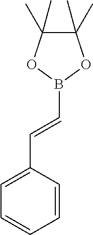 | 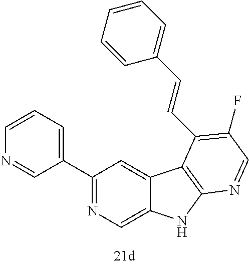 | 3-fluoro-4-[(E)-2- phenylethenyl]-6-(pyrid- 3-yl)-9H-pyrrolo[2,3- b:5,4-c′]dipyridine | 75% | 1H NMR (400 MHz, DMSO-d6) δ ppm: 7.42 (t, J = 7.5 Hz, 1 H) 7.51 (m, 3 H) 7.68 (d, J = 16.6 Hz, 1 H) 7.93 (d, J = 7.5 Hz, 2 H) 7.97 (d, J = 16.6 Hz, 1H) 8.45 (dt, J = 7.9, 2.0 Hz, 1 H) 8.58 (dd, J = 4.7, 2.0 Hz, 1H) 8.64 (d, J = 1.0 Hz, 1 H) 8.67 (d, J = 3.6 Hz, 1 H) 9.07 (s, 1 H) 9.29 (d, J = 2.0 Hz, 1 H) 12.50 (broad m, 1 H) |
| 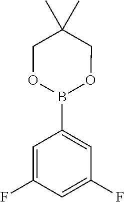 | 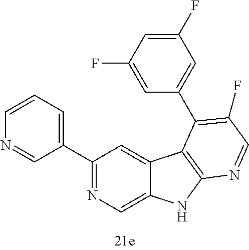 | 4-(3,5-difluorophenyl)-3- fluoro-6-(pyrid-3-yl)-9H- pyrrolo [2,3-b:5,4-c′]- dipyridine | 80% | 1H NMR (400 MHz, DMSO-d6) δ ppm: 7.49 (dd, J = 8.1, 4.9 Hz, 1 H) 7.56-7.66 (m, 3 H) 7.74 (s, 1 H) 8.18 (dt, J = 8.0, 2.0 Hz, 1 H) 8.55 (dd, J = 4.9, 2.0 Hz, 1 H) 8.78 (d, J = 2.4 Hz, 1 H) 8.98 (broad d, J = 2.0 Hz, 1 H) 9.09 (d, J = 1.0 Hz, 1 H) 12.67 (broad m, 1 H) |
| 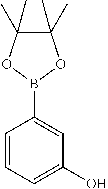 | 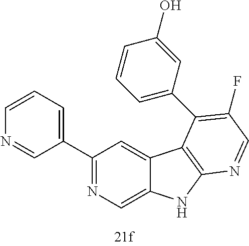 | 3-(3-fluoro-6-(pyrid-3- yl)-9H-pyrrolo[2,3-b:5,4- c′)(dipyrid-4-yl]phenol | 82% | 1H NMR (400 MHz, DMSO-d6) δ ppm: 7.00-7.19 (m, 3 H) 7.48 (dd, J = 7.9, 4.9 Hz, 1 H) 7.52 (t, J = 7.9 Hz, 1 H) 7.82 (s, 1 H) 8.17 (dt, J = 7.9, 2.0 Hz, 1 H) 8.54 (dd, J = 4.9, 2.0 Hz, 1H) 8.72 (d, J = 2.4 Hz, 1 H) 8.96 (d, J = 2.0 Hz, 1 H) 9.06 (s, 1 H) 9.92 (broad m, 1 H) 12.56 (broad m, 1 H) |
| 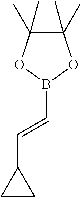 | 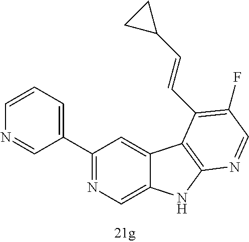 | 4-[(E)-2-cyclopropyl- ethenyl]-3-fluoro-6- (pyrid-3-yl)-9H-pyrrolo- [2,3-b:5,4-c′]dipyridine | 60% | 1H NMR (400 MHz, DMSO-d6) δ ppm: 0.73 (m, 2 H) 1.01 (m, 2 H) 2.15 (m, 1 H) 6.38 (dd, J = 15.8, 9.5 Hz, 1 H) 7.39 (d, J = 15.8 Hz, 1 H) 7.53 (dd, J = 8.1, 4.7 Hz, 1 H) 8.52-8.56 (m, 2 H) 8.60 (dd, J = 4.7, 2.0 Hz, 1 H) 8.71 (s, 1 H) 9.04 (s, 1 H) 9.39 (d, J = 2.0 Hz, 1 H) 12.39 (broad m, 1 H) |
| 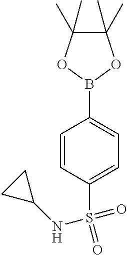 | 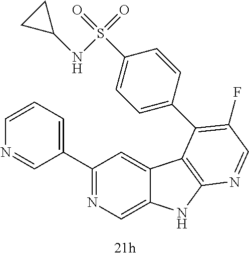 | N-cyclopropyl-4-(3- fluoro-6-(pyrid-3-yl)-9H- pyrrolo[2,3-b:5,4-c′]- dipyrid-4-yl]benzene- sulfonamide | 47% | 1H NMR (400 MHz, DMSO-d6) δ ppm: 0.47 (m, 2 H) 0.57 (m, 2 H) 2.24 (m, 1 H) 7.53 (dd, J = 8.1, 4.9 Hz, 1 H) 7.65 (s, 1 H) 8.03 (d, J = 8.5 Hz, 2 H) 8.13 (masked m, 1 H) 8.14 (d, J = 8.5 Hz, 2 H) 8.22 (broad d, J = 8.1 Hz, 1 H) 8.59 (dd, J = 4.9, 2.0 Hz, 1 H) 8.81 (d, J = 2.4 Hz, 1 H) 8.97 (broad d, J = 2.0 Hz, 1H) 9.11 (d, J = 1.0 Hz, 1 H) 12.71 (s, 1 H) |
| 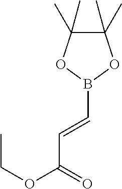 | 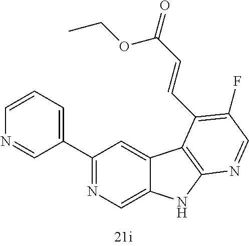 | ethyl (2E)-3-[3-fluoro-6- (pyrid-3-yl)-9H-pyrrolo- [2,3-b:5,4-c′]dipyrid-4- yl]prop-2-enoate | 48% | 1H NMR (400 MHz, DMSO-d6) δ ppm: 1.37 (t, J = 7.1 Hz, 3 H) 4.33 (q, 7 = 7.1 Hz, 2 H) 6.95 (d, J = 16.2 Hz, 1 H) 7.63 (dd, J = 7.7, 5.1 Hz, 1 H) 8.36 (d, J = 16.2 Hz, 1 H) 8.53 (broad d, J = 7.7 Hz, 1 H) 8.55 (broad s, 1 H) 8.66 (broad d, J = 5.1 Hz, 1 H) 8.77 (d, J = 3.3 Hz, 1H) 9.13 (d, J = 1.2 Hz, 1 H) 9.32 (broad s, 1H) 12.72 (s, 1 H) |
| 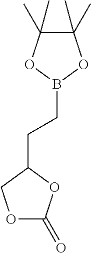 | 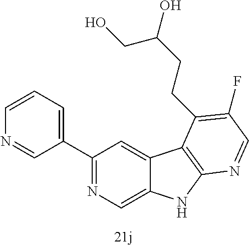 | 4-(3-fluoro-6-(pyrid-3- yl)-9H-pyrrolo[2,3-b:5,4- c′]dipyrid-4-yl]butane- 1,2-diol | 54% | 1H NMR (400 MHz, DMSO-d6) δ ppm: 1.74 (m, 1 H) 1.99 (m, 1 H) 3.23- 3.39 (masked m, 2 H) 3.45 (m, 2H) 3.67 (m, 1 H) 4.63 (broad s, 1 H) 5.11 (broad s, 1 H) 7.51 (dd, J = 8.1, 4.8 Hz, 1 H) 8.50-8.60 (m, 3 H) 8.88 (s, 1 H) 9.05 (s, 1 H) 9.39 (broad d, J = 2.0 Hz, 1 H) 12.42 (broad m, 1 H) |
| 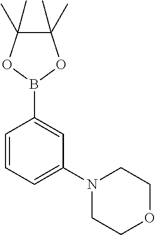 | 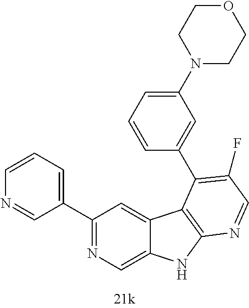 | 3-fluoro-4-[3-(morpholin- 4-yl)phenyl]-6-(pyrid-3- yl)-9H-pyrrolo [2,3-b:5,4- c′]dipyridine | 55% | 1H NMR (400 MHz, DMSO-d6) δ ppm: for this batch, all the signals are broad, with: 3.21 (m, 4 H) 3.73 (m, 4 H) 7.15 (d, J = 8.1 Hz, 1 H) 7.27 (d, J = 8.1 Hz, 1 H) 7.32 (s, 1 H) 7.49 (dd, J = 7.8, 4,8 Hz, 1 H) 7.57 (t, J = 8.1 Hz, 1 H) 7.83 (s, 1 H) 8.17 (d, J = 7.8 Hz, 1 H) 8.56 (d, J = 4.8 Hz, 1H) 8.74 (s, 1 H) 8.96 (s, 1 H) 9.06 (s, 1 H) 12.59 (broad m, 1 H) |

### Example 32

### Synthesis of 4-cyclopropyl-fluoro-6-(pyrid-3-yl)-9H-pyrrolo[2,3-b:5,4-c′]dipyridine 22

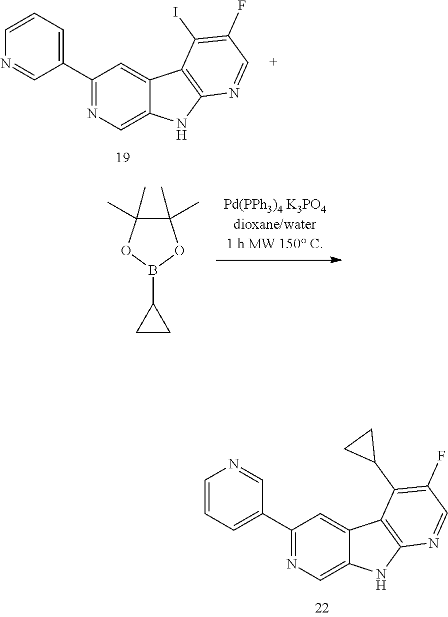

100 mg of 19, 129.2 mg of (4,4,5,5-tetramethyl-1,3,2-dioxaborolan)cyclopropane, 26.6 mg of tetrakis(triphenylphosphine)palladium(0), 81.6 mg of potassium phosphate, 2 mL of dioxane and 0.500 mL of water are introduced into a microwave reactor of suitable size. The mixture is irradiated for 1 hour at 150° C. The suspension obtained is taken up in water and ethyl acetate, and the solid is filtered off by suction through a sinter funnel (0.45 μm), washed with ethyl acetate and dried. 68 mg (87%) of pale yellow solid 4-cyclopropyl-3-fluoro-6-(pyrid-3-yl)-9H-pyrrolo[2,3-b:5,4-c′]dipyridine 22 are obtained.

1H NMR (400 MHz, DMSO-d6) δ ppm: 1.13 (m, 2 H) 1.40 (m, 2 H) 2.70 (m, 1 H) 7.73 (dd, J=7.8, 5.2 Hz, 1 H) 8.51 (d, J=4.0 Hz, 1 H) 8.70 (broad d, J=5.2 Hz, 1 H) 8.78 (broad d, J=7.8 Hz, 1 H) 8.88 (s, 1 H) 9.08 (d, J=1.0 Hz, 1 H) 9.46 (d, J=1.7 Hz, 1 H) 12.46 (broad s, 1 H)

### Example 33

### Synthesis of 3-fluoro-4-(morpholin-4-yl)-6-(pyrid-3-yl)-9H-pyrrolo[2,3-b:5,4-c′]-dipyridine; trifluoroacetic acid salt 23

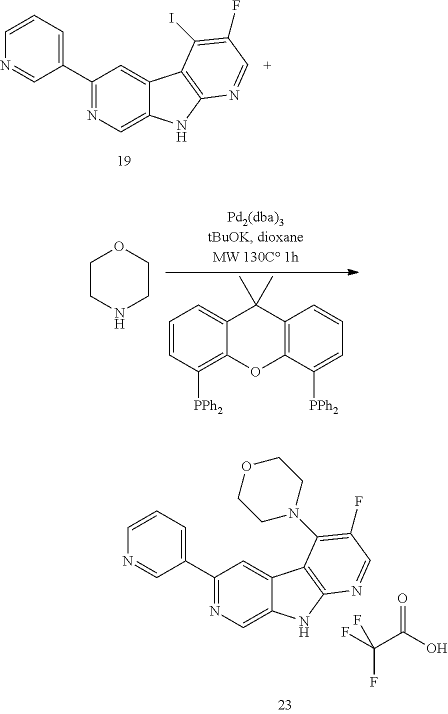

To a mixture of 100 mg of 19 and 28 mg of potassium tert-butoxide in 1 mL of dioxane is added a solution, stirred beforehand under argon for 15 minutes, of 37 mg of 9,9-dimethyl-4,5-bis(diphenylphosphino)xanthene, 23.4 mg of tris(dibenzylideneacetone)dipalladium and 1 ml of anhydrous dioxane. 1 mL of dioxane is added to rinse the glassware containing the Xantphos/Pd(OAc)₂. 120 μL of morpholine are then added. The mixture obtained is then irradiated in a microwave oven for 1 hour at 130° C. The reaction medium is concentrated under reduced pressure and then purified by preparative HPLC (VP240/50 mm NUCLEODUR 100-10 C18ec column) using a gradient of acetonitrile in water (MilliQ water supplemented with 0.07% trifluoroacetic acid). The fractions containing the expected product are combined and concentrated under reduced pressure to give 36 mg (30%) of yellow solid 3-fluoro-4-(morpholin-4-yl)-6-(pyrid-3-yl)-9H-pyrrolo[2,3-b:5,4-c′]dipyridine in the form of the trifluoroacetic acid salt.

1H NMR (400 MHz, DMSO-d6) δ ppm: 3.56 (m, 4 H) 3.96 (m, 4 H) 7.76 (broad m, 1 H) 8.37 (s, 1 H) 8.50 (d, J=6.0 Hz, 1 H) 8.66-8.74 (m, 2 H) 9.03 (s, 1 H) 9.38 (s, 1 H) 12.42 (broad s, 1 H)

### Synthesis of 3-fluoro-6-pyrid-3-yl-9-(toluene-4-sulfonyl)-4-trimethylstannyl-9H-pyrrolo[2,3-b:5,4-c′]-dipyridine 24

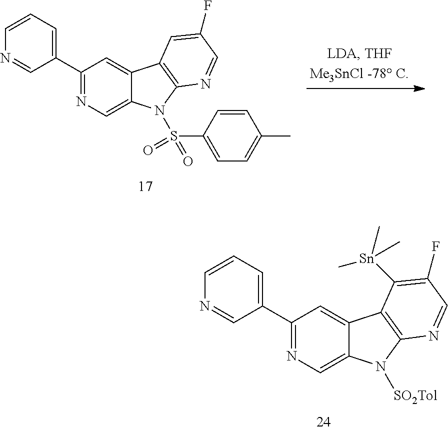

2.14 mL of diisopropylamine in 60 mL of THF are placed in a dry one-necked flask under argon. The mixture is stirred and cooled to −78° C., followed by addition of 5.73 mL of n-butyllithium. After stirring for 15 minutes, 4 g of compound 17 predissolved in 240 mL of THF are added. The reaction medium is stirred for 2 hours, followed by addition of 3.04 g of chloro(trimethyl)stannane in 20 mL of THF. After cooling to 25° C., the reaction medium is hydrolysed with 250 ml of aqueous 10% ammonium chloride solution and 250 mL of water, and the aqueous phase is then extracted twice with 300 mL of ethyl acetate. The combined organic phases are washed with saturated aqueous KF solution, dried over magnesium sulfate, filtered and concentrated under reduced pressure. The crude residue obtained is purified by chromatography on silica gel (600 g of silica) using a gradient of 1% to 6% methanol in dichloromethane. The fractions containing the expected product are combined and concentrated under reduced pressure. 2.79 g of product are obtained, and are again purified by chromatography on silica gel (200 g of silica) using a gradient of 0% to 6% methanol in dichloromethane, to give 1.3 g (22%) of 3-fluoro-6-pyrid-3-yl-9-(toluene-4-sulfonyl)-4-trimethyl stannyl-9H-pyrrolo[2,3-b:5,4-c′]dipyridine 24.

UPLC-MS-DAD-ELSD: 583(+)=(M+H)(+) (isotope profile corresponding to a tin derivative) Rt (min)=1.43

### Examples 34 to 36 (27a-27c)

### General Procedure for the Synthesis of Ketone and Amine Via the Derivative with Trimethylstannyl at 4 24

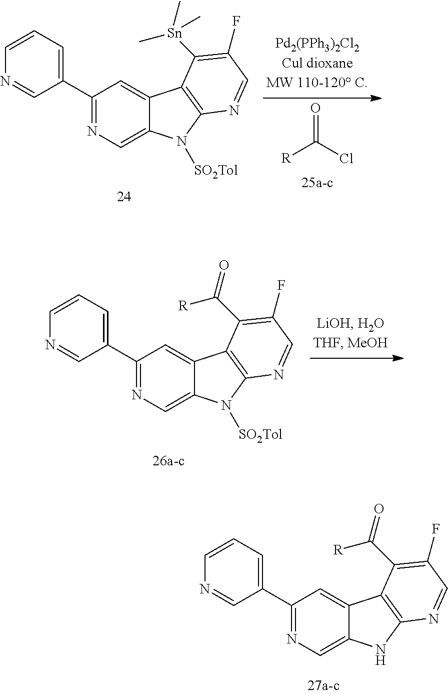

0.2 mmol of 3-fluoro-6-pyrid-3-yl-9-(toluene-4-sulfonyl)-4-trimethylstannyl-9H-pyrrolo[2,3-b:5,4-c′]-dipyridine 24, 0.03 mmol of dichlorobis(triphenylphosphine)palladium(II), 3 mL of toluene, 0.2 mmol of copper iodide and 0.5 mmol of chloride 25a-c are introduced into a microwave reactor of suitable size. The mixture is irradiated for 1 hour between 110 and 120° C. The reaction medium is hydrolysed with 25 mL of water, and the aqueous phase is then extracted twice with 50 mL of ethyl acetate. The combined organic phases are dried over magnesium sulfate, filtered and concentrated under reduced pressure. The crude residue obtained is purified by chromatography on silica gel (25 g of silica) using a gradient of 0% to 6% methanol in dichloromethane. The tosyl intermediates 26a-c are thus obtained. Product 26a-c is taken up in 4 mL of a methanol/tetrahydrofuran mixture (1/1 by volume), followed by addition of aqueous lithium hydroxide solution. After stirring for 2 hours, the reaction medium is neutralized with aqueous ammonium chloride solution and extracted with twice 40 mL of ethyl acetate. The combined organic phases are dried over magnesium sulfate, filtered and concentrated under reduced pressure. The residues are purified by chromatography on silica gel (25 g of silica, elution gradient: 98/2 to 95/5 dichloromethane/methanol). The products 27a-c obtained are described in Table 4.

TABLE 4

| Reagent 25 | Structure 21 | Name | Yield | Analysis |
|---|---|---|---|---|
| 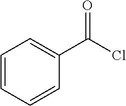 | 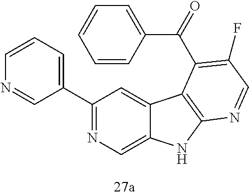 | [3-fluoro-6-(pyrid-3-yl)- 9H-pyrrolo[2,3-b:5,4-c′]- dipyrid-4-yl](phenyl)- methanone | 18% | 1H NMR (400 MHz, DMSO-d6) δ ppm: 7.46 (ddd, J = 8.1,4.8, 0.7 Hz. 1 H) 7.63 (t, J = 7.7 Hz, 2 H) 7.67 (d, J = 1.2 Hz, 1 H) 7.81 (tt, J = 7.7, 1.2 Hz, 1 H) 8.03 (dd, J = 7.7, 1.2 Hz, 2 H) 8.12 (dt, J = 8.1, 2.0 Hz, 1 H) 8.54 (dd, J = 4.8, 2.0 Hz, 1 H) 8.87 (d, J = 2.2 Hz, 1 H) 8.92 (dd, J = 2.0, 0.7 Hz, 1 H) 9.11 (d, J = 1.2 Hz, 1 H) 12.77 (broad m, 1 H) UPLC-MS-DAD- ELSD: 367(−) = (M − H)(−); 369 (+) = (M + H)(+)(Rt = 0.67 min) |
| 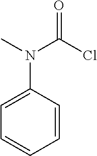 | 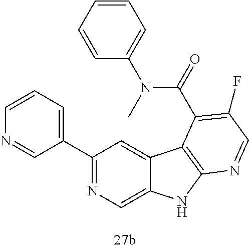 | 3-fluoro-N-methyl-N- phenyl-6-(pyrid-3-yl)-9H- pyrrolo[2,3-b:5,4-c′]- dipyridine-4-carboxamide | 12% | 1H NMR (400 MHz, DMSO-d6) δ ppm: 3.64 (s, 3 H) 7.07-7.22 (m, 5 H) 7.57 (dd, J = 7.8, 4.8 Hz, 1 H) 8.37 (d, J = 1.2 Hz, 1 H) 8.42-8.49 (m, 2 H) 8.63 (dd, J = 4.8, 2.0 Hz, 1 H) 9.09 (d, J = 1.2 Hz, 1 H) 9.31 (d, J = 2.0 Hz, 1 H) 12.56 (m, 1 H) UPLC-MS-DAD-ELSD: 396(−) = (M − H)(−); 398(+) = (M + H)(+) (Rt = 0.60 min) |
| 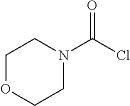 | 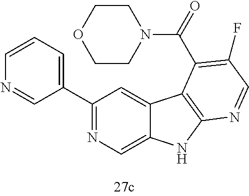 | [3-fluoro-6-(pyrid-3-yl)- 9H-pyrrolo[2,3-b:5,4-c′]- dipyrid-4-yl](morpholin-4- yl)methanone | 13% | 1H NMR (400 MHz, DMSO-d6) δ ppm: 3.22- 3.47 (partially masked m, 4 H) 3.77-4.07 (m, 4 H) 7.55 (dd, J = 8.0, 4.6 Hz, 1 H) 8.19 (d, J = 0.8 Hz, 1 H) 8.38 (dt, J = 8.0, 2.0 Hz, 1 H) 8.62 (dd, J = 4.6, 2.0 Hz, 1 H) 8.77 (d, J = 1.9 Hz, 1 H) 9.12 (d, J = 0.8 Hz, 1 H) 9.23 (d, J = 2.0 Hz, 1 H) 12.70 (broads, 1 H) UPLC-MS-DAD-ELSD: 376(−) = (M + H)(−); 378(+) = (M + H)(+) (Rt = 0.4 min) |

### Example 37

### Synthesis of 4-chloro-3-fluoro-6-(pyrid-3-yl)-9H-pyrrolo[2,3-b:5,4-c′]dipyridine 29

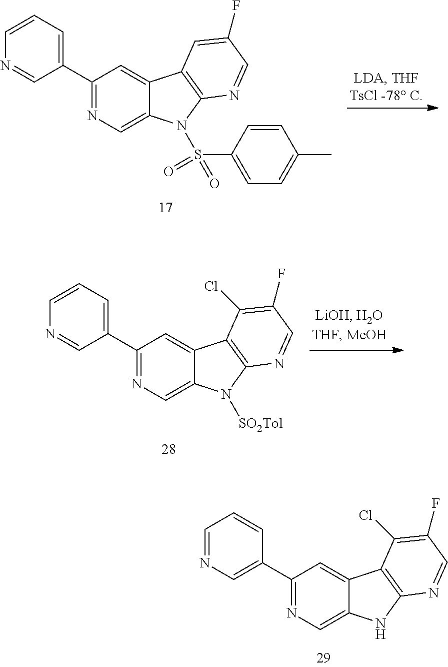

### Step 1

105 μl of diisopropylamine in 3 mL of THF are placed in a dry one-necked flask under argon. The mixture is stirred and cooled to −78° C., followed by addition of 280 μl of n-butyllitium. After stirring for 15 minutes, 210 mg of 3-fluoro-6-pyrid-3-yl-9-(toluene-4-sulfonyl)-9H-pyrrolo[2,3-b:5,4-c′]-dipyridine 17 in 4 mL of THF are added. The reaction medium is stirred for 2 hours, followed by addition of 191 mg of toluenesulfonyl chloride in 1 mL of THF. After stirring for 1 hour, the reaction medium is hydrolysed with 50 mL of aqueous 10% ammonium chloride solution and 50 mL of water, and the aqueous phase is then extracted twice with 50 mL of ethyl acetate. The combined organic phases are dried over magnesium sulfate, filtered and concentrated under reduced pressure. The crude residue obtained is purified by chromatography on silica gel (25 g of silica), using a gradient of 10% to 66% ethyl acetate in dichloromethane. The fractions containing the expected product are combined and concentrated under reduced pressure to give 81 mg (35%) of 3-fluoro-4-chloro-6-pyrid-3-yl-9-(toluene-4-sulfonyl)-9H-pyrrolo[2,3-b:5,4-c′]dipyridine 28.

LC-MS-DAD-ELSD: 453(+)=(M+H)(+) (isotope profile corresponding to a chloro derivative) Rt (min)=4.53

### Step 2

80 mg 3-fluoro-4-chloro-6-pyrid-3-yl-9-(toluene-4-sulfonyl)-9H-pyrrolo[2,3-b:5,4-c′]dipyridine, 2 mL of methanol, 2 ml of THF and 41 mg of lithium hydroxide dissolved in 0.500 mL of water are placed in a one-necked flask. The reaction mixture is stirred overnight at 25° C., then the methanol and the THF are evaporated off under reduced pressure. The crude residue obtained is taken up in 5 mL of water and 2 ml of aqueous 10% ammonium chloride solution, triturated, filtered and then purified by preparative HPLC (Macherey-Nagel 250×40 mm phase inverse C18 Nucleodur 10μ column). Elution is performed on a gradient (acetonitrile containing 0.07% TFA and H₂O containing 0.07% TFA). The fractions containing the expected compound are combined, the acetonitrile is evaporated off under reduced pressure, and the water is removed with a lyophilizer, to give 6 mg of 4-chloro-3-fluoro-6-(pyrid-3-yl)-9H-pyrrolo[2,3-b:5,4-c′]dipyridine 29.

1H NMR (400 MHz, DMSO-d6) δ ppm: 7.54 (dd, J=8.1, 4.8 Hz, 1 H) 8.52 (dt, J=8.1, 1.8 Hz, 1 H) 8.60 (dd, J=4.8, 1.8 Hz, 1 H) 8.79 (d, J=2.4 Hz, 1 H) 8.80 (d, J=1.0 Hz, 1 H) 9.13 (d, J=1.0 Hz, 1 H) 9.36 (d, J=1.8 Hz, 1 H) 12.23 (broad m, 1 H)

UPLC-MS-DAD-ELSD: 297/ . . . (−)=(M−H)/ . . . (−); 299(+)/ . . . =(M+H)/ . . . (+)(1 C1 present) (Rt32 2.74 min)

### Example 38

### Synthesis of 4-methyl-3-fluoro-6-(pyrid-3-yl)-9H-pyrrolo[2,3-b:5,4-c′]dipyridine 31

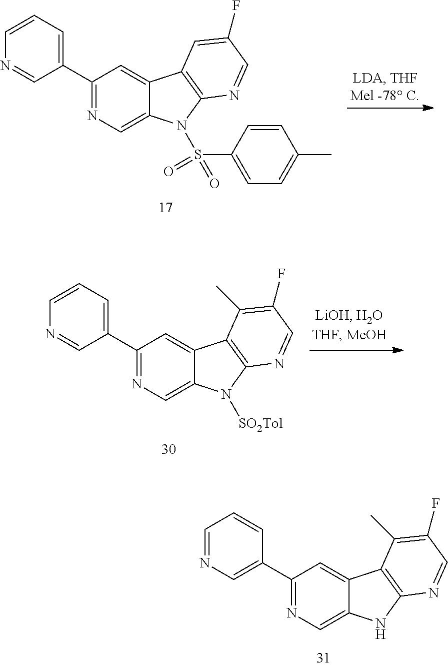

### Step 1

106 μl of diisopropylamine in 3 mL of THF are placed in a dry one-necked flask under argon. The mixture is stirred and cooled to −78° C., followed by addition of 280 μl of n-butyllitium. After stirring for 15 minutes, 210 mg of 3-fluoro-6-pyrid-3-yl-9-(toluene-4-sulfonyl)-9H-pyrrolo[2,3-b:5,4-c′]-dipyridine 17 in 4 mL of THF are added. The reaction medium is stirred for 2 hours, followed by addition of 142.5 mg of iodomethane in 1 mL of THF. After stirring for 1 hour, the reaction medium is hydrolysed with 50 mL of aqueous 10% ammonium chloride solution and 50 mL of water, and the aqueous phase is then extracted twice with 50 mL of ethyl acetate. The combined organic phases are dried over magnesium sulfate, filtered and concentrated under reduced pressure. The crude residue obtained is purified by chromatography on silica gel (25 g of silica, using a gradient of 10% to 66% ethyl acetate in dichloromethane. The fractions containing the expected product are combined and concentrated under reduced pressure. The 60 mg obtained are purified by semi-preparative HPLC (Kromasil C18 5 μm, 2×25 cm column), eluting at 18 ml/min with a mixture composed of 70% acetonitrile and 30% water. 20 mg (28%) of 3-fluoro-4-methyl-6-pyrid-3-yl-9-(toluene-4-sulfonyl)-9H-pyrrolo[2,3-b:5,4-c′]dipyridine 30 are obtained.

LC-MS-DAD-ELSD: 433(+)=(M+H)(+) Rt (min)=4.78

### Step 2

16 mg of 3-fluoro-4-methyl-6-pyrid-3-yl-9-(toluene-4-sulfonyl)-9H-pyrrolo[2,3-b:5,4-c′]dipyridine, 1 mL of methanol, 0.500 mL of THF and 16 mg of lithium hydroxide dissolved in 0.500 mL of water are placed in a one-necked flask. The reaction mixture is heated for 1 hour at 45° C., followed by dropwise addition of aqueous 10% ammonium chloride solution until a precipitate forms. After filtering off the precipitate by suction and washing three times with 5 mL of distilled water, 5 mg of 4-methyl-3-fluoro-6-(pyrid-3-yl)-9H-pyrrolo[2,3-b:5,4-c′]dipyridine are obtained, the characteristics of which are as follows:

1H NMR (400 MHz, DMSO-d6) δ ppm 2.91 (d, J=1.5 Hz, 3 H) 7.52 (dd, J=8.1, 4.6 Hz, 1 H) 8.53-8.61 (m, 3 H) 8.71 (s, 1 H) 9.05 (s, 1 H) 9.41 (d, J=2.2 Hz, 1 H) 12.23 (broad m, 1 H)

### Synthesis of 6-methoxy-9H-pyrrolo[2,3-b:5,4-c′]dipyridine 35

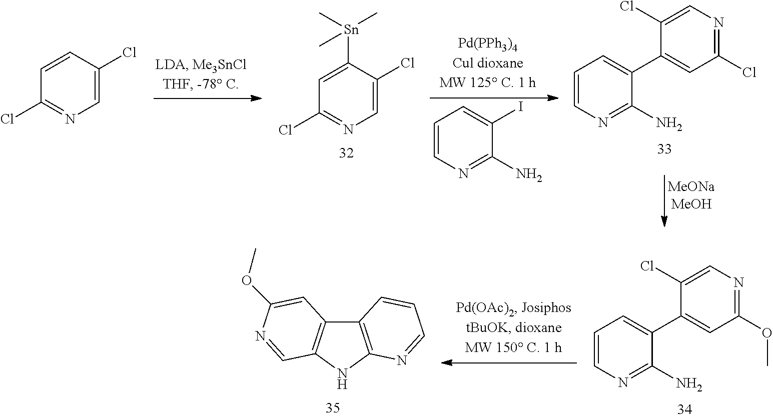

### Step 1

10.5 mL of diisopropylamine in 40 mL of THF are placed in a dry one-necked flask under argon. The mixture is stirred and cooled to −78° C., followed by addition of 29.73 mL of n-butyllitium dropwise over 45 minutes, and then addition of 10 g of 2,5-dichloropyridine dissolved in 170 mL of THF, over 20 minutes. The medium turns yellow and then brown. After stirring for 2 hours, 17.5 g of chloro(trimethyl)stannane dissolved in THF are added over 20 minutes at −78° C., and the mixture is then allowed to warm to −10° C. overnight. The reaction medium is hydrolysed with 1 litre of ammonium chloride solution and 300 mL of water, and the aqueous phase is then extracted with ethyl acetate. The organic phase is dried over magnesium sulfate, filtered and concentrated under reduced pressure. The crude residue obtained is purified by chromatography on silica gel using a gradient of 0 to 25% ethyl acetate in heptane. The fractions containing the expected product are combined and concentrated under reduced pressure to give 18 g of 2,5-dichloro-4-trimethylstannylpyridine 32 in the foam of a white solid.

LC-MS-DAD-ELSD: 309(+)=(M+H)(+) (isotope profile corresponding to a tin derivative) Rt (min)=5.09

### Step 2

220 mg of 2-amino-3-iodopyridine, 311 mg of 2,5-dichloro-4-trimethylstannylpyridine 32, 80.89 mg of tetrakis(triphenylphosphine)palladium(0), 40 mg of copper iodide and 3 mL of dioxane are introduced into a microwave reactor of suitable size. The reaction medium is irradiated for 1 hour at 125° C., and then hydrolysed with 75 ml of aqueous 10% sodium bicarbonate solution and 5 mL of water. The aqueous phase is extracted twice with 50 mL of ethyl acetate, and the combined organic phases are then dried over sodium sulfate, filtered and concentrated under reduced pressure. The crude residue obtained is purified by chromatography on silica gel (70 g of silica) using a gradient of 0% to 5% methanol in ethyl acetate. The fractions containing the expected product are combined and concentrated under reduced pressure to give 133 mg of 2′,5′-dichloro-[3,4′]bipyridyl-2-ylamine 33.

LC-MS-DAD-ELSD: 239.9(+)=(M+H)(+) (isotope profile corresponding to a dichloro derivative) Rt (min)=1.94

### Step 3

1 g of 2′,5′-dichloro-[3,4′]bipyridyl-2-ylamine, 10 mL of methanol and 202.5 mg of sodium methoxide are introduced into a microwave reactor of suitable size. The mixture is irradiated for three times 1 hour at 100° C., and the suspension obtained is then filtered and washed with dichloromethane. This product is purified by preparative HPLC (acidic eluent). The fractions containing the expected product are combined and concentrated under reduced pressure to give 2.3 g of 5′-chloro-2′-methoxy-[3,4′]bipyridyl-2-ylamine 34 in the form of a white solid.

### Step 4

To a yellow suspension of 100 mg of 5′-chloro-2′-methoxy-[3,4′]bipyridyl-2-ylamine ethyl ester, 66.61 mg of potassium tert-butoxide and 5 mL of dioxane under argon is added an orange-brown solution, stirred beforehand under argon for 10 minutes, of 25.86 mg of (R)-(−)-1-[(S)-2-(dicyclohexylphosphino)ferrocenyl]ethyldi-tert-butylphosphine, 9.52 mg of palladium(II) acetate and 1 mL of anhydrous dioxane. 1 mL of dioxane is added to rinse the glassware containing the Josiphos/Pd(OAc)₂ preparation. The reaction medium is then irradiated for 1 hour at 150° C. The insoluble matter of the suspension obtained is separated out by filtration and washed with dichloromethane, and the filtrate is then concentrated under reduced pressure. The crude residue is purified by preparative acidic HPLC (VP 240/50 mm Nucleodur 100-10 C18ec column) using a gradient of acetonitrile in MilliQ water containing 0.07% trifluoroacetic acid. The fractions containing the expected product are combined and concentrated under reduced pressure to give 70 mg of beige-coloured solid 6-methoxy-9H-pyrrolo[2,3-b:5,4-c′]dipyridine 35 in the form of the trifluoroacetic acid salt.

LC-MS-DAD-ELSD: 200(+)=(M+H)(+) Rt (min)=2.45

1H NMR (400 MHz, DMSO-d6) δ ppm: 3.93 (s, 3 H) 7.24 (dd, J=7.8, 4.9 Hz, 1 H) 7.63 (s, 1 H) 8.48 (s, 1 H) 8.56 (dd, J=4.9, 1.7 Hz, 1 H) 8.63 (dd, J=7.8, 1.7 Hz, 1 H) 11.84 (broad s, 1 H)

### Examples 39 to 41 (39a-39c)

### Preparation of the Triflate and Suzuki Coupling

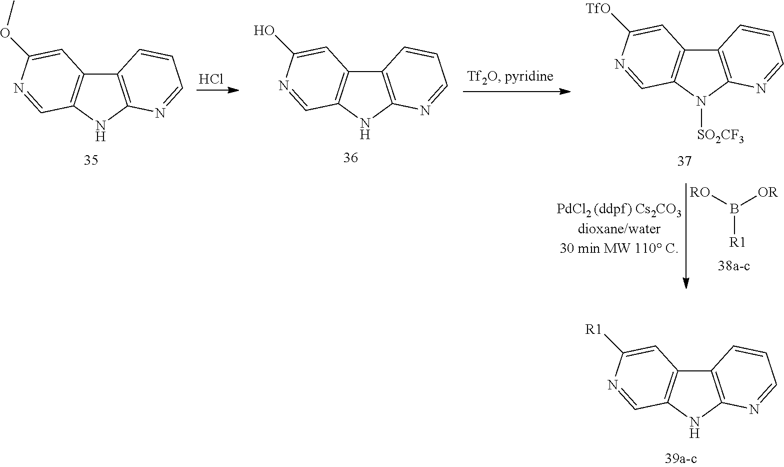

### Step 1

To a solution of 305 mg of 6-methoxy-9H-pyrrolo[2,3-b:5,4-c′]dipyridine 35 in 7.5 mL of acetic acid are added 1.5 mL of 37% hydrochloric acid solution. The mixture is heated by microwave for 3 hours at 150° C., and the insoluble matter formed is filtered off by suction and washed with diethyl ether to give 312 mg of 9H-dipyrido[2,3-b:4′,3′-d]pyrrol-6-ol 36.

UPLC-MS-DAD-ELSD: 186(+)=(M+H)(+) Rt (min)=0.32

### Step 2

To a suspension of 280 mg of 9H-dipyrido[2,3-b:4′,3′-d]pyrrol-6-ol 36 in 8 mL of pyridine is added 1 ml of trifluoromethanesulfonic anhydride, and then 0.5 ml after stirring for 1 hour at 25° C. The reaction medium is stirred overnight and then concentrated under reduced pressure. The residue is taken up in dichloromethane, and the organic phase obtained is washed with saturated aqueous sodium bicarbonate solution, and then concentrated under reduced pressure. The brown solid obtained is purified by chromatography on silica gel (30 g of silica) using a gradient of 0% to 100% ethyl acetate in heptane. The fractions containing the expected product are combined and concentrated under reduced pressure to give 305 mg of 9-[(trifluoromethyl)sulfonyl]-9H-pyrrolo[2,3-b:5,4-c′]dipyrid-6-yl trifluoromethanesulfonate, in the form of a beige-coloured solid 37.

UPLC-MS-DAD-ELSD: 450(+)=(M+H)(+) Rt (min)=1.39

### Step 3

0.1 mmol of 9-[(trifluoromethyl)sulfonyl]-9H-pyrrolo[2,3-b:5,4-c′]dipyrid-6-yl trifluoromethane-sulfonate 37, 5 μmol of 1,1′-bis(diphenylphosphino)ferrocenedichloropalladium(II), 0.3 mmol of caesium carbonate, 2 mL of dioxane, 0.500 mL of water and 0.15 mmol of the boronic derivative 38a-c are introduced into a microwave reactor of suitable size. The mixture obtained is then irradiated for 30 minutes at 120° C., and then taken up in ethyl acetate and water. After settling, separation and washing of the two phases, the organic phases are combined and concentrated under reduced pressure. The crude residue is triturated in acetonitrile, and the solid in suspension thus obtained is filtered off by suction under vacuum and washed with ethyl ether to give the expected compounds 39a-c (see Table 5).

TABLE 5

| Boronic | | | | |
|---|---|---|---|---|
| precursor | | | | |
| 38 | Structure 39 | Name | Yield | Analysis |
| 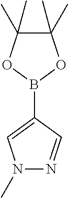 | 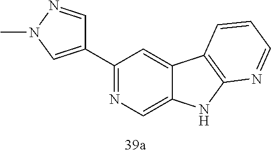 | 6-(1-Methyl-1H- pyrazol-4-yl)-9H- pyrrolo[2,3-b:5,4- c′]dipyridine | 76% | 1H NMR (400 MHz, DMSO-d6) δ ppm: 3.91 (s, 3 H) 7.30 (dd, J = 7.8, 4.9 Hz, 1 H) 8.00 (s, 1 H) 8.22 (s, 1 H) 8.42 (d, J = 1.0 Hz, 1 H) 8.57 (dd, J = 4.9, 2.0 Hz, 1 H) 8.61 (dd, J = 7.8, 2.0 Hz, 1 H) 8.84 (d, J = 1.0 Hz, 1 H) 12.04 (broad s, 1 H) UPLC-MS-DAD-ELSD: 250(+) = (M + H)(+) (Rt = 2.01 min) |
| 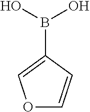 | 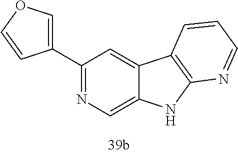 | 6-furan-3-yl-9H- pyrrolo[2,3-b:5,4- c′]dipyridine | 80% | 1H NMR (400 MHz, DMSO-d6) δ ppm: 7.12 (dd, J = 1.8, 0.8 Hz, 1 H) 7.32 (dd, J = 7.7, 4.9 Hz, 1 H) 7.77 (t, J = 1.8 Hz, 1 H) 8.26 (dd, J = 1.8, 0.8 Hz, 1 H) 8.49 (d, J = 1.2 Hz, 1 H) 8.59 (dd, J = 4.9, 1.7 Hz, 1 H) 8.63 (dd, J =7.7, 1.7 Hz, 1 H) 8.88 (d, J = 1.2 Hz, 1 H) 12.12 (broads, 1H) LC-MS-DAD-ELSD: 236(+) = (M + H)(+) (Rt = 2.30 min) |
| 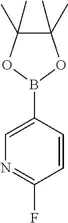 | 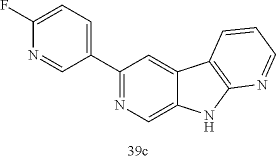 | 6-(6-fluoropyrid-3- yl)-9H-pyrrolo[2,3- b:5,4-c′]dipyridine | 88% | 1H NMR (400 MHz, DMSO-d6) δ ppm: 7.28-7.38 (m, 2 H) 8.62 (dd, J = 4.8, 1.8 Hz, 1 H) 8.70 (m, 2 H) 8.90 (d, J = 0.8 Hz, 1 H) 9.00 (d, J = 2.0 Hz, 1 H) 9.01 (d, J = 0.8 Hz, 1 H) 12.29 (broad s, 1 H) LC-MS-DAD-ELSD: 263(−) = (M − H)(−); 265(+) = (M + H)(+) ( Rt = 2.75 min) |

### Synthesis of 6-methylsulfanyl-9H-pyrrolo[2,3-b:5,4-c′]dipyridine 41

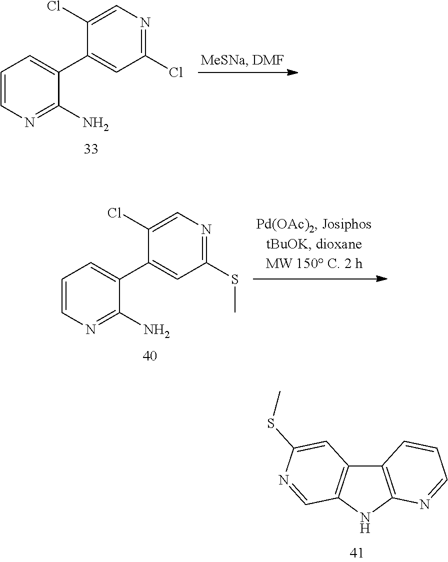

### Step 1

A mixture of 740 mg of 2′,5′-dichloro-[3,4′]bipyridyl-2-ylamine (product described in step 2 of Example 43) and 216 mg of sodium methanethiolate in 12 mL of DMF is irradiated in a microwave oven for five times 1 hour at 100° C. The brown suspension obtained is filtered and washed with ethyl acetate, and the filtrate is concentrated under reduced pressure. The crude residue is taken up in dimethyl sulfoxide, and then purified by preparative Basic HPLC (VP 240/50 mm Nucleodur 100-10 C18ec column) using a gradient of acetonitrile in water (MilliQ water supplemented with 0.07% trifluoroacetic acid). The fractions containing the expected product are combined and concentrated under reduced pressure to give 495 mg of 5′-chloro-2′-methylsulfanyl[3,4]bipyridyl-2-ylamine 40 in the form of a brown solid.

LC-MS-DAD-ELSD: 251(+)=(M+H)(+) (isotope profile corresponding to a chloro derivative) Rt (min)=2.44

### Step 2

To a suspension of 450 mg of 5′-chloro-2′-methylsulfanyl[3,4′]bipyridyl-2-ylamine 40 and 177.9 mg of potassium tert-butoxide in 10 mL of dioxane, under argon, is added an orange-brown solution, stirred beforehand under argon for 10 minutes, of 69 mg of (R)-(−)-1-[(S)-2-(dicyclohexylphosphino)ferrocenyl]ethyldi-tert-butylphosphine, 25.4 mg of palladium(II) acetate and 2 mL of anhydrous dioxane. 2 mL of dioxane are added to rinse the glassware containing the Pd(OAc)₂ preparation. The reaction medium is then irradiated for 2 hours at 150° C. The insoluble matter of the suspension obtained is separated out by filtration and the filtrate is taken up in dichloromethane and then washed with water. The organic phase is concentrated under reduced pressure, and the crude residue thus obtained is dissolved in dimethyl sulfoxide and purified by preparative basic HPLC (VP 240/50 mm Nucleodur 100-10 C18ec column) using a gradient of acetonitrile in water (MilliQ water supplemented with 0.07% trifluoroacetic acid). The fractions containing the expected product are combined and concentrated under reduced pressure to give 287 mg of beige-coloured solid 6-methylsulfanyl-9H-pyrrolo[2,3-b:5,4-c′]dipyridine 41, the characteristics of which are as follows:

1H NMR (400 MHz, DMSO-d6) δ ppm: 2.60 (s, 3 H) 7.28 (dd, J=7.8, 4.9 Hz, 1 H) 8.11 (d, J=1.5 Hz, 1H) 8.57 (dd, J=4.9, 1.5 Hz, 1 H) 8.65 (dd, J=7.8, 1.5 Hz, 1 H) 8.78 (d, J=1.5 Hz, 1 H) 11.96 (broad m, 1H)

### Example 42

### Synthesis of 6-(5-fluoropyrid-3-yl)-9H-pyrrolo[2,3-b:5,4-c′]dipyridine 42

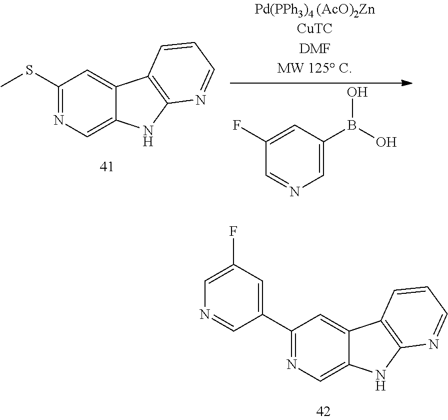

60 mg of 6-methylsulfanyl-9H-pyrrolo[2,3-b:5,4-c′]dipyridine 41, 78.5 mg of 5-fluoropyridine-3-boronic acid, 150 mg of copper thiophene-2-carboxylate, 32.2 mg of tetrakis(triphenyl-phosphine)palladium(0) and 76.7 mg of zinc acetate are introduced into a microwave reactor of suitable size, followed by addition of 3 mL of DMF under argon. The air present in the reactor is removed under vacuum and replaced with argon. The mixture thus obtained is irradiated for three times 1 hour at 150° C., and then taken up in ethyl acetate and aqueous sodium bicarbonate solution, and filtered through 0.42 μm and 0.22 μm membranes. The organic phase is concentrated under vacuum and the yellow oil obtained is purified by preparative acidic HPLC (VP 240/50 mm Nucleodur 100-10 C18ec column) using a gradient of acetonitrile supplemented with 0.07% of trifluoroacetic acid in MilliQ water supplemented with 0.07% trifluoroacetic acid. The fractions containing the expected product are combined and concentrated under reduced pressure. The residue is repurified to give 3.5 mg (5%) of 6-(5-fluoropyrid-3-yl)-9H-pyrrolo[2,3-b:5,4-c′]dipyridine in the form of a beige-coloured solid 42.

1H NMR (400 MHz, DMSO-d6) δ ppm: 7.38 (dd, J=7.8, 4.9 Hz, 1 H) 8.41 (ddd, J=10.3, 2.4, 1.5 Hz. 1H) 8.60 (d, J=2.4 Hz, 1 H) 8.64 (dd, J=4.9, 1.7 Hz, 1 H) 8.71 (dd, J=7.8, 1.7 Hz, 1 H) 9.02 (s, 1 H) 9.04 (s, 1 H) 9.28 (broad s, 1 H) 12.36 (broad s, 1 H)

### Example 43 (45) and Example 44 (46)

### Synthesis of N-[4-(3-methoxy-6-(pyrid-3-yl)-9H-pyrrolo[2,3-b:5,4-c′]dipyrid-4-yl)phenyl]methane-sulfonamide 46

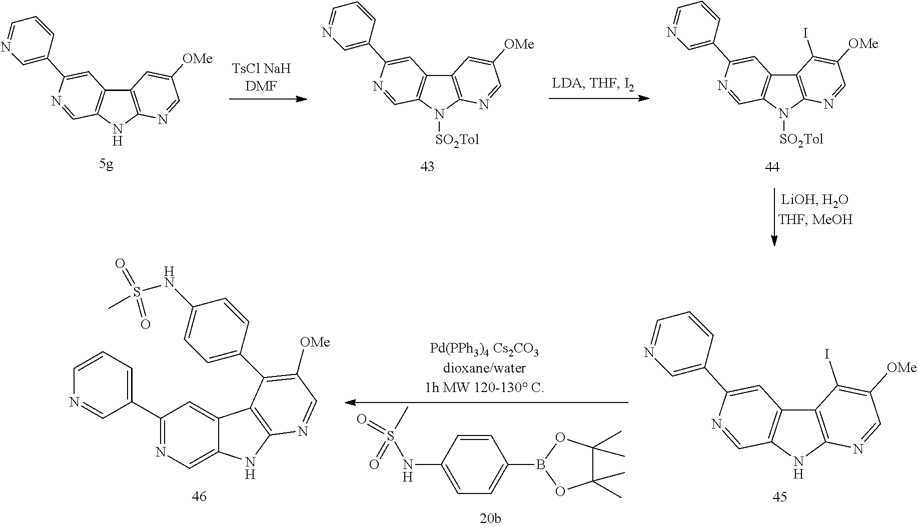

### Step 1:

0.5 g of 5 g and 12 mL of dimethylformamide are placed in a 150 mL three-necked flask. The mixture is stirred, and 126 mg of sodium hydride are then added under argon. After two hours, 690 mg of tosyl chloride in 2 mL of dimethylformamide are added. After stirring for two hours at room temperature, 100 mL of a 10% sodium bicarbonate solution and 100 mL of water are added, the mixture is extracted with 150 mL of ethyl acetate, and the extracts are dried over magnesium sulfate and filtered. The filtrate is purified by chromatography on silica gel (70 g of silica, gradient: 100/0 to 95/5 dichloromethane/methanol). 721 mg (93%) of the intermediate 3-methoxy-6-pyrid-3-yl-9-(toluene-4-sulfonyl)-9H-pyrrolo[2,3-b:5,4-c′]dipyridine 43 are obtained.

### Step 2:

0.31 mL of diisopropylamine in 10 mL of THF is placed in a dry round-bottomed flask under an argon atmosphere. The solution is cooled to −78° C., followed by addition of 1.06 mL of n-butyllithium (2.5 M in hexane). This mixture is stirred for 15 minutes, followed by dropwise addition of 600 mg of 43 predissolved in 40 mL of tetrahydrofuran. After stirring for 2 hours at −78° C., 566 mg of diiodine predissolved in 5 mL of tetrahydrofuran are added dropwise. The mixture is stirred for 10 minutes. The reaction medium is poured into 250 mL of ammonium chloride solution and the resulting mixture is extracted with 500 mL of ethyl acetate. The organic phase is washed with 200 mL of aqueous sodium thiosulfate solution and then dried over magnesium sulfate, filtered and concentrated to dryness under reduced pressure. The residue is purified by chromatography on silica gel (90 g of silica, gradient: 100/0 to 95/5 dichloromethane/methanol) to give 270 mg (35%) of the expected compound 3-methoxy-4-iodo-6-pyrid-3-yl-9-(toluene-4-sulfonyl)-9H-pyrrolo[2,3-b:5,4-c′]dipyridine 44.

### Step 3:

250 mg of 44, 6 ml, of methanol and 10 mL of tetrahydrofuran are placed in a round-bottomed flask. Aqueous lithium hydroxide solution (194 mg of LiOH.H₂O dissolved in 5 mL of water) is added. The mixture is stirred for 2 hours. 10 mL of water are added and the reaction medium is then neutralized with 4 mL of aqueous 2M hydrochloric acid solution. The precipitate is filtered off and then dried. 107 mg of 3-methoxy-4-iodo-6-(pyrid-3-yl)-9H-pyrrolo[2,3-b:5,4-c′]dipyridine 45 are thus obtained.

### Step 4:

100 mg (0.25 mmol) of 3-methoxy-4-iodo-6-(pyrid-3-yl)-9H-pyrrolo[2,3-b:5,4-c′]dipyridine 46, 0.75 mmol of the boronate 20b, 28 mg of tetrakis(triphenylphosphine)palladium(0), 121 mg of caesium carbonate, 2 mL of dioxane and 0.7 mL of water are introduced into a microwave reactor of suitable size. The mixture is irradiated for 1 hour at 120. 1 mL of methanol is added and the mixture is then poured into water (25 mL) and ethyl acetate (50 mL), the phases are separated and the aqueous phase is again extracted with 50 mL of ethyl acetate. The organic phases are combined and dried over magnesium sulfate, filtered and then concentrated under reduced pressure. The residue is purified by chromatography on silica gel (30 g of silica, gradient: 100/0 to 90/10 dichloromethane/methanol) to give 68 mg (61%) of the expected compound N-[4-(3-methoxy-6-(pyrid-3-yl)-9H-pyrrolo[2,3-b:5,4-c′]dipyrid-4-yl)phenyl]methanesulfonamide 46.

1H NMR (400 MHz, DMSO-d6) δ ppm: 3.10 (s, 3 H) 3.87 (s, 3 H) 7.42 (partially masked dd, J=8.0.4.9 Hz, 1 H) 7.45 (d, J=8.0 Hz, 2 H) 7.57 (m, 3 H) 8.09 (dt, J=8.0, 2.0 Hz, 1 H) 8.52 (dd, J=4.9, 2.0 Hz, 1H) 8.57 (s, 1 H) 8.93 (d, J=2.0 Hz, 1 H) 8.98 (s, 1 H) 10.14 (broad m, 1 H) 12.20 (broad s, 1 H)

UPLC-SQD: Retention time Rt (min)=0.53; MH+=446+; MH−=444−; Purity: 98%

### Process for Synthesizing the Comparative Compounds (not Claimed)

### Synthesis of the comparative molecule 3-methoxy-6-(pyrid-3-yl)-9H-pyrrol[2,3-c:5,4-c′]dipyridine 51

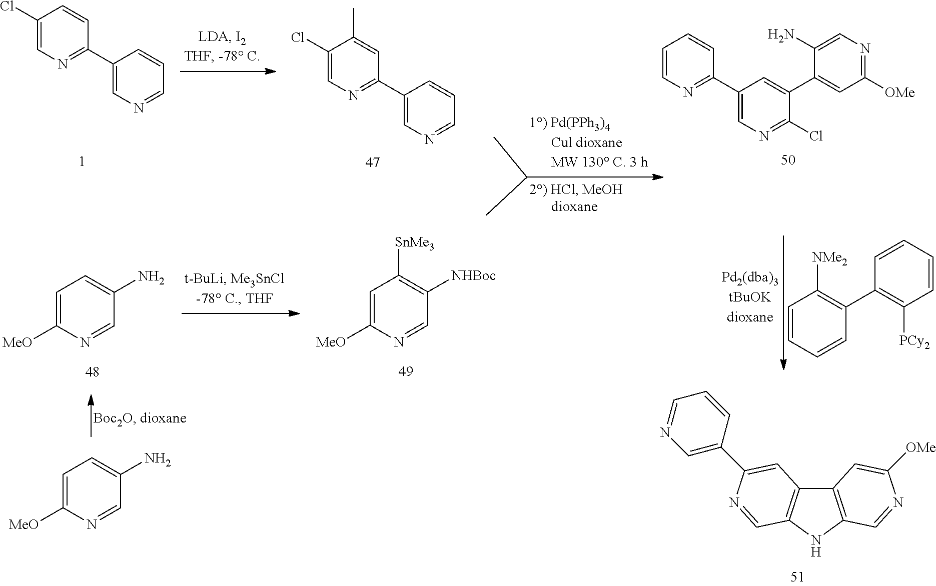

Step 1 synthesis of 47:

1.2 mL of diisopropylamine and 5 mL of tetrahydrofuran are introduced into a dry round-bottomed flask under an argon atmosphere, equipped with a magnetic stirrer. The solution is cooled to −78° C., followed by addition of 3.24 mL of n-butyllithium (2.5 M in hexane). After stirring for 15 minutes, 1.47 g of 1 predissolved in 20 mL of tetrahydrofuran are added. After stirring for 2 hours, 2.15 g of diiodine dissolved in 2.5 mL of THF are added. The mixture is thus stirred for 1 hour at −78° C. The reaction medium is hydrolysed with 120 mL of 10% ammonium chloride solution and 30 mL of water. The resulting mixture is extracted twice with 50 mL of ethyl acetate, and the combined organic phases are washed with aqueous sodium thiosulfate solution and then dried over sodium sulfate, filtered and concentrated to dryness under reduced pressure. 2.25 g of a crude product are obtained, and are purified by chromatography on silica gel with a gradient of heptane and ethyl acetate eluent (from 100/0 to 60/40 by volume), thus giving 1.58 (66%) g of 5-chloro-4-iodo-2-(3′-pyridyl)pyridine 47.

LC-MS-DAD-ELSD: 316.89(+)=(M+H)(+) Rt (min)=3.44

Step 2 synthesis of 49:

1.0 g of 5-amino-2-methoxypyridine is placed in a one-necked flask and dissolved in 40 mL of dioxane. 1.79 g of di-tert-butyl dicarbonate are added and the mixture is refluxed overnight. After cooling, the solvent is evaporated off under reduced pressure and the residue is purified by chromatography on silica gel with a gradient of heptane and ethyl acetate eluent (from 90/10 to 70/30 by volume). 1.58 g of compound 48 (97%) are obtained.

A solution of 4 mmol of 48 in dry tetrahydrofuran (20 mL) is introduced by syringe into a dry one-necked flask under argon. The solution is cooled to −78° C. and 10 mmol of tert-butyllithium (1.5 M in pentane) are then added over 15 minutes. The temperature is allowed to rise to −10° C. and the mixture is left stirring for 3 hours. The reaction mixture is again cooled to −78° C., followed by addition of a solution of 6 mmol of trimethyltin chloride in 4 mL of dry tetrahydrofuran. The reaction mixture is then poured into aqueous ammonium chloride solution and the mixture is extracted with ethyl acetate. The organic phase is dried over magnesium sulfate and concentrated under reduced pressure. The crude product is purified by chromatography on silica gel with a gradient of heptane and ethyl acetate eluent (from 95/5 to 70/30 by volume). 1.01 g (65%) of compound 49 are obtained.

LC-MS-DAD-ELSD: 389(+)=(M+H)(+) (isotope profile corresponding to a tin derivative) Rt (min)=4.69

Step 3 synthesis of 50:

453 mg (1.43 mmol) of 47, 554 mg (1.43 mmol) of the tin derivative 49, 165 mg of tetrakis(triphenylphosphine)palladium(0), 81 mg of copper iodide and 3.5 mL of dioxane are placed in a 5 mL microwave reactor. The mixture is irradiated for 1 hour at 150° C. After cooling, the mixture is poured into aqueous sodium bicarbonate solution (55 mL) and ethyl acetate (50 mL), the phases are separated and the aqueous phase is again extracted with 50 mL of ethyl acetate. The organic phases are combined and dried over magnesium sulfate, filtered and then concentrated under reduced pressure. The residue is purified by chromatography on silica gel (30 g of silica, gradient: 1/1 heptane/ethyl acetate 1/1 to pure ethyl acetate) to give 367 mg (62%) of the expected compound.

LC-MS-DAD-ELSD: 413(+)=(M+H)(+) Rt (min)=3.59

The product is redissolved in 10 mL of methanol, followed by addition of 50 mL of 4M hydrochloric acid in dioxane. After two hours, the solvents are evaporated off, the residue is dissolved in 100 mL of ethyl acetate and this phase is washed with aqueous sodium bicarbonate solution (100 mL). The organic phase is dried over magnesium sulfate and concentrated under reduced pressure. Compound 50 is obtained quantitatively, and may be used without further purification.

LC-MS-DAD-ELSD: 313(+)=(M+H)(+) Rt (min)=2.60

Step 4 synthesis of 51:

Product 50 (400 mg, 1.28 mmol) is placed in a 20 mL microwave tube with 58 mg (0.064 mmol) of tris(dibenzylideneacetone)dipalladium, 55 mg (0.14 mmol) of 2-dicyclohexylphosphino-2-(N,N-dimethylamino)biphenyl and 1.8 mmol of potassium tert-butoxide. The tube is sealed and placed under an argon atmosphere, followed by addition of 7 mL of 1,4-dioxane. The mixture is heated by microwave for 1 hour at 130° C. After cooling, the reaction mixture is poured into 50 mL of a sodium bicarbonate solution, extracted twice with 50 mL of ethyl acetate, dried over sodium sulfate, filtered and concentrated to dryness under reduced pressure. The residue is purified by chromatography on silica gel (30 g of silica, gradient: 100/0 to 90/10 ethyl acetate/methanol) to give 261 mg (74%) of the expected compound 3-methoxy-6-(pyrid-3-yl)-9H-pyrrolo[2,3-c:5,4-c′]dipyridine 51.

LC-MS-DAD-ELSD: 275(−)=(M−H)(−) Rt (min)=2.11

### Synthesis of the comparative molecule 3-(pyrid-3-yl)-9H-pyrrolo[2,3-b:5,4-c′]dipyridine 56

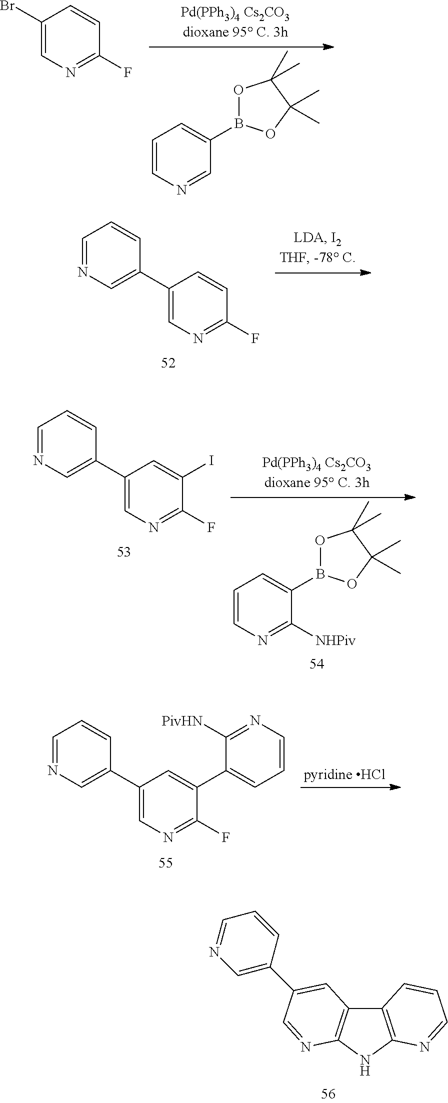

### Step 1:

0.52 g of 5-bromo-2-fluoropyridine, 646 mg of 3-(4,4,5,5-tetramethyl-1,3,2-dioxaborolan-2-yl)-pyridine, 173 mg tetrakis(triphenylphosphine)palladium(0) and 1.46 g of caesium carbonate, and then 3.8 ml, of 1,4-dioxane and 0.2 ml of water, are placed in a 5 mL microwave tube, under argon. The mixture is heated by microwave for 1 hour at 125° C. After cooling, the reaction mixture is poured into 50 mL of a 10% sodium bicarbonate solution and 25 mL of water, extracted twice with 60 mL of ethyl acetate, dried over sodium sulfate, filtered and concentrated to dryness under reduced pressure. 552 mg of a crude product are obtained, and are purified by chromatography on silica gel, eluting with a mixture of heptane and ethyl acetate (gradient: 100/0 to 60/40 by volume), thus giving 220 mg (42%) of 2-fluoro-5-(3′-pyridyl)pyridine 52.

LC-MS-DAD-ELSD: 175(+)=(M+H)(+) Rt (min)=1.84

### Step 2:

0.23 mL of diisopropylamine and 1 mL of tetrahydrofuran are introduced into a dry round-bottomed flask under an argon atmosphere, equipped with a magnetic stirrer. The solution is cooled to −78° C., followed by addition of 0.63 mL of n-butyllithium (2.5 M in hexane). After stirring for 15 minutes, 220 mg of 52 predissolved in 3 mL of tetrahydrofuran are added. After stirring for 1 hour, 417 mg of diiodine dissolved in 1 mL of THF are added. The mixture is thus stirred for 1 hour at −78° C. The reaction medium is hydrolysed with 50 mL of 10% ammonium chloride solution and 10 mL of water. The resulting mixture is extracted twice with 50 mL of ethyl acetate, and the combined organic phases are washed with aqueous sodium thiosulfate solution and then dried over sodium sulfate, filtered and concentrated to dryness under reduced pressure. The residue is purified by chromatography on silica gel with a gradient of heptane and ethyl acetate eluent (from 95/5 to 75/25 by volume), thus giving 258 mg (68%) of 2-fluoro-3-iodo-5-(3′-pyridyl)pyridine 53.

LC-MS-DAD-ELSD: 301(+)=(M+H)(+) Rt (min)=3.13

### Step 3:

250 mg (0.83 mmol) of 2-fluoro-3-iodo-5-(3′-pyridyl)pyridine 53, 278 mg (0.91 mmol) of the boronate 54, 96 mg of tetrakis(triphenylphosphine)palladium(0), 543 mg of caesium carbonate, 2.5 mL of dioxane and 0.3 mL of water are placed in a 5 mL microwave reactor. The mixture is irradiated for 1 hour at 120 C. After cooling, the reaction mixture is poured into 40 mL of a 10% sodium bicarbonate solution and 5 mL of water, extracted twice with 50 mL of ethyl acetate, dried over sodium sulfate, filtered and concentrated to dryness under reduced pressure. 289 mg of a crude product are obtained, and are purified by chromatography on silica gel (30 g of silica, gradient: 100/0 to 90/10 ethyl acetate/methanol), thus giving 202 mg (59%) of 55.

LC-MS-DAD-ELSD: 351(+)=(M+H)(+) Rt (min)=2.68

### Step 4:

175 mg of 55 preground in 2.5 g of pyridinium hydrochloride are placed in a 5 mL microwave reactor. The tube is sealed and the powders are heated by microwave for 30 minutes at 220° C. After cooling, the solid is dissolved in ethyl acetate and this phase is then washed with aqueous sodium bicarbonate solution. After drying and evaporating off the solvent, the residue is purified by preparative HPLC (phase Chiralcel OD-I 20 μm) to give 8 mg (6.5%) of the expected compound 3-(pyrid-3-yl)-9H-pyrrolo[2,3-b:5,4-c′]dipyridine 56.

LC-MS-DAD-ELSD: 247(+)=(M+H)(+) Rt (min)=2.14

### Example 45

### 1-chloro-N-{4-[3-fluoro-6-(pyrid-3-yl)-9H-pyrrolo[2,3-b:5,4-c′]dipyrid-4-yl]-phenyl}-methanesulfonamide 59

Step 1: 4-{3-fluoro-9-[(4-methylphenyl)sulfonyl]-6-(pyrid-3-yl)-9H-pyrrolo[2,3-b:5,4-c′]dipyrid-4-yl}aniline

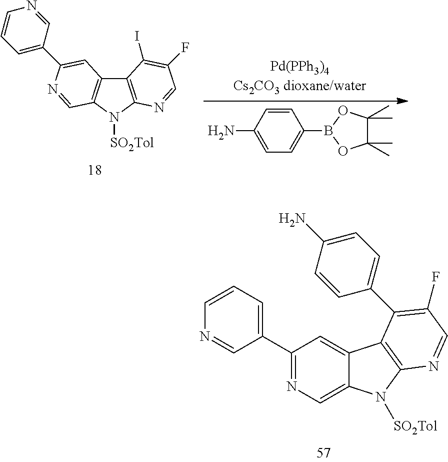

330 mg of 3-fluoro-4-iodo-6-pyrid-3-yl-9-(toluene-4-sulfonyl)-9H-pyrrolo[2,3-b:5,4-c′]dipyridine 18, 398 mg of 4-(4,4,5,5-tetramethyl-1,3,2-dioxaborolan-2-yl)aniline, 70 mg of tetrakis(triphenyl-phosphine)palladium(0), 296 mg of caesium carbonate in 8.5 mL of 1,4-dioxane and 1.5 mL of water are placed in a reactor, and the tube is sealed and subjected to microwave irradiation at 125° C. for 1 hour. 200 mL of water are added to the reaction medium, which is then extracted with twice 250 mL of ethyl acetate. The combined organic phases are concentrated to dryness under reduced pressure. The residue is purified by chromatography on a column of silica, eluting with a 100/0 to 97/3 dichloromethane/methanol mixture to give 793 mg of 4-{3-fluoro-9-[(4-methylphenyl)sulfonyl]-6-(pyrid-3-yl)-9H-pyrrolo[2,3-b:5,4-c′]dipyrid-4-yl}aniline 57.

UPLC-MS-DAD-ELSD (LS): Rt (min)=1.25; (M+H)(+): 510(+)

Step 2: 1-chloro-N-(4-{3-fluoro-9-[(4-methylphenyl)sulfonyl]-6-(pyrid-3-yl)-9H-pyrrolo[2,3-b:5,4-c′]dipyrid-4-yl}phenyl)methanesulfonamide 58

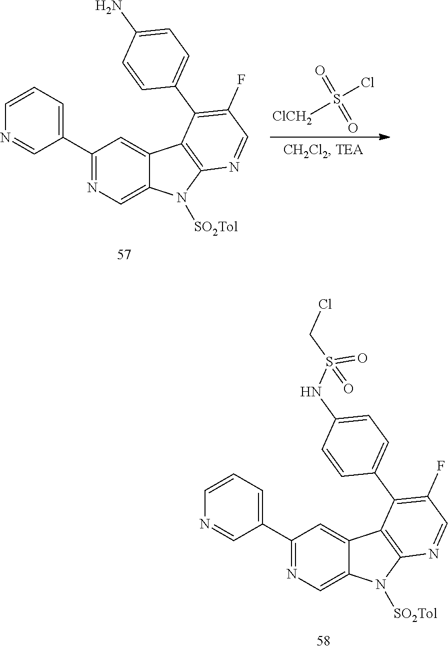

60 mg of 4-{3-fluoro-9-[(4-methylphenyl)sulfonyl]-6-(pyrid-3-yl)-9H-pyrrolo[2,3-b:5,4-c′]dipyrid-4-yl}aniline 57 in 6 mL of tetrahydrofuran and 3 mL of dichloromethane, 83 μl of triethylamine and then 35 mg of chloromethanesulfonyl chloride are placed in a reactor, and the tube is sealed and subjected to microwave irradiation for 20 minutes at 100° C. A further 35 mg of chloromethanesulfonyl chloride and 83 μl of triethylamine are added and the reaction mixture is again subjected to microwave irradiation for 30 minutes at 110° C. 300 mL of water are added to the reaction medium, which is then extracted with twice 300 mL of ethyl acetate. The combined organic phases are concentrated to dryness under reduced pressure.

The reaction medium is diluted with 5 mL of DMSO and purified by preparative HPLC, on a reverse phase in acidic medium, eluting with a gradient of the mixture: water containing 0.07% trifluoroacetic acid/acetonitrile containing 0.07% trifluoroacetic acid, to give 34 mg of 1-chloro-N-(4-{3-fluoro-9-[(4-methylphenyl)sulfonyl]-6-(pyrid-3-yl)-9H-pyrrolo[2,3-b:5,4-c′]dipyrid-4-yl}phenyl)methanesulfonamide 58 in the form of the trifluoroacetic acid salt as a white lyophilizate.

UPLC-MS-DAD-ELSD (LS): Rt (min)=1.29; (M+H)(+): 622(+)/ . . . (presence of a chlorine atom).

Step 3: 1-chloro-N-{-4-[3-fluoro-6-(pyrid-3-yl)-9H-pyrrolo[2,3-b:5,4-c′]dipyrid-4-yl]phenyl}methane-sulfonamide 59

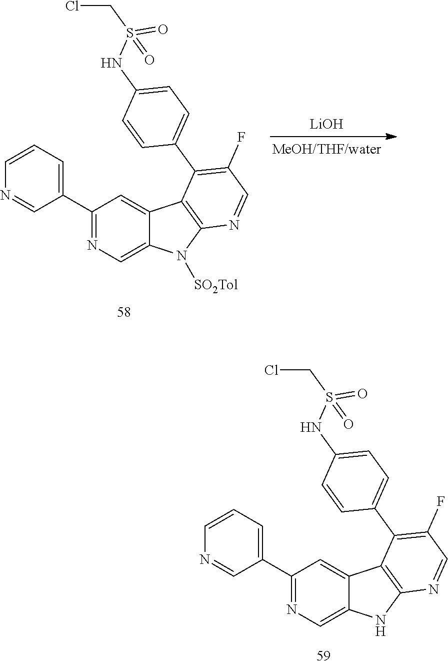

34 mg of 1-chloro-N-(4-{3-fluoro-9-[(4-methylphenyl)sulfonyl]-6-(pyrid-3-yl)-9H-pyrrolo[2,3-b:5,4-c′]dipyrid-4-yl}phenyl)methanesulfonamide 58 in 1 mL of tetrahydrofuran and then 6.88 mg of lithium hydroxide monohydrate dissolved in 0.11 mL of water are placed in a round-bottomed flask. The reaction mixture is stirred for 18 hours at room temperature and then concentrated to dryness under reduced pressure. The residue is diluted with 5 mL of DMSO and purified by preparative HPLC, on a reverse phase in acidic medium, eluting with a gradient of water containing 0.07% trifluoroacetic acid/acetonitrile containing 0.07% trifluoroacetic acid, to give 13 mg of 1-chloro-N-{-4-[3-fluoro-6-(pyrid-3-yl)-9H-pyrrolo[2,3-b:5,4-c′]dipyrid-4-yl]phenyl}methanesulfonamide 59 in the form of the trifluoroacetic acid salt as a white lyophilizate.

UPLC-MS-DAD-ELSD: Rt (min)=0.66; [M+H]+: m/z 468.

1H NMR (400 MHz, DMSO-d6) δ ppm: 5.20 (s, 2 H) 7.52 (dd, J=7.8, 4.9 Hz, 1 H) 7.57 (d, J=8.8 Hz, 2 H) 7.76 (d, J=8.3 Hz, 2 H) 7.82 (s, 1 H) 8.23 (d, J=7.8 Hz, 1 H) 8.59 (d, J=4.4 Hz, 1 H) 8.74 (d, J=2.4 Hz, 1H) 9.04-9.09 (m, 2 H) 10.76 (s, 1 H) 12.60 (s, 1 H).

### Example 46

### N-{4-[3-fluoro-6-(pyrid-3-yl)-9H-pyrrolo[2,3-b:5,4-c′]dipyrid-4-yl]phenyl}cyclo-propanesulfonamide 61

Step 1: N-(4-{3-fluoro-9-[(4-methylphenyl)sulfonyl]-6-(pyrid-3-yl)-9H-pyrrolo[2,3-b:5,4-c′]dipyrid-4-yl}phenyl)cyclopropanesulfonamide 60

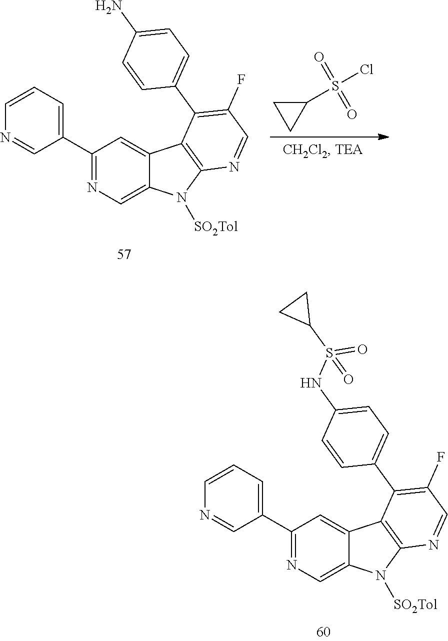

196 mg of 4-{3-fluoro-9-[(4-methylphenyl)sulfonyl]-6-(pyrid-3-yl)-9H-pyrrolo[2,3-b:5,4-c′]dipyrid-4-yl}aniline 57 in 10 mL of tetrahydrofuran and 5 mL of dichloromethane, 0.138 mL of triethylamine and then 55 mg of cyclopropanesulfonyl chloride are placed in a reactor, and the tube is sealed and subjected to microwave irradiation for 20 minutes at 100° C. The reaction medium is treated with 300 mL of water and then extracted with three times 300 mL of ethyl acetate. The combined organic phases are concentrated to dryness under reduced pressure to give 148 mg of N-(4-{3-fluoro-9-[(4-methylphenyl)sulfonyl]-6-(pyrid-3-yl)-9H-pyrrolo[2,3-b:5,4-c′]dipyrid-4-yl}phenyl)cyclopropanesulfonamide 60, which is used in crude form in the following step.

Step 2: N-{4-[3-fluoro-6-(pyrid-3-yl)-9H-pyrrolo[2,3-b:5,4-c′]dipyrid-4-yl]phenyl}cyclopropanesulfonamide 61

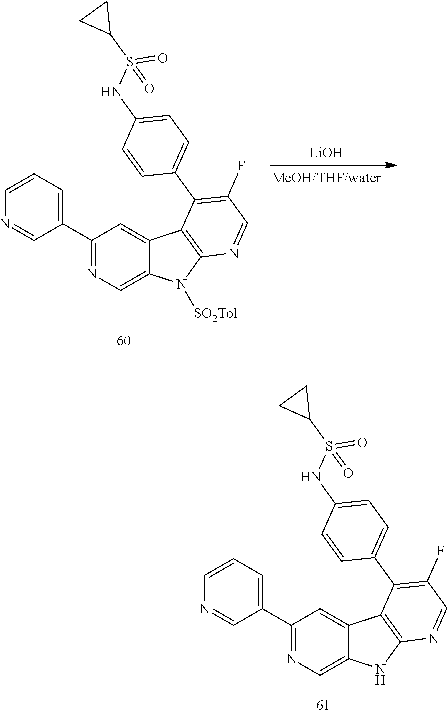

148 mg of N-(4-{3-fluoro-9-[(4-methylphenyl)sulfonyl]-6-(pyrid-3-yl)-9H-pyrrolo[2,3-b:5,4-c′]-dipyrid-4-yl}phenyl)cyclopropanesulfonamide 60 in 4.7 mL of tetrahydrofuran and then 30 mg of lithium hydroxide monohydrate dissolved in 0.47 mL of water are placed in a round-bottomed flask. The reaction mixture is stirred for 4 hours at room temperature, followed by addition of a further 30 mg of lithium hydroxide monohydrate. The reaction mixture is stirred for 42 hours at room temperature and then concentrated to dryness under reduced pressure. The residue is diluted with 5 mL of DMSO and purified by preparative HPLC, on a reverse phase in basic medium, eluting with a gradient of water+10 mM ammonium formate+aqueous ammonia (pH of between 9 and 10)/acetonitrile to give 37 mg of N-{-4-[3-fluoro-6-(pyrid-3-yl)-9H-pyrrolo[2,3-b:5,4-c′]dipyrid-4-yl]-phenyl}cyclopropanesulfonamide 61 in the form of a beige-coloured powder.

UPLC-MS-DAD-ELSD: Rt (min)=0.64; [M+H]⁺: m/z 460; [M−H]⁻: m/z 458.

1H NMR (400 MHz, DMSO-d6) δ ppm: 1.01-1.07 (m, 4 H) 2.74-2.82 (m, 1 H) 7.44 (dd, J=7.8, 4.6 Hz. 1 H) 7.57 (d, J=8.8 Hz, 2 H) 7.73 (d, J=8.1 Hz, 2 H) 7.78 (d, J=1.0 Hz, 1 H) 8.18 (dt, J=8.1, 2.0 Hz, 1 H) 8.54 (dd, J=4.8, 1.6 Hz, 1 H) 8.73 (d, J=2.4 Hz, 1 H) 8.94 (d, J=1.7 Hz, 1 H) 9.06 (d, J=1.0 Hz, 1 H) 10.14 (br. s., 1 H) 12.55 (br. s., 1 H).

### Example 47

### N-{-4-[3-fluoro-6-(pyrid-3-yl)-9H-pyrrolo[2,3-b:5,4-c′]dipyrid-4-yl]-2-methoxyphenyl}-methanesulfonamide 64

Step 1: 4-{3-fluoro-9-[(4-methylphenyl)sulfonyl]-6-(pyrid-3-yl)-9H-pyrrolo[2,3-b:5,4-c′]dipyrid-4-yl}-2-methoxyaniline 62

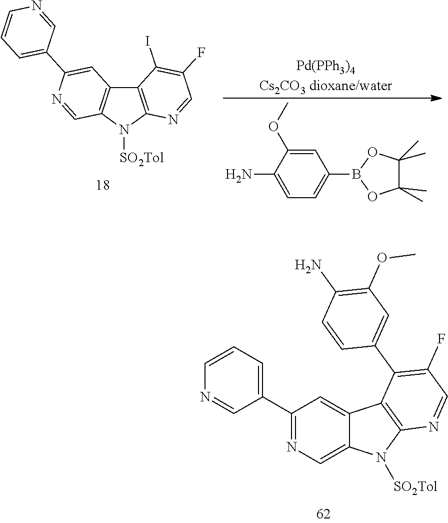

250 mg of 3-fluoro-4-iodo-6-pyrid-3-yl-9-(toluene-4-sulfonyl)-9H-pyrrolo[2,3-b:5,4-c′]dipyridine 18, 343 mg of 4-amino-3-methoxyphenylboronic acid pinacol ester, 53 mg of tetrakis(triphenyl-phosphine)palladium(0), 224 mg of caesium carbonate in 4 mL of 1,4-dioxane and 1 mL of water are placed in a reactor, and the tube is sealed and subjected to microwave irradiation at 125° C. for 1 hour. After 18 hours at room temperature, 300 ml of water are added to the reaction medium, which is then extracted with twice 300 mL of ethyl acetate. The combined organic phases are concentrated to dryness under reduced pressure. The residue is purified by chromatography on a column of silica, eluting with a 100/0 to 98/2 dichloromethane/methanol mixture to give 113 mg of 4-{3-fluoro-9-[(4-methylphenyl)sulfonyl]-6-(pyrid-3-yl)-9H-pyrrolo[2,3-b:5,4-c′]dipyrid-4-yl}-2-methoxyaniline 62.

UPLC-MS-DAD-ELSD (LS): Rt (min)=1.28; (M+H)(+): 540(+).

Step 2: N-(4-{3-fluoro-9-[(4-methylphenyl)sulfonyl]-6-(pyrid-3-yl)-9H-pyrrolo[2,3-b:5,4-c′]dipyrid-4-yl}-2-methoxyphenyl)methanesulfonamide 63

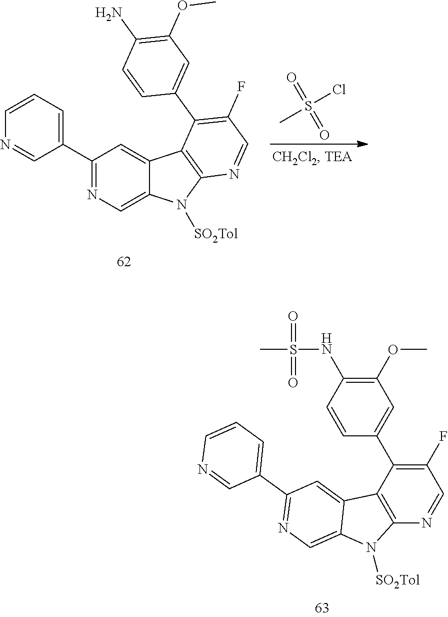

113 mg of 4-{3-fluoro-9-[(4-methylphenyl)sulfonyl]-6-(pyrid-3-yl)-9H-pyrrolo[2,3-b:5,4-c′]dipyrid-4-yl}-2-methoxyaniline 62 in 10 mL, of tetrahydrofuran and 5 mL of dichloromethane, 0.456 mL, of triethylamine and then 55 mg of methanesulfonyl chloride are placed in a reactor, and the tube is sealed and subjected to microwave irradiation for 20 minutes at 100° C. 300 mL of water are added to the reaction medium, which is then extracted with twice 300 mL of ethyl acetate. The combined organic phases are concentrated to dryness under reduced pressure to give 210 mg of N-(4-[3-fluoro-9-[(4-methylphenyl)sulfonyl]-6-(pyrid-3-yl)-9H-pyrrolo[2,3-b:5,4-c′]dipyrid-4-yl]-2-methoxy-phenyl)methane sulfonamide 63.

UPLC-MS-DAD-ELSD (LS): Rt (min)=1.27; (M+H)(+): 618(+); (M−H)(−): 616(−).

Step 3: N-{-4-[3-fluoro-6-(pyrid-3-yl)-9H-pyrrolo[2,3-b:5,4-c′]dipyrid-4-yl]-2-methoxyphenyl}-methanesulfonamide 64

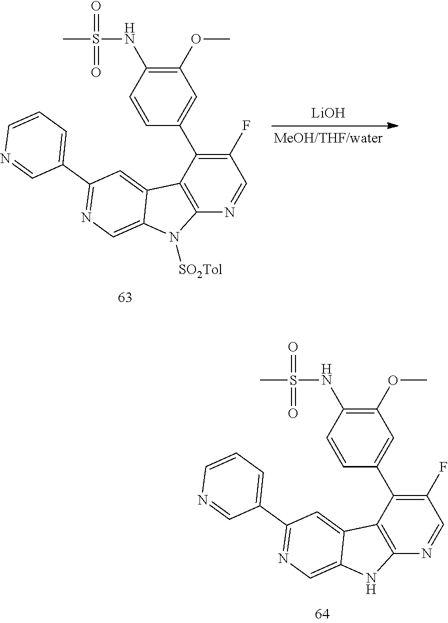

129 mg of N-(4-{3-fluoro-9-[(4-methylphenyl)sulfonyl]-6-(pyrid-3-yl)-9H-pyrrolo[2,3-b:5,4-c′]-dipyrid-4-yl}-2-methoxyphenyl)methanesulfonamide 63 in 4 mL of tetrahydrofuran and then 26 mg of lithium hydroxide monohydrate dissolved in 0.67 mL of water are placed in a round-bottomed flask. The reaction mixture is stirred for 16 hours at room temperature, followed by addition of 300 mL of water, which is then extracted with twice 300 mL of ethyl acetate. The combined organic phases are concentrated to dryness under reduced pressure and the residue is then diluted with 5 mL of DMSO and purified by preparative HPLC, on a reverse phase in basic medium, eluting with a gradient of water+10 mM ammonium formate+aqueous ammonia (pH between 9 and 10)/acetonitrile to give 61 mg of N-{-4-[3-fluoro-6-(pyrid-3-yl)-9H-pyrrolo[2,3-b:5,4-c′]dipyrid-4-yl]-2-methoxyphenyl}-methanesulfonamide 64 in the form of a yellow lyophilizate.

UPLC-MS-DAD-ELSD: Rt (min)=0.59; [M+H]⁺: m/z 464; [M−H]⁻: m/z 462.

1H NMR (400 MHz, DMSO-d6) δ ppm: 3.11 (s, 3 H) 3.85 (s, 3 H) 7.32 (d, J=8.1 Hz, 1 H) 7.42-7.47 (m, 2 H) 7.62 (d, J=8.1 Hz, 1 H) 7.76 (d, J=0.7 Hz, 1 H) 8.12 (dt, J=8.1, 1.7 Hz, 1 H) 8.54 (dd, J=4.6, 1.5 Hz, 1 H) 8.74 (d, J=2.4 Hz, 1 H) 9.00 (d, J=1.7 Hz, 1 H) 9.07 (d, J=0.7 Hz, 1 H) 9.36 (br. s., 1 H) 12.58 (br. s., 1 H).

### Example 48

### N-{-4-[3-fluoro-6-(pyrid-3-yl)-9H-pyrrolo[2,3-b:5,4-c′]dipyrid-4-yl]-phenyl}-N-methyl-methanesulfonamide 66

Step 1: N-methyl-N-[4-(4,4,5,5-tetramethyl-1,3,2-dioxaborolan-2-yl)phenyl]methanesulfonamide

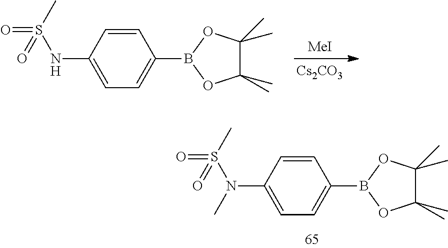

600 mg of 4-methanesulfonylaminophenylboronic acid pinacol ester, 1.32 g of caesium carbonate in 40 mL of dimethylformamide and then 0.25 ml of iodomethane are placed in a reactor, and the tube is sealed and subjected to microwave irradiation for 20 minutes at 90° C. The reaction mixture is poured into 2 1 of water and 500 ml, of ethyl acetate. After separation of the phases by settling, the organic phase is concentrated under vacuum to give 513 mg of N-methyl-N-[4-(4,4,5,5-tetramethyl-1,3,2-dioxaborolan-2-yl)phenyl]methanesulfonamide 65 in the form of a beige-coloured oil.

UPLC-MS-DAD-ELSD (LS): Rt (min)=1.24; (M+H)(+): 312(+).

Step 2: N-{-4-[3-fluoro-6-(pyrid-3-yl)-9H-pyrrolo[2,3-b:5,4-c′]dipyrid-4-yl]phenyl}-N-methyl-methanesulfonamide 66

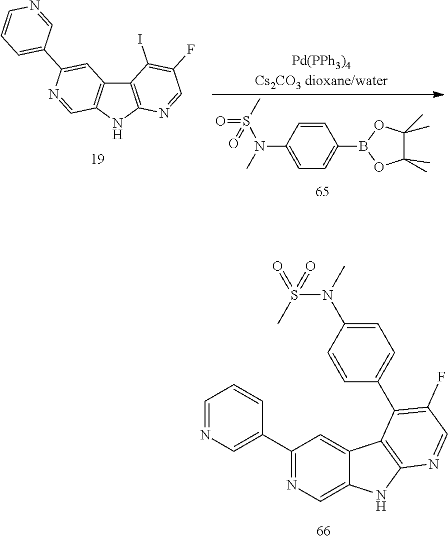

500 mg of 3-fluoro-4-iodo-6-(pyrid-3-yl)-9H-pyrrolo[2,3-b:5,4-c′]dipyridine 19, 518 mg of N-methyl-N-[4-(4,4,5,5-tetramethyl-1,3,2-dioxaborolan-2-yl)phenyl]methanesulfonamide dissolved in 10 mL of 1,4-dioxane, 407 mg of caesium carbonate, 101 mg of tetrakis(triphenylphosphine)palladium(0) in 7 mL, of 1,4-dioxane and 2 mL of water are placed in a reactor, and the tube is sealed and subjected to microwave irradiation for 1 hour at 125° C. After 18 hours at room temperature, 1 1 of water and 1 1 of ethyl acetate are added to the reaction mixture and the whole is then stirred for 30 minutes at room temperature. The precipitate formed is filtered off by suction under vacuum and then washed with 50 mL of water and 50 mL of ethyl acetate and dried again under vacuum to give 334 mg of N-{4-[3-fluoro-6-(pyrid-3-yl)-9H-pyrrolo[2,3-b:5,4-c′]dipyrid-4-yl]phenyl}-N-methylmethanesulfonamide 66.

UPLC-MS-DAD-ELSD: Rt (min)=0.64; [M+H]⁺: m/z 448; [M−H]⁻: m/z 446.

1H NMR (400 MHz, DMSO-d6) δ □ppm: 3.10 (s, 3 H) 3.41 (s, 3 H) 7.44 (dd, J=7.1, 4.9 Hz, 1 H) 7.64 (s, 1H) 7.74-7.83 (m, 4 H) 8.10 (d, J=7.8 Hz, 1 H) 8.54 (d, J=3.4 Hz, 1 H) 8.76 (s, 1 H) 9.02 (s, 1 H) 9.07 (s, 1 H) 12.57 (br. s., 1 H).

### Example 49

### N-[3-(dimethylamino)propyl]-N-{4-[3-fluoro-6-(pyrid-3-yl)-9H-pyrrolo[2,3-b:5,4-c′]-dipyrid-4-yl]phenyl}methanesulfonamide 68

Step 1: N-[3-(dimethylamino)propyl]-N-[4-(4,4,5,5-tetramethyl-1,3,2-dioxaborolan-2-yl)phenyl]-methanesulfonamide 67

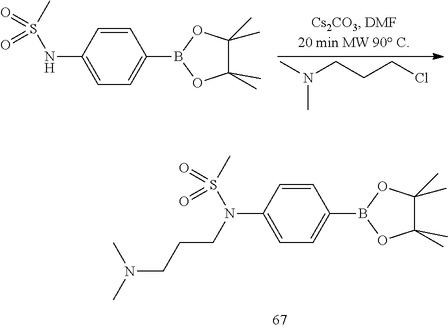

300 mg of 4-methanesulfonylaminophenylboronic acid pinacol ester, 1.15 g of caesium carbonate in 18 mL of dimethylformamide and then 160 mg of 3-dimethylaminopropyl chloride hydrochloride are introduced into a microwave reactor of suitable size. The tube is sealed and subjected to microwave irradiation for 20 minutes at 90° C. and then for 1 hour at 60° C. The reaction mixture is poured into 500 mL of water and extracted with 300 mL of ethyl acetate. The organic phase is concentrated under vacuum to give 481 mg of N-[3-(dimethylamino)propyl]-N-[4-(4,4,5,5-tetramethyl-1,3,2-dioxaborolan-2-yl)phenyl]methanesulfonamide 67 in the form of a colourless oil.

UPLC-MS-DAD-ELSD (LS): Rt (min)=0.69; (M+H)(+): 383(+).

### Step 2:

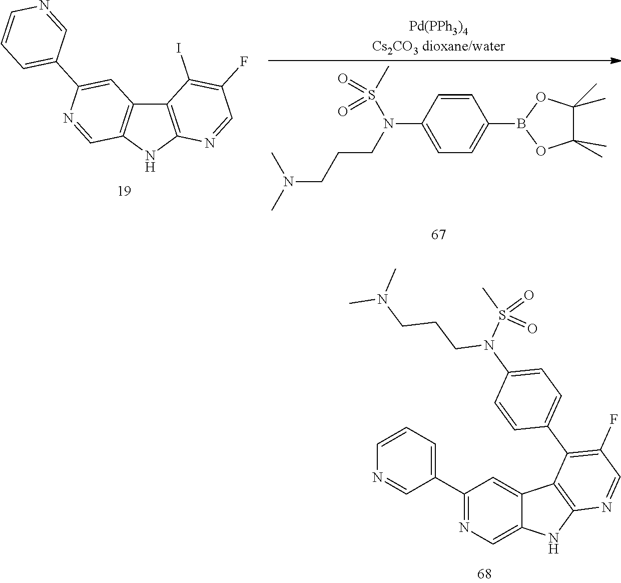

100 mg of 3-fluoro-4-iodo-6-(pyrid-3-yl)-9H-pyrrolo[2,3-b:5,4-c′]dipyridine 19, 294 mg of N-[3-(dimethylamino)propyl]-N-[4-(4,4,5,5-tetramethyl-1,3,2-dioxaborolan-2-yl)phenyl]methanesulfon-amide 67 prepared in step 1, 125 mg of caesium carbonate, 30 mg of tetrakis(triphenyl-phosphine)palladium(0), 3.6 mL of 1,4-dioxane and 0.6 mL of water are introduced into a microwave reactor of suitable size. The tube is sealed and subjected to microwave irradiation for 1 hour at 125° C. The reaction mixture is poured into 200 mL of water and extracted with three times 200 mL of ethyl acetate. The combined organic phases are concentrated under vacuum to give a brown solid. This product is purified by chromatography on a column of silica, eluting with a 100/0/0 to 95/4.5/0.5 dichloromethane/methanol/concentrated aqueous ammonia mixture, to give 16 mg of N-[3-(dimethylamino)propyl]-N-{4-[3-fluoro-6-(pyrid-3-yl)-9H-pyrrolo[2,3-b:5,4-c′]dipyrid-4-yl]phenyl}-methanesulfonamide 68 in the form of a yellow solid.

1H NMR (400 MHz, DMSO-d6) δ □ppm 1.55 to 1.67 (m, 2 H); 2.04 (s, 6 H); 2.27 (t, J=6.9 Hz, 2 H); 3.12 (s, 3 H); 3.82 (t, J=6.9 Hz, 2 H); 7.42 (ddd, J=0.8 and 4.9 and 8.1 Hz, 1 H); 7.62 (d, J=1.0 Hz, 1 H); 7.76 (d, J=8.3 Hz, 2 H); 7.82 (d, J=8.3 Hz, 2 H); 8.07 (ddd, J=1.7 and 2.2 and 8.1 Hz, 1 H); 8.54 (dd, J=1.7 and 4.9 Hz, 1 H); 8.77 (d, J=2.2 Hz, 1 H); 9.01 (dd, J=0.8 and 2.2 Hz, 1 H); 9.07 (d, J=1.0 Hz, 1 H); 12.36 to 12.83 (broad m, 1 H).

LC-MS (7 min). Rt (min)=2.27; [M+H]⁺, m/z 519; [M+2H]²⁺: m/z 260 (base peak); [M−H]⁻: m/z 517.

### Example 50

### 4-[3-fluoro-6-(pyrid-3-yl)-9H-pyrrolo[2,3-b:5,4-c′]dipyrid-4-yl]-N-(prop-2-en-1-yl)-aniline 70

Step 1: N-[4-(4,4,5,5-tetramethyl-1,3,2-dioxaborolan-2-yl)phenyl]prop-2-ene-1-sulfonamide 69

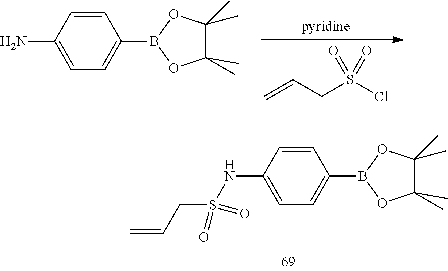

To 438 mg of 4-(4,4,5,5-tetramethyl-1,3,2-dioxaborolan-2-yl)aniline in 10 mL of pyridine at 25° C. are added 309 mg of prop-2-ene-1-sulfonyl chloride. The reaction medium is stirred for 1 hour at 25° C. and then concentrated. The residue is taken up in ethyl acetate, and the organic phase is washed twice with water, dried over magnesium sulfate, filtered and then concentrated to dryness under reduced pressure to give 625 mg of N-[4-(4,4,5,5-tetramethyl-1,3,2-dioxaborolan-2-yl)phenyl]prop-2-ene-1-sulfonamide 69 in the form of a beige-coloured solid.

### Step 2:

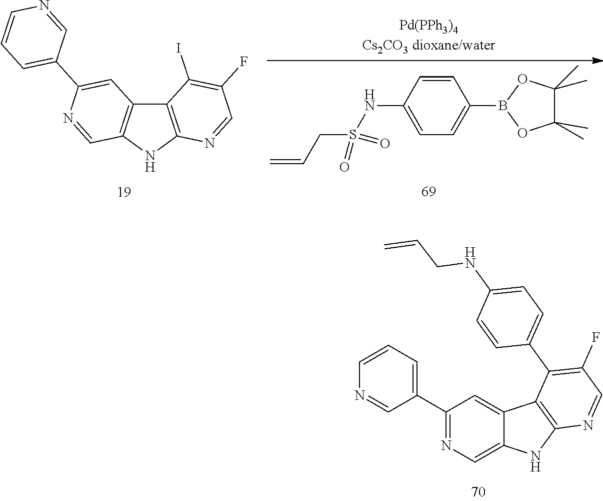

100 mg of 3-fluoro-4-iodo-6-(pyrid-3-yl)-9H-pyrrolo[2,3-b:5,4-c′]dipyridine 19 and 207 mg of N-methyl-N-[4-(4,4,5,5-tetramethyl-1,3,2-dioxaborolan-2-yl)phenyl]methanesulfonamide 69 in 1.0 mL of 1,4-dioxane are introduced into a microwave reactor of suitable size, followed by addition of 0.34 mL of aqueous 1.5 M caesium carbonate solution and 30 mg of tetrakis(triphenyl-phosphine)palladium(0), and the mixture is subjected to microwave irradiation for 1 hour at 150° C. The suspension is filtered and the filtrate concentrated. The residue is purified by chromatography on a column of silica, eluting with a 0 to 10% dichloromethane/isopropanol gradient to give 15 mg of 4-[3-fluoro-6-(pyrid-3-yl)-9H-pyrrolo[2,3-b:5,4-c′]dipyrid-4-yl]-N-(prop-2-en-1-yl)aniline 70 in the form of a yellow solid.

1H NMR (400 MHz, DMSO-d6) δ □ppm 3.81 to 3.87 (m, 2 H); 5.22 (qd, J=1.7 and 10.3 Hz, 1 H); 5.33 (qd, J=1.7 and 17.5 Hz, 1 H); 5.90 to 6.07 (m, 1 H); 6.48 (t, J=6.1 Hz, 1 H); 6.87 (d, J=8.8 Hz, 2 H); 7.44 to 7.55 (m, 3 H); 8.02 (d, J=1.0 Hz, 1 H); 8.20 (dt, J=2.2 and 8.1 Hz, 1 H); 8.56 (dd, J=1.7 and 4.8 Hz, 1 H); 8.63 (d, J=2.9 Hz, 1 H); 9.00 (d, J=2.2 Hz, 1 H); 9.03 (d, J=1.0 Hz, 1 H); 12.23 to 12.58 (broad m, 1 H).

LC-MS (7 min): Rt (min)=3.44; [M+H]⁺: m/z 396; m/z 356 (base peak); [M−H]⁻: m/z 394.

### Examples 51 to 74 (71a-71×)

### General Procedure

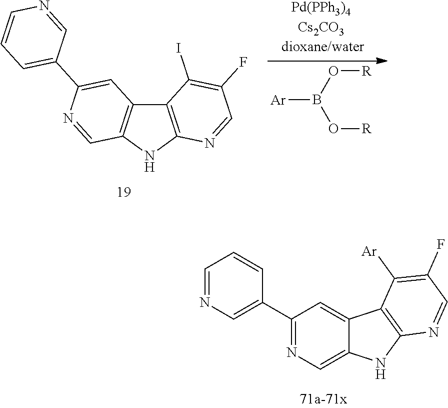

0.2 mmol of 3-fluoro-4-iodo-6-(pyrid-3-yl)-9H-pyrrolo[2,3-b:5,4-c′]dipyridine 19, 0.4 mmol of the boronic derivative (acid or ester) in 2 mL of 1,4-dioxane, and 0.4 mmol of caesium carbonate in 0.5 mL of water are placed in a reactor, followed by addition, under argon, of 0.02 mmol of tetrakis(triphenylphosphine)palladium(0) in 0.5 mL of dimethylformamide, and the tube is sealed and stirred at 110° C. for 18 hours. After cooling, the reaction mixture is diluted with 6 mL of 1,4-dioxane, 2 mL of methanol and 0.1 ml trifluoroacetic acid and then treated for 4 hours at room temperature with 150 mg of resin of propanethiol type grafted onto silica. The reaction mixture is filtered and then washed twice with a 4/1 1,4-dioxane/methanol mixture. After evaporating under reduced pressure, the residue is dissolved in 2 mL of dimethylformamide and 0.1 ml trifluoroacetic acid, filtered and then purified by preparative HPLC, eluting with a 90/10 to 5/95 water+0.1% trifluoroacetic acid/acetonitrile+0.1% trifluoroacetic acid gradient.

The products 71a to 71× are detailed in Table 6.

TABLE 6

| Boronic acid or | | Name of the neutral | |
|---|---|---|---|
| ester reagent | Structure obtained | compound | Analysis |
| 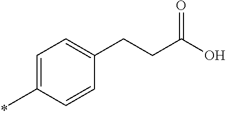 | 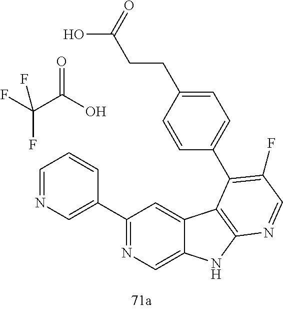 | 3-{4-[3-fluoro-6- (pyrid-3-yl)-9H- pyrrolo[2,3-b:5,4-c′]- dipyrid-4-yl]phenyl}- propanoic acid | 1H NMR (400 MHz, DMSO- d6) δ ppm: 2.71 (t, J = 7.3 Hz, 2 H); 2.97 to 3.13 (m, 2 H); 7.59 (d, J = 8.1 Hz, 2H); 7.63 to 7.71 (m, 3 H); 7.74 (s, 1 H); 8.28 (d, J = 8.3 Hz, 1 H); 8.59 to 8.68 (m, J = 5.9 Hz, 1 H); 8.75 (d, J = 2.4 Hz, 1 H); 9.07 (broad s, 1 H); 9.08 (s, 1 H); 12.63 (s, 1 H) LC-TOF-MS: Rt (min) = 2.50; [M + H]^{+:} m/z = 413 |
| 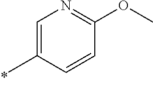 | 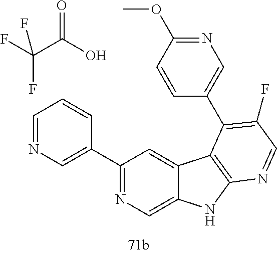 | 3-fluoro-4-(6-meth- oxypyrid-3-yl)-6- (pyrid-3-yl)-9H- pyrrolo[2,3-b:5,4-c′]- dipyridine | 1H NMR (400 MHz, DMSO- d6) δ ppm: 4.02 (s, 3 H); 7.20 (d, J = 8.6 Hz, 1 H); 7.65 (dd, J = 5.4 and 8.3 Hz, 1 H); 7.89 (s, 1 H); 8.17 (dd, J = 2.7 and 8.6 Hz, 1 H); 8.38 (d, J = 8.3 Hz, 1 H); 8.60 (s, 1 H); 8.64 (d, J = 5.4 Hz, 1 H); 8.77 (d, J = 2.4 Hz, 1 H); 9.07 to 9.10 (m, 1 H); 9.11 (s, 1 H); 12.67 (s, 1 H) LC-TOF-MS: Rt (min) = 2.43; [M + H]^{+:} m/z = 372 |
| 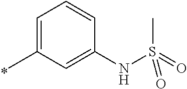 | 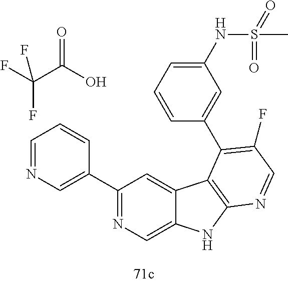 | N-{3-[3-fluoro-6- (pyrid-3-yl)-9H- pyrrolo[2,3-b:5,4-c′]- dipyrid-4-yl]phenyl}- methanesulfonamide | 1H NMR (400 MHz, DMSO- d6) δ ppm: 3.08 (s, 3 H); 7.47 to 7.57 (m, 3 H); 7.63 to 7.72 (m, 2 H); 7.86 (s, 1 H); 8.39 (d, J = 8.3 Hz, 1 H); 8.65 (dd, J = 0.7 and 5.1 Hz, 1 H); 8.77 (d, J = 2.4 Hz, 1 H); 9.08 (s, 1 H); 9.10 (s, 1 H); 10.09 (s, 1 H); 12.69 (s, 1 H) LC-TOF-MS: Rt (min) = 2.43; [M + H]^{+:} m/z = 434 (ES+) |
| 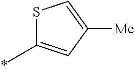 | 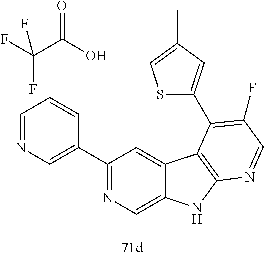 | 3-fluoro-4-(4- methylthiophen-2- yl)-6-(pyrid-3-yl)- 9H-pyrrolo[2,3-b:5,4- c′]dipyridine | 1H NMR (400 MHz, DMSO- d6) δ ppm: 2.43 (s, 3 H); 7.61 (d, J = 1.5 Hz 1 H); 7.64 (s, 1 H); 7.68 (dd, J = 5.0 and 8.2 Hz, 1 H); 8.23 (s, 1 H); 8.42 (d, J = 8.1 Hz, 1 H); 8.66 (d, J = 5.4 Hz, 1 H); 8.75 (d, J = 2.7 Hz, 1 H); 9.10 (s, 1 H); 9.13 (broad s, 1 H); 12.69 (s, 1 H) LC-TOF-MS: Rt (min) = 2.67; [M + H]^{+:} m/z = 361 (ES+) |
| 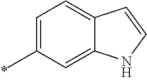 | 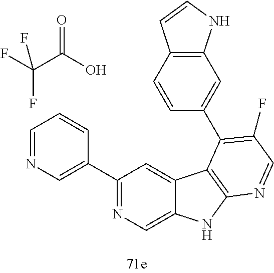 | 3-(fluoro-4-(1H-indol- 6-yl)-6-(pyrid-3-yl)- 9H-pyrrolo[2,3-b:5,4- c′]dipyridine | 1H NMR (400 MHz, DMSO- d6) δ ppm: 6.62 to 6.66 (m, 1 H); 7.40 (d, J = 8.1 Hz, 1 H); 7.58 (t, J = 2.8 Hz, 1 H); 7.64 (dd, J = 5.0 and 8.2 Hz, 1 H); 7.82 (s, 1 H); 7.88 (d, J = 8.1 Hz, 1 H); 7.94 (s, 1 H); 8.31 (d, J = 8.3 Hz, 1 H); 8.61 (dd, J = 1.7 and 4.9 Hz, 1 H); 8.75 (d, J = 2.7 Hz, 1 H); 8.96 (d, J = 2.0 Hz, 1 H); 9.09 (s, 1 H); 11.44 (broads, 1 H); 12.63 (s, 1 H) LC-TOF-MS: Rt (min) = 2.60; m/z = 380(ES+) |
| 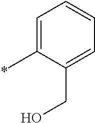 | 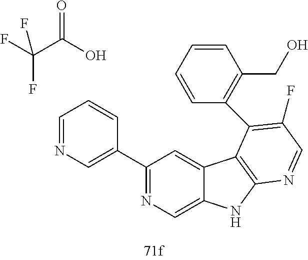 | {2-(3-fluoro-6- (pyrid-3-y1)-9H- pyrrolo[2,3-b:5,4-c′]- dipyrid-yl]phenyl}- methanol | 1H NMR (400 MHz, DMSO- d6) δ ppm: 4.2l to 4.29 (m, 1 H); 4.31 to 4.39 (m, 1 H); 7.23 (s, 1 H); 7.48 (d, J = 7.1 Hz, 1 H); 7.57 (td, J = 1.2 and 7.5 Hz, 1 H); 7.65 (dd, J = 5.0 and 8.2 Hz, 1 H); 7.71 (td, J = 1.5 and 7.6 Hz, 1 H); 7.82 (d, J = 7.3 Hz, 1 H); 8.22 (d, J = 8.3 Hz, 1 H); 8.63 (d, J = 5.1 Hz, 1 H); 8.77 (d, J = 2.2 Hz, 1 H); 8.90 (s, 1 H); 9.08 (s, 1 H); 12.64 (s, 1 H) LC-TOF-MS: Rt (min) = 2.30; [M + H]^{+:} m/z = 371 (ES+) |
| 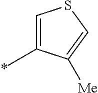 | 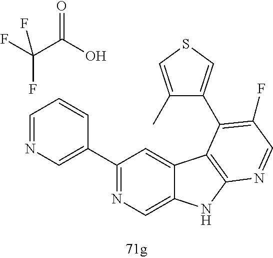 | 3-fluoro-4-(4-methyl- thiophen-3-yl)-6- (pyrid-3-yl)-9H- pyrrolo[2,3-b:5,4-c′]- dipyridine | 1H NMR (400 MHz, DMSO- d6) δ ppm: 2.06 (s, 3 H); 7.60 (s, 1 H); 7.62 (dd, J = 1.1 and 3.3 Hz, 1 H); 7.69 (dd, J = 4.8 and 8.2 Hz, 1 H); 7.96 (d, J = 3.2 Hz, 1 H); 8.36 (d, J = 8.6 Hz, 1 H); 8.66 (d, J = 5.0 Hz, 1 H); 8.77 (d, J = 2.4 Hz, 1 H); 9.05 (s, 1 H); 9.10 (s, 1 H); 12.65 (s, 1 H) LC-TOF-MS: Rt (min) = 2.57; [M+H]^{+:} m/z = 361 (ES+) |
| 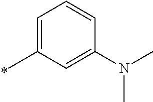 | 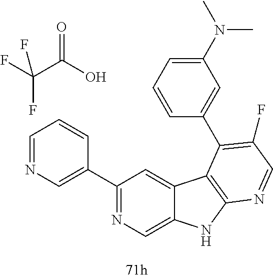 | 3-[3-fluoro-6-(pyrid- 3-yl)-9H-pyrrolo[2,3- b:5,4-c′]dipyrid-4- yl]-N,N- dimethylaniline | 1H NMR (400 MHz, DMSO- d6) δ ppm: 2.97 (s, 6 H); 6.96 to 7.10 (m, 3 H); 7.53 (t, J = 7.8 Hz, 1 H); 7.70 (dd, J =5.3 and 8.2 Hz, 1 H); 7.88 (s, 1 H); 8.34 (d, J = 7.6 Hz, 1 H); 8.66 (d, J = 4.9 Hz, 1 H); 8.74 (d, J = 2.4 Hz, 1 H); 9.02 (broad s, 1 H); 9.09 (s, 1 H); 12.63 (s, 1H) LC-TOF-MS: Rt (min) = 2.32; [M + H]^{:+} m/z = 384 (ES+) |
| 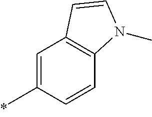 | 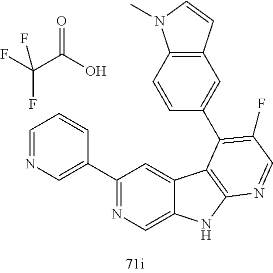 | 3-fluoro-4-(1-methyl- 1H-indol-5-yl)-6- (pyrid-3-yl)-9H- pyrrolo[2,3-b:5,4-c′]- dipyridine | 1H NMR (400 MHz, DMSO- d6) δ ppm: 3.94 (s, 3 H); 6.65 (d, J = 2.9 Hz, 1 H); 7.45 to 7.61 (m, 3 H); 7.77 (d, J = 8.3 Hz, 1 H); 7.89 (s, 1 H); 8.01 (s, 1 H); 8.22 (d, J = 8.3 Hz, 1 H); 8.56 (d, J = 4.6 Hz, 1 H); 8.73 (d, J = 2.7 Hz, 1 H); 8.91 (s, 1 H); 9.07 (s, 1 H); 12.57 (s, 1 H) LC-TOF-MS: Rt (min) = 2.67; m/z = 394 (ES+) |
| 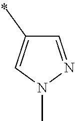 | 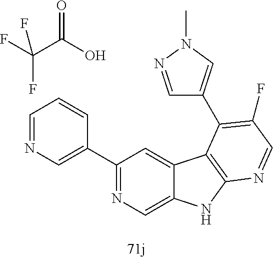 | 3-fluoro-4-(1-methyl- 1H-pyrazol-4-yl)-6- (pyrid-3-yl)-9H- pyrrolo[2,3-b:5,4-c′]- dipyridine | 1H NMR (400 MHz, DMSO- d6) δ ppm: 4.07 (s, 3 H); 7.69 to 7.81 (m, J = 6.2 and 7.0 Hz, 1 H); 8.12 (s, 1 H); 8.46 (s, 1H); 8.48 (s, 1 H); 8.60 (d, J = 8.1 Hz, 1 H); 8.69 (d, J = 2.7 Hz, 1 H); 8.71 (broad s, 1 H); 9.09 (s, 1 H); 9.27 (broad s, 1 H); 12.60 (s, 1 H) LC-TOF-MS: Rt (min) = 2.30; [M + H]^{+:} m/z = 345 (ES+) |
| 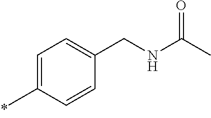 | 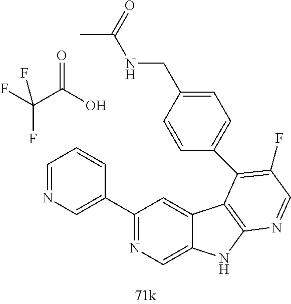 | N-{4-[3-fluoro-6- (pyrid-3-yl)-9H- pyrrolo[2,3-b:5,4-c′]- dipyrid-4-y]benzyl}- acetamide | 1H NMR (400 MHz, DMSO- d6) δ ppm: 1.95 (s, 3 H); 4.44 (d, J = 6.1 Hz, 2 H); 7.60 (d, J = 8.3 Hz, 2 H); 7.67 to 7.70 (m, 1 H); 7.72 (d, J = 7.8 Hz, 2 H); 7.76 (s, 1 H); 8.32 (d, J = 8.8 Hz, 1 H); 8.52 (t, J = 5.9 Hz, 1 H); 8.66 (broad s, 1 H); 8.76 (d, J = 2.4 Hz, 1 H); 9.03 to 9.13 (m, 2 H); 12.66 (s, 1 H) LC-TOF-MS: Rt (min) = 2.32; [M + H]^{+:} m/z = 412 (ES+) |
| 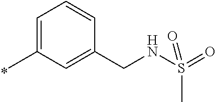 | 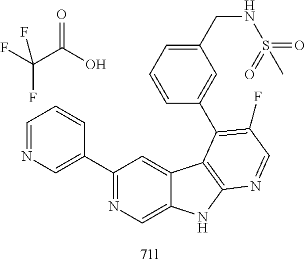 | N-{3-[3-fluoro-6- (pyrid-3-yl)-9H- pyrrolo[2,3-b:5,4-c′]- dipyrid-4-yl]benzyl}- methanesulfonamide | 1H NMR (400 MHz, DMSO- d6) δ ppm: 2.90 (s, 3 H); 4.34 (d, J = 6.1 Hz, 2 H); 7.50 to 7.80 (m, 7 H); 8.28 (d, J = 9.0 Hz, 1 H); 8.59 to 8.65 (m, J = 5.6 Hz, 1 H); 8.77 (d, J = 2.2 Hz, 1 H); 9.02 (broad s, 1 H); 9.09 (s, 1 H); 12.66 (s, 1 H) LC-TOF-MS: Rt (min) = 2.40; [M + H]^{+:} m/z = 448 (ES+) |
| 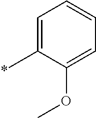 | 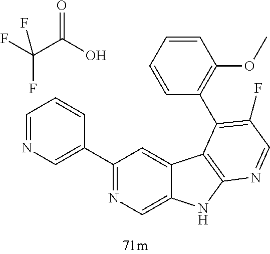 | 3-fluoro-4-(2- methoxyphenyl)-6- (pyrid-3-yl)-9H- pyrrolo[2,3-b:5,4-c′]- dipyridine | 1H NMR (400 MHz, DMSO- d6) δ ppm: 3.72 (s, 3 H); 7.27 (t, J = 7.3 Hz, 1 H); 7.39 (d, J = 8.1 Hz, 1 H); 7.49 (s, 1 H); 7.56 (dd, J = 1.3 and 7.9 Hz, 1 H); 7.58 to 7.64 (m, 1 H); 7.70 (t, J = 7.6 Hz, 1 H); 8.24 (d, J = 8.8 Hz, 1 H); 8.61 (d, J = 4.9 Hz, 1H); 8.72 (d, J = 2.4 Hz, 1 H); 8.95 (s, 1 H); 9.07 (s, 1 H); 12.57 (s, 1 H) LC-TOF-MS: Rt (min) = 2.50; [M + H]^{+:} m/z = 371 (ES+) |
| 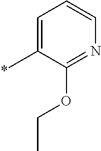 | 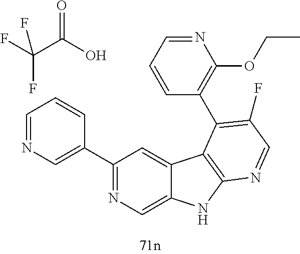 | 4-(2-ethoxypyrid-3- yl)-3-fluoro-6-(pyrid- 3-yl)-9H-pyrrolo[2,3- b:5,4-c′]dipyridine | 1H NMR (400 MHz, DMSO- d6) δ ppm: 1.03 (t, J = 7.0 Hz, 3 H); 4.23 to 4.42 (m, 2 H); 7.32 (dd, J = 4.9 and 7.3 Hz, 1 H); 7.59 (s, 1 H); 7.65 (t, J = 7.3 Hz, 1 H); 8.09 (dd, J = 2.1 and 7.2 Hz, 1 H); 8.32 (d, J = 8.1 Hz, 1 H); 8.51 (dd, J = 2.0 and 4.9 Hz, 1 H); 8.63 (d, J = 5.1 Hz, 1 H); 8.77 (d, J = 2.4 Hz, 1 H); 8.99 to 9.05 (m, 1 H); 9.10 (s, 1 H); 12.64 (s, 1 H) LC-TOF-MS: Rt (min) = 2.48; [M + H]^{+:} m/z = 386 (ES+) |
| 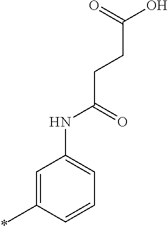 | 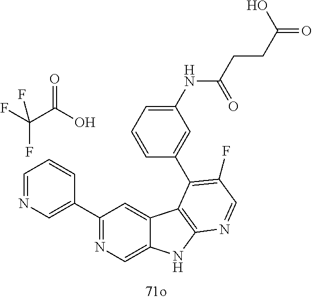 | acide 4-({3-[3-fluoro- 6-(pyrid-3-yl)-9H- pyrrolo[2,3-b:5,4-c′]- dipyrid-4-yl]- phenyl}amino)-4- oxobutanoic acid | 1H NMR (400 MHz, DMSO- d6) δ ppm: 2.53 to 2.57 (m, 2 H); 2.59 to 2.65 (m, J = 6.6 Hz, 2 H); 7.42 (d, J = 8.1 Hz, 1 H); 7.61 to 7.68 (m, 2 H); 7.77 (d, J = 8.3 Hz, 1 H); 7.93 (s, 1 H); 8.12 (s, 1 H); 8.34 (d, J = 8.1 Hz, 1 H); 8.62 (broads, 1 H); 8.77 (d, J = 2.7 Hz, 1 H); 9.04 (broad s, 1 H); 9.10 (s, 1 H); 10.30 (s, 1 H); 12.67 (s, 1 H) LC-TOF-MS: Rt (min) = 2.37; [M + H]^{+:} m/z = 456 (ES+) |
| 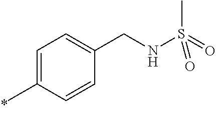 | 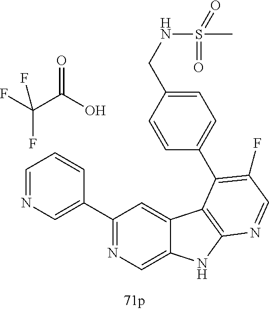 | N-{4-[3-fluoro-6- (pyrid-3-yl)-9H- pynolo[2,3-b:5,4-c′]- dipyrid-4-yl]benzyl}- methanesulfonamide | 1H NMR (400 MHz, DMSO- d6) δ ppm: 2.97 (s, 3 H); 4.37 (d, J = 6.1 Hz, 2 H); 7.63 to 7.72 (m, 3 H); 7.76 (d, J = 8.1 Hz, 4 H); 8.30 (d, J = 8.1 Hz, 1 H); 8.66 (broad s, 1 H); 8.76 (d, J = 2.4 Hz, 1 H); 9.09 (s, 2 H); 12.67 (s, 1 H) LC-TOF-MS: Rt (min) = 2.39; [M + H]^{+:} m/z = 448 (ES+) |
| 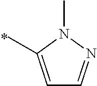 | 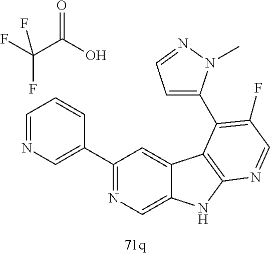 | 3-fluoro-4-(1-methyl- 1H-pyrazol-5-yl)-6- (pyrid-3-yl)-9H- pyrrolo[2,3-b:5,4-c']- dipyridine | 1H NMR (400 MHz, DMSO- d6) δ ppm: 3.74 (s, 3 H); 6.86 (d, J = 2.0 Hz, 1 H); 7.61 (broad s, 1 H); 7.68 (s, 1 H); 7.84 (d, J = 2.0 Hz, 1 H); 8.31 (d, J = 7.6 Hz, 1 H); 8.59 to 8.66 (m, 1 H); 8.85 (d, J = 2.2 Hz, 1 H); 9.07 (broad s, 1 H); 9.13 (s, 1 H); 12.76 (s, 1H) LC-TOF-MS: Rt (min) = 2.25; [M + H]^{+:} m/z = 345 (ES+) |
| 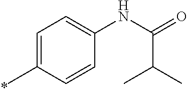 | 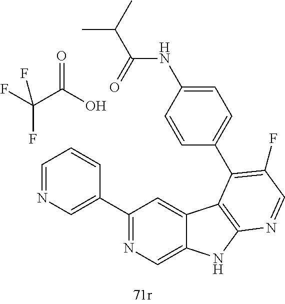 | N-{4-[3-fluoro-6- (pyrid-3-yl)-9H- pyrrolo[2,3-b:5,4-c']- dipyrid-4-yl]phenyl}- 2-methylpropanamide | 1H NMR (400 MHz, DMSO- d6) δ ppm: 1.17 (d, J = 6.8 Hz, 6H); 2.69 (quin, J = 6.9 Hz, 1 H); 7.63 (dd, J = 5.0 and 8.2 Hz, 1 H); 7.73 (d, J = 8.3 Hz, 2 H); 7.91 (s, 1 H); 7.94 (d, J = 8.6 Hz, 2 H); 8.36 (d, J = 8.1 Hz, 1 H); 8.63 (dd, J = 1.6 and 5.0 Hz, 1 H); 8.73 (d, J = 2.7 Hz, 1 H); 9.04 (s, 1 H); 9.09 (d, J = 1.0 Hz, 1 H); 10.17 (s, 1 H); 12.63 (s, 1 H) LC-TOF-MS: Rt (min) = 2.54; [M + H]^{+:} m/z = 426 (ES+) |
| 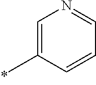 | 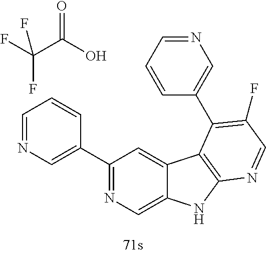 | 3-fluoro-4,6-di(pyrid- 3-yl)-9H-pyrrolo[2,3- b:5,4-c′]dipyridine | 1H NMR (400 MHz, DMSO- d6) δ ppm: 7.58 to 7.67 (m, 1 H); 7.71 (s, 1 H); 7.74 to 7.84 (m, 1 H); 8.20 to 8.38 (m, 2 H); 8.55 to 8.74 (m, 2 H); 8.82 (d, J = 2.7 Hz, 1 H); 8.99 (d, J = 7.6 Hz, 1 H); 9.02 to 9.06 (m, 1 H); 9.12 (s, 1 H); 12.73 (s, 1 H) LC-TOF-MS: Rt (min) = 2.04; [M + H]^{+:} m/z = 342 (ES+) |
| 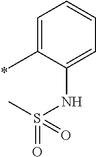 | 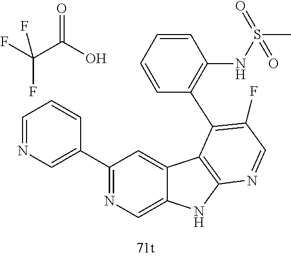 | N-{2-[3-fluoro-6- (pyrid-3-yl)-9H- pyrrolo[2,3-b:5,4-c′]- dipyrid-4-yl]phenyl}- methanesulfonamide | 1H NMR (400 MHz, DMSO- d6) δ ppm: 2.93 (s, 2 H); 6.72 to 6.81 (m, 1 H); 6.85 to 6.91 (m, 1 H); 6.98 to 7.07 (m, 1 H); 7.18 (dd, J = 1.7 and 7.8 Hz, 1 H); 7.42 (s, 1 H); 7.57 (d, J = 7.6 Hz, 1 H); 8.26 (d, J = 8.3 Hz, 1 H); 8.56 to 8.66 (m, 2 H); 8.73 (d, J = 2.4 Hz, 1 H); 8.93 to 9.01 (m, 1 H); 9.06 (s, 1 H); 12.53 (s, 1 H) LC-TOF-MS: Rt (min) = 2.30; [M + H]^{+:} m/z = 434 (ES+) |
| 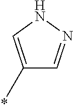 | 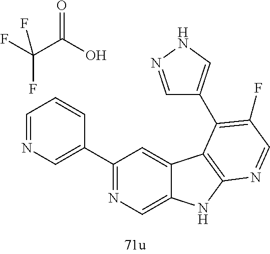 | 3-fluoro-4-(1H- pyrazol-4-yl)-6- (pyrid-3-yl)-9H- pyrrolo[2,3-b:5,4-c′]- dipyridine | LC-TOF-MS: Rt (min) = 2.12; [M + H]^{+:} m/z = 331 (ES+) |
| 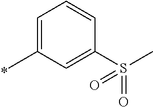 | 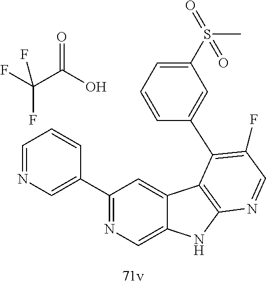 | 3-fluoro-4-[3- (methylsulfonyl) phenyl]-6-(pyrid-3- yl)-9H-pyrrolo[2,3- b:5,4-c′]dipyridine | 1H NMR (400 MHz, DMSO- d6) δ ppm: 3.25 to 3.39 (m, 3 H); 7.62 (dd, J = 5.4 and 8.3 Hz, 1 H); 7.78 (s, 1 H); 8.01 (t, J = 7.8 Hz, 1 H); 8.17 (dd, J = 1.3 and 7.9 Hz, 1 H); 8.25 (dt, J = 1.6 and 7.9 Hz, 1 H); 8.40 (d, J = 7.8 Hz, 1 H); 8.43 (s, 1 H); 8.63 (d, J = 5.4 Hz, 1 H); 8.83 (d, J = 2.4 Hz, 1 H); 9.10 (s, 1 H); 9.13 (s, 1 H); 12.75 (s, 1 H) LC-TOF-MS: Rt (min) = 2.25; [M + H]^{+:} m/z = 419 (ES+) |
| 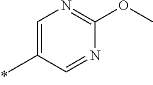 | 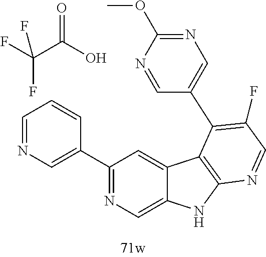 | 3-fluoro-4-(2- methoxypyrimidin-5- yl)-6-(pyrid-3-yl)- 9H-pyrrolo[2;3-b:5,4- c′]dipyridine | 1H NMR (400 MHz, DMSO- d6) δ ppm: 4.10 (s, 3 H); 7.65 to 7.73 (m, 1 H); 8.03 (s, 1 H); 8.51 (d, J = 8.3 Hz, 1 H); 8.64 to 8.68 (m, 1 H); 8.81 (d, J = 2.2 Hz, 1 H); 9.08 (d, J = 1.2 Hz, 2 H); 9.12 (s, 1 H); 9.19 (broad s, 1 H); 12.74 (s, 1 H) LC-TOF-MS: Rt (min) = 2.25; [M +H]^{+:} m/z = 373 (ES+) |
| 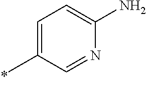 | 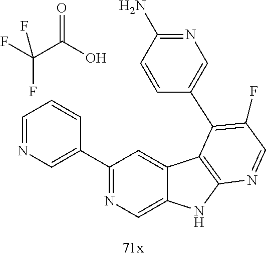 | 5-[3-fluoro-6-(pyrid- 3-yl)-9H-pyrrolo[2,3- b,-c′]dipyrid-4- yl]pyrid-2-amine | 1H NMR (400 MHz, DMSO- d6) δ ppm:7.18 d, J = 9.3 Hz, 1 H); 7.49 to 7.64 (m, 1 H); 8.13 (s, 1 H); 8.21 (d, J = 9.3 Hz, 1 H); 8.40 to 8.52 (m, 2 H); 8.56 to 8.69 (m,J = 4.6 Hz, 1 H); 8.78 (d, J = 2.7 Hz, 1 H); 9.11 (s, 1 H); 9.20 (broad s, 1 H); 12.69 (s, 1H) LC-TOF-MS: Rt (min) = 2.23; [M + H] m/z = 357 (ES+) |

### Examples 75 to 89 (72a-72o)

### General Procedure for the Aryl Amination Reaction (Hartwig-Buchwald)

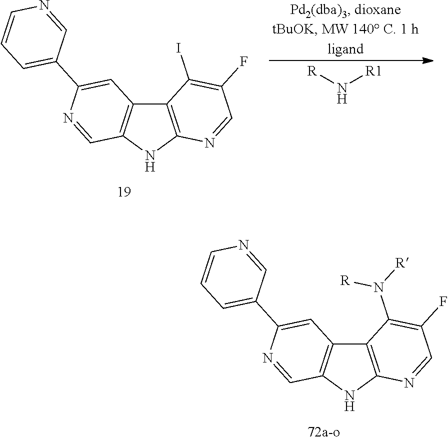

19 mg of R-(+)-2.2′-bis(diphenylphosphino)-1,1′-binaphthyl and 6.0 mg of tris(dibenzylidene-acetone)dipalladium(0) in 1.25 mL of anhydrous 1,4-dioxane are placed in a tube, under an argon atmosphere.

100 mg of 3-fluoro-4-iodo-6-(pyrid-3-yl)-9H-pyrrolo[2,3-b:5,4-c′]dipyridine 19, 65 mg of potassium tert-butoxide and 5 equivalents of amine, the whole in 1.25 mL of anhydrous 1,4-dioxane, are placed in a microwave reactor, under argon, the solution prepared previously is then added and the reactor is sealed and subjected to microwave irradiation for 1 hour at 140° C.

The reaction mixture is poured into a mixture of 150 mL of ethyl acetate, 75 mL of water and 75 mL of saturated aqueous sodium bicarbonate solution. After separation of the phases by settling, the organic phase is dried over magnesium sulfate, filtered and evaporated under reduced pressure. The residue is purified by chromatography on a column of silica, eluting with a 100/0 to 80/20 dichloromethane/methanol mixture, depending on the substrate.

The products are detailed in Table 7 (yield of between 31% and 75% depending on the reagents).

TABLE 7

| Amine | Structure obtenue | Name | Analysis |
|---|---|---|---|
| 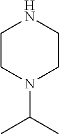 | 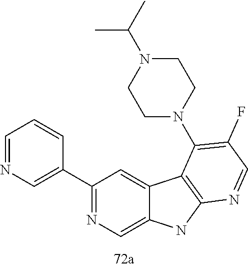 | 3-fluoro-4-[4- (propan-2-yl)- piperazin-1-yl]-6- (pyrid-3-yl)-9H- pyrrolo[2,3-b:5,4- c′]dipyridine | UPLC-MS-DAD-ELSD: Rt (min) = 0.31; [M + H]^{+:} m/z 391; [M − H]^{-:} m/z 389 1H NMR (300 MHz, DMSO-d6) δ□ ppm: 1.08 (d, J = 6.5 Hz, 6 H) 2.74-2.86 (m, 5 H) 3.53 (br. s., 4 H) 7.55 (dd, J = 8.1, 4.8 Hz, 1 H) 8.37 (s, 1 H) 8.41-8.48 (m, 2H) 8.60 (dd, J = 4.8, 1.6 Hz, 1 H) 8.99 (s, 1 H) 9.27 (d, J = 1.7 Hz, 1 H) 12.36 (br. s., 1 H) |
| 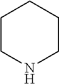 | 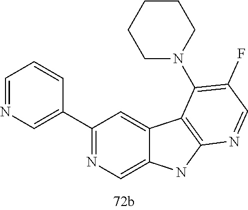 | 3-fluoro-4- (piperid-1-yl)-6- (pyrid-3-yl)-9H- pyrrolo[2,3-b:5,4- c′]dipyridine | UPLC-MS-DAD-ELSD: Rt (min) = 0.31; [M + H]^{+:} m/z 391; [M − H]^{-:} m/z 389 1H NMR (400 MHz, DMSO-d6) δ□ ppm: 1.75 (br. s., 2 H) 1.85 (br. s., 4 H) 3.50 (br. s., 4 H) 7.55 (dd, J = 7.9, 4.6 Hz, 1 H) 8.34 (s, 1 H) 8.41-8.47 (m, 2H) 8.60 (dd, J = 4.8, 1.5 Hz, 1 H) 8.99 (s, 1 H) 9.28 (d, J = 1.8 Hz, 1 H) 12.21 (br. s., 1 H) |
| 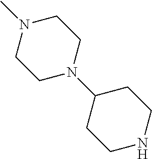 | 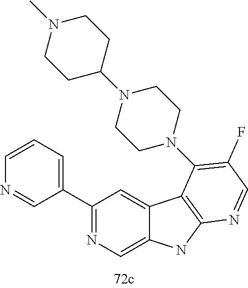 | 3-fluoro-4-[4-(1- methylpiperid-4- yl)piperazin-1- yl]-6-(pyrid-3- yl)-9H-pyrrolo- [2,3-b:5,4-c′]- dipyridine | UPLC-MS-DAD-ELSD: Rt (min) = 0.26; [M + H]^{+:} m/z 446; [M − H]^{-:} m/z 444 1H NMR (400 MHz, DMSO-d6) δ□ ppm: 1.47- 1.58 (m, 2 H) 1.80 (d, J = 10.1 Hz, 2 H) 1.89 (t, J = 11.5 Hz, 2 H) 2.16 (s, 3 H) 2.25-2.32 (m, 1 H) 2.52-2.56 (m, 2 H) 2.82 (br. s., 4 H) 3.53 (br. s., 4 H) 7.54 (dd, J = 7.6, 4.7 Hz, 1 H) 8.36 (s, 1 H) 8.41- 8.47 (m, 2 H) 8.59 (dd, J = 4.8, 1.3 Hz, 1 H) 8.99 (s, 1 H) 9.27 (d, J = 2.0 Hz, 1 H) |
| 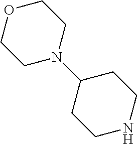 | 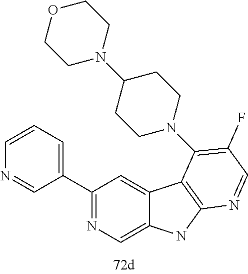 | 3-fluoro-4-[4- (morpholin-4- yl)piperid-1-yl]- 6-(pyrid-3-yl)- 9H-pyrrolo[2,3- b:5,4-c′]- dipyridine | UPLC-MS-DAD-ELSD: Rt (min) = 0.47; [M + H]^{+:} m/z 433; [M − H]^{-:} m/z 431 1H NMR (400 MHz, DMSO-d6) δ□ ppm: 1.68- 1.83 (m, 1 H) 2.05 (d, J = 12.1 Hz, 2 H) 2.57 (t, J = 3.9 Hz, 4 H) 3.34-3.46 (m, 4 H) 3.63 (t, J = 4.4 Hz, 4 H) 3.75 (d, J = 12.5 Hz, 2 H) 7.54 (dd, J = 7.9, 4.8 Hz, 1 H) 8.33 (d, J = 0.4 Hz, 1 H) 8.40-8.47 (m, 2 H) 8.60 (dd, J = 4.8, 1.5 Hz, 1 H) 8.99 (d, J = 0.7 Hz, 1 H) 9.27 (d, J = 2.0 Hz, 1 H) 12.28 (br. s., 1 H) |
| 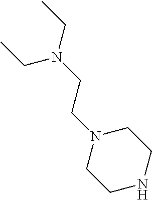 | 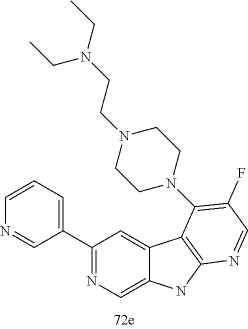 | N,N-diethyl-2- {4-[3-fluoro-6- (pyrid-3-yl)- 9H-pyrrolo[2,3- b:5,4-c′]dipyrid- 4-yl]-piperazin- 1-yl}-ethanamine | UPLC-MS-DAD-ELSD: Rt (min) = 0.56; [M + H]^{+:} m/z 448; [M − H]^{-:} m/z 446 1H NMR (400 MHz, DMSO-d6) δ□ ppm: 0.98 (t, J = 7.1 Hz, 6 H) 2.51-2.62 (m, 8 H) 2.76 (br. s., 4 H) 3.53 (br. s., 4 H) 7.54 (dd, J = 7.9, 4.8 Hz, 1 H) 8.34 (s, 1 H) 8.41-8.47 (m, 2 H) 8.60 (dd, J = 4.8, 1.5 Hz, 1 H) 9.00 (s, 1 H) 9.28 (d, J = 2.4 Hz, 1 H) 12.36 (br. s., 1 H) |
| 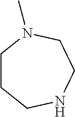 | 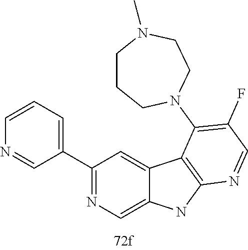 | 3-fluoro-4-(4- methyl-1,4- diazepan-1-yl)-6- (pyrid-3-yl)-9H- pyrrolo[2,3-b:5,4- c′]dipyridine | UPLC-MS-DAD-ELSD: Rt (min) = 0.41; [M + H]^{+:} m/z 377; [M − H]^{-:} m/z 375 1H NMR (400 MHz, DMSO-d6) δ□ ppm: 1.99 (quin, J = 5.4 Hz, 2 H) 2.41 (s, 3 H) 2.72-2.80 (m, 4 H) 3.69 (t, J = 4.8 Hz, 4 H) 7.54 (dd, J = 8.0, 4.7 Hz, 1 H) 8.44 (d, J = 5.7 Hz, 1 H) 8.49 (dt, J = 8.0, 1.9 Hz, 1 H) 8.58 (s, 1 H) 8.59 (dd, J = 4.8, 1.5 Hz, 1 H) 8.98 (d, J = 0.7 Hz, 1 H) 9.34 (d, J = 2.0 Hz, 1 H) 12.26 (br. s.. 1 H) |
| 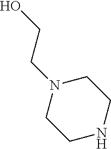 | 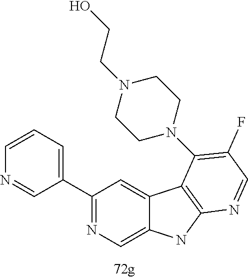 | 2-{4-[3-fluoro-6- (pyrid-3-yl)-9H- pyrrolo[2,3- b:5,4-c′]dipyrid- 4-yl]piperazin-1- yl}ethanol | UPLC-MS-DAD-ELSD: Rt (min) = 0.33; [M + H]^{+:} m/z 393; [M − H]^{-:} m/z 391 1H NMR (400 MHz, DMSO-d6) δ□ ppm: 2.56 (t, J = 6.4 Hz, 2 H) 2.77 (br. s., 4 H) 3.54 (br. s., 4 H) 3.60 (q, J = 5.9 Hz, 2 H) 4.48 (t, J = 5.5 Hz, 1 H) 7.55 (dd, J = 8.0, 4.7 Hz, 1 H) 8.35 (s, 1 H) 8.42- 8.47 (m, 2 H) 8.60 (dd, J = 4.8, 1.5 Hz, 1 H) 9.00 (d, J = 0.7 Hz, 1 H) 9.28 (d, J = 2.0 Hz, 1 H) 12.30 (br. s., 1 H) |
| 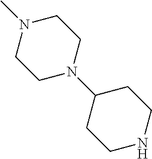 | 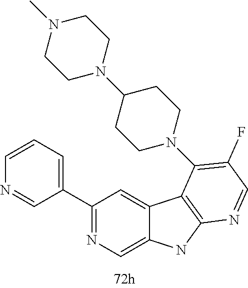 | 3-fluoro-4-[4-(4- methylpiperazin- 1-yl)piperid-1- yl]-6-(pyrid-3- yl)-9H-pyrrolo- [2,3-b:5,4-c′]- dipyridine | UPLC-MS-DAD-ELSD: Rt (min) = 0.31; [M + H]^{+:} m/z 446 1H NMR (400 MHz, DMSO-d6) δ□ ppm: 1.69-1.85 (m, 2 H) 2.01 (d, J = 11.8 Hz, 2 H) 2.17 (s, 3 H) 2.36 (br. s., 4 H) 2.50 (s, 2 H) 2.59 (br. s., 4 H) 3.35-3.48 (m, 1 H) 3.74 (d, J = 11.8 Hz, 2 H) 7.54 (dd, J = 8.1, 4.6 Hz, 1 H) 8.33 (s, 1 H) 8.40-8.48 (m, 2 H) 8.60 (dd, J = 4.8, 1.5 Hz, 1 H) 8.99 (s, 1 H) 9.27 (d, J = 2.0 Hz, 1 H) 12.24 (br. s., 1 H) |
| 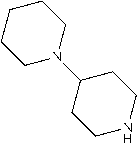 | 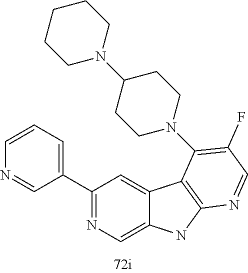 | 4-(1.4′-bipiperid- 1′-yl)-3-fluoro-6- (pyrid-3-yl)-9H- pyrrolo[2,3- b:5,4-c′]- dipyridine | UPLC-MS-DAD-ELSD: Rt (min) = 0.61; [M + H]^{+:} m/z 431 1H NMR (400 MHz, DMSO-d6 + CD3COOD) δ□ ppm: 1.61 (br. s., 2 H) 1.83 (br. s., 4 H) 1.93-2.08 (m, 2 H) 2.29 (d, J = 11.8 Hz, 2 H) 3.28 (br. s, 4 H) 3.40-3.56 (m, 3 H) 3.85 (d, J = 12.5 Hz, 2 H) 7.55 (dd, J = 8.1, 4.8 Hz, 1 H) 8.32 (s, 1 H) 8.44 (d, J = 5.9 Hz, 1 H) 8.50 (dt, J = 8.2, 1.8 Hz, 1 H) 8.60 (d, J = 4.6 Hz, 1 H) 9.02 (s, 1 H) 9.30 (s, 1 H) |
| 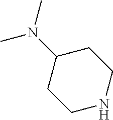 | 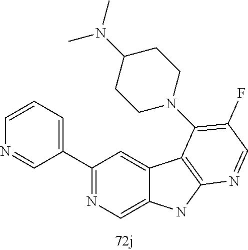 | 1-[3-fluoro-6- (pyrid-3-yl)-9H- pyrrolo[2,3- b:5,4-c′]dipyrid- 4-yl]-N,N- dimethylpiperid- 4-amine | UPLC-MS-DAD-ELSD: Rt (min) = 0.46; [M + H]^{+:} m/z 391 1H NMR (400 MHz, DMSO-d6) δ□ ppm: 1.66-1.81 (m, 2 H) 2.01 (d, J = 11.2 Hz, 2 H) 2.28 (s, 6 H) 2.37-2.47 (m, 1 H) 3.35- 3.43 (m, 2 H) 3.73 (d, J = 12.3 Hz, 2 H) 7.54 (dd, J = 8.0, 4.7 Hz, 1 H) 8.33 (s, 1 H) 8.41- 8.47 (m, 2 H) 8.60 (dd, J = 4.7, 1.4 Hz, 1 H) 8.99 (s, 1 H) 9.27 (d, J = 2.2 Hz, 1 H) 12.27 (br. s., 1 H) |
| 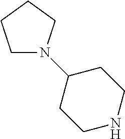 | 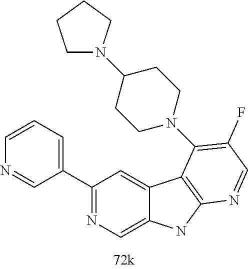 | 3-fluoro-6- (pyrid-3-yl)-4-[4- (pyrrolidin-1- yl)piperid-1-yl]- 9H-pyrrolo[2,3- b:5,4-c′]- dipyridine | UPLC-MS-DAD-ELSD: Rt (min) = 0.53; [M + H]^{+:} m/z 417; [M − H]^{-:} m/z 415 1H NMR (400 MHz, DMSO-d6) δ□ ppm: 1.72-1.87 (m, 6 H) 2.15 (d, J = 12.1 Hz, 2 H) 2.72 (br. s., 4 H) 3.42 (br. s., 3 H) 3.73 (d, J = 12.1 Hz, 2 H) 7.56 (dd, J = 8.0, 4.7 Hz, 1 H) 8.31 (s, 1 H) 8.42-8.49 (m, 2 H) 8.61 (d, J = 4.2 Hz, 1 H) 9.00 (s, 1 H) 9.27 (s, 1 H) 12.30 (s, 1 H) |
| 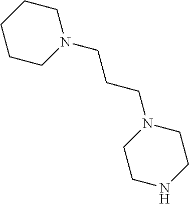 | 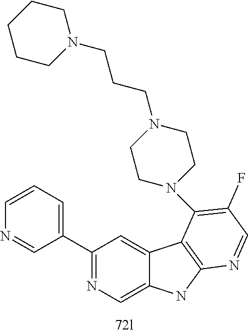 | 3-fluoro-4-{4-[3- (piperid-1- yl)propyl]piper- azin-1-yl}-6-(pyrid- 3-yl)-9H-pyrrolo- [2,3-b:5,4-c′]- dipyridine | UPLC-MS-DAD-ELSD: Rt (min) = 0.25; [M + H]^{+:} m/z 474; [M − H]^{-:} m/z 472 1H NMR (400 MHz, DMSO-d6) δ□ ppm: 1.33-1.42 (m, 2 H) 1.49 (quin, J = 5.5 Hz, 4 H) 1.65 (quin, J = 7.3 Hz, 2 H) 2.25-2.37 (m, 6 H) 2.44 (t, J = 7.3 Hz, 2 H) 2.70 (br. s., 4 H) 3.54 (br. s., 4 H) 7.55 (dd, J = 7.8, 4.5 Hz, 1 H) 8.34 (s, 1 H) 8.41-8.48 (m, 2 H) 8.60 (dd, J = 4.6, 1.5 Hz, 1 H) 9.00 (s, 1 H) 9.27 (d, J = 1.8 Hz, 1 H) 12.33 (br. s., 1 H) |
| 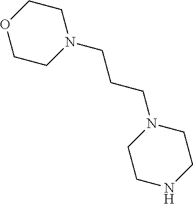 | 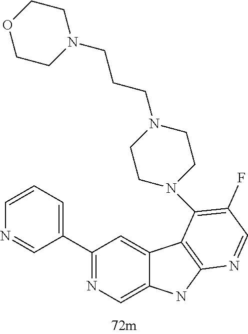 | 3-fluoro-4-{4-[3- (morpholin-4- yl)propyl]piper- azin-1-yl}-6-(pyrid- 3-yl)-9H-pyrrolo- [2,3-b:5,4-c′]- dipyridine | UPLC-MS-DAD-ELSD: Rt (min) = 1.43; [M + H]^{+:} m/z 490; [M − H]^{-:} m/z 488 1H NMR (400 MHz, DMSO-d6) δ□ ppm: 1.67 (quin, J = 7.1 Hz, 2 H) 2.30-2.40 (m, 6 H) 2.43- 2.49 (m, 2 H) 2.71 (br. s, 4 H) 3.54 (br. s., 4 H) 3.58 (t, J = 4.6 Hz, 4 H) 7.55 (dd, J = 7.9, 4.6 Hz, 1 H) 8.35 (s, 1 H) 8.41- 8.48 (m, 2 H) 8.60 (dd, J = 4.7, 1.4 Hz, 1 H) 9.00 (s, 1 H) 9.27 (d, J = 2.2 Hz, 1 H) 12.30 (br. s., 1H) |
| 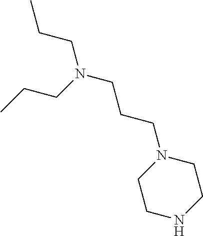 | 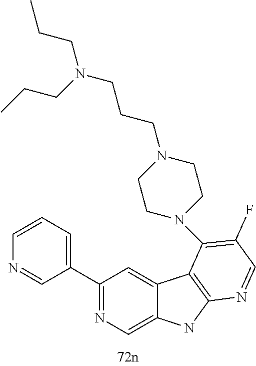 | 3-{4-[3-fluoro-6- (pyrid-3-yl)-9H- pyrrolo[2,3- b:5,4-c′]dipyrid- 4-yl]piperazin-1- yl}-N,N- dipropylpropan- 1-amine | UPLC-MS-DAD-ELSD: Rt (min) = 0.31; [M + H]^{+:} m/z 391; [M − H]^{-:} m/z 389 1H NMR (400 MHz, DMSO-d6) δ□ ppm: 0.85 (t, J = 7.3 Hz, 6 H) 1.40 (sxt, J = 7.3 Hz, 4 H) 1.61 (quin, J = 7.1 Hz, 2 H) 2.32 (t, J = 7.1 Hz, 4 H) 2.40-2.48 (m, 4 H) 2.69 (br. s., 4 H) 3.54 (br. s, 4 H) 7.54 (dd, J = 7.8, 4.7 Hz, 1 H) 8.34 (s, 1 H) 8.40-8.47 (m, 2 H) 8.60 (dd, J = 4.7, 1.4 Hz, 1 H) 9.00 (s, 1 H) 9.27 (d, J = 2.0 Hz, 1 H) 12.31 (br. s., 1 H) |
| 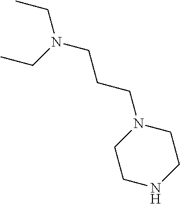 | 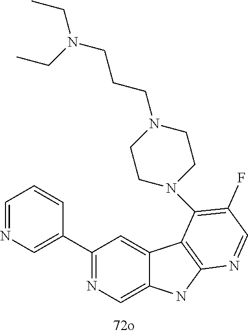 | N,N-diethyl-3- {4-[3-fluoro-6- (pyrid-3-yl)-9H- pyrrolo[2,3- b:5,4-c′]dipyrid- 4-yl]piperazin-1- yl}propan-1- amine | UPLC-MS-DAD-ELSD: Rt (min) = 0.31; [M + H]^{+:} m/z 391; [M − H]^{-:} m/z 389 1H NMR (400 MHz, DMSO-d6) δ□ ppm: 0.98 (t, J = 6.9 Hz, 6 H) 1.58-1.68 (m, 2 H) 2.42-2.49 (m, 8 H) 2.70 (br. s., 4 H) 3.54 (br. s., 4 H) 7.55 (dd, J = 7.8, 4.7 Hz, 1 H) 8.35 (s, 1 H) 8.42- 8.48 (m, 2 H) 8.60 (d, J = 4.6 Hz, 1 H) 9.00 (s, 1 H) 9.27 (s, 1 H) 12.31 (br. s., 1 H) |

### Example 90

### 4-[3-fluoro-6-(pyrid-3-yl)-9H-pyrrolo[2,3-b:5,4-c′]dipyrid-4-yl]-2-methylbut-3-yn-2-amine 73

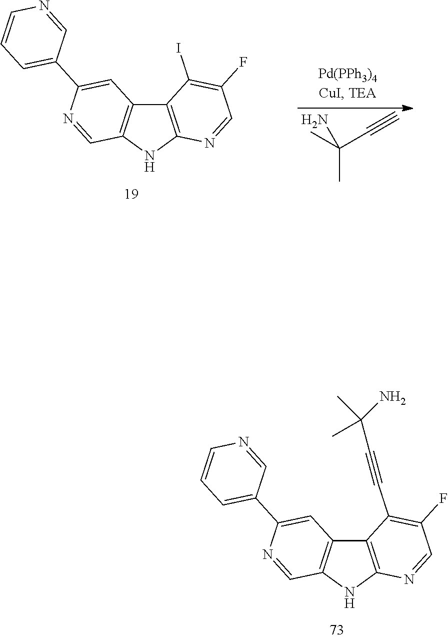

158 mg of 3-fluoro-4-iodo-6-(pyrid-3-yl)-9H-pyrrolo[2,3-b:5,4-c′]dipyridine 19, 50 mg of 1,1-dimethylprop-2-ynylamine, 23 mg of tetrakis(triphenylphosphine)palladium(0) and 3.86 mg of copper (I) iodide in 10 mL of triethylamine are placed in a reactor, and the tube is sealed and subjected to microwave irradiation for 1 hour at 120° C. After 18 hours at room temperature, the reaction mixture is concentrated under reduced pressure and then dissolved in a 50/50 dichloromethane/methanol mixture and 5 g of silica are added. After concentrating under reduced pressure, the solid deposit is purified by chromatography on a column of silica, eluting with a 100/0 to 90/10 dichloromethane/methanol mixture to give 28 mg of 4-[3-fluoro-6-(pyrid-3-yl)-9H-pyrrolo[2,3-b:5,4-c′]dipyrid-4-yl]-2-methylbut-3-yn-2-amine 73 in the form of a beige-coloured powder.

UPLC-MS-DAD-ELSD: Rt (min)=2.10; [M+H]⁺: m/z 346; [M−H]⁻: m/z 344.

1H NMR (400 MHz, DMSO-d6) δ □ppm: 1.59 (s, 6 H) 7.53 (dd, J=7.9, 4.8 Hz, 1 H) 8.48 (dt, J=8.1, 2.0 Hz, 1 H) 8.60 (dd, J=4.6, 1.5 Hz, 1 H) 8.70 (d, J=2.4 Hz, 1 H) 8.83 (d, J=1.0 Hz, 1 H) 9.10 (d, J=1.0 Hz, 1 H) 9.31 (d, J=1.7 Hz, 1 H) 12.56 (br. s., 1 H).

### Example 91

### 4-[3-fluoro-6-(pyrid-3-yl)-9H-pyrrolo[2,3-b:5,4-c′]dipyrid-4-yl]-2-methylbut-3-yn-2-ol 74

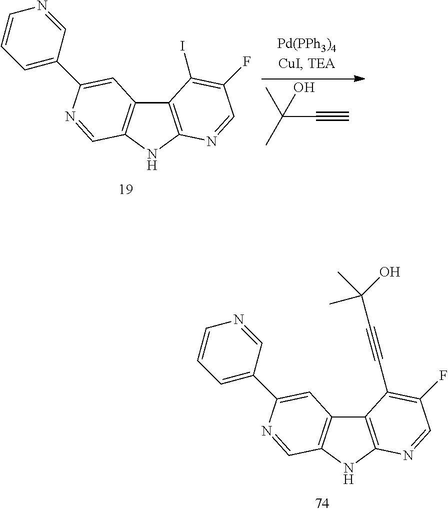

In a manner similar to that for compound 73, 38 mg of 4-[3-fluoro-6-(pyrid-3-yl)-9H-pyrrolo[2,3-b:5,4-c′]dipyrid-4-yl]-2-methylbut-3-yn-2-ol 74 are obtained from 158 mg of 3-fluoro-4-iodo-6-(pyrid-3-yl)-9H-pyrrolo[2,3-b:5,4-c′]dipyridine 19 and 51 mg of 2-methyl-3-butyn-2-ol.

UPLC-MS-DAD-ELSD: Rt (min)=2.80; [M+H]⁺: m/z 347; [M−H]⁻: m/z 345.

1H NMR (400 MHz, DMSO-d6) δ □ppm: 1.68 (s, 6 H) 6.05 (s, 1 H) 7.54 (dd, J=8.0, 4.6 Hz, 1 H) 8.48 (d, J=8.0 Hz, 1 H) 8.62 (br. s., 1 H) 8.72 (d, J=2.4 Hz, 1 H) 8.87 (d, J=1.0 Hz, 1 H) 9.11 (d, =1.2 Hz, 1 H) 9.34 (br. s., 1 H) 12.59 (s, 1 H).

### Example 92

### 4-[3-(4-ethylpiperazin-1-yl)-3-methylbut-1-yn-1-yl]-3-fluoro-6-(pyrid-3-yl)-9H-pyrrolo-[2,3-b:5,4-c′]dipyridine 75

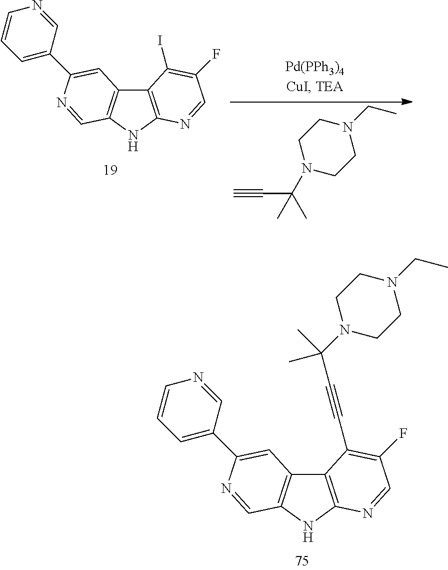

In a manner similar to that for compound 73, 29 mg of 4-[3-(4-ethylpiperazin-1-yl)-3-methylbut-1-yn-1-yl]-3-fluoro-6-(pyrid-3-yl)-9H-pyrrolo[2,3-b:5,4-c′]dipyridine 75 are obtained from 158 mg of 3-fluoro-4-iodo-6-(pyrid-3-yl)-9H-pyrrolo[2,3-b:5,4-c′]dipyridine 19 and 109 mg of 1-(1,1-dimethyl-2-propynyl)-4-ethylpiperazine.

UPLC-MS-DAD-ELSD: Rt (min)=2.42; [M+H]⁺: m/z 443; [M−H]⁻: m/z 441.

1H NMR (400 MHz, DMSO-d6) δ □ppm: 0.90 (t, J=7.2 Hz, 3 H) 1.62 (s, 6 H) 2.25 (q, J=7.2 Hz, 2 H) 2.41 (br. s., 4 H) 2.77 (br. s., 4 H) 7.55 (dd, J=7.8, 4.9 Hz, 1 H) 8.39 (dt, J=8.1, 1.8 Hz, 1 H) 8.61 (dd, J=4.8.1.1 Hz, 1 H) 8.71 (d, J=2.2 Hz, 1 H) 8.73 (d, J=0.7 Hz, 1 H) 9.12 (d, J=0.7 Hz, 1 H) 9.23 (d, J=1.5 Hz, 1H) 12.60 (br. s., 1 H).

### Example 93

### N,N-diethyl-2-({-4-[3-fluoro-6-(pyrid-3-yl)-9H-pyrrolo[2,3-b:5,4-c′]dipyrid-4-yl]-2-methylbut-3-yn-2-yl}oxy)ethanamine 77

Step 1: N,N-diethyl-2-[(2-methylbut-3-yn-2-yl)oxy]ethanamine 76

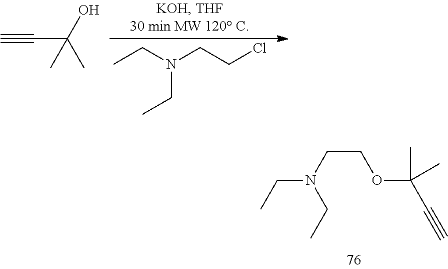

1.42 g of 2-methyl-3-butyl-2-ol, 2.91 g of 2-chlorotriethylamine hydrochloride, 4.75 g of potassium hydroxide and 17 mL of tetrahydrofuran are introduced into a microwave reactor of suitable size. The mixture is stirred for 5 minutes at 25° C. and then irradiated for 30 minutes at 120° C. The medium is diluted with ethyl acetate, washed with water and then treated with aqueous 1N hydrochloric acid solution. The aqueous phase is basified by addition of aqueous sodium hydroxide and then extracted with ethyl acetate. The combined organic phases are dried over magnesium sulfate, filtered and concentrated; the product is taken up in ethyl ether, the suspension filtered and the filtrate concentrated to give 525 mg of N,N-diethyl-2-[(2-methylbut-3-yn-2-yl)oxy]ethanamine 76 in the form of a yellow liquid.

UPLC-SQD: Rt (min)=0.30; [M+H]⁺: m/z 184.

### Step 2:

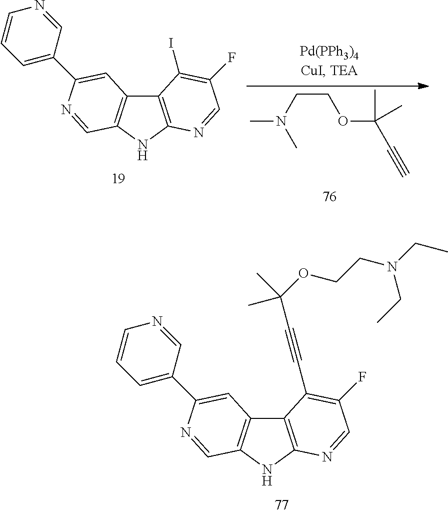

103 mg of 3-fluoro-4-iodo-6-(pyrid-3-yl)-9H-pyrrolo[2,3-b:5,4-c′]dipyridine 19, 35 mg of N,N-diethyl-2-[(2-methylbut-3-yn-2-yl)oxy]ethanamine 76 prepared in step 1, 16 mg of tetrakis(triphenyl-phosphine)palladium(0), 3 mg of copper iodide, 1.5 mL of triethylamine and 0.5 mL of DMF are introduced into a microwave reactor of suitable size. The mixture is irradiated for 1 hour at 120° C. A further 3 mg of copper iodide, 10 mg of tetrakis(triphenylphosphine)palladium(0), 35 mg of the same alkyne as previously and 0.5 mL of DMF are added and the mixture is irradiated again for 1 hour at 120° C. The suspension obtained is diluted with ethyl acetate and water and then filtered. The organic phase is washed twice with water and then dried over magnesium sulfate, filtered and concentrated under reduced pressure. The residue is purified by chromatography on a column of silica, eluting with a 90/10 to 80/20 dichloromethane/methanol mixture to give 44 mg of N,N-diethyl-2-({4-[3-fluoro-6-(pyrid-3-yl)-9H-pyrrolo[2,3-b:5,4-c′]dipyrid-4-yl]-2-methylbut-3-yn-2-yl}oxy)ethanamine 77 in the form of a yellow solid.

1H NMR (400 MHz, DMSO-d6) δ □ppm 0.63 to 1.33 (broad m, 6 H); 1.75 (s, 6 H); 2.18 to 3.44 (partially masked broad m, 6 H); 3.71 to 3.99 (broad m, 2 H); 7.55 (dd, J=4.9 and 8.0 Hz, 1 H); 8.41 (dt, J=1.8 and 8.0 Hz, 1 H); 8.59 to 8.65 (broad m, 1 H); 8.68 (broad s, 1 H); 8.76 (d, J=2.4 Hz, 1 H); 9.13 (d, J=1.0 Hz, 1 H); 9.20 to 9.28 (broad m, 1 H); 12.67 (broad s, 1 H).

LC-MS (7 min): Rt (min)=2.67; [M+H]⁺: m/z 446; [M+2H]²⁺ m/z 223.5 (base peak); [M−H]⁻: m/z 444.

### Example 94

### 3-{-4-[3-fluoro-6-(pyrid-3-yl)-9H-pyrrolo[2,3-b:5,4-c′]dipyrid-4-yl]phenoxy}-N,N-dimethylpropan-1-amine 78

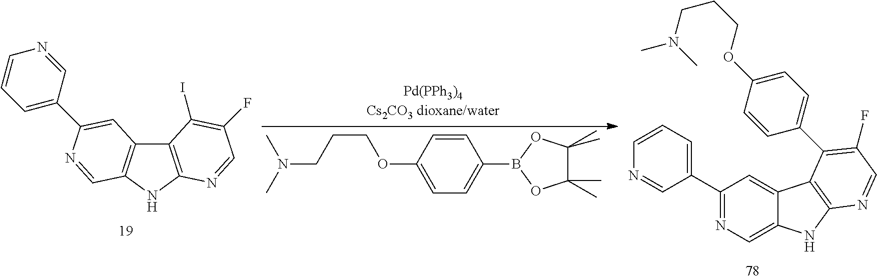

75 mg of 3-fluoro-4-iodo-6-(pyrid-3-yl)-9H-pyrrolo[2,3-b:5,4-c′]dipyridine 19, 176 mg of boronate, 22 mg of tetrakis(triphenylphosphine)palladium(0), 125 mg, 1.25 mL of dioxane and 02.5 mL of aqueous 1.5 M caesium carbonate solution are introduced into a microwave reactor of suitable size. The mixture is irradiated for 45 minutes at 150° C. The suspension obtained is diluted with ethyl acetate and washed three times with water. The organic phase is dried over magnesium sulfate, filtered and then concentrated under reduced pressure. The residue is purified by chromatography on a column of silica, eluting with a 90/10 to 80/20 dichloromethane/methanol mixture to give 39 mg of 3-{4-[3-fluoro-6-(pyrid-3-yl)-9H-pyrrolo[2,3-b:5,4-c′]dipyrid-4-yl]phenoxy}-N,N-dimethylpropan-1-amine 78 in the fond of a yellow solid.

1H NMR (400 MHz, DMSO-d6) δ □ppm 1.90 to 1.98 (m, 2 H); 2.18 (s, 6 H); 2.42 (t, J=6.6 Hz, 2 H); 4.17 (t, J=6.6 Hz, 2 H); 7.27 (d, J=8.3 Hz, 2 H); 7.48 (ddd, J=0.8 and 4.8 and 8.1 Hz, 1 H); 7.70 (d, J=8.3 Hz, 2 H); 7.84 (d, J=1.0 Hz, 1 H); 8.17 (ddd, J=1.7 and 2.2 and 8.1 Hz, 1 H); 8.55 (dd, J=1.7 and 4.8 Hz, 1 H); 8.70 (d, J=2.7 Hz, 1 H); 8.97 (dd, J=0.8 and 2.2 Hz, 1 H); 9.06 (d, J=1.0 Hz, 1 H); 12.21 to 12.69 (broad m, 1 H).

LC-MS (7 min): Rt (min)=2.41; [M+H]⁺: m/z 442; [M+2H]²⁺ m/z 221.5 (base peak); [M−H]⁻: m/z 440.

### Example 95

### 4-[3-fluoro-6-(pyrid-3-yl)-9H-pyrrolo[2,3-b:5,4-c′]dipyrid-4-yl]phenol 79

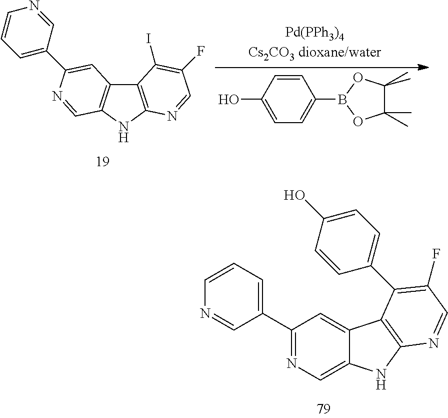

In a manner similar to that for compound 78, starting with 200 mg of 3-fluoro-4-iodo-6-(pyrid-3-yl)-9H-pyrrolo[2,3-b:5,4-c′]dipyridine 19 and 339 mg of 4-(4,4,5,5-tetramethyl-1,3,2-dioxaborolan-2-yl)phenol, 32 mg of 4-[3-fluoro-6-(pyrid-3-yl)-9H-pyrrolo[2,3-b:5,4-c′]dipyrid-4-yl]phenol 79 are obtained in the form of a yellow solid.

1H NMR (400 MHz, DMSO-d6) δ □ppm 7.10 (d, J=8.3 Hz, 2 H); 7.49 (ddd, J=0.8 and 4.8 and 8.1 Hz, 1 H); 7.60 (d, J=8.3 Hz, 2 H); 7.89 (d, J=1.0 Hz, 1 H); 8.19 (ddd, J=1.7 and 2.2 and 8.1 Hz, 1 H); 8.55 (dd, J=1.7 and 4.8 Hz, 1 H); 8.68 (d, J=2.7 Hz, 1 H); 8.97 (dd, J=0.8 and 2.2 Hz, 1 H); 9.06 (d, J=1.0 Hz, 1 H); 9.91 to 10.10 (broad m, 1 H); 12.03 to 12.72 (broad m, 1 H).

LC-MS (7 min): Rt (min)=2.74; [M+H]⁺: m/z 357; [M−H]⁻: m/z 355.

### Example 96

### 2-{4-[3-fluoro-6-(pyrid-3-yl)-9H-pyrrolo[2,3-b:5,4-c′]dipyrid-4-yl]-phenoxy}-N,N-dimethylethanamine 81

Step 1: N,N-dimethyl-2-[4-(4,4,5,5-tetramethyl-1,3,2-dioxaborolan-2-yl)phenoxy]ethanamine 80

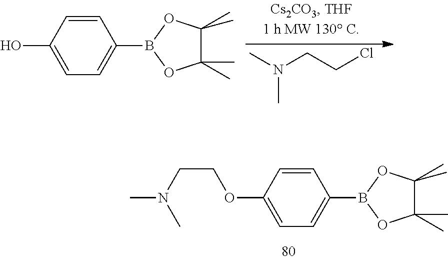

220 mg of 4-(4,4,5,5-tetramethyl-1,3,2-dioxaborolan-2-yl)phenol, 164 mg of 2-dimethylaminoethyl chloride hydrochloride, 1.3 g of caesium carbonate and 4 mL of tetrahydrofuran are introduced into a microwave reactor of suitable size. The mixture is irradiated for 1 hour at 130° C. The medium is diluted with ethyl acetate and washed three times with water. The organic phase is dried over magnesium sulfate, treated with carbon black, filtered and then concentrated under reduced pressure to give 244 mg of N,N-dimethyl-2-[4-(4,4,5,5-tetramethyl-1,3,2-dioxaborolan-2-yl)phenoxy]ethanamine 80 in the form of a brown oil, which is used in crude form in the following step.

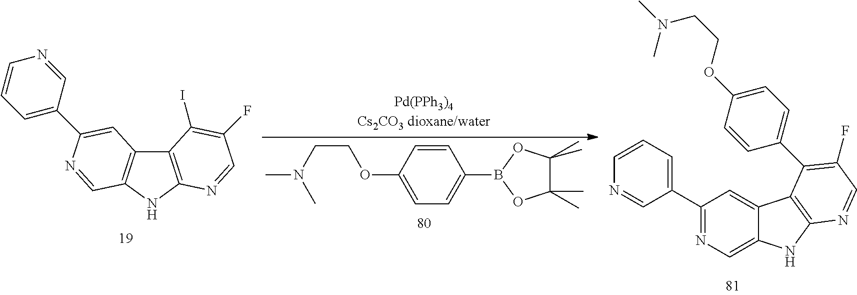

### Step 2:

In a manner similar to that for compound 78, starting with 75 mg of 3-fluoro-4-iodo-6-(pyrid-3-yl)-9H-pyrrolo[2,3-b:5,4-c′]dipyridine 19 and 176 mg of N,N-dimethyl-2-[4-(4,4,5,5-tetramethyl-1,3,2-dioxaborolan-2-yl)phenoxy]ethanamine prepared in step 1, 43 mg of 2-{4-[3-fluoro-6-(pyrid-3-yl)-9H-pyrrolo[2,3-b:5,4-c′]dipyrid-4-yl]phenoxy}-N,N-dimethylethanamine 81 are obtained in the form of a brown solid.

1H NMR (400 MHz, DMSO-d6) δ □ppm 2.42 (s, 6 H); 2.84 to 2.98 (m, 2 H); 4.29 (t, J=5.6 Hz, 2 H); 7.31 (d, J=8.6 Hz, 2 H); 7.48 (dd, J=4.8 and 8.1 Hz, 1 H); 7.72 (d, J=8.6 Hz, 2 H); 7.84 (d, J=1.0 Hz, 1 H); 8.18 (dt, J=2.0 and 8.1 Hz, 1 H); 8.55 (dd, J=2.0 and 4.8 Hz, 1 H); 8.71 (d, J=2.7 Hz, 1 H); 8.96 (d, J=2.0 Hz, 1 H); 9.07 (d, J=1.0 Hz, 1 H); 12.54 (s, 1 H).

UPLC-SQD: Rt (min)=0.42; [M+H]⁺: m/z 428; [M+2H]²⁺ m/z 214.5 (base peak); [M−H]⁻: m/z 426.

### Example 97

### 3-fluoro-6-(pyrid-3-yl)-4-{4-[2-(pyrrolidin-1-yl)ethoxy]phenyl}-9H-pyrrolo[2,3-b:5,4-c′]dipyridine 83

Step 1: 1-{2-[4-(4,4,5,5-tetramethyl-1,3,2-dioxaborolan-2-yl)phenoxy]ethyl}pyrrolidine 82

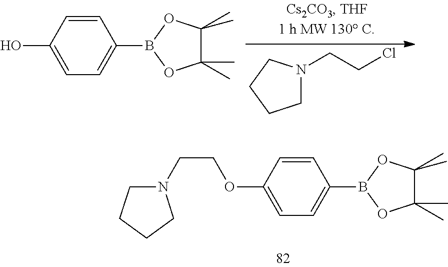

In a manner similar to that for compound 80, starting with 220 mg of 4-(4,4,5,5-tetramethyl-1,3,2-dioxaborolan-2-yl)phenol and 244 mg of 2-pyrrolidinoethyl bromide hydrochloride, 239 mg of 1-{2-[4-(4,4,5,5-tetramethyl-1,3,2-dioxaborolan-2-yl)phenoxy]ethyl}pyrrolidine 82 are obtained in the form of an ochre-coloured oil, which is used in crude form in the following step.

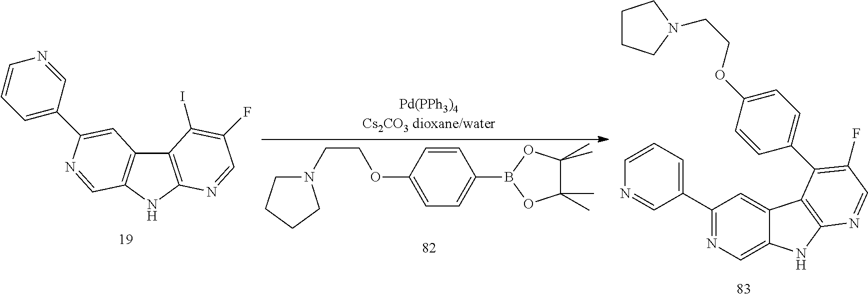

### Step 2:

In a manner similar to that for compound 78, starting with 75 mg of 3-fluoro-4-iodo-6-(pyrid-3-yl)-9H-pyrrolo[2,3-b:5,4-c′]dipyridine 19 and 183 mg of 1-{2-[4-(4,4,5,5-tetramethyl-1,3,2-dioxaborolan-2-yl)phenoxy]ethyl}pyrrolidine prepared in step 1, 32 mg of 3-fluoro-6-(pyrid-3-yl)-4-{4-[2-(pyrrolidin-1-yl)ethoxy]phenyl}-9H-pyrrolo[2,3-b:5,4-c′]dipyridine 83 are obtained in the form of a yellow solid.

1H NMR (400 MHz, DMSO-d6) δ □ppm 1.83 to 1.88 (broad m, 4 H); 2.76 to 3.59 (partially masked broad m, 6 H); 4.33 to 4.39 (broad m, 2 H); 7.33 (d, J=8.5 Hz, 2 H); 7.48 (dd, J=4.7 and 8.1 Hz, 1 H); 7.74 (d, J=8.5 Hz, 2 H); 7.84 (s, 1 H); 8.20 (dt, J=1.7 and 8.1 Hz, 1 H); 8.55 (dd, J=1.7 and 4.7 Hz, 1 H); 8.72 (d, J=2.6 Hz, 1 H); 8.95 (d, J=1.7 Hz, 1 H); 9.07 (s, 1 H); 12.56 (broad s, 1 H).

UPLC-SQD: Rt (min)=0.47; [M+H]⁺: m/z 454; [M−H]⁻: m/z 452.

### Example 98

### 3-{4-[3-fluoro-6-(pyrid-3-yl)-9H-pyrrolo[2,3-b:5,4-c′]dipyrid-4-yl]phenoxy}-N,N,2-trimethylpropan-1-amine 85

Step 1: N,N,2-trimethyl-3-[4-(4,4,5,5-tetramethyl-1,3,2-dioxaborolan-2-yl)phenoxy]propan-1-amine 84

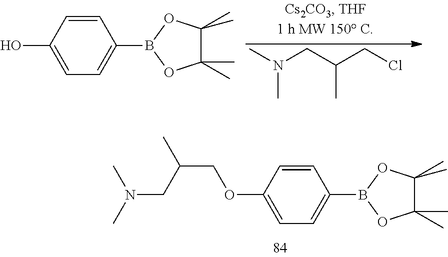

In a manner similar to that for compound 80, starting with 440 mg of 4-(4,4,5,5-tetramethyl-1,3,2-dioxaborolan-2-yl)phenol and 344 mg of 3-dimethylamino-2-methylpropyl chloride hydrochloride (microwave irradiation for 1 hour at 150° C.), 594 mg of N,N,2-trimethyl-3-[4-(4,4,5,5-tetramethyl-1,3,2-dioxaborolan-2-yl)phenoxy]propan-1-amine 84 are obtained in the form of a colourless oil, which is used in crude form in the following step.

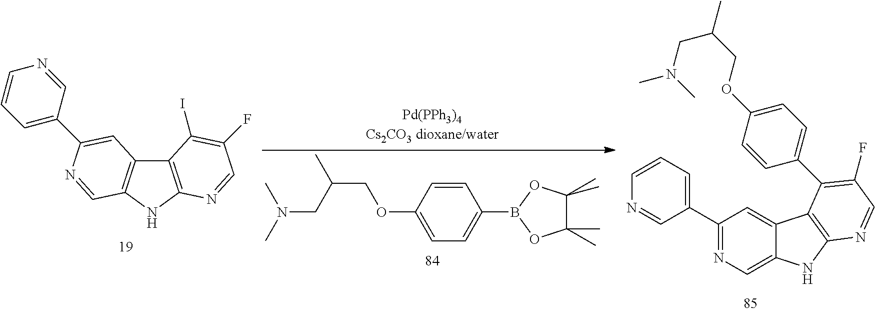

### Step 2:

In a manner similar to that for compound 78, starting with 75 mg of 3-fluoro-4-iodo-6-(pyrid-3-yl)-9H-pyrrolo[2,3-b:5,4-c′]dipyridine 19 and 184 mg of N,N,2-trimethyl-3-[4-(4,4,5,5-tetramethyl-1,3,2-dioxaborolan-2-yl)phenoxy]propan-1-amine prepared in step 1, 26 mg of 3-{4-[3-fluoro-6-(pyrid-3-yl)-9H-pyrrolo[2,3-b:5,4-c′]dipyrid-4-yl]phenoxy}-N,N,2-trimethylpropan-1-amine 85 are obtained in the form of a yellow solid.

1H NMR (400 MHz, DMSO-d6) δ □ppm 1.09 (d, J=6.5 Hz, 3 H); 1.99 to 2.92 (partially masked broad m, 9 H); 3.96 to 4.06 (m, 1 H); 4.11 (dd, J=5.6 and 9.5 Hz, 1 H); 7.29 (d, J=8.5 Hz, 2 H); 7.48 (dd, J=4.7 and 8.1 Hz, 1 H); 7.72 (d, J=8.5 Hz, 2 H); 7.84 (broad s, 1 H); 8.19 (dt, J=1.7 and 8.1 Hz, 1 H); 8.55 (dd, J=1.7 and 4.7 Hz, 1 H); 8.71 (d, J=2.6 Hz, 1 H); 8.95 (d, J=1.7 Hz, 1 H); 9.07 (d, J=1.0 Hz, 1 H); 12.54 (broad s, 1 H).

LC-MS (7 min): Rt (min)=2.50; [M+H]⁺: m/z 456; [M+2H]²⁺: m/z 228.5 (base peak); [M−H]⁻: m/z 454.

### Example 99

### 3-fluoro-4-{4-[2-(morpholin-4-yl)ethoxy]phenyl}-6-(pyrid-3-yl)-9H-pyrrolo[2,3-b:5,4-c′]dipyridine 87

Step 1: 4-{2-[4-(4,4,5,5-tetramethyl-1,3,2-dioxaborolan-2-yl)phenoxy]ethyl}morpholine 86

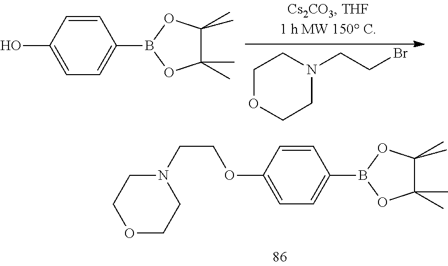

In a manner similar to that for compound 84, starting with 220 mg of 4-(4,4,5,5-tetramethyl-1,3,2-dioxaborolan-2-yl)phenol and 372 mg of 4-(2-chloroethyl)morpholine hydrochloride (microwave irradiation for 1 hour at 150° C.), 356 mg of 4-{2-[4-(4,4,5,5-tetramethyl-1,3,2-dioxaborolan-2-yl)phenoxy]ethyl}morpholine 86 are obtained in the form of a white solid, which is used in crude form in the following step.

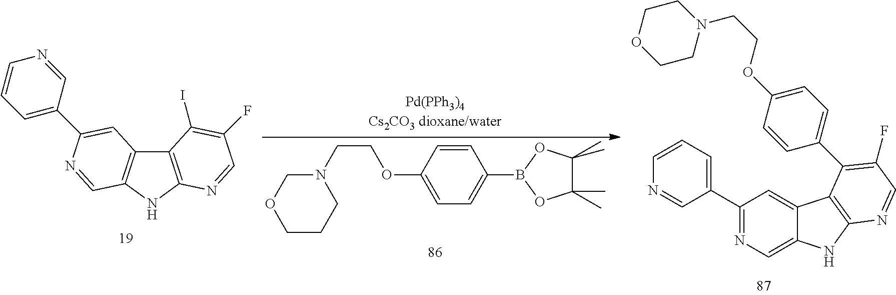

### Step 2:

In a manner similar to that for compound 78, starting with 75 mg of 3-fluoro-4-iodo-6-(pyrid-3-yl)-9H-pyrrolo[2,3-b:5,4-c′]dipyridine 19 and 160 mg of 4-{2-[4-(4,4,5,5-tetramethyl-1,3,2-dioxaborolan-2-yl)phenoxy]ethyl}morpholine prepared in step 1, 40 mg of 3-fluoro-4-{4-[2-(morpholin-4-yl)ethoxy]phenyl}-6-(pyrid-3-yl)-9H-pyrrolo[2,3-b:5,4-c′]dipyridine 87 are obtained in the form of a yellow solid.

1H NMR (400 MHz, DMSO-d6) δ □ppm 2.51 to 2.56 (m, 4 H); 2.79 (t, J=5.6 Hz, 2 H); 3.58 to 3.64 (m, 4 H); 4.27 (t, J=5.6 Hz, 2 H); 7.30 (d, J=8.4 Hz, 2 H); 7.48 (dd, J=4.7 and 8.1 Hz, 1 H); 7.70 (d, J=8.4 Hz, 2 H); 7.83 (d, J=1.2 Hz, 1 H); 8.18 (dt, J=2.0 and 8.1 Hz, 1 H); 8.55 (dd, J=1.7 and 4.7 Hz, 1 H); 8.71 (d, J=2.7 Hz, 1 H); 8.96 (broad d, J=2.0 Hz, 1 H); 9.06 (d, J=1.2 Hz, 1 H); 12.19 to 12.72 (broad m, 1 H).

### Example 100

### N,N-diethyl-2-{4-[3-fluoro-6-(pyrid-3-yl)-9H-pyrrolo[2,3-b:5,4-c′]dipyrid-4-yl]-phenoxy}ethanamine 89

Step 1: N,N-diethyl-2-[4-(4,4,5,5-tetramethyl-1,3,2-dioxaborolan-2-yl)phenoxy]ethanamine 88

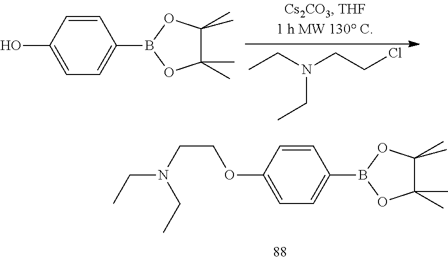

440 mg of 4-(4,4,5,5-tetramethyl-1,3,2-dioxaborolan-2-yl)phenol, 688 mg of 2-chlorotriethylamine hydrochloride, 2.6 g of caesium carbonate and 8 mL of tetrahydrofuran are introduced into a microwave reactor of suitable size. The mixture is irradiated for 1 hour at 150° C. The medium is diluted with ethyl acetate and washed three times with water. The organic phase is dried over magnesium sulfate, filtered and then concentrated under reduced pressure to give 640 mg of N,N-diethyl-2-[4-(4,4,5,5-tetramethyl-1,3,2-dioxaborolan-2-yl)phenoxy]ethanamine 88 in the form of a colourless oil, which is used in crude form in the following step.

LC-MS (7 min): Rt (min)=2.95; [M+H]⁺: m/z 320.

### Step 2:

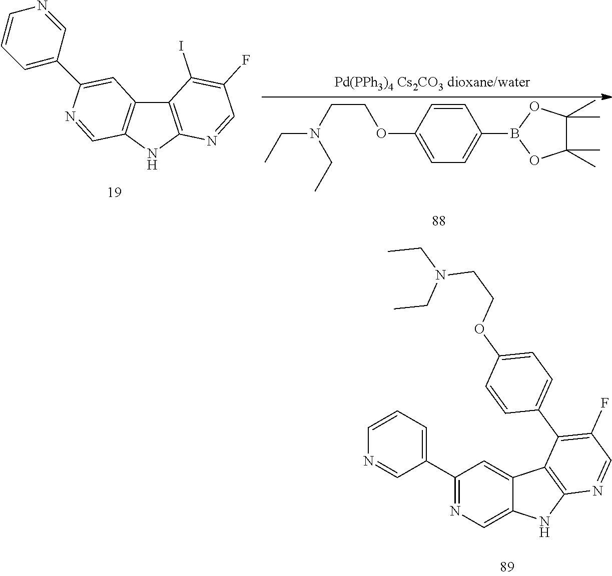

In a manner similar to that for compound 78, starting with 75 mg of 3-fluoro-4-iodo-6-(pyrid-3-yl)-9H-pyrrolo[2,3-b:5,4-c′]dipyridine 19 and 184 mg of N,N-diethyl-2-[4-(4,4,5,5-tetramethyl-1,3,2-dioxaborolan-2-yl)phenoxy]ethanamine prepared in step 1, 20 mg of N,N-diethyl-2-{4-[3-fluoro-6-(pyrid-3-yl)-9H-pyrrolo[2,3-b:5,4-c′]dipyrid-4-yl]phenoxy}ethanamine 89 are obtained in the form of a beige-coloured solid.

1H NMR (400 MHz, DMSO-d6) δ □ppm 0.80 to 1.53 (broad m, 6 H); 2.19 to 3.71 (partially masked broad m, 6 H); 4.05 to 4.46 (broad m, 2 H); 7.31 (broad d, J=8.5 Hz, 2 H); 7.47 (dd, J=4.8 and 8.1 Hz, 1 H); 7.73 (broad d, J=8.5 Hz, 2 H); 7.84 (s, 1 H); 8.19 (broad d, J=8.1 Hz, 1 H); 8.55 (d, J=4.8 Hz, 1 H); 8.71 (d, J=2.6 Hz, 1 H); 8.93 to 8.98 (broad m, 1 H); 9.07 (s, 1 H); 12.52 (broad s, 1 H).

LC-MS (7 min): Rt (min)=2.43; [M+H]⁺: m/z 456; [M+2H]²⁺: m/z 228.5 (base peak); [M−H]⁻: m/z 454.

### Example 101

### 1-{4-[3-fluoro-6-(pyrid-3-yl)-9H-pyrrolo[2,3-b:5,4-c′]dipyrid-4-yl]phenoxy}-3-(morpholin-4-yl)propan-2-ol 90

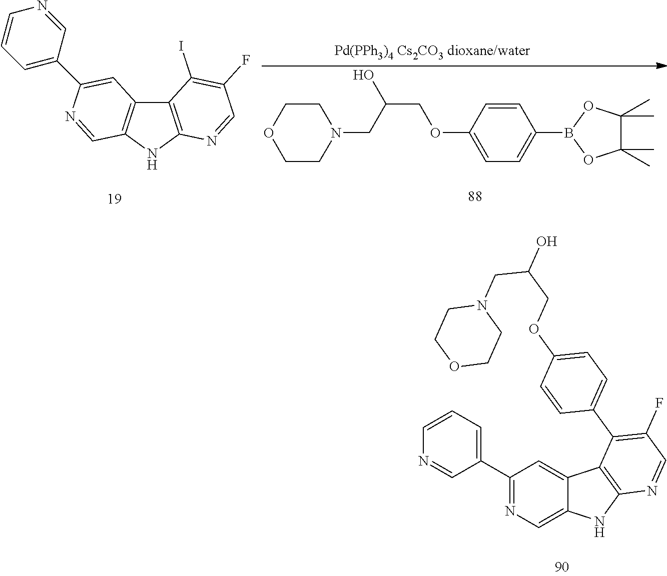

In a manner similar to that for compound 78, starting with 75 mg of 3-fluoro-4-iodo-6-(pyrid-3-yl)-9H-pyrrolo[2,3-b:5,4-c′]dipyridine 19 and 210 mg of boronate, 48 mg of 1-{4-[3-fluoro-6-(pyrid-3-yl)-9H-pyrrolo[2,3-b:5,4-c′]dipyrid-4-yl]phenoxy}-3-(morpholin-4-yl)propan-2-ol 90 are obtained.

1H NMR (400 MHz, DMSO-d6) δ □ppm 2.42 to 2.52 (partially masked m, 6 H); 3.55 to 3.60 (m, 4 H); 4.02 to 4.11 (m, 2 H); 4.13 to 4.20 (m, 1 H); 4.94 to 5.00 (broad m, 1 H); 7.30 (d, J=8.6 Hz, 2 H); 7.48 (ddd, J=0.8 and 4.9 and 8.1 Hz, 1 H); 7.71 (d, J=8.6 Hz, 2 H); 7.85 (d, J=1.0 Hz, 1 H); 8.18 (dt, J=1.7 and 8.1 Hz, 1 H); 8.55 (dd, J=1.7 and 4.9 Hz, 1 H); 8.71 (d, J=2.7 Hz, 1 H); 8.97 (broad d, J=2.0 Hz, 1 H); 9.06 (d, J=1.0 Hz, 1 H); 12.43 (s, 1 H).

UPLC-SQD: Rt (min)=0.44; [M+H]⁺: m/z 500; [M−H]⁻: m/z 498.

### Example 102

### N-ethyl-3-{-4-[3-fluoro-6-(pyrid-3-yl)-9H-pyrrolo[2,3-b:5,4-c′]dipyrid-4-yl]-phenoxy}propan-1-amine 92

Step 1: N-ethyl-3-[4-(4,4,5,5-tetramethyl-1,3,2-dioxaborolan-2-yl)phenoxy]propan-1-amine 91

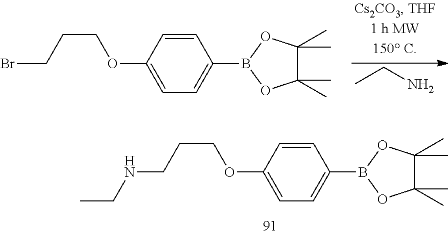

682 mg of 4-(3-bromopropoxy)benzeneboronic acid pinacolate, 2.0 mL of a 2 M solution of ethylamine in tetrahydrofuran, 2.6 g of caesium carbonate and 6 mL of tetrahydrofuran are introduced into a microwave reactor of suitable size. The mixture is irradiated for 1 hour at 150° C. The medium is diluted with ethyl acetate and washed three times with water. The organic phase is dried over magnesium sulfate, filtered and then concentrated under reduced pressure to give 525 mg of N-ethyl-3-[4-(4,4,5,5-tetramethyl-1,3,2-dioxaborolan-2-yl)phenoxy]propan-1-amine 91 in the form of a brown oil, which is used in crude form in the following step.

UPLC-SQD: Rt (min)=0.68; [M+H]⁺: m/z 306.

Step 2: N-ethyl-3-{4-[3-fluoro-6-(pyrid-3-yl)-9H-pyrrolo[2,3-b:5,4-c′]dipyrid-4-yl]phenoxy}propan-1-amine 92

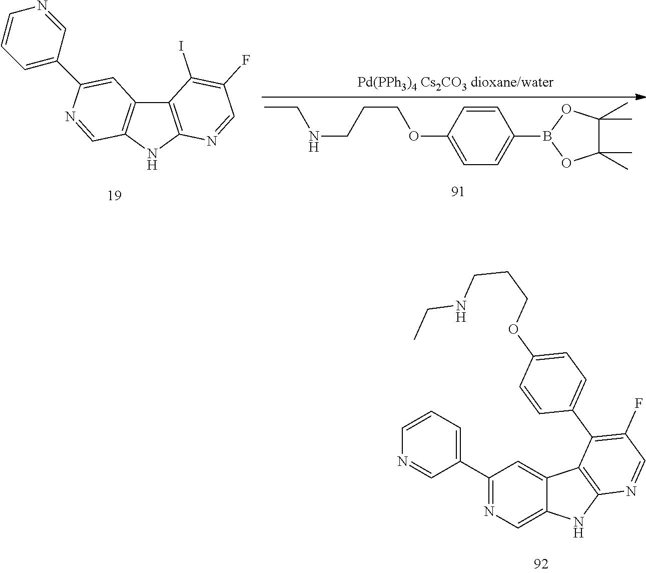

In a manner similar to that for compound 78, starting with 125 mg of 3-fluoro-4-iodo-6-(pyrid-3-yl)-9H-pyrrolo[2,3-b:5,4-c′]dipyridine 19 and 293 mg of boronate prepared in step 1, 107 mg of N-ethyl-3-{-4-[3-fluoro-6-(pyrid-3-yl)-9H-pyrrolo[2,3-b:5,4-c′]dipyrid-4-yl]phenoxy}propan-1-amine 92 are obtained in the form of a yellow solid.

1H NMR (400 MHz, DMSO-d6) δ □ppm 1.16 (t, J=7.2 Hz, 3 H); 2.03 to 2.14 (m, 2 H); 2.82 to 2.93 (m, 3 H); 3.02 (t, J=7.2 Hz, 2 H); 4.24 (t, J=6.1 Hz, 2 H); 7.30 (d, J=8.8 Hz, 2 H); 7.49 (dd, J=4.8 and 8.0 Hz, 1 H); 7.73 (d, J=8.8 Hz, 2 H); 7.85 (d, J=0.5 Hz, 1 H); 8.22 (dt, J=2.0 and 8.0 Hz, 1 H); 8.56 (dd, J=2.0 and 4.8 Hz, 1 H); 8.72 (d, J=2.4 Hz, 1 H); 8.95 (d, J=2.0 Hz, 1 H); 9.07 (d, J=0.5 Hz, 1 H).

UPLC-SQD: Rt (min)=0.50; [M+H]⁺: m/z 442; [M+2H]²⁺: m/z 221.5; [M−H]⁻: m/z 440.

### Example 103 (94) and Example 104

### (2E)-N-[4-(dimethylamino)butyl]-3-[3-fluoro-6-(pyrid-3-yl)-9H-pyrrolo[2,3-b:5,4-c′]dipyrid-4-yl]prop-2-enamide 95

### Step 1

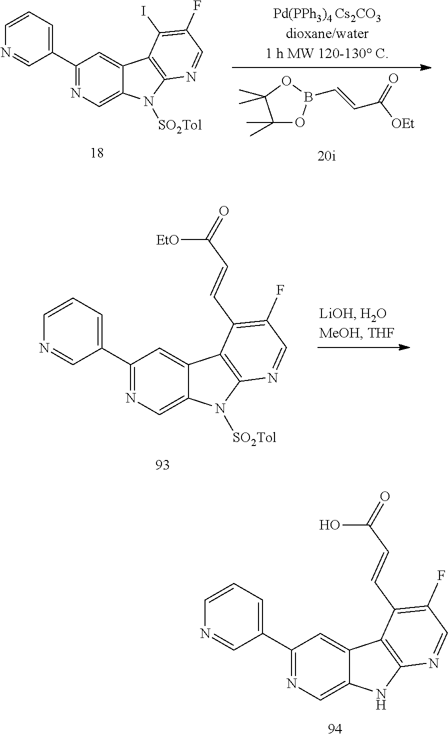

1 g of 3-fluoro-4-iodo-6-pyrid-3-yl-9-(toluene-4-sulfonyl)-9H-pyrrolo[2,3-b:5,4-c′]dipyridine 18, 1.24 g of the boronate 20i, 212 mg of tetrakis(triphenylphosphine)palladium(0), 898 mg of caesium carbonate, 20 mL of dioxane and 5 mL of water are introduced into a microwave reactor of suitable size. The mixture is irradiated for 1 hour at 120° C. The reaction medium is poured into a mixture of ethyl acetate and water with vigorous stirring. After separation of the phases, the organic phase is dried over MgSO₄, filtered and then concentrated under reduced pressure. The residue is dissolved in 10 mL of THF and 10 mL of methanol, and then 1.065 g of lithium hydroxide monohydrate dissolved in 5 mL of water are added. After stirring overnight, 100 mL of water are added and the pH is brought to 5 by addition of aqueous hydrochloric acid solution. The expected compound is recovered by filtration. 552 mg (80%) of (2E)-3-[3-fluoro-6-(pyrid-3-yl)-9H-pyrrolo[2,3-b:5,4-c′]dipyrid-4-yl]prop-2-enoic acid 94 are obtained.

UPLC-MS-DAD-ELSD (LS): Rt (min)=0.56; (M+H)(+): 335(+); (M−H)(−): 333(−).

### Step 2

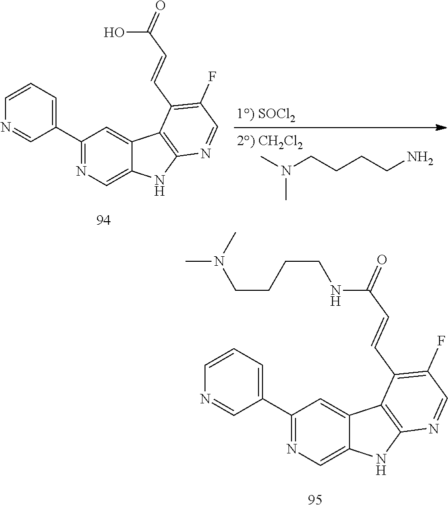

110 mg of (2E)-3-[3-fluoro-6-(pyrid-3-yl)-9H-pyrrolo[2,3-b:5,4-c′]dipyrid-4-yl]prop-2-enoic acid 94 and 10 mL of thionyl chloride are placed in a one-necked flask. The mixture is refluxed overnight with stirring and then concentrated under reduced pressure. The crude product is taken up in 10 mL of dichloromethane, and 456 μl of 4-dimethylaminobutylamine are then added. After 1 hour, the reaction medium is concentrated under vacuum. The residue is purified by chromatography on silica gel (25 g of silica, gradient: 100/0 to 90/10 dichloromethane/2N ammoniacal methanol) to give 82 mg (58%) of the expected compound (2E)-N-[4-(dimethylamino)butyl]-3-[3-fluoro-6-(pyrid-3-yl)-9H-pyrrolo[2,3-b:5,4-c′]dipyrid-4-yl]prop-2-enamide 95.

1H NMR (400 MHz, DMSO-d6) δ ppm: 1.45 to 1.58 (m, 4 H); 2.21 (m, 6 H); 2.30 to 2.38 (m, 2 H); 3.25 to 3.33 (masked m, 2 H); 7.15 (d, J=15.9 Hz, 1 H); 7.56 (ddd, J=1.0 and 4.7 and 8.1 Hz, 1 H); 8.14 (d, J=15.9 Hz, 1 H); 8.41 (dt, J=2.2 and 8.1 Hz, 1 H); 8.50 (d, J=1.0 Hz, 1 H); 8.59 (t, J=6.1 Hz, 1 H); 8.62 (dd, J=1.5 and 4.7 Hz, 1 H); 8.71 (d, J=2.9 Hz, 1 H); 9.10 (d, J=1.0 Hz, 1 H); 9.26 (broad d, J=2.2 Hz, 1 H); 12.08 to 13.05 (broad m, 1 H)

UPLC-SQD: Rt (min)=0.36; [M+H]⁺: m/z 433; [M−H]⁻: m/z 431

### Example 105

### 3-fluoro-4-methoxy-6-(pyrid-3-yl)-9H-pyrrolo[2,3-b:5,4-c′]dipyridine 96

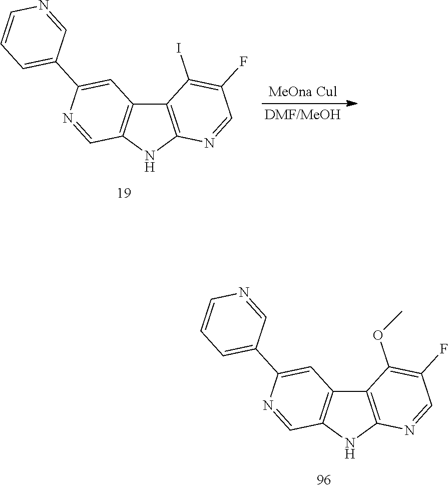

30 mg of 3-fluoro-4-iodo-6-(pyrid-3-yl)-9H-pyrrolo[2,3-b:5,4-c′]dipyridine 19, 166 mg of sodium methoxide and 37 mg of copper (I) iodide in 0.75 mL of dimethylformamide and 0.45 mL of methanol are placed in a reactor, and the tube is sealed and subjected to irradiation for 1 hour at 60° C. The reaction mixture is poured into 50 mL of ethyl acetate and a mixture of 15 mL of water and 15 mL of saturated aqueous ammonium chloride solution. After separation of the phases by settling, the aqueous phase is extracted with 30 mL of ethyl acetate and the combined organic phases are washed with 40 mL of distilled water, dried over sodium sulfate, filtered and concentrated under reduced pressure. The residue is purified by chromatography on a column of silica, eluting with a 100/0 to 95/5 dichloromethane/methanol mixture to give 12 mg of 3-fluoro-4-methoxy-6-(pyrid-3-yl)-9H-pyrrolo-[2,3-b:5,4-c′]dipyridine 96.

UPLC-MS-DAD-ELSD: Rt (min)=2.48; [M+H]⁺: m/z 295; [M−H]⁻: m/z 293.

1H NMR (400 MHz, DMSO-d6) δ □ppm: 4.43 (d, J=5.0 Hz, 3 H) 7.52 (dd, J=8.0, 4.7 Hz, 1 H) 8.49 (d, J=7.9 Hz, 1 H) 8.55-8.61 (m, 3 H) 9.00 (s, 1 H) 9.33 (d, J=2.2 Hz, 1 H) 12.31 (br. s., 1 H).

### Example 106

### 3-(4-methylpiperazin-1-yl)-6-(pyrid-3-yl)-9H-pyrrolo[2,3-b:5,4-c′]dipyridine 97

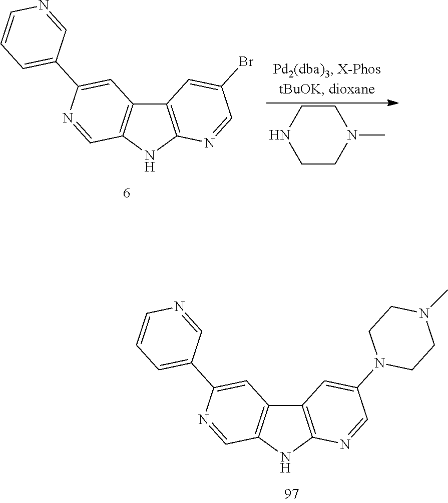

28.2 mg of tris(dibenzylideneacetone)dipalladium(0), 36.7 mg of 2-dicyclohexylphosphino2′,4′,6′-triisopropylbiphenyl and 86.3 g of potassium tert-butoxide in 6 mL of 1,4-dioxane are placed in a reactor. After stirring for 5 minutes under argon, 100 mg of 3-bromo-6-(pyrid-3-yl)-9H-pyrrolo[2,3-b:5,4-c′]dipyridine 6 and 1 mL of 1-methylpiperazine are added. The reaction mixture is stirred for 5 minutes at room temperature and then irradiated by microwave for 1 hour at 140° C. 28.2 mg of tris(dibenzylideneacetone)dipalladium(0), 36.7 mg of 2-dicyclohexylphosphino-2′,4′,6′-triisopropyl-biphenyl and 86.3 g of potassium tert-butoxide again added and the mixture is then irradiated by microwave for one hour at 140° C.

The reaction mixture is concentrated under reduced pressure and then poured into 10 mL of water and 5 ml ethyl acetate. After separation of the phases by settling, the aqueous phase is extracted with four times 5 mL of ethyl acetate and the combined organic phases are then washed with aqueous sodium chloride solution, dried over magnesium sulfate, filtered and concentrated under reduced pressure. The residue is purified by chromatography on a column of silica, eluting with a 100/0 to 50/50 dichloromethane/isopropanol mixture to give 50 mg of 3-(4-methylpiperazin-1-yl)-6-(pyrid-3-yl)-9H-pyrrolo[2,3-b:5,4-c′]dipyridine 97 in the form of a yellow solid.

UPLC-MS-DAD-ELSD: Rt (min)=0.82; [M+H]⁺: m/z 345; [M−H]⁻: m/z 343.

1H NMR (400 MHz, DMSO-d6) δ □ppm: 2.27 (s, 3 H) 2.56 (t, J=5.1 Hz, 4 H) 3.22 (t, J=4.6 Hz, 4 H) 7.52 (dd, J=7.9, 4.5 Hz, 1 H) 8.33 (d, J=2.9 Hz, 1 H) 8.46-8.50 (m, 2 H) 8.57 (dd, J=4.8, 1.6 Hz, 1 H) 8.88 (s, 1 H) 8.95 (d, J=1.0 Hz, 1 H) 9.34 (d, J=2.2 Hz, 1 H) 11.94 (s, 1 H).

### Example 107 (98) and Example 108

### 3-(piperazin-1-yl)-6-(pyrid-3-yl)-9H-pyrrolo[2,3-b:5,4-c′]-dipyridine 99

Step 1: 2-methyl-2-propyl 4-[6-(pyrid-3-yl)-9H-pyrrolo[2,3-b:5,4-c′]dipyrid-3-yl]piperazine-1-carboxylate 98

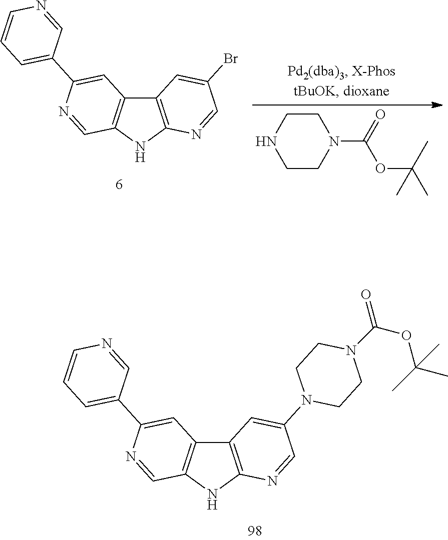

In a manner similar to that for 97, 40 mg of 2-methyl-2-propyl 4-[6-(pyrid-3-yl)-9H-pyrrolo[2,3-b:5,4-c′]dipyrid-3-yl]piperazine-1-carboxylate 98 are obtained from 200 mg of 3-bromo-6-(pyrid-3-yl)-9H-pyrrolo[2,3-b:5,4-c′]dipyridine 6 and 0.468 g of 1-Boc-piperazine. The ligand used for this experiment is 4,5-bis(diphenylphosphino)-9,9-dimethylxanthene.

UPLC-MS-DAD-ELSD: Rt (min)=3.08; [M+H]⁺: m/z 431; [M−H]⁻: m/z 429.

1H NMR (400 MHz, DMSO-d6) δ □ppm: 1.45 (s, 9 H) 3.17 (t, J=4.9 Hz, 4 H) 3.56 (t, J=4.6 Hz, 4 H) 7.53 (ddd, J=8.0, 4.7, 0.7 Hz, 1 H) 8.37 (d, J=2.7 Hz, 1 H) 8.46-8.51 (m, 2 H) 8.58 (dd, J=4.8, 1.6 Hz, 1 H) 8.87 (s, 1 H) 8.97 (d, J=1.0 Hz, 1 H) 9.34 (d, J=2.2 Hz, 1 H) 12.00 (s, 1 H).

Step 2: 3-(piperazin-1-yl)-6-(pyrid-3-yl)-9H-pyrrolo[2,3-b:5,4-c′]dipyridine 99

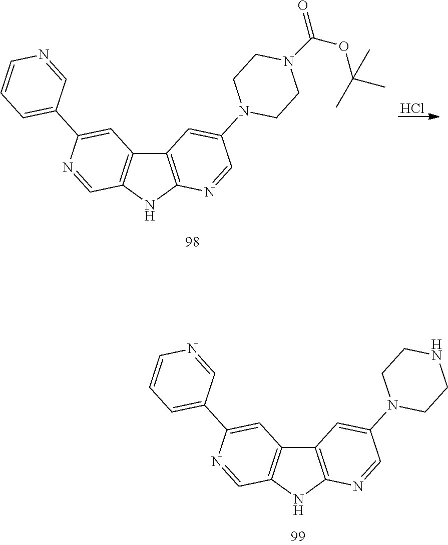

A mixture of 40 mg of 98 and 4 mL of a 4N solution of hydrochloric acid in 1,4-dioxane is stirred for 1 hour at room temperature. After concentrating, the reaction mixture is diluted with 100 μl of acetic acid and 350 μl of water and then purified by preparative LCMS to give 8.8 mg of 3-(piperazin-1-yl)-6-(pyrid-3-yl)-9H-pyrrolo[2,3-b:5,4-c′]dipyridine in the form of the trifluoroacetic acid salt 99.

UPLC-MS-DAD-ELSD: Rt (min)=0.28; [M+H]⁺: m/z 331.

1H NMR (300 MHz, DMSO-d6) δ □ppm: 3.40 (masked m, 8 H) 7.63 (dd, J=7.8, 4.9 Hz, 1 H) 8.41 (d, J=2.6 Hz, 1 H) 8.55 (d, J=2.8 Hz, 1 H) 8.58 (dt, J=8.1, 2.0 Hz, 1 H) 8.64 (dd, J=4.8, 1.6 Hz, 1 H) 8.79 (br. s., 2 H) 8.91 (s, 1 H) 9.00 (d, J=1.1 Hz, 1 H) 9.36 (d, J=2.2 Hz, 1 H) 12.13 (s, 1 H).

### Example 109

### 6-(pyrid-3-yl)-9H-pyrrolo[2,3-b:5,4-c′]dipyrid-3-amine 102

Step 1: 5′-chloro-5″-nitro-3,2′:4′,3″-terpyrid-2″-amine 100

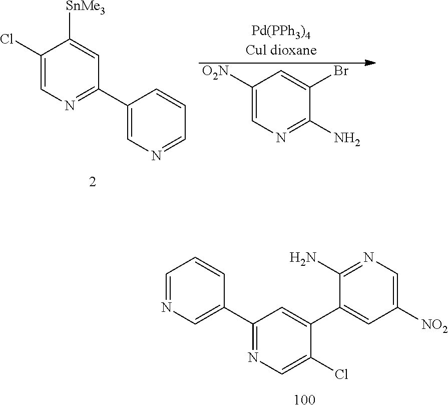

4 g of 2-amino-3-bromo-5-nitropyridine, 6.8 g of 5-chloro-4-trimethylstannyl-2-(3′-pyridyl)pyridine 2, 1.49 g of tetrakis(triphenylphosphine)palladium(0) and 734 mg of copper (I) iodide in 80 mL of 1,4-dioxane are placed in a reactor under argon, and the tube is sealed. After stirring for 5 minutes under argon, the reactor is subjected to microwave irradiation for 2 hours at 120° C. The reaction medium is concentrated under reduced pressure and then taken up in a 50/50 dichloromethane/methanol mixture and filtered through Clarcel. After concentrating under reduced pressure, 7.11 g of 5′-chloro-5″-nitro-3,2′:4′,3″-terpyrid-2″-amine 100 are obtained in the form of a brown-yellow powder.

UPLC-MS-DAD-ELSD (LS): Rt (min)=0.88; (M+H)(+): 328(+)/ . . . ; (M−H)(−): 326(−)/ . . . (presence of a chlorine atom).

Step 2: 5′-chloro-3,2′:4′,3″-terpyridine-2″,5″-diamine 101

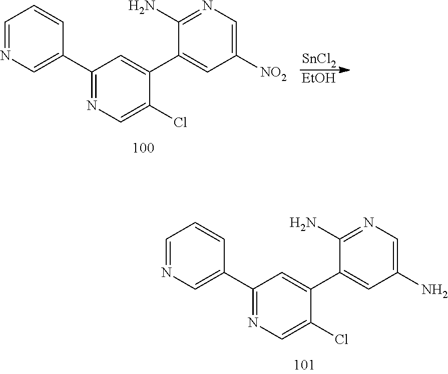

A mixture of 7.11 g of 5′-chloro-5″-nitro-3,2′:4′,3″-terpyrid-2″-amine 100 and 24.48 g of tin (II) chloride dihydrate in 300 ml of ethanol is refluxed for 2.5 hours. After concentrating under reduced pressure, the reaction mixture is diluted with 1 litre of ethyl acetate and 1 litre of water and is then stirred for 18 hours at room temperature. After filtering through Clarcel, the mixture is separated by settling and the aqueous phase is then brought to pH 8 with aqueous sodium hydrogen carbonate solution and extracted with five times 1 litre of ethyl acetate. The organic phases are combined and then evaporated under reduced pressure to give 3.67 g of 5′-chloro-3,2′:4′,3″-terpyridine-2″,5″-diamine 101 in the form of a black powder.

UPLC-MS-DAD-ELSD (LS): Rt (min)=0.32; (M+H)(+): 298(+)/ . . . (presence of a chlorine atom).

Step 3: 6-(pyrid-3-yl)-9H-pyrrolo[2,3-b:5,4-c′]dipyrid-3-amine 102

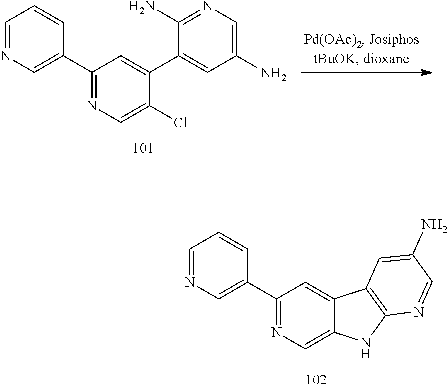

752 mg of (R)-(−)-1-[(S)-2-(dicyclohexylphosphino)ferrocenyl]ethyldi-tert-butylphosphine and 277 mg of palladium (II) acetate in 3 mL of anhydrous 1,4-dioxane are placed in a tube, under an argon atmosphere, and stirred for 10 minutes at 40° C.

3.97 g of 5′-chloro-3,2′:4′,3″-terpyridine-2″,5″-diamine 101 and 2.1 g of potassium tert-butoxide in 35 mL of anhydrous 1,4-dioxane are placed in a reactor under argon, the solution prepared previously is then added, and the tube is sealed and subjected to microwave irradiation for 2 hours at 125° C. After leaving to stand at room temperature for 18 hours, the reaction mixture is poured into 500 mL of water and 500 mL of ethyl acetate, a greenish precipitate appears. This precipitate is filtered off by suction under vacuum and is taken up in 50 mL of water acidified with aqueous 1N hydrochloric acid solution and then neutralized with sodium hydrogen carbonate powder. After filtering and drying under vacuum, the solid obtained is taken up in a 50/50 dichloromethane/methanol mixture, 15 g of silica are added, and the mixture is concentrated under reduced pressure and purified by chromatography on a column of silica, eluting with a 100/0 to 90/10 dichloromethane/methanol mixture to give 911 mg of 6-(pyrid-3-yl)-9H-pyrrolo[2,3-b:5,4-c′]dipyrid-3-amine 102 in the form of a yellow powder.

1H NMR (400 MHz, DMSO-d6) δ □ppm 5.10 (s, 2 H) 7.50 (dd, J=7.9, 4.8 Hz, 1 H) 7.81 (d, J=2.4 Hz, 1 H) 8.12 (d, J=2.4 Hz, 1 H) 8.50 (dt, J=8.0, 1.9 Hz, 1 H) 8.56 (dd, J=4.8, 1.3 Hz, 1 H) 8.73 (s, 1 H) 8.90 (s, 1H) 9.36 (d, J=2.2 Hz, 1 H) 11.68 (s, 1 H).

UPLC-SQD: Rt (min)=0.24; [M+H]⁺: m/z 262.

### Example 110

### N-propyl-6-(pyrid-3-yl)-9H-pyrrolo[2,3-b:5,4-c′]dipyrid-3-amine 103

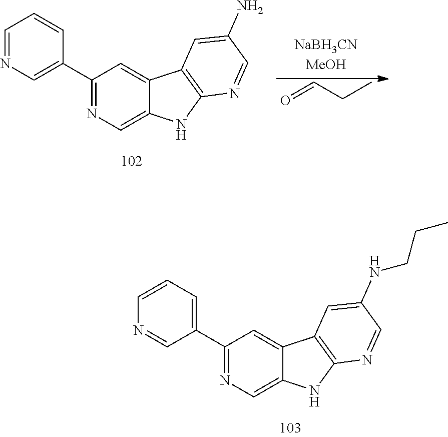

A mixture of 100 mg of 6-(pyrid-3-yl)-9H-pyrrolo[2,3-b:5,4-c′]dipyrid-3-amine 102, 55.8 μl of propionaldehyde and 29 mg of sodium cyanoborohydride in 5 mL of methanol is stirred at room temperature for 18 hours. 55.8 μl of propionaldehyde and 29 mg of sodium cyanoborohydride are added and the mixture is stirred for 4 hours at room temperature. A further 55.8 μl of propionaldehyde and 29 mg of sodium cyanoborohydride are added to the reaction mixture, which is then stirred for 18 hours at room temperature. The reaction mixture is diluted with 10 ml of saturated aqueous sodium hydrogen carbonate solution and then concentrated under reduced pressure. This residue is taken up in 200 mL of ethyl acetate and 200 mL of water. After separation of the phases by settling, the organic phase is concentrated under reduced pressure. The residue obtained is taken up in a 50/50 dichloromethane/methanol mixture, 2 g of silica are added, and the mixture is concentrated under reduced pressure to give a solid deposit, which is purified by chromatography on a column of silica, eluting with a 100/0 to 95/5 dichloromethane/methanol mixture to give 65 mg of N-propyl-6-(pyrid-3-yl)-9H-pyrrolo[2,3-b:5,4-c′]dipyrid-3-amine 103 in the form of a beige-coloured powder.

1H NMR (400 MHz, DMSO-d6) ε □ppm: 1.02 (t, J=7.5 Hz, 3 H) 1.67 (sext., J=7.2 Hz, 2 H) 3.10 (q, J=6.8 Hz, 2 H) 5.66 (t, J=5.5 Hz, 1 H) 7.50 (ddd, J=8.0, 4.7, 0.7 Hz, 1 H) 7.80 (d, J=2.7 Hz, 1 H) 8.17 (d, J=2.7 Hz, 1 H) 8.50 (dt, J=8.1, 1.7 Hz, 1 H) 8.56 (dd, J=4.6, 1.7 Hz, 1 H) 8.81 (d, J=1.0 Hz, 1 H) 8.90 (d, J=1.2 Hz, 1 H) 9.35 (dd, J=2.2, 0.7 Hz, 1 H) 11.70 (br. s., 1 H).

LC-MS (7 min): Rt (min)=2.28; [M+H]⁺: m/z 304.

### Example 111

### 6-(pyrid-3-yl)-3-(2,2,2-trifluoroethoxy)-9H-pyrrolo[2,3-b:5,4-c′]dipyridine 104

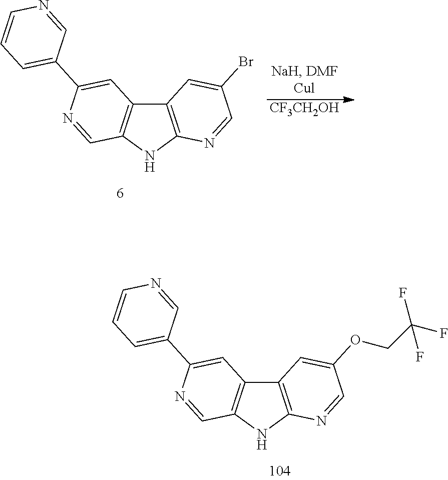

5 mL of trifluoroethanol in 0.5 mL of dimethylformamide are placed in a tube and 115 mg of 60% sodium hydride in oil are added at 0° C. After stirring for 1 hour at room temperature, the solution is poured into a reactor containing a mixture of 160 mg of 3-bromo-6-(pyrid-3-yl)-9H-pyrrolo[2,3-b:5,4-c′]dipyridine 6 and 290 mg of copper (I) iodide, and the tube is sealed and subjected to microwave irradiation for 30 minutes at 140° C. The reaction mixture is diluted with 5 mL of dimethylformamide, filtered through Celite and then washed with 20 mL of dimethylformamide. After concentrating under reduced pressure, the residue is purified by preparative HPLC to give, after freeze-drying, 17.5 mg of 6-(pyrid-3-yl)-3(2,2,2-trifluoroethoxy)-9H-pyrrolo[2,3-b:5,4-c′]dipyridine 104 in the form of the trifluoroacetic acid salt as a pale yellow lyophilizate.

1H NMR (400 MHz, DMSO-d6) δ □ppm 4.94 (q, J=8.8 Hz, 2 H) 7.74-7.80 (m, 1 H) 8.54 (s, 2 H) 8.68-8.77 (m, 2 H) 8.94 (s, 1 H) 9.05 (d, J=1.0 Hz, 1 H) 9.42 (br. s., 1 H) 12.34 (s, 1 H).

UPLC-SQD: Rt (min)=0.59; [M+H]⁺: m/z 345; [M−H]⁻: m/z 343.

### Example 112

### 3-ethoxy-6-(pyrid-3-yl)-9H-pyrrolo[2,3-b:5,4-c′]dipyridine 105

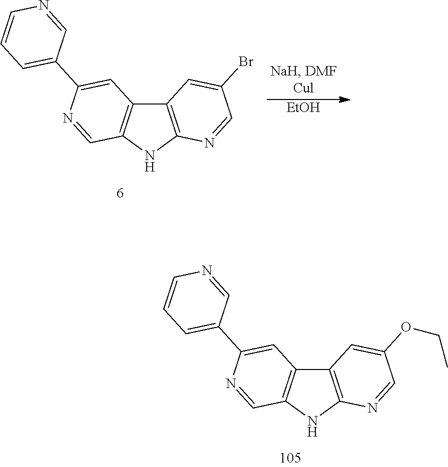

492 mg of 60% sodium hydride in oil, washed beforehand with three times 2 mL of pentane, and 1.3 ml of ethanol are placed in a round-bottomed flask. After stirring for 1 hour at room temperature, this solution is poured into a reactor containing 100 mg of 3-bromo-6-(pyrid-3-yl)-9H-pyrrolo[2,3-b:5,4-c′]dipyridine 6, 146 mg of copper iodide (I) and 0.65 mL of dimethylformamide. The reaction mixture is subjected to microwave irradiation for 1 hour at 120° C. and then poured into a mixture of 50 mL of ethyl acetate and aqueous ammonium chloride solution with vigorous stirring. After separation of the phases by settling, the organic phase is dried over sodium sulfate, filtered and concentrated to dryness. The residue is purified by chromatography on a column of silica, eluting with a 100/0 to 95/5 dichloromethane/methanol mixture to give 21 mg of 3-ethoxy-6-(pyrid-3-yl)-9H-pyrrolo[2,3-b:5,4-c′]dipyridine 105.

1H NMR (400 MHz, DMSO-d6) δ ppm 1.43 (t, J=7.0 Hz, 3 H) 4.21 (q, J=7.1 Hz, 2 H) 7.53 (dd, J=7.9, 4.8 Hz, 1 H) 8.38 (s, 2 H) 8.48 (dt, J=8.1, 2.0 Hz, 1 H) 8.58 (dd, J=4.6, 1.5 Hz, 1 H) 8.88 (s, 1 H) 8.98 (d, J=0.7 Hz, 1 H) 9.34 (d, J=2.0 Hz, 1 H) 12.07 (br. s., 1 H).

LC-MS (7 min): Rt (min)=2.53; [M+H]⁺: m/z 291; [M−H]⁻: m/z 289.

### Example 113

### 3-(2-methoxyethoxy)-6-(pyrid-3-yl)-9H-pyrrolo[2,3-b:5,4-c′]dipyridine 106

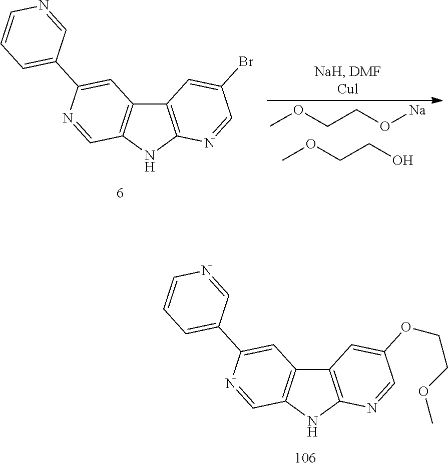

180 mg of 3-bromo-6-(pyrid-3-yl)-9H-pyrrolo[2,3-b:5,4-c′]dipyridine 6, 169 mg of copper iodide (I), 4.1 mL of 21% sodium methoxyethanoate dissolved in methoxyethanol and 0.4 mL of dimethylformamide are placed in a reactor, and the tube is sealed and subjected to microwave irradiation for 45 minutes at 120° C.

The rest of the protocol is the same as that for compound 105. After purification, 17 mg of 3-(2-methoxyethoxy)-6-(pyrid-3-yl)-9H-pyrrolo[2,3-b:5,4-c′]dipyridine 106 are obtained.

1H NMR (400 MHz, DMSO-d6) δ □ppm 3.36 (s, 3 H) 3.76 (t, J=4.9 Hz, 2 H) 4.27 (t, J=4.4 Hz, 2 H) 7.53 (dd, J=7.9, 4.8 Hz, 1 H) 8.39-8.41 (m, 2 H) 8.48 (dt, J=8.0, 2.0 Hz, 1 H) 8.58 (dd, J=4.6, 1.7 Hz, 1 H) 8.87 (d, J=1.0 Hz, 1 H) 8.99 (d, J=1.0 Hz, 1 H) 9.34 (d, J=2.2 Hz, 1H) 12.11 (br. s., 1 H).

LC-MS (7 min): Rt (min)=2.31; [M+H]⁺: m/z 321; [M−H]⁻: m/z 319.

### Example 114

### 3-iodo-6-(pyrid-3-yl)-9H-pyrrolo[2,3-b:5,4-c′]dipyridine 107

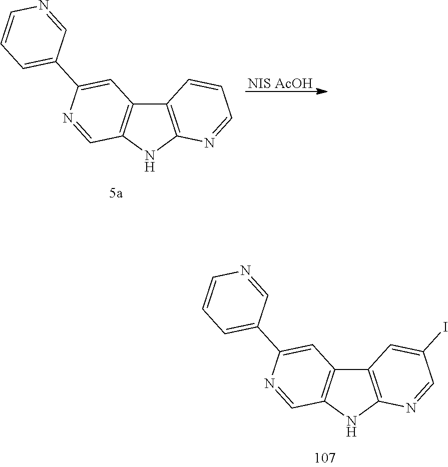

A mixture of 500 mg of 6-(pyrid-3-yl)-9H-pyrrolo[2,3-b:5,4-c′]dipyridine 5a in 5 ml of acetic acid and 502 mg of N-iodosuccinimide is stirred at room temperature for 4 hours and then heated for 1 hour at 80° C. After concentrating under reduced pressure, the residue is purified by chromatography on a column of silica, eluting with a 100/0 to 0/100 heptane/ethyl acetate mixture to give 150 mg of 3-iodo-6-(pyrid-3-yl)-9H-pyrrolo[2,3-b:5,4-c′]dipyridine 107 in the form of a dark brown solid.

1H NMR (400 MHz, DMSO-d6) δ □ppm 7.54 (dd, J=8.2, 4.8 Hz, 1 H) 8.48 (dt, J=7.9, 1.8 Hz, 1 H) 8.59 (dd, J=4.6, 1.5 Hz, 1 H) 8.77 (d, J=2.0 Hz, 1 H) 8.92 (d, J=0.5 Hz, 1 H) 9.03 (d, J=1.0 Hz, 1 H) 9.13 (d, J=2.0 Hz, 1 H) 9.33 (d, J=2.4 Hz, 1 H) 12.38 (br. s., 1 H).

### Examples 115 to 127 (111a-111m)

Step 1: 1-methyl-4-{3-[4-(4,4,5,5-tetramethyl-1,3,2-dioxaborolan-2-yl)-1H-pyrazol-1-yl]propyl}-piperazine 108

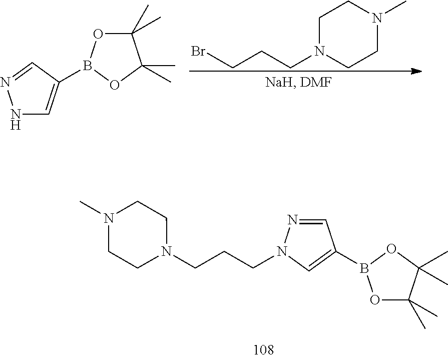

330 mg of 60% sodium hydride in oil and 1 mL of dimethylformamide are placed in a reactor, under an argon atmosphere, followed by dropwise addition of 500 mg of 4-(4,4,5,5-tetramethyl-1,3,2-dioxaborolan-2-yl)-1H-pyrazole dissolved in 4 mL of dimethylformamide (temperature at the end of addition in the region of 32° C.). Once the evolution of gas has ceased, 987 mg of 3-(N-methylpiperazine)propyl bromide dihydrobromide suspended in 20 mL of dimethylformamide are added. The reaction mixture is stirred for 24 hours at room temperature and then poured into a mixture of 100 mL of water and 100 mL of ethyl acetate. After separation of the phases by settling, the aqueous phase is extracted with four times 100 mL of ethyl acetate and the combined organic phases are washed once with water, dried over magnesium sulfate, filtered and then concentrated under reduced pressure to give 2 g of 1-methyl-4-{3-[4-(4,4,5,5-tetramethyl-1,3,2-dioxaborolan-2-yl)-1H-pyrazol-1-yl]propyl}piperazine 108 in the form of a colourless oil.

LC (4 min)-MS-DAD-ELSD (LS): Rt (min)=0.76; (M+H)(+): 335(+).

Step 1 bis: 2-[3,5-dimethyl-4-(4,4,5,5-tetramethyl-1,3,2-dioxaborolan-2-yl)-1H-pyrazol-1-yl]-N,N-diethylethanamine 109

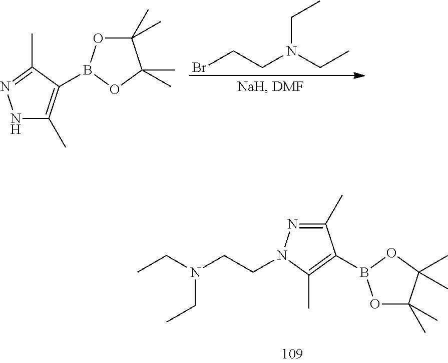

In a manner similar to that for 108, 1.65 g of 2-[3,5-dimethyl-4-(4,4,5,5-tetramethyl-1,3,2-dioxaborolan-2-yl)-1H-pyrazol-1-yl]-N,N-diethylethanamine 109 in the form of a colourless oil is obtained from 1 g of 3,5-dimethyl-4-(4,4,5,5-tetramethyl-1,3,2-dioxaborolan-2-yl)-1H-pyrazole and 1.18 g of 2-bromo-N,N-diethylethylamine hydrobromide.

1H NMR (400 MHz, DMSO-d6) δ □ppm 0.89 (t, J=7.1 Hz, 6 H) 1.24 (s, 12 H) 2.16 (s, 3 H) 2.33 (s, 3 H) 2.45 (q, J=7.2 Hz, 4 H) 2.63-2.68 (m, 2 H) 3.92 (t, J=7.0 Hz, 2 H).

Step 1 ter: N,N-diethyl-3-[4-(4,4,5,5-tetramethyl-1,3,2-dioxaborolan-2-yl)-1H-pyrazol-1-yl]propan-1-amine 110

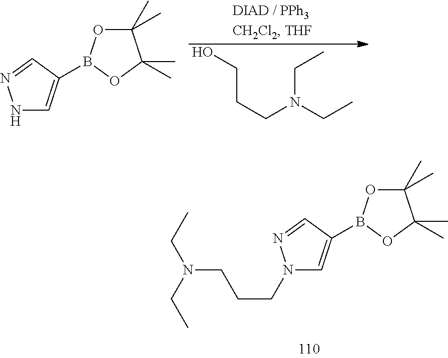

500 mg of 4-(4,4,5,5-tetramethyl-1,3,2-dioxaborolan-2-yl)-1H-pyrazole, 338 mg of 3-diethylamino-1 propanol and 1.91 g of triphenylphosphine supported on resin at 1.57 mmol/g in 10 mL of dichloromethane are placed in a reactor, under an argon atmosphere, followed by dropwise addition of 0.61 mL of diisopropyl azodicarboxylate (temperature at the end of the addition in the region of 32° C.). After stirring for 1 hour at room temperature and adding 5 mL of tetrahydrofuran, followed by refluxing for 6 hours, the reaction mixture is filtered under vacuum, rinsed with tetrahydrofuran and then concentrated under reduced pressure to give 1.42 g of N,N-diethyl-3-[4-(4,4,5,5-tetramethyl-1,3,2-dioxaborolan-2-yl)-1H-pyrazol-1-yl]propan-1-amine 110 in the form of a yellow oil, which is used in crude form for the rest of the synthesis.

LC (4 min)-MS-DAD-ELSD (LS): Rt (min)=0.95; (M+H)(+): 308(+).

### Procedure Procedure—Suzuki Coupling in Position 3

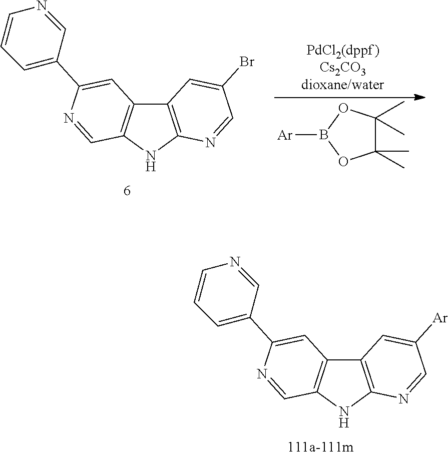

145 mg of 3-bromo-6-(pyrid-3-yl)-9H-pyrrolo[2,3-b:5,4-c′]dipyridine 6 in 0.5 mL of 1,2-dimethoxyethane, 1.45 mL of aqueous 2N sodium carbonate solution, 0.03 equivalent of tetrakis(triphenylphosphine)palladium(0) or 0.15 equivalent of 1,1′-bis(diphenylphosphino)ferrocene-dichloropalladium(II) and 1.6 equivalents of boronate (commercial or prepared during step 1) are placed in a reactor, and the tube is sealed and subjected to microwave irradiation for 10 to 30 minutes from 120 to 180° C. After concentrating under reduced pressure, the reaction mixture is purified by chromatography on a column of silica, eluting with a 100/0 to 90/10 dichloromethane/methanol mixture. The product 111b was also purified by preparative HPLC in acidic medium using a 95/5 to 40/60 gradient of water+0.07% trifluoroacetic acid/acetonitrile+0.07% trifluoroacetic acid. The products 111a to 111m obtained are detailed in Table 8 (yield of between 8% and 55% depending on the reagents).

| Reagent | | Name of the | |
|---|---|---|---|
| Boronic acid | | neutral | |
| or ester | Structure obtained | compound | Analysis |
| 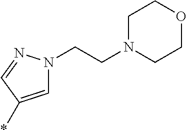 | 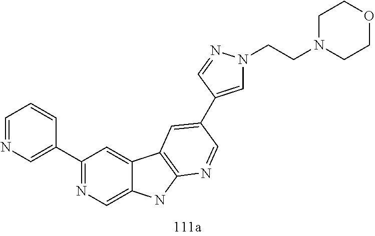 | 3-{1-[2- (morpholin-4- yl)ethyl]-1H- pyrazol-4-yl}- 6-(pyrid-3-yl)- 9H-pyrrolo [2,3-b:5,4- c′]dipyridine | 1H NMR (400 MHz, DMSO-d6) δ □ ppm 2.43-2.47 (m, 4H) 2.79 (t, J = 6.7 Hz, 2H) 3.55-3.60 (m, 4H) 4.30 (t, J = 6.6 Hz, 2H) 7.54 (dd, J = 8.1, 4.9 Hz, 1H) 7.99 (s, 1H) 8.30 (s, 1H) 8.50 (dt, J = 8.1, 1.9 Hz, 1H) 8.59 (dd, J = 4.6, 1.5 Hz, 1H) 8.86-8.89 (m, 2H) 8.91 (d, J = 2.2 Hz, 1H) 9.02 (d, J = 1.0 Hz, 1H) 9.35 (d, J = 1.7 Hz, 1H) 12.24 (br. s., 1H). |
| 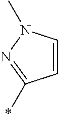 | 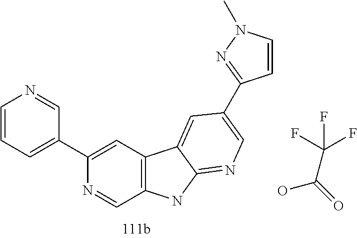 | 3-(1-methyl- 1H-pyrazol-3- yl)-6- (pyrid-3-yl)- 9H-pyrrolo [2,3-b:5,4- c′]dipyridine | UPLC-SQD: Rt (min) = 0.59; [M + H]^{+:} m/z 345; [M − H]^{-:} m/z 343. 1H NMR (400 MHz, DMSO-d6) δ □ ppm 3.96 (s, 3H) 6.57 (d, J = 2.0 Hz, 1H) 7.56 (d, J = 1.7 Hz, 1H) 7.77 (dd, J = 7.8, 4.9 Hz, 1H) 8.71 (d, J = 4.9 Hz, 1H) 8.74 (d, J = 7.6 Hz, 1H) 8.80 (d, J = 2.2 Hz, 1H) 8.92 (d, J = 2.0 Hz, 1H) 9.05 (s, 1H) 9.10 (d, J = 1.0 Hz, 1H) 9.42 (d, J = 2.0 Hz, 1H) 12.59 (s, 1H). |
| 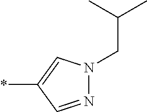 | 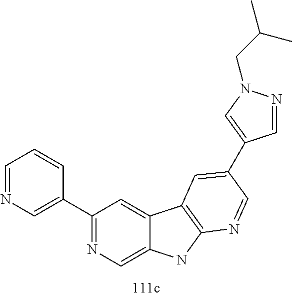 | 3-[1-(2-methyl- propyl)-1H- pyrazol-4-yl]- 6-(pyrid-3- yl)-9H- pyrrolo[2,3- b:5,4-c′] dipyridine | UPLC-SQD: Rt (min) = 0.61; [M + H]^{+:} m/z 369; [M − H]^{-:} m/z 367. 1H NMR (400 MHz, DMSO-d6) δ □ ppm 0.91 (d, J = 6.6 Hz, 6H) 2.19 (spt, J = 6.6 Hz, 1H) 3.99 (d, J = 6.6 Hz, 2H) 7.54 (dd, J = 8.1, 4.6 Hz, 1H) 7.99 (s, 1H) 8.26 (s, 1H) 8.50 (dt, J = 8.0, 1.9 Hz, 1H) 8.59 (dd, J = 4.8, 1.6 Hz, 1H) 8.87 (d, J = 0.7 Hz, 1H) 8.89 (d, J = 2.2 Hz, 1H) 8.92 (d, J = 2.2 Hz, 1H) 9.02 (d, J = 1.0 Hz, 1H) 9.35 (d, J = 1.7 Hz, 1H) 12.23 (br. s., 1H). |
| 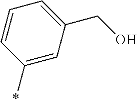 | 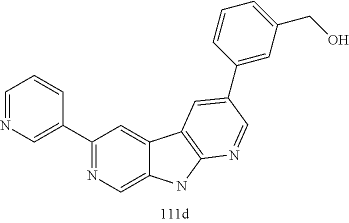 | {3-[6-(pyrid-3- yl)-9H-pyrrolo- [2,3-b:5,4-c′]- dipyrid-3-yl]- phenyl} methanol | UPLC-SQD: Rt (min) = 0.50; [M + H]^{+:} m/z 353; [M − H]^{-:} m/z 351. 1H NMR (400 MHz, DMSO-d6) δ □ ppm 4.63 (d, J = 4.4 Hz, 2H) 5.28 (t, J = 5.1 Hz, 1H) 7.38 (d, J = 7.3 Hz, 1H) 7.48-7.57 (m, 2H) 7.70 (d, J = 8.1 Hz, 1H) 7.78 (s, 1H) 8.52 (dt, J = 8.1, 1.9 Hz, 1H) 8.59 (dd, J = 4.8, 1.6 Hz, 1H) 8.94 (d, J = 2.2 Hz, 1H) 9.02 (d, J = 1.0 Hz, 1H) 9.05 (d, J = 1.0 Hz, 1H) 9.06 (d, J = 2.2 Hz, 1H) 9.38 (d, J = 1.7 Hz, 1H) 12.34 (br. s., 1H). |
| 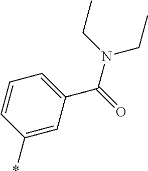 | 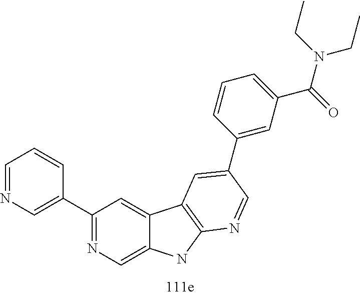 | N,N-diethyl-3- [6-(pyrid-3-yl)- 9H-pyrrolo [2,3-b:5,4-c′] dipyrid-3-yl]- benzamide | LC-MS (7 min): Rt (min) = 2.96; [M + H]^{+:} m/z 422; [M − H]^{-:} m/z 420. 1H NMR (400 MHz, DMSO-d6) δ □ ppm 1.06-1.28 (m, 6H) 3.21-3.32 (m, 2H) 3.42-3.56 (m, 2H) 7.38 (d, J = 7.3 Hz, 1H) 7.55 (dd, J = 7.7, 4.8 Hz, 1H) 7.61 (t, J = 7.7 Hz, 1H) 7.78 (s, 1H) 7.92 (d, J = 8.3 Hz, 1H) 8.51 (dt, J = 8.1, 1.8 Hz, 1H) 8.60 (dd, J = 4.8, 1.6 Hz, 1H) 9.00 (d, J = 2.4 Hz, 1H) 9.03 (d, J = 1.0 Hz, 1H) 9.05 (d, J = 1.0 Hz, 1H) 9.14 (d, J = 2.2 Hz, 1H) 9.37 (d, J = 1.5 Hz, 1H)12.42 (br. s., 1H). |
| 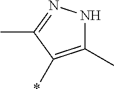 | 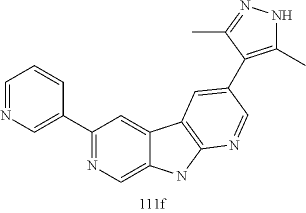 | 3-(3,5- dimethyl- 1H-pyrazol-4- yl)-6-(pyrid-3- yl)-9H- pyrrolo[2,3- b:5,4-c′]- dipyridine | UPLC-SQD: Rt (min) = 0.41; [M + H]^{+:} m/z 341. 1H NMR (400 MHz, DMSO-d6) δ □ ppm 2.28 (br. s., 6H) 7.54 (dd, J = 7.7, 5.0 Hz, 1H) 8.51 (dt, J = 7.9, 1.9 Hz, 1H) 8.54 (d, J = 2.2 Hz, 1H) 8.59 (dd, J = 4.6, 1.7 Hz, 1H) 8.65 (d, J = 2.0 Hz, 1H) 8.98 (d, J = 0.7 Hz, 1H) 9.03 (d, J = 1.0 Hz, 1H) 9.37 (d, J = 1.7 Hz, 1H) 12.31 (br. s., 1H) 12.42 (br. s., 1H). |
| 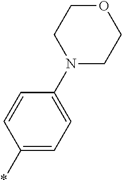 | 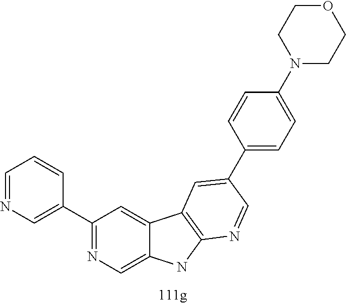 | 3-[4- (morpholin- 4-yl)phenyl]- 6-(pyrid-3- yl)-9H- pyrrolo[2,3- b:5,4-c′]- dipyridine | 1H NMR (400 MHz, DMSO- d6) δ □ ppm 3.18-3.22 (m, 4H) 3.76-3.80 (m, 4H) 7.12 (d, J = 8.8 Hz, 2H) 7.54 (dd, J = 8.1, 4.6 Hz, 1H) 7.71 (d, J = 8.8 Hz, 2H) 8.51 (dt, J = 8.1, 2.0 Hz, 1H) 8.59 (dd, J = 4.8, 1.6 Hz, 1H) 8.89 (d, J = 2.2 Hz, 1H) 8.96-8.99 (m, 2H) 9.02 (d, J = 1.0 Hz, 1H) 9.37 (d, J = 2.2 Hz, 1H) 12.26 (br. s., 1H). |
| 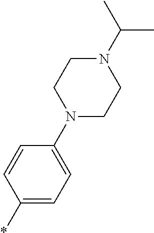 | 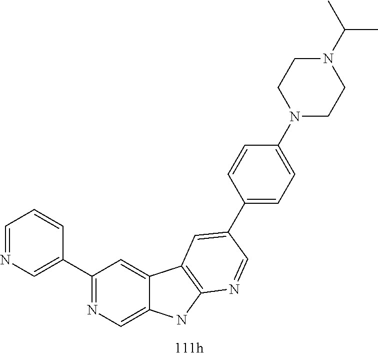 | 3-{4-[4- (propan- 2-yl) piperazin-1- yl]phenyl}-6- (pyrid-3-yl)- 9H-pyrrolo [2,3-b:5,4-c′]- dipyridine | 1H NMR (400 MHz, DMSO- d6) δ □ ppm 1.03 (d, J = 6.6 Hz, 6H) 2.59-2.63 (m, 4H) 2.65-2.74 (m, 1H) 3.19-3.23 (m, 4H) 7.09 (d, J = 8.8 Hz, 2H) 7.54 (dd, J = 8.1, 4.6 Hz, 1H) 7.68 (d, J = 8.8 Hz, 2H) 8.51 (dt, J = 8.1, 2.0 Hz, 1H) 8.59 (dd, J = 4.8, 1.6 Hz, 1H) 8.88 (d, J = 2.2 Hz, 1H) 8.95-8.99 (m, 2H) 9.02 (d, J = 1.0 Hz, 1H) 9.37 (d, J = 2.2 Hz, 1H) 12.25 (s, 1H). |
| 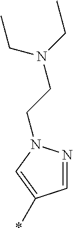 | 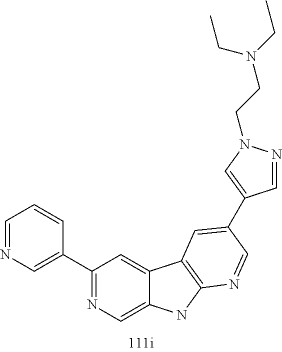 | N,N-diethyl-2- {4-[6-(pyrid-3- yl)-9H-pyrrolo- [2,3-b:5,4-c′]- dipyrid-3-yl]- 1H-pyrazol- 1-yl}- ethanamine | 1H NMR (400 MHz, DMSO- d6) δ □ ppm 0.95 (t, J = 7.1 Hz, 6H) 2.51-2.57 (m, 4H) 2.85 (t, J = 6.7 Hz, 2H) 4.21 (t, J = 6.6 Hz, 2H) 7.54 (dd, J = 7.9, 4.8 Hz, 1H) 7.98 (s, 1H) 8.28 (s, 1H) 8.50 (dt, J = 8.1, 1.8 Hz, 1H) 8.59 (dd, J = 4.8, 1.6 Hz, 1H) 8.87-8.89 (m, 2H) 8.91 (d, J = 2.0 Hz, 1H) 9.02 (d, J = 0.7 Hz, 1H) 9.35 (d, J = 2.2 Hz, 1H) 12.23 (br. s., 1H). |
| 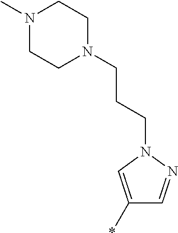 | 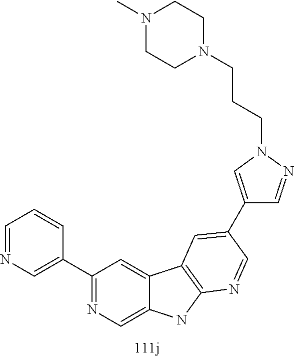 | 3-{1-[3-(4- methyl- piperazin- 1-yl)propyl]- 1H-pyrazol-4- yl}-6- (pyrid-3-yl)- 9H-pyrrolo [2,3-b:5,4-c′]- dipyridine | UPLC-SQD: Rt (min) = 0.32; [M + H]^{+:} m/z 4.53; [M − H]^{-:} m/z 451. 1H NMR (400 MHz, DMSO-d6) δ □ ppm 1.99 (quin, J = 6.8 Hz, 2H) 2.15 (s, 3H) 2.25-2.43 (m, 10H) 4.19 (t, J = 7.1 Hz, 2H) 7.54 (dd, J = 7.9, 4.8 Hz, 1H) 7.98 (d, J = 0.5 Hz, 1H) 8.27 (s, 1H) 8.50 (dt, J = 8.1, 1.8 Hz, 1H) 8.59 (dd, J = 4.8, 1.6 Hz, 1H) 8.86 (d, J = 1.0 Hz, 1H) 8.88 (d, J = 2.2 Hz, 1H) 8.91 (d, J = 2.2 Hz, 1H) 9.02 (d, J = 1.0 Hz, 1H) 9.35 (d, J = 1.7 Hz, 1H) 12.24 (br. s., 1H). |
| 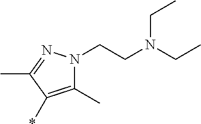 | 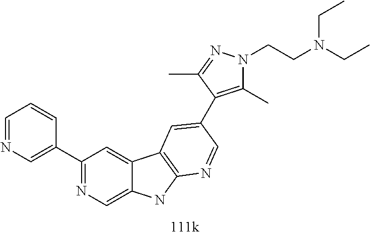 | 2-{3,5- dimethyl- 4-[6-(pyrid-3- yl)-9H- pyrrolo[2,3- b:5,4-c′] dipyrid- 3-yl]-1H- pyrazol-1-yl}- N,N-diethyl- ethanamine | UPLC-SQD: Rt (min) = 0.39; [M + H]^{+:} m/z 440; [M − H]^{-:} m/z 438. 1H NMR (400 MHz, DMSO-d6) δ □ ppm 0.95 (t, J = 7.1 Hz, 6H) 2.21 (s, 3H) 2.32 (s, 3H) 2.51-2.57 (m, 4H) 2.76 (t, J = 7.0 Hz, 2H) 4.08 (t, J = 6.8 Hz, 2H) 7.54 (dd, J = 7.9, 4.8 Hz, 1H) 8.48- 8.54 (m, 2H) 8.59 (dd, J = 4.6, 1.5 Hz, 1H) 8.63 (d, J = 2.2 Hz, 1H) 8.99 (s, 1H) 9.03 (d, J = 1.2 Hz, 1H) 9.37 (d, J = 1.7 Hz, 1H) 12.33 (s, 1H). |
| 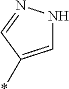 | 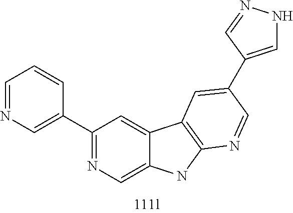 | 3-(1H-pyrazol- 4-yl)-6-(pyrid- 3-yl)-9H- pyrrolo- [2,3-b:5,4-c′]- dipyridine | UPLC-SQD: Rt (min) = 0.39; [M − H]^{-:} m/z 311. 1H NMR (400 MHz, DMSO-d6) δ □ ppm 7.54 (ddd, J = 7.9, 4.8, 0.7 Hz, 1H) 8.05 (br. s., 1H) 8.30 (br. s., 1H) 8.50 (dt, J = 8.1, 2.0 Hz, 1H) 8.59 (dd, J = 4.6, 1.5 Hz, 1H) 8.87 (s, 1H) 8.93 (d, J = 2.2 Hz, 1H) 8.94 (d, J = 2.2 Hz, 1H) 9.01 (d, J = 1.0 Hz, 1H) 9.36 (d, J = 1.7 Hz, 1H) 12.22 (s, 1H) 13.03 (br. s., 1H). |
| 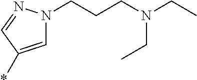 | 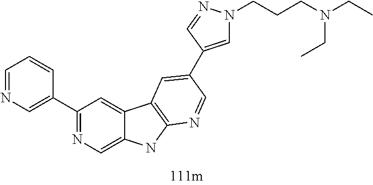 | N,N-diethyl-3- {4-[6-(pyrid-3- yl)-9H-pyrrolo- [2,3-b:5,4-c′]- dipyrid-3-yl]- 1H-pyrazol-1- yl}-propan-l- amine | 1H NMR (400 MHz, DMSO- d6) δ □ ppm 0.95 (t, J = 7.1 Hz, 6H) 1.97 (quint, J = 7.0 Hz, 2H) 2.38-2.49 (m, 6H) 4.19 (t, J = 7.0 Hz, 2H) 7.54 (ddd, J = 8.1, 4.6, 0.7 Hz, 1H) 7.99 (d, J = 0.5 Hz, 1H) 8.28 (s, 1H) 8.50 (dt, J = 8.1, 2.2 Hz, 1H) 8.59 (dd, J = 4.6, 1.5 Hz, 1H) 8.87 (d, J = 1.0 Hz, 1H) 8.88 (d, J = 2.2 Hz, 1H) 8.91 (d, J = 2.2 Hz, 1H) 9.02 (d, J = 1.2 Hz, 1H) 9.35 (d, J = 2.4 Hz, 1H) 12.24 (br. s., 1H). UPLC-SQD: Rt (min) = 0.37; [M + H]^{+:} m/z 426; [M − H]^{-:} m/z 424. |

### Table 8

### Example 128

### 3-{1-[(1-ethylpyrrolidin-2-yl)methyl]-1H-pyrazol-4-yl}-6-(pyrid-3-yl)-9H-pyrrolo[2,3-b:5,4-c′]dipyridine 113

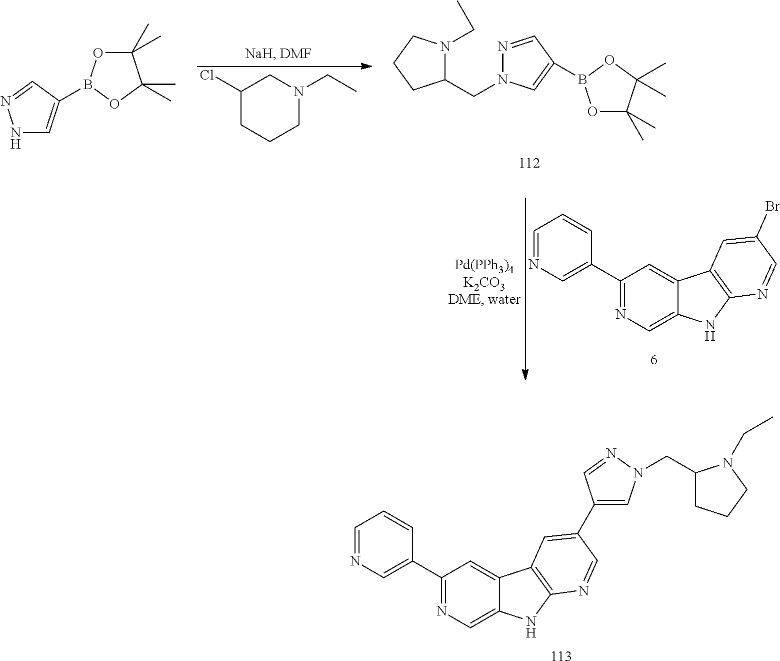

To 96 mg of 60% sodium hydride in oil in 5 mL of dimethylformamide under argon at 25° C. is added dropwise a solution of 500 mg of 4-(4,4,5,5-tetramethyl-1,3,2-dioxaborolan-2-yl)-1H-pyrazole in 7 mL of dimethylformamide. After stirring for 30 minutes at 25° C., a solution of 222 mg of N-ethyl-3-chloropiperidine hydrochloride in 7 mL of dimethylformamide is added. The reaction medium is stirred for 2 hours at 25° C., then for 1 hour at 70° C. and then for 8 hours at reflux. The reaction medium is treated with 20 mL of water and then extracted with three times 20 mL of ethyl acetate. The organic phases are combined, washed once with water, dried over magnesium sulfate, filtered and then concentrated under reduced pressure to give 0.8 g of a brown oil, which is used in crude form in the following reaction (112).

524 mg of 3-bromo-6-(pyrid-3-yl)-9H-pyrrolo[2,3-b:5,4-c′]dipyridine 6 and 786 mg of boronate 112 in 24 mL of 1,2-dimethoxyethane are placed in a microwave reactor of suitable size, followed by addition of 6.5 mL of aqueous 2 M sodium carbonate solution and 65 mg of tetrakis(triphenyl-phosphine)palladium(0), and the mixture is subjected to microwave irradiation for 10 minutes at 150° C. A further 40 mg of tetrakis(triphenylphosphine)palladium(0) are added and the mixture is irradiated for 5 minutes at 180° C. The reaction mixture is filtered and rinsed with ethanol, and the filtrate concentrated. The crude product is purified by chromatography on a column of silica, eluting with a 100/0 to 80/20 dichloromethane/methanol mixture and then by preparative HPLC in acidic medium using a 95/5 to 20/80 gradient of water+0.07% trifluoroacetic acid/acetonitrile+0.07% trifluoroacetic acid, to give 7 mg of 3-{1-[(1-ethylpyrrolidin-2-yl)methyl]-1H-pyrazol-4-yl}-6-(pyrid-3-yl)-9H-pyrrolo[2,3-b:5,4-c′]dipyridine 113 in the form of the trifluoroacetic acid salt as a yellow solid.

1H NMR (400 MHz, DMSO-d6) δ □ppm: 1.22 (t, J=7.2 Hz, 3 H); 1.68 to 2.28 (m, 4 H); 3.02 to 3.30 (m, 4 H); 3.61 to 3.72 (m, 1 H); 4.55 (dd, J=6.2 and 14.7 Hz, 1 H); 4.68 (dd, J=6.6 and 14.7 Hz, 1 H); 7.68 to 7.76 (m, 1 H); 8.16 (s, 1 H); 8.43 (s, 1 H); 8.67 to 8.73 (m, 2 H); 8.89 to 8.99 (m, 3 H); 9.06 (d, J=1.0 Hz, 1 H); 9.40 (broad s, 1 H); 9.48 to 9.59 (broad m, 1 H); 12.38 (broad s, 1 H).

LC-MS (7 min): Rt (min)=2.02; [M+H]⁺: m/z 424; [M−H]⁻: m/z 422.

### Example 129

### 2-methyl-2-propyl 4-{4-[6-(pyrid-3-yl)-9H-pyrrolo[2,3-b:5,4-c′]dipyrid-3-yl]phenyl}-piperazine-1-carboxylate 114

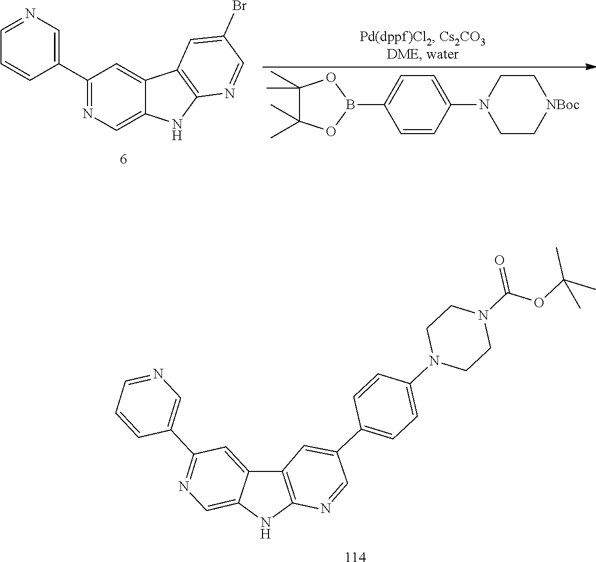

3-Bromo-6-(pyrid-3-yl)-9H-pyrrolo[2,3-b:5,4-c′]dipyridine 6 (1.5 g, 4.62 mmol), tert-butyl 4-[4-(4,4,5,5-tetramethyl[1,3,2]dioxaborolan-2-yl)phenyl]piperazine-1-carboxylate (2.1 g, 6.86 mmol), PdCl₂(dppf) (190 mg, 0.233 mmol) and caesium carbonate (3.0 g, 9.21 mmol) are placed in a sealed tube. The tube is flushed with a stream of nitrogen, followed by addition of 1,2-dimethoxyethane (25 ml) and water (2.5 ml). The tube is then sealed and heated at 110° C. for 7 hours. The reaction mixture is then cooled, diluted with water and extracted with a mixture of methylene chloride containing 10% tetrahydrofuran (60 ml) and saturated aqueous ammonium chloride solution (100 ml). The organic phases are evaporated to dryness and chromatographed on silica with a dichloromethane/methanol mixture. The yellow-brown solid obtained is triturated from methanol to give, after drying, a pale yellow solid 114 (1.75 g, 75%).

MS: m/z=507 (ES+).

1H NMR (300 MHz, DMSO-d6) δ □ppm: 9.37 (s, 1H), 9.02 (s, 1H), 8.97 (s, 2H), 8.90 (s, 1H), 8.60 (d, 1H), 8.51 (d, 1H), 7.70 (d, 2H), 7.54 (dd, 1H), 7.13 (d, 2H), 3.50 (t, 4H), 3.28 (t, 4H), 1.44 (s, 9H).

### Example 130

### 3-[4-(piperazin-1-yl)phenyl]-6-(pyrid-3-yl)-9H-pyrrolo[2,3-b:5,4-c′]dipyridine 115

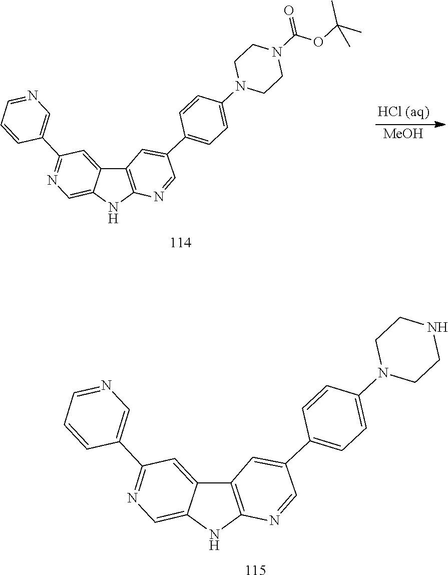

Compound 114 (600 mg, 1.19 mmol) suspended in methanol (18 ml) with HCl (4 N, 12 ml) is heated at 55° C. for 5 hours. The reaction mixture is cooled to 0-4° C. overnight. The suspension is then filtered and the solid is rinsed with a small amount of cold methanol. The orange solid is dried at 50° C. under reduced pressure to give compound 115 in the form of the hydrochloride (563 mg, 92%).

MS: m/z=407 (ES+).

1H NMR (300 MHz, DMSO-d6) δ □ppm 12.73 (s, 1H), 9.53 (s, 1H), 9.24 (s, 3H), 9.11 (s, 1H), 9.08 (d, 1H), 8.99 (d, 2H), 8.87 (d, 1H), 8.08 (dd, 1H), 7.76 (d, 2H), 7.18 (d, 2H), 3.48 (t, 4H), 3.22 (m, 4H).

### Example 131

### 2-methyl-2-propyl 4-{3-[6-(pyrid-3-yl)-9H-pyrrolo[2,3-b:5,4-c′]dipyrid-3-yl]phenyl}piperazine-1-carboxylate 116

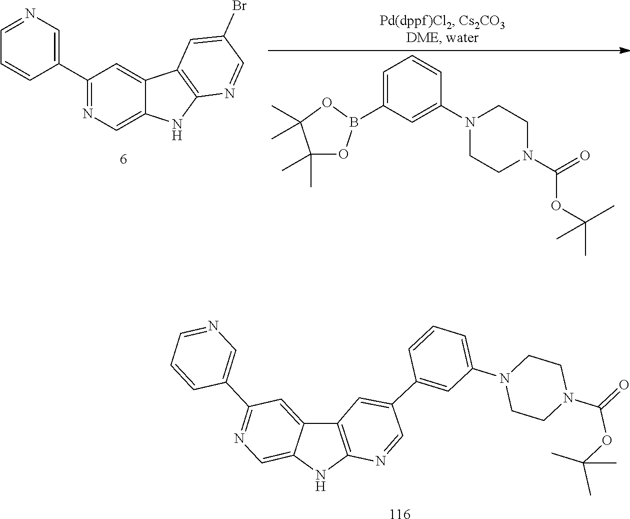

Compound 116 is prepared in the same manner as compound 114 starting with 3-bromo-6-(pyrid-3-yl)-9H-pyrrolo[2,3-b:5,4-c′]dipyridine 6 (600 mg, 1.85 mmol) and tert-butyl 4-[3-(4,4,5,5-tetramethyl[1,3,2]dioxaborolan-2-yl)phenyl]piperazine-1-carboxylate (930 mg, 2.39 mmol) to give 116 in the form of a brown solid (824 mg, 80%).

MS: m/z=507 (ES+).

1H NMR (300 MHz, DMSO-d6) δ □ppm 12.37 (s, 1H), 9.38 (s, 1H), 9.05 (s, 1H), 9.04 (s, 1H), 8.98 (s, 1H), 8.95 (s, 1H), 8.60 (d, 1H), 8.52 (d, 1H), 7.55 (dd, 1H), 7.40 (t, 1H), 7.37 (s, 1H), 7.26 (d, 1H), 7.02 (d, 1H), 3.51 (t, 4H), 3.25 (t, 4H), 1.44 (s, 9H).

### Example 132

### 3-[3-(piperazin-1-yl)phenyl]-6-(pyrid-3-yl)-9H-pyrrolo[2,3-b:5,4-c′]dipyridine 117

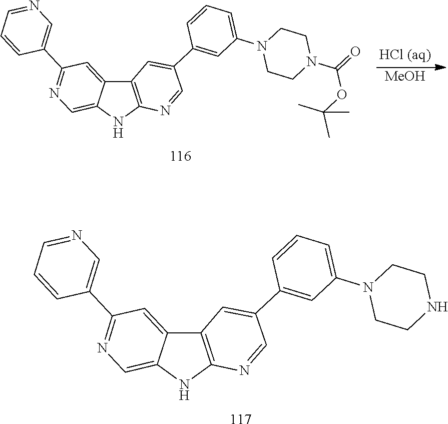

Compound 117 is prepared from 116 (670 mg, 1.32 mmol) as for compound 115 to give a yellow solid in the form of the hydrochloride (636 mg, 93%).

MS: m/z=407 (ES+).

1H NMR (300 MHz, DMSO-d6) δ □ppm 12.63 (s, 1H), 9.51 (s, 1H), 9.19 (s, 1H), 9.13 (d, 1H), 9.11 (s, 1H), 9.06 (s, 1H), 9.01 (s, 1H), 8.54 (d, 1H), 8.01 (dd, 1H), 7.45 (t, 1H), 7.42 (s, 1H), 7.32 (d, 1H), 7.08 (d, 1H), 3.52 (t, 4H), 3.27 (m, 4H).

### Example 133

### N,N-4-triethyl-5-[6-(pyrid-3-yl)-9H-pyrrolo[2,3-b:5,4-c′]dipyrid-3-yl]pyrid-2-amine 118

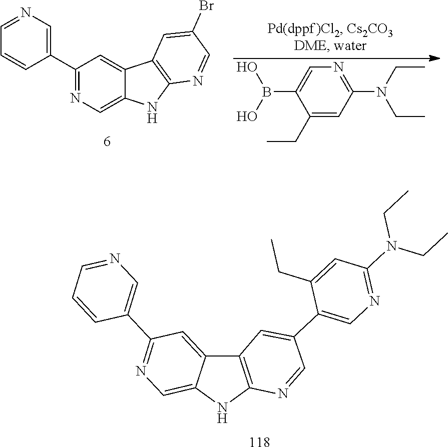

Compound 118 is prepared according to the procedure for compound 114 starting with 3-bromo-6-(pyrid-3-yl)-9H-pyrrolo[2,3-b:5,4-c′]dipyridine 6 (120 mg, 0.369 mmol) and 6-diethylamino-4-ethylpyrid-3-ylboronic acid (160 mg, 0.721 mmol). After purification by preparative HPLC (acetonitrile/H₂O containing 0.1% trifluoroacetic acid) 50 mg of product 118 are obtained in the form of the trifluoroacetic acid salt.

MS: m/z=423 (ES+).

1H NMR (300 MHz, DMSO-d6) δ □ppm: 12.58 (s, 1H), 9.42 (s, 1H), 9.11 (s, 1H), 9.02 (s, 1H), 8.76 (s, 1H), 8.73 (d, 1H), 8.71 (d, 1H), 8.63 (s, 1H), 7.96 (s, 1H), 7.75 (dd. 1H), 7.09 (s, 1H), 3.65 (q, 4H), 2.72 (q, 2H), 1.23 (t, 6H), 1.06 (t, 3H).

### Example 134

### 2-(dimethylamino)-1-(4-{3-[6-(pyrid-3-yl)-9H-pyrrolo[2,3-b:5,4-c′]dipyrid-3-yl]-phenyl}piperazin-1-yl)ethanone 119

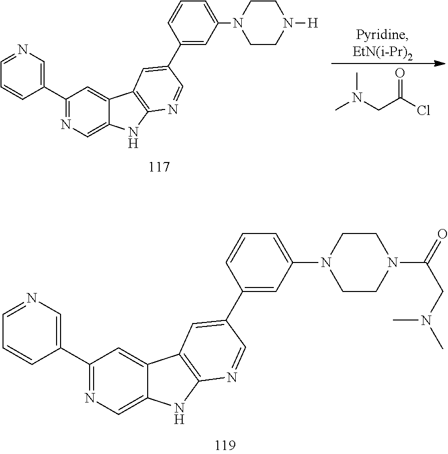

Compound 117 (70 mg, 0.136 mmol) and N,N-dimethylglycyl chloride hydrochloride (49 mg, 0.310 mmol) are placed in a Keller tube. Anhydrous pyridine (1 ml) is added under nitrogen, followed by N,N-diisopropylethylamine (148 mg, 1.15 mmol). The orange suspension is then stirred for 1.5 hours, and then diluted with saturated aqueous sodium bicarbonate solution and extracted with dichloromethane containing 10% tetrahydrofuran (5×30 ml). The combined organic phases are dried over MgSO₄ and concentrated to dryness. The solid obtained is dried under reduced pressure at 50° C. to give compound 119 in the form of a cream-white solid (56 mg, 84%).

MS: m/z=492 (ES+).

1H NMR (300 MHz, DMSO-d6) δ □ppm 12.37 (s, 1H), 9.38 (s, 1H), 9.06 (s, 1H), 9.05 (s, 1H), 8.99 (s, 1H), 8.96 (s, 1H), 8.60 (t, 1H), 8.52 (dt, 1H), 7.55 (dd, 1H), 7.41 (t, 1H), 7.38 (s, 1H), 7.27 (d, 1H), 7.03 (d, 1H), 3.68 (m, 4H), 3.52 (s, 2H), 3.32 (m, 4H), 2.41 (s, 61-1).

### Example 135

### 2-(dimethylamino)-1-(4-{4-[6-(pyrid-3-yl)-9H-pyrrolo[2,3-b:5,4-c′]dipyrid-3-yl]-phenyl}piperazin-1-yl)ethanone 120

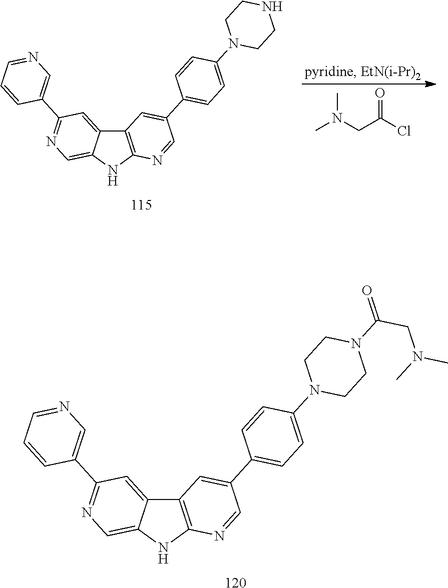

Compound 120 is prepared as for 119 using compound 115 (70 mg, 0.136 mmol) to give a bright white solid (42 mg, 63%).

MS: m/z=492 (ES+).

1H NMR (300 MHz, DMSO-d6) δ □ppm 12.30 (s, 1H), 9.37 (s, 1H), 9.03 (s, 2H), 8.99 (s, 1H), 8.90 (s, 1H), 8.60 (d, 1H), 8.51 (dd, 1H), 7.72 (d, 2H), 7.55 (dd, 1H), 7.15 (d, 2H), 3.71 (t, 2H), 3.64 (t, 2H), 3.25 (m, 4H), 3.22 (s, 2H), 2.25 (s, 6H).

### Example 136

### 1-(4-{3-[6-(pyrid-3-yl)-9H-pyrrolo[2,3-b:5,4-c′]dipyrid-3-yl]phenyl}piperazin-1-yl)-ethanone 121

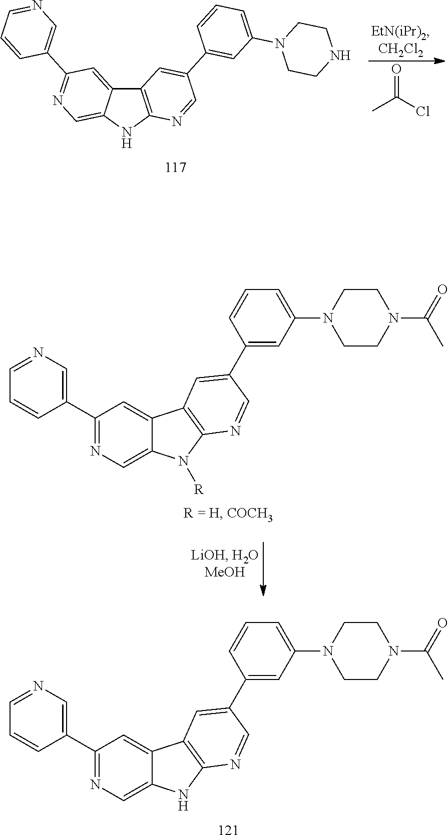

To a suspension of compound 117 (70 mg, 0.136 mmol) in dichloromethane (1.5 ml) is added N,N-diisopropylethylamine (74 mg, 0.1 ml, 0.574 mmol). The reaction is stirred at 25° C. for 15 minutes, followed by addition of acetyl chloride (22 mg, 0.28 mmol). After stirring at 25° C. for 1 hour, a further portion of acetyl chloride (22 mg) and N,N-diisopropylethylamine (0.2 ml) is added. After a further 15 minutes, methanol (0.5 ml) is added and the mixture is concentrated to dryness. The residue is partitioned between saturated aqueous sodium bicarbonate solution and dichloromethane, and the aqueous phase is then re-extracted with dichloromethane (5×30 ml). The organic phases are dried over MgSO₄ and concentrated to dryness. The residue is stirred with LiOH (5 mg) in methanol (5 ml+a few % of water) for 30 minutes. The solution is evaporated to dryness and the residue obtained is again partitioned between saturated aqueous sodium bicarbonate solution and dichloromethane, and the aqueous phase is then re-extracted with dichloromethane. The organic phases are dried over MgSO₄ and concentrated to dryness, and the solid obtained is dried under reduced pressure at 50° C. to give compound 121 in the form of an orange-red solid (38 mg, 62%).

MS: m/z=449 (ES+).

1H NMR (300 MHz, DMSO-d6) δ □ppm 9.38 (s, 1H), 9.04 (s, 2H), 8.97 (s, 1H), 8.94 (s, 1H), 8.59 (d, 1H), 8.52 (d, 1H), 7.54 (dd, 1H), 7.40 (t, 1H), 7.37 (s, 1H), 7.26 (d, 1H), 7.02 (d, 1H), 3.60 (m, 4H), 3.26 (m, 4H), 2.07 (s, 3H).

### Example 137

### 1-(4-{4-[6-(pyrid-3-yl)-9H-pyrrolo[2,3-b:5,4-c′]dipyrid-3-yl]phenyl}piperazin-1-yl)-ethanone 122

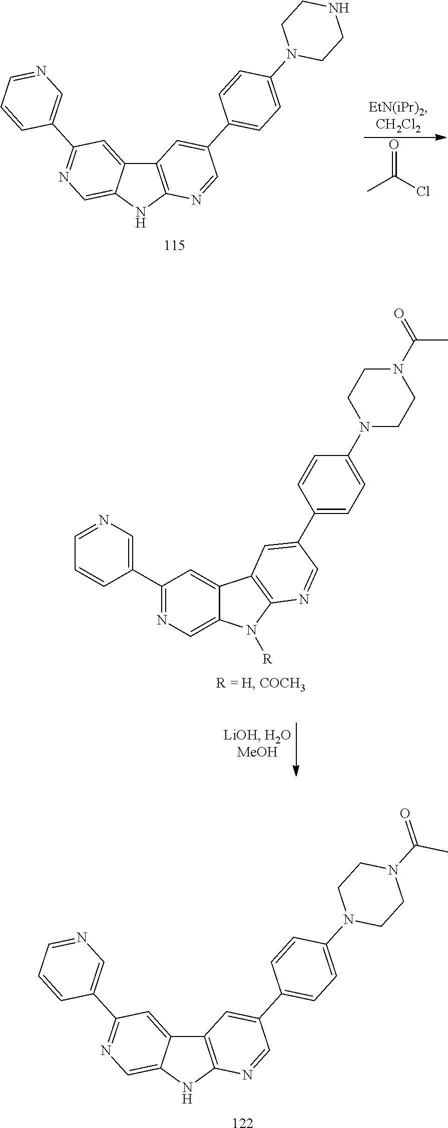

Compound 122 is prepared according to the procedure for compound 121 starting with compound 115 (70 mg, 0.136 mmol). The crude product (26 mg) is purified by SPE chromatography (SCX cartridge eluted with 7N ammoniacal methanol solution, in dichloromethane) followed by trituration in methanol to give 9 mg (15%) of product in the form of an orange-coloured solid.

MS: m/z=449 (ES+).

1H NMR (300 MHz, DMSO-d6) δ □ppm 12.30 (s, 1H), 9.38 (s, 1H), 9.03 (s, 1H), 8.99 (s, 2H), 8.90 (s, 1H), 8.60 (s, 1H), 8.52 (d, 1H), 7.72 (d, 2H), 7.55 (dd, 1H), 7.14 (d, 2H), 3.60 (t, 4H), 3.26 (t, 2H), 3.20 (t, 2H), 2.07 (s, 3H).

### Example 138

### 3-[4-(4-methylpiperazin-1-yl)phenyl]-6-(pyrid-3-yl)-9H-pyrrolo[2,3-b:5,4-c′]dipyridine 123

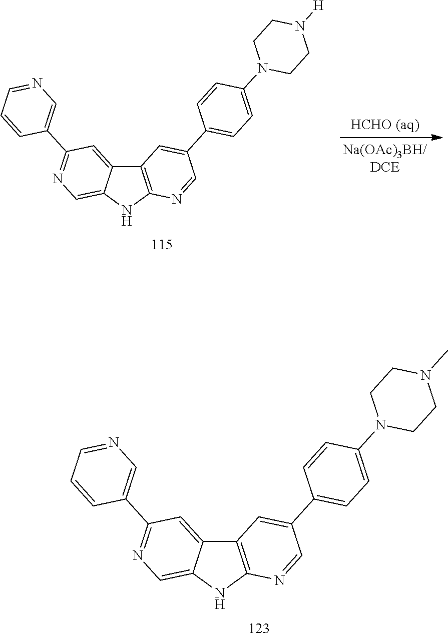

Compound 115 (70 mg, 0.136 mmol) and sodium triacetoxyborohydride (370 mg, 1.75 mmol) in 1,2-dichloroethane (4 ml) are stirred at 25° C. for 20 minutes, followed by addition of an aqueous solution of HCHO (0.026 ml, 37%, 0.347 mmol). The mixture is stirred overnight and then diluted with a mixture of saturated aqueous sodium bicarbonate solution and dichloromethane. The aqueous phases are re-extracted with dichloromethane (12×30 ml). The combined organic phases are dried over MgSO₄, concentrated and dried at 50° C. under reduced pressure to give compound 123 in the form of a yellow solid (54 mg, 74%).

MS: m/z=421 (ES+).

1H NMR (300 MHz, DMSO-d6) δ □ppm 12.32 (br, 1H), 9.38 (s, 1H), 9.03 (s, 1H), 9.02 (s, 1H), 8.97 (d, 1H), 8.89 (s, 1H), 8.60 (t, 1H), 8.52 (t, 1H), 7.70 (d, 2H), 7.55 (dt, 1H), 7.11 (d, 2H), 3.24 (m, 8H), 2.24 (s, 3H).

### Example 139

### 3-[3-(4-methylpiperazin-1-yl)phenyl]-6-(pyrid-3-yl)-9H-pyrrolo[2,3-b:5,4-c′]dipyridine 124

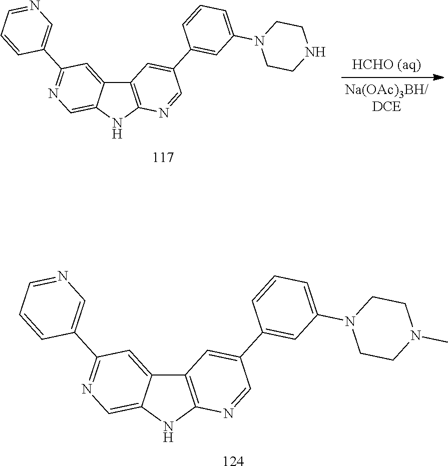

Compound 124 is prepared according to the procedure for compound 123, starting with compound 117 (70 mg, 0.136 mmol), to give a white solid (49 mg, 86%).

MS: m/z=421 (ES+).

1H NMR (300 MHz, DMSO-d6) δ □ppm 12.35 (s, 1H), 9.38 (s, 1H), 9.04 (s, 2H), 9.00 (s, 1H), 8.94 (s, 1H), 8.60 (d, 1H), 8.52 (d, 1H), 7.55 (dd, 1H), 7.35 (t, 1H), 7.33 (s, 1H), 7.21 (d, 1H), 7.00 (d, 1H), 3.28 (m, 8H), 2.25 (s, 3H).

### Example 140

### 3-{3-[4-(propan-2-yl)piperazin-1-yl]phenyl}-6-(pyrid-3-yl)-9H-pyrrolo[2,3-b:5,4-c′]dipyridine 125

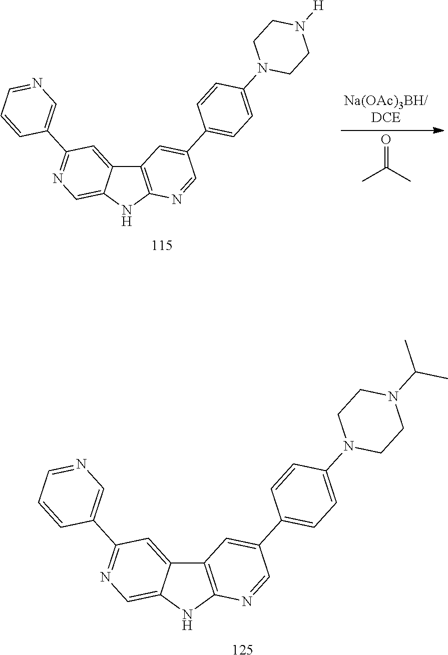

Compound 125 is prepared according to the procedure for compound 123 starting with compound 115 (70 mg, 0.136 mmol) and acetone (78 mg, 1.35 mmol) to give a white solid (52 mg, 85%).

MS: m/z=449 (ES+).

1H NMR (300 MHz, DMSO-d6) δ □ppm 12.36 (s, 1H), 9.38 (s, 1H), 9.04 (s, 2H), 9.00 (s, 1H), 8.94 (s, 1H), 8.60 (d, 1H), 8.52 (d, 1H), 7.55 (dd, 1H), 7.37 (t, 1H), 7.32 (s, 1H), 7.22 (d, 1H), 7.00 (s, 1H), 3.26 (m, 4H), 2.70 (m, 1H), 2.63 (m, 4H), 1.08 (t, 6H).

### Example 141

### 3-[4-(4-cyclopropylpiperazin-1-yl)phenyl]-6-(pyrid-3-yl)-9H-pyrrolo[2,3-b:5,4-c′]-dipyridine 126

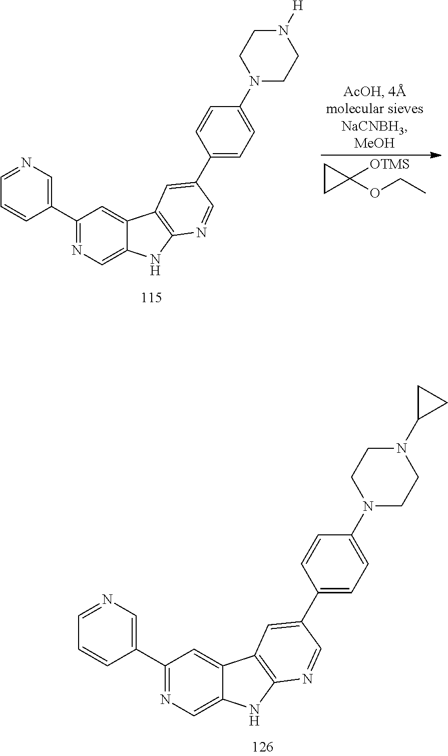

To a suspension of compound 115 (100 mg, 0.194 mmol) and ground 4 Å molecular sieves (200 mg) in methanol (5 ml) are added (1-ethoxycyclopropoxy)trimethylsilane (200 mg, 1.15 mmol), acetic acid (115 mg, 1.92 mmol) and sodium cyanoborohydride (1 M solution in tetrahydrofuran, 0.87 ml, 0.87 mmol). After heating at 60° C. for 7 hours, the reaction mixture is cooled and partitioned between saturated aqueous sodium bicarbonate solution and dichloromethane. The aqueous phases are re-extracted with dichloromethane (30 ml×6). The combined organic phases are concentrated to dryness and dried at 50° C. under reduced pressure to give 66 mg of a beige-coloured solid. This solid is purified by SPE chromatography (10% SCX 7N NH₃/methanol in dichloromethane). 44 mg of a yellow solid are obtained, which is triturated in methanol to give 40 mg of a yellow solid 126 (29%).

MS: m/z=447 (ES+).

1H NMR (300 MHz, DMSO-d6) δ □ppm 12.28 (s, 1H), 9.37 (s, 1H), 9.03 (s, 1H), 8.99 (s, 1H), 8.97 (s, 1H), 8.89 (s, 1H), 8.60 (d, 1H), 8.51 (d, 1H), 7.69 (d, 2H), 7.55 (dd, 1H), 7.10 (d, 2H), 3.19 (t, 4H), 2.71 (t, 4H), 1.68 (m, 1H), 0.45 (m, 2H), 0.37 (m, 2H).

### Example 142

### 3-[3-(4-cyclopropylpiperazin-1-yl)phenyl]-6-(pyrid-3-yl)-9H-pyrrolo[2,3-b:5,4-c′]-dipyridine 127

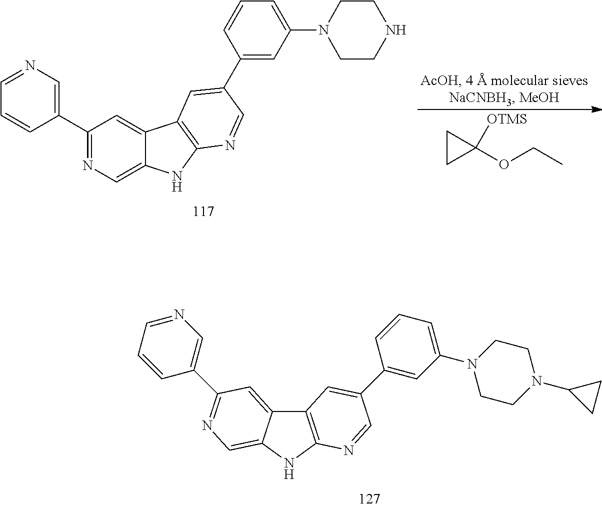

Compound 127 is prepared according to the procedure for compound 126 starting with compound 117 (100 mg, 0.194 mmol). The crude product obtained (97 mg) is purified by SPE chromatography (SCX, 10% of 7N ammoniacal methanol solution, in dichloromethane) to give 58 mg (42%) of an off-white solid.

MS: m/z=447 (ES+).

1H NMR (300 MHz, DMSO-d6) δ □ppm 12.35 (s, 1H), 9.38 (s, 1H), 9.04 (s, 2H), 8.99 (s, 1H), 8.93 (s, 1H), 8.60 (d, 1H), 8.52 (d, 1H), 7.55 (dd, 1H), 7.37 (t, 1H), 7.33 (s, 1H), 7.22 (d, 1H), 6.99 (d, 1H), 3.24 (t, 4H), 2.73 (t, 4H), 1.70 (m, 1H), 0.44 (m, 2H), 0.38 (m, 2H).

### Example 143

### 2-methyl-2-propyl 4-{4-[6-(pyrid-3-yl)-9H-pyrrolo[2,3-b:5,4-c′]dipyrid-3-yl]pyrid-2-yl}piperazine-1-carboxylate 128

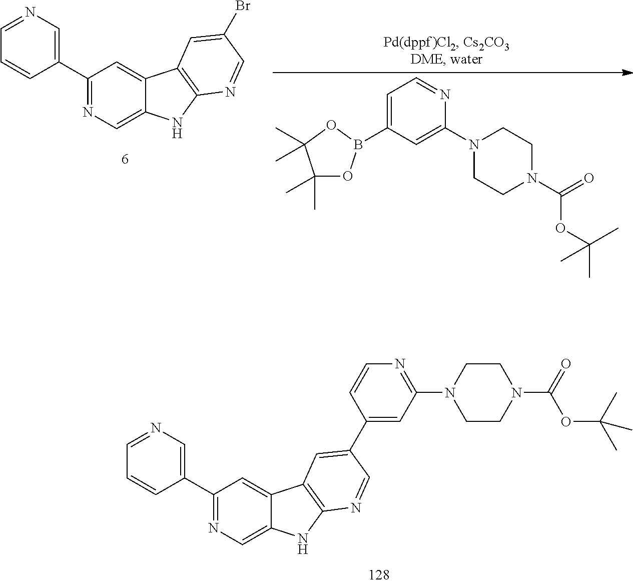

Compound 128 is prepared according to the procedure for compound 116 from 3-bromo-6-(pyrid-3-yl)-9H-pyrrolo[2,3-b:5,4-c′]dipyridine 6 (150 mg, 0.462 mmol) and text-butyl 4-[4-(4,4,5,5-tetramethyl[1,3,2]dioxaborolan-2-yl)pyrid-2-yl]piperazine-1-carboxylate (270 mg, 0.694 mmol), to give a white solid (160 mg, 68%).

MS: m/z=508 (ES+).

1H NMR (300 MHz, DMSO-d6) δ □ppm 12.50 (s, 1H), 9.38 (s, 1H), 9.18 (s, 1H), 9.07 (s, 2H), 8.97 (s, 1H), 8.61 (d, 1H), 8.52 (d, 1H), 8.26 (d, 1H), 7.55 (dd, 1H), 7.28 (s, 1H), 7.17 (d, 1H), 3.64 (m, 4H), 3.50 (m, 4H), 1.44 (s, 9H).

### Example 144

### 3-[2-(piperazin-1-yl)pyrid-4-yl]-6-(pyrid-3-yl)-9H-pyrrolo[2,3-b:5,4-c′]dipyridine 129

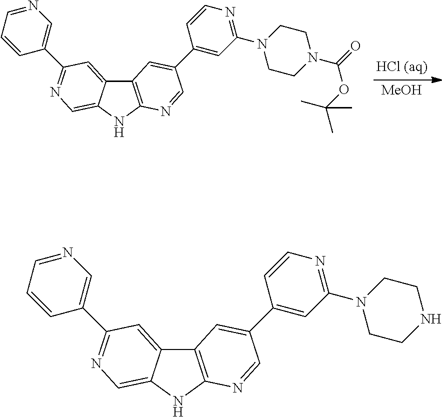

Compound 129 is prepared according to the procedure for compound 117 starting with compound 128 (108 mg, 0.213 mmol), to give the expected compound (hydrochloride, 4 HCl, 100 mg, 86%) in the form of a yellow solid.

MS: m/z=408 (ES+).

1H NMR (300 MHz, DMSO-d6) δ □ppm 12.95 (s, 1H), 9.63 (br, 2H), 9.53 (s, 1H), 9.39 (s, 1H), 9.28 (s, 1H), 9.25 (s, 1H), 9.16 (t, 1H), 9.14 (s, 1H), 8.93 (d, 1H), 8.27 (d, 1H), 8.16 (dd, 1H), 7.72 (s, 1H), 7.46 (d, 1H), 4.10 (br, 4H), 3.30 (br, 4H).

### Example 145

### N,N-dimethyl-3-({5-[6-(pyrid-3-yl)-9H-pyrrolo[2,3-b:5,4-c′]dipyrid-3-yl]pyrid-2-yl}-oxy)propan-1-amine 130

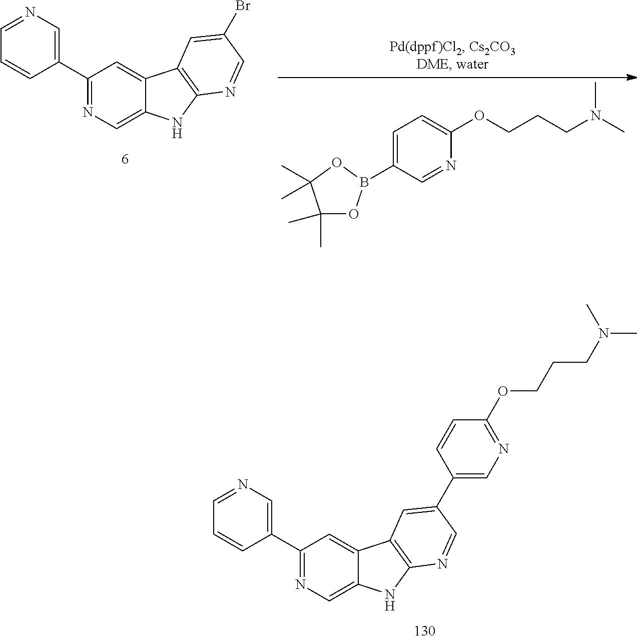

Compound 130 is prepared according to the procedure for compound 114 from 3-bromo-6-(pyrid-3-yl)-9H-pyrrolo[2,3-b:5,4-c′]dipyridine 6 (150 mg, 0.462 mmol) and dimethyl-{3-[5-(4,4,5,5-tetramethyl[1,3,2]dioxaborolan-2-yl)pyrid-2-yloxy]propyl}amine (184 mg, 0.60 mmol), to give a beige-coloured solid (80 mg, 41%).

MS: m/z=425 (ES+).

1H NMR (300 MHz, DMSO-d6) δ □ppm 12.40 (s, 1H), 9.36 (s, 1H), 9.05 (s, 2H), 8.96 (s, 1H), 8.93 (s, 1H), 8.61 (s, 1H), 8.60 (d, 1H), 8.51 (d, 1H), 8.16 (d, 1H), 7.55 (dd, 1H), 6.99 (d, 1H), 4.36 (t, 2H), 2.38 (t, 2H), 1.99 (s, 6H), 1.90 (m, 2H).

### Example 146

### N,N-dimethyl-3-{4-[6-(pyrid-3-yl)-9H-pyrrolo[2,3-b:5,4-c′]dipyrid-3-yl]phenoxy}-propan-1-amine 131

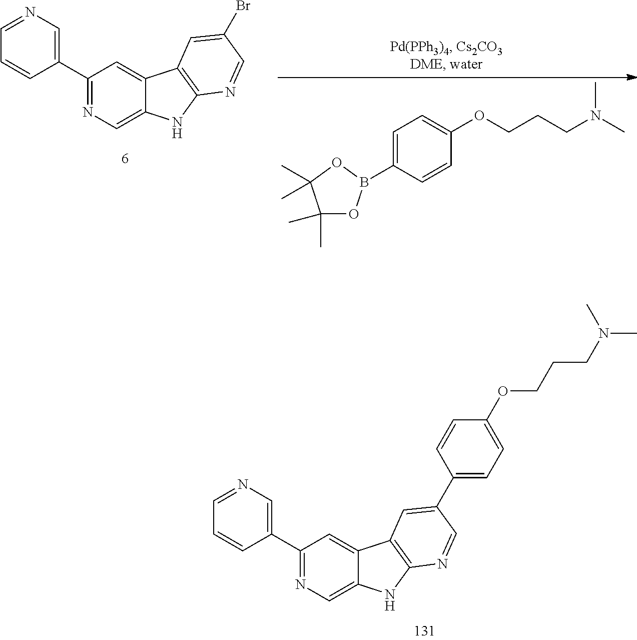

A suspension of 3-bromo-6-(pyrid-3-yl)-9H-pyrrolo[2,3-b:5,4-c′]dipyridine 6 (250 mg, 0.77 mmol), dimethyl-{3-[4-(4,4,5,5-tetramethyl[1,3,2]dioxaborolan-2-yl)phenoxy]propyl}amine (250 mg, 0.82 mmol), Pd(PPh₃)₄ (44 mg, 0.039 mmol) and caesium carbonate (627 mg, 1.9 mmol) in 1,2-dimethoxyethane (2.5 ml) and water (0.25 ml) is heated at 105° C. under nitrogen in a sealed tube for 23 hours. The reaction mixture is diluted with dichloromethane/tetrahydrofuran/methanol and filtered through Celite. The filtrate is concentrated to dryness and the residue is chromatographed (silica gel treated beforehand with 1% triethylamine in dichloromethane, and then eluted with dichloromethane/methanol) to give the product 131 in the form of a white solid (41 mg, 13%).

MS: m/z=424 (ES+).

1H NMR (300 MHz, DMSO-d6) δ □ppm 12.32 (s, 1H), 9.37 (s, 1H), 9.03 (s, 1H), 9.00 (s, 1H), 8.99 (s, 1H), 8.90 (s, 1H), 8.59 (d, 1H), 8.51 (d, 1H), 7.75 (d, 2H), 7.55 (dd, 1H), 7.10 (d, 2H), 4.08 (t, 2H), 2.39 (t, 2H), 2.17 (s, 6H), 1.89 (t, 2H).

### Example 147

### 3-{4-[3-(piperid-1-yl)propoxy]phenyl}-6-(pyrid-3-yl)-9H-pyrrolo[2,3-b:5,4-c′]-dipyridine 132

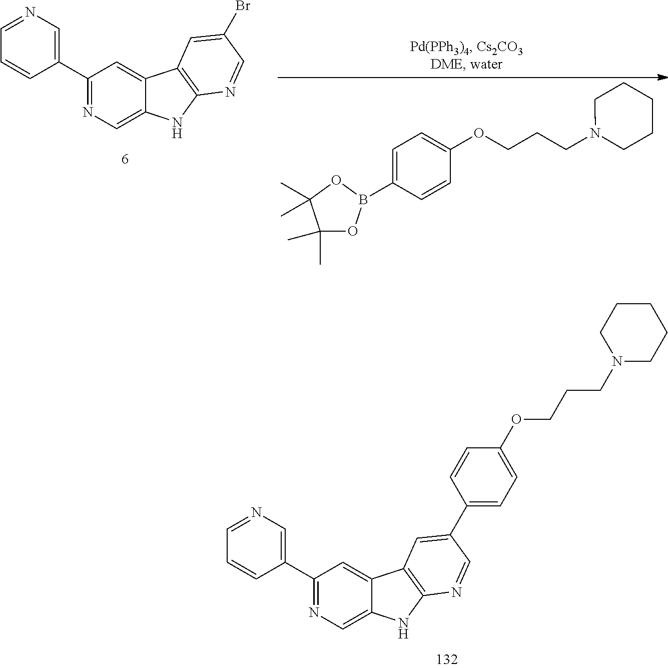

Compound 132 is prepared according to the procedure for compound 131 starting with 1-{3-[4-(4,4,5,5-tetramethyl[1,3,2]dioxaborolan-2-yl)phenoxy]propyl}piperidine (397 mg, 1.15 mmol) and 3-bromo-6-(pyrid-3-yl)-9H-pyrrolo[2,3-b:5,4-c′]dipyridine 6 (250 mg, 0.77 mmol), to give the expected product (92 mg, 26%) in the form of a white solid.

MS: m/z=464 (ES+).

1H NMR (300 MHz, DMSO-d6) δ □ppm 12.32 (s, 1H), 9.37 (s, 1H), 9.03 (s, 1H), 9.00 (s, 1H), 8.99 (s, 1H), 8.89 (s, 1H), 8.59 (d, 1H), 8.51 (d, 1H), 7.75 (d, 2H), 7.55 (dd, 1H), 7.10 (d, 2H), 4.08 (t, 2H), 2.42-2.36 (m, 6H), 1.90 (p, 2H), 1.51 (m, 4H), 1.39 (m, 2H).

### Example 148

### 3-{4-[2-(morpholin-4-yl)ethoxy]phenyl}-6-(pyrid-3-yl)-9H-pyrrolo[2,3-b:5,4-c′]-dipyridine 133

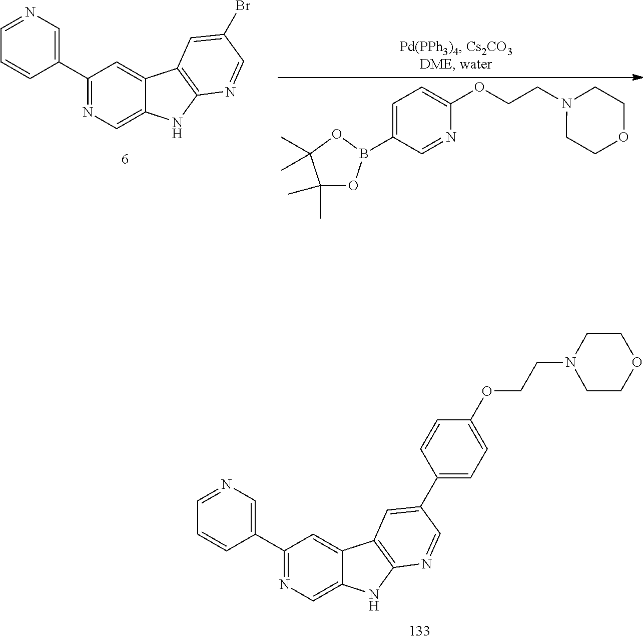

Compound 133 is prepared according to the procedure for compound 131 starting with 4-{2-[4-(4,4,5,5-tetramethyl[1,3,2]dioxaborolan-2-yl)phenoxy]ethyl}morpholine (383 mg, 1.15 mmol) and 3-bromo-6-(pyrid-3-yl)-9H-pyrrolo[2,3-b:5,4-c′]dipyridine 6 (250 mg, 0.77 mmol), to give the expected product (45 mg, 13%) in the form of a white solid.

MS: m/z=452 (ES+).

1H NMR (300 MHz, DMSO-d6) δ □ppm 12.36 (s, 1H), 9.39 (s, 1H), 9.04 (s, 1H), 9.01 (s, 1H), 8.99 (s, 1H), 8.91 (s, 1H), 8.62 (m, 1H), 8.52 (d, 1H), 7.83 (d, 2H), 7.56 (m, 1H), 7.16 (d, 2H), 4.30 (m, 2H), 3.71 (m, 4H), 3.20-2.50 (m, 6H).

### Example 149

### 3-{4-[3-(morpholin-4-yl)propoxy]phenyl}-6-(pyrid-3-yl)-9H-pyrrolo[2,3-b:5,4-c′]-dipyridine 134

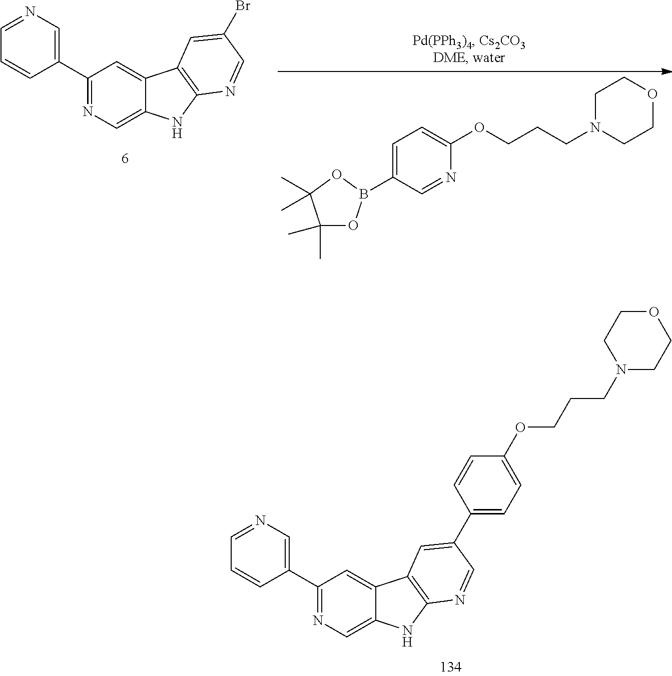

Compound 134 is prepared according to the procedure for compound 131 starting with 4-{3-[4-(4,4,5,5-tetramethyl[1,3,2]dioxaborolan-2-yl)phenoxy]propyl}morpholine (399 mg, 1.15 mmol) and 3-bromo-6-(pyrid-3-yl)-9H-pyrrolo[2,3-b:5,4-c′]dipyridine 6 (250 mg, 0.77 mmol), to give the expected product (125 mg, 35%) in the form of a white solid.

MS: m/z=466 (ES+).

1H NMR (300 MHz, DMSO-d6) δ □ppm 12.32 (s, 1H), 9.37 (s, 1H), 9.03 (s, 1H), 9.00 (s, 1H), 8.99 (s, 1H), 8.90 (s, 1H), 8.59 (d, 1H), 8.51 (d, 1H), 7.75 (d, 2H), 7.55 (dd, 1H), 7.11 (d, 2H), 4.09 (t, 2H), 3.60 (t, 4H), 2.47-2.40 (m, 6H), 1.92 (p, 2H).

### Example 150

### 3-{4-[2-(1H-imidazol-1-yl)ethoxy]phenyl}-6-(pyrid-3-yl)-9H-pyrrolo[2,3-b:5,4-c′]-dipyridine 135

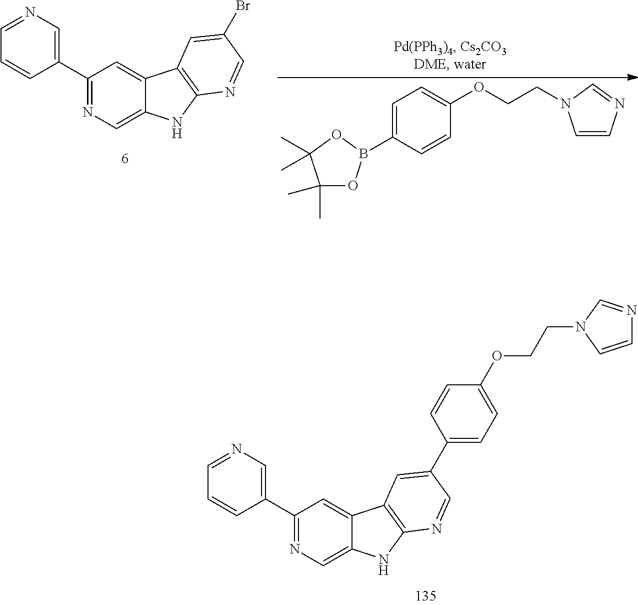

Compound 135 is prepared according to the procedure for compound 131 starting with 1-{2-[4-(4,4,5,5-tetramethyl[1,3,2]dioxaborolan-2-yl)phenoxy]ethyl}-1H-imidazole (250 mg, 1.08 mmol) and 3-bromo-6-(pyrid-3-yl)-9H-pyrrolo[2,3-b:5,4-c′]dipyridine 6 (250 mg, 0.77 mmol), to give the expected product (72 mg, 22%) in the form of a white solid.

MS: m/z=433 (ES+).

1H NMR (300 MHz, DMSO-d6) δ □ppm 12.30, 9.37 (s, 1H), 9.03 (s, 1H), 9.00 (s, 1H), 8.98 (s, 1H), 8.89 (s, 1H), 8.59 (d, 1H), 8.51 (d, 1H), 7.76 (d, 2H), 7.55 (dd, 1H), 7.29 (s, 1H), 7.12 (d, 2H), 6.93 (s, 1H), 4.41 (t, 2H), 4.34 (t, 2H).

### Example 151

### 4-[6-(pyrid-3-yl)-9H-pyrrolo[2,3-b:5,4-c′]dipyrid-3-yl]phenol 136

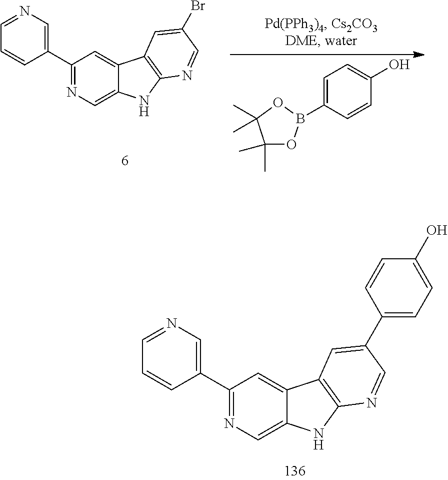

Compound 136 is prepared according to the procedure for compound 131 starting with 4-(4,4,5,5-tetramethyl[1,3,2]dioxaborolan-2-yl)phenol (305 mg, 1.39 mmol) and 3-bromo-6-(pyrid-3-yl)-9H-pyrrolo[2,3-b:5,4-c′]dipyridine 6 (200 mg, 0.62 mmol), to give the expected product (37 mg, 18%) in the form of a white solid.

MS: m/z=339 (ES+).

1H NMR (300 MHz, DMSO-d6) δ □ppm 12.27 (s, 1H), 9.60 (s, 1H), 9.36 (s, 1H), 9.02 (s, 1H), 8.88 (s, 1H), 8.86 (s, 1H), 8.83 (d, 1H), 8.60 (d, 1H), 8.50 (d, 1H), 7.62 (d, 2H), 7.55 (dd, 1H), 6.93 (d, 2H).

### Example 152

### 3-(4-{3-[4-(methylsulfonyl)piperazin-1-yl]propoxy}phenyl)-6-(pyrid-3-yl)-9H-pyrrolo-[2,3-b:5,4-c′]dipyridine 138

Step 1: 1-(methylsulfonyl)-4-{3-[4-(4,4,5,5-tetramethyl-1,3,2-dioxaborolan-2-yl)phenoxy]propyl}-piperazine 137

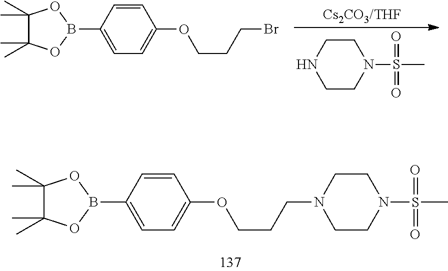

A suspension of 2-[4-(3-bromo-propoxy)phenyl]-4,4,5,5-tetramethyl[1,3,2]dioxaborolane (350 mg, 1.03 mmol), 1-(methylsulfonyl)piperazine (185 mg, 1.13 mmol) and caesium carbonate (336 mg, 1.03 mmol) in tetrahydrofuran (3.0 ml) is heated at 150° C. by microwave for 1 hour. The reaction medium is then concentrated to dryness and the residue is washed with water and triturated with toluene and then with ether to give the expected product 137 (520 mg) in the form of a whitish gel.

MS: m/z=424.2 (ES+).

1H NMR (300 MHz, CDCl₃) δ □ppm 7.75 (d, 2H), 7.25 (s, 1H), 6.85 (d, 2H), 4.04 (t, 2H), 3.25 (t, 4H), 2.79 (s, 3H), 3.62-3.50 (m, 6H), 1.94 (p, 2H), 1.32 (s, 12H).

### Step 2:

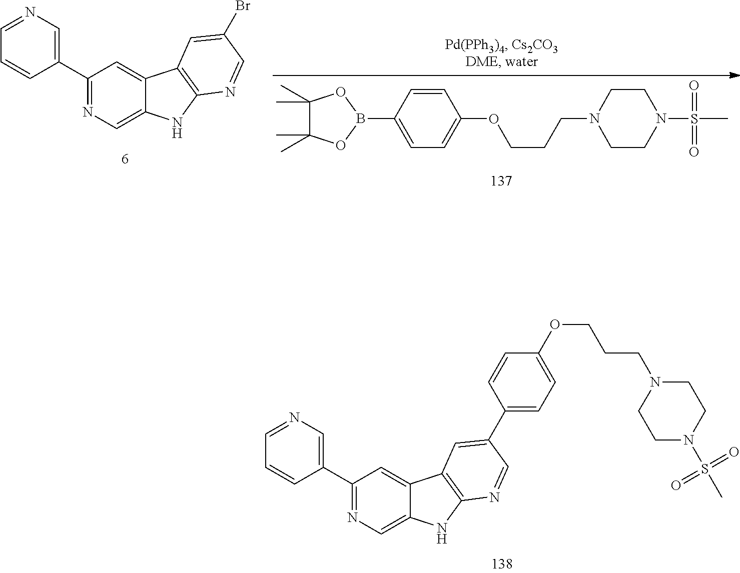

Compound 138 is prepared according to the procedure for compound 131 starting with 137 (used in crude form, 520 mg, 1.03 mmol) and 3-bromo-6-(pyrid-3-yl)-9H-pyrrolo[2,3-b:5,4-c′]dipyridine 6 (250 mg, 0.77 mmol), to give the expected product (118 mg, 28%) in the form of a white solid.

MS: m/z=543 (ES+).

1H NMR (300 MHz, CDCl₃+methanol-d₄) δ □ppm 9.21 (s, 1H), 9.04 (s, 1H), 8.78 (s, 1H), 8.68 (s, 1H), 8.58 (d, 1H), 8.46 (s, 1H), 8.43 (d, 1H), 7.62 (d, 2H), 7.50 (dd, 1H), 7.06 (d, 2H), 4.12 (t, 2H), 3.29 (m, 4H), 2.83 (s, 3H), 2.65 (m, 6H), 2.05 (t, 2H).

### Example 153

### N,N-diethyl-2-{3-[6-(pyrid-3-yl)-9H-pyrrolo[2,3-b:5,4-c′]dipyrid-3-yl]phenoxy}ethan-amine 140

Step 1: N,N-diethyl-2-[3-(4,4,5,5-tetramethyl-1,3,2-dioxaborolan-2-yl)phenoxy]ethanamine 139

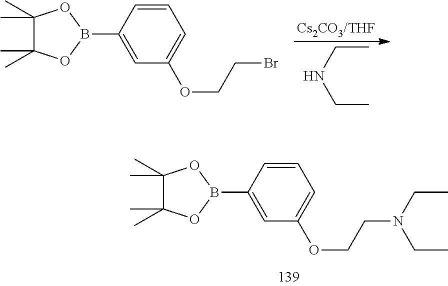

139 is prepared according to the procedure for compound 137 starting with 2-[3-(2-bromo-ethoxy)phenyl]-4,4,5,5-tetramethyl[1,3,2]dioxaborolane (300 mg, 0.92 mmol) and diethylamine (104 mg, 1.43 mmol), to give the expected product 139 (262 mg, 89%) in the form of a yellow oil.

MS: m/z=320 (ES+).

1H NMR (300 MHz, CDCl₃) δ □ppm 7.46-7.26 (m, 3H), 7.03 (s, 1H), 4.10 (t, 2H) 2.89 (t, 2H), 2.64 (q, 4H), 1.35 (s, 12H), 1.10 (t, 6H).

### Step 2:

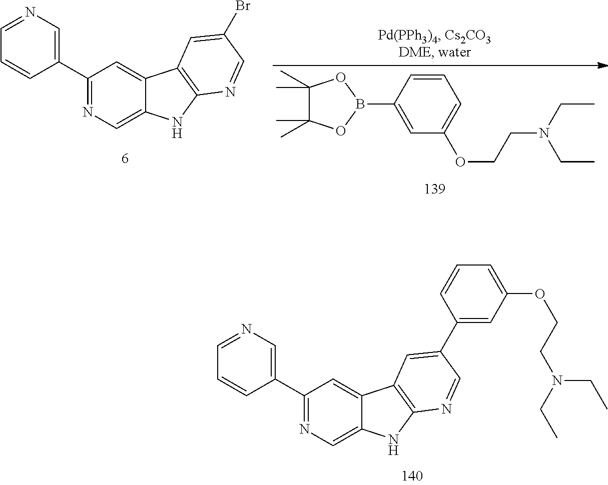

Compound 140 is prepared according to the procedure for compound 131 starting with 139 (used in crude form, 259 mg, 0.81 mmol) and 3-bromo-6-(pyrid-3-yl)-9H-pyrrolo[2,3-b:5,4-c′]dipyridine 6 (200 mg, 0.62 mmol), to give the expected product (56 mg, 21%) in the form of a white solid.

MS: m/z=438 (ES+).

1H NMR (300 MHz, CDCl₃+methanol-d₄) δ □ppm 9.24 (s, 1H), 9.05 (s, 1H), 8.80 (s, 1H), 8.75 (s, 1H), 8.58 (d, 1H), 8.50 (s, 1H), 8.42 (d, 1H), 7.48 (dd, 1H), 7.44 (t, 1H), 7.33 (d, 1H), 7.32 (s, 1H), 6.97 (dd, 1H), 4.58 (t, 2H), 3.50 (t, 2H), 3.28 (q, 4H), 1.45 (t, 6H).

### Example 154 (142) and Example 155

### 3-{4-[6-(pyrid-3-yl)-9H-pyrrolo[2,3-b:5,4-c′]dipyrid-3-yl]-phenoxy}propan-1-amine 143

Step 1: 2-methyl-2-propyl {3-[4-(4,4,5,5-tetramethyl-1,3,2-dioxaborolan-2-yl)phenoxy]propyl}-carbamate 141

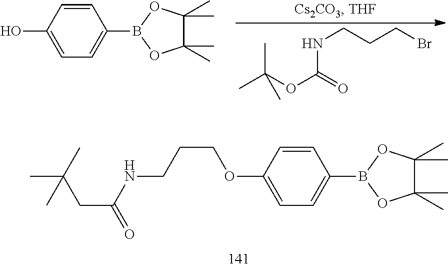

141 is prepared according to the procedure for 137 starting with 4-(4,4,5,5-tetramethyl-[1,3,2]dioxaborolan-2-yl)phenol (264 mg, 1.2 mmol) and 2-methyl-2-propyl (3-bromopropyl)-carbamate (450 mg, 1.89 mmol), to give a brown oil (500 mg).

MS: m/z=378 (ES+).

1H NMR (300 MHz, CDCl₃) δ □ppm 7.75 (d, 2H), 6.89 (d, 2H), 4.05 (t, 2H), 3.33 (q, 2H), 1.99 (p, 2H), 1.42 (s, 9H), 1.35 (s, 12H).

Step 2: 2-methyl-2-propyl (3-{4-[6-(pyrid-3-yl)-9H-pyrrolo[2,3-b:5,4-c′]dipyrid-3-yl]phenoxy}-propyl)carbamate 142

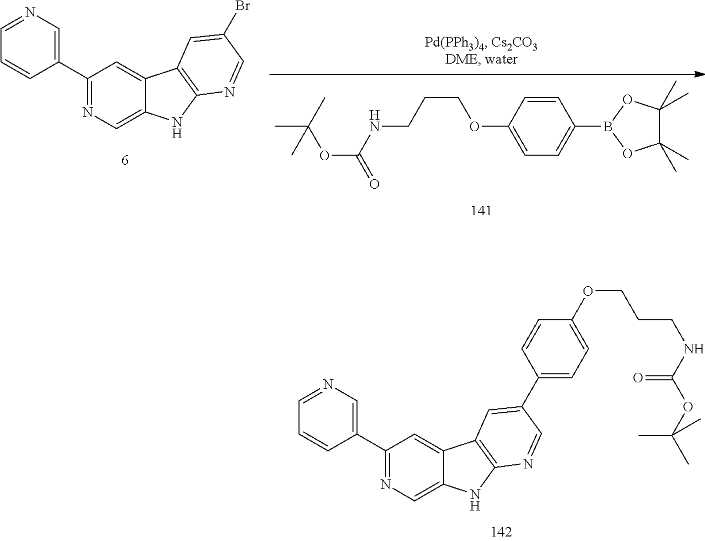

142 is prepared according to the procedure for compound 131 starting with 141 (used in crude form, 500 mg, 1.32 mmol) and 3-bromo-6-(pyrid-3-yl)-9H-pyrrolo[2,3-b:5,4-c′]dipyridine 6 (200 mg, 0.62 mmol), to give the expected product (46 mg, 15%) in the form of a pale yellow solid.

1H NMR (300 MHz, CDCl₃) δ □ppm 9.31 (s, 1H), 9.11 (s, 1H), 8.83 (s, 1H), 8.66 (d, 1H), 8.62 (s, 1H), 8.44 (s, 1H), 8.42 (d, 1H), 7.61 (d, 2H), 7.44 (dd, 1H), 7.05 (d, 2H), 4.82 (br, 1H), 4.11 (t, 2H), 3.39 (q, 2H), 2.04 (p, 2H), 1.47 (s, 9H).

### Step 3:

Compound 142 (45 mg, 0.09 mmol) is treated with 0.5 ml of trifluoroacetic acid in dichloromethane (3 ml) at 0° C. and then stirred at 25° C. overnight. The mixture is then cooled to 0° C. and neutralized with saturated aqueous sodium bicarbonate solution to give a suspension, which is filtered; the precipitate is washed with water and then with dichloromethane, and then dried to give 3-{4-[6-(pyrid-3-yl)-9H-pyrrolo[2,3-b:5,4-c′]dipyrid-3-yl]phenoxy}propan-1-amine 143 (35 mg, 97%) in the form of an amber-yellow solid.

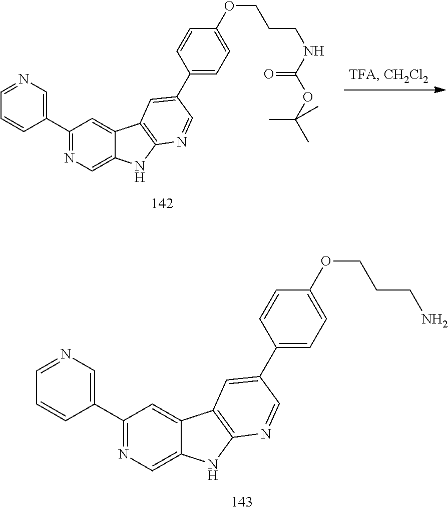

MS: m/z=396 (ES+)

1H NMR (300 MHz, DMSO-d6) δ □ppm 9.37 (s, 1H), 9.03 (s, 1H), 8.98 (s, 2H), 8.89 (s, 1H), 8.59 (d, 1H), 8.51 (d, 1H), 7.74 (d, 2H), 7.54 (dd, 1H), 7.10 (d, 2H), 4.09 (t, 2H), 3.14 (m, 2H), 2.73 (br, 1H), 1.86 (m, 2H).

### Example 156

### 2-methyl-2-propyl 4-{4-methyl-5-[6-(pyrid-3-yl)-9H-pyrrolo[2,3-b:5,4-c′]dipyrid-3-yl]-pyrid-2-yl}piperazine-1-carboxylate 144

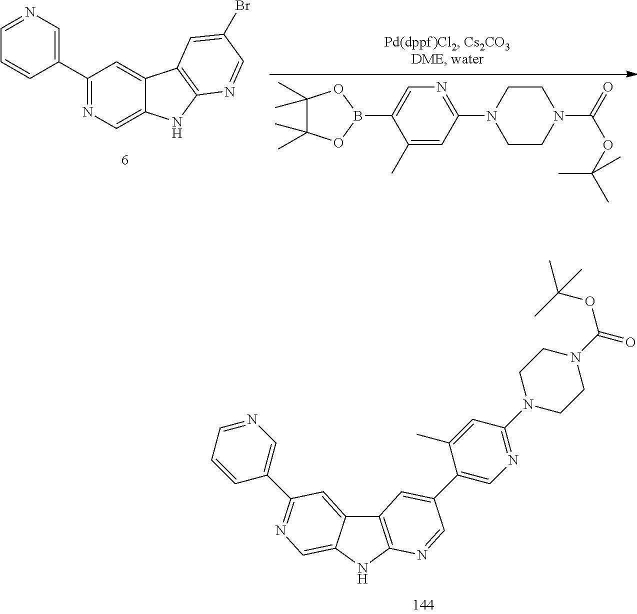

Compound 144 is prepared according to the procedure for compound 114 starting with 3-bromo-6-(pyrid-3-yl)-9H-pyrrolo[2,3-b:5,4-c′]dipyridine 6 (150 mg, 0.462 mmol) and tert-butyl 4-[4-methyl-5-(4,4,5,5-tetramethyl[1,3,2]dioxaborolan-2-yl)pyrid-2-yl]piperazine-1-carboxylate (242 mg, 0.60 mmol), to give the expected compound (120 mg, 50%) in the form of a brown solid.

MS: m/z=522 (ES+).

1H NMR (300 MHz, DMSO-d6) δ □ppm 12.37 (s, 1H), 9.36 (s, 1H), 9.05 (s, 1H), 8.95 (s, 1H), 8.73 (s, 1H), 8.60 (d, 1H), 8.58 (s, 1H), 8.50 (m, 1H), 8.09 (s, 1H), 7.54 (dd, 1H), 6.89 (s, 1H), 3.56 (m, 4H), 3.46 (m, 4H), 2.30 (s, 3H), 1.44 (s, 9H).

### Example 157

### 3-[4-methyl-6-(piperazin-1-yl)pyrid-3-yl]-6-(pyrid-3-yl)-9H-pyrrolo[2,3-b:5,4-c′]-dipyridine 145

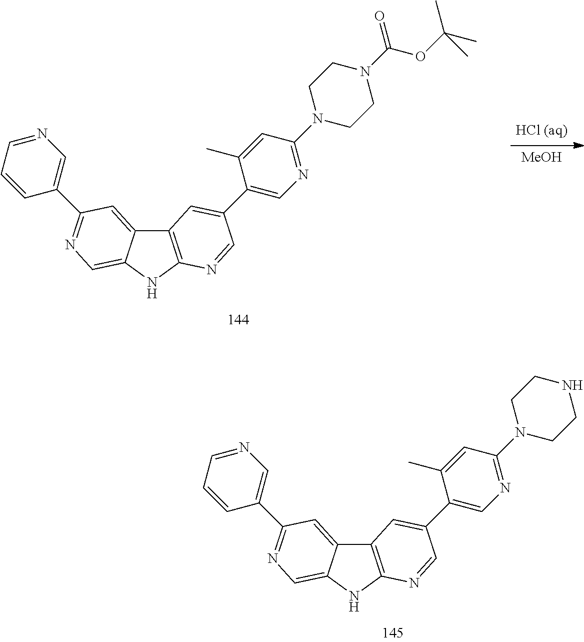

Compound 145 is prepared according to the procedure for compound 115 starting with compound 144 (60 mg, 0.115 mmol), to give the expected product (hydrochloride, 4 HCl), 65 mg, 100%) in the form of a yellow solid.

MS: m/z=422 (ES+).

1H NMR (300 MHz, DMSO-d6) δ □ppm 12.78 (s, 1H), 9.54 (s, 1H), 9.45 (br, 2H), 9.26 (s, 1H), 9.17 (s, 1H), 9.14 (s, 1H), 8.90 (d, 1H), 8.81 (s, 1H), 8.69 (s, 1H), 8.12 (t, 2H), 7.23 (s, 1H), 3.93 (br, 4H), 3.25 (br, 4H), 2.38 (s, 3H).

### Example 158

### 3-[6-(piperazin-1-yl)pyrid-3-yl]-6-(pyrid-3-yl)-9H-pyrrolo[2,3-b:5,4-c′]dipyridine 146

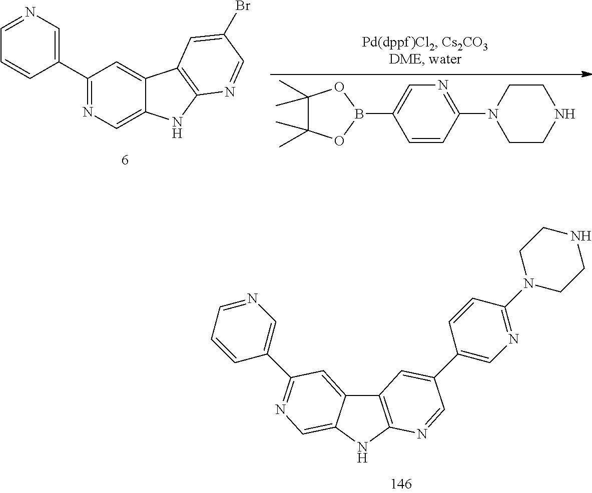

Compound 146 is prepared according to the procedure for compound 114 starting with 3-bromo-6-(pyrid-3-yl)-9H-pyrrolo[2,3-b:5,4-c′]dipyridine 6 (150 mg, 0.462 mmol) and 1-[5-(4,4,5,5-tetra-methyl[1,3,2]dioxaborolan-2-yl)pyrid-2-yl]-piperazine (175 mg, 0.605 mmol), to give the expected compound (172 mg, 92%) in the form of a brown solid.

MS: m/z=408 (ES+).

1H NMR (300 MHz, DMSO-d6) δ □ppm 12.32 (br, 1H), 9.37 (s, 1H), 9.03 (s, 1H), 8.99 (s, 1H), 8.95 (s, 1H), 8.90 (s, 1H), 8.60 (t, 1H), 8.58 (s, 1H), 8.51 (m, 1H), 8.00 (d, 1H), 7.55 (dd, 1H), 6.98 (d, 1H), 3.49 (t, 4H), 2.81 (t, 4H).

### Example 159

### 3-fluoro-6-(5-methoxypyrid-3-yl)-9H-pyrrolo[2,3-b:5,4-c′]dipyridine 152

Step 1: 2′,5′-dichloro-5-fluoro-[3,4′]bipyridyl-2-ylamine 147

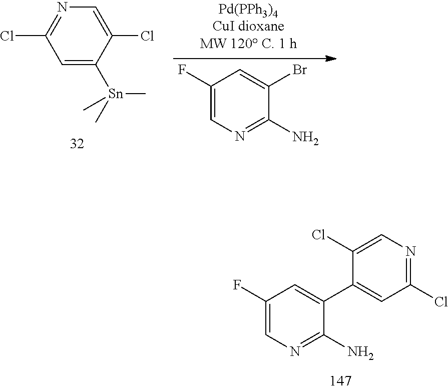

580 mg of 2-amino-3-bromo-5-fluoropyridine, 1.04 g of 2,5-dichloro-4-trimethylstannylpyridine 32, 246 mg of tetrakis(triphenylphosphine)palladium(0) and 122 mg of copper (I) iodide in 10 mL of 1,4-dioxane are placed in a reactor, and the tube is sealed and subjected to microwave irradiation for 1 hour at 125° C. The reaction mixture is filtered through a 0.45 μm sinter funnel and then washed with dichloromethane. After concentrating under reduced pressure, the residue obtained is purified by chromatography on a column of silica, eluting with a 100/0 to 95/5 dichloromethane/methanol mixture, and then taken up in ethyl acetate. After filtering through a 0.45 μm sinter funnel and washing with diethyl ether, 712 mg of 2′,5′-dichloro-5-fluoro[3,4′]bipyridyl-2-ylamine 147 are obtained in the form of a beige-coloured solid.

UPLC-MS-DAD-ELSD (LS): Rt (min)=0.96; (M+H)(+): 258(+)/260(+)/ . . . (presence of two chlorine atoms).

Step 3: 5′-chloro-5-fluoro-2′-methoxy-[3,4′]bipyridyl-2-ylamine 148

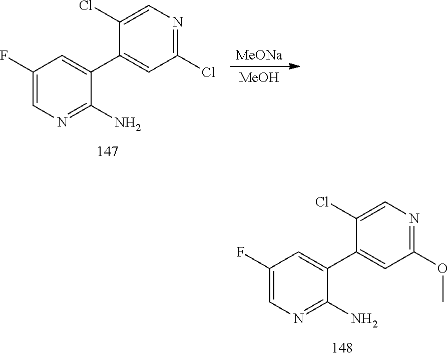

1.6 g of 2′,5′-dichloro-5-fluoro[3,4′]bipyridyl-2-ylamine 147 in 10 mL of methanol and then 670 mg of sodium methoxide are placed in a reactor, and the tube is sealed and subjected to microwave irradiation for 1 hour at 100° C. The reaction mixture is filtered through a 0.45 μm sinter funnel and then washed with dichloromethane and concentrated under reduced pressure. The residue is taken up in dichloromethane and water. After separation of the phases by settling, the organic phase is dried over magnesium sulfate, filtered and then concentrated under reduced pressure to give 1.3 g of 5′-chloro-5-fluoro-2′-methoxy[3,4′]bipyridyl-2-ylamine 148.

UPLC-MS-DAD-ELSD (LS): Rt (min)=0.93; (M+H)(+): 254(+)/ . . . (presence of a chlorine atom).

Step 4: 3-fluoro-6-methoxy-9H-pyrrolo[2,3-b:5,4-c′]dipyridine 149

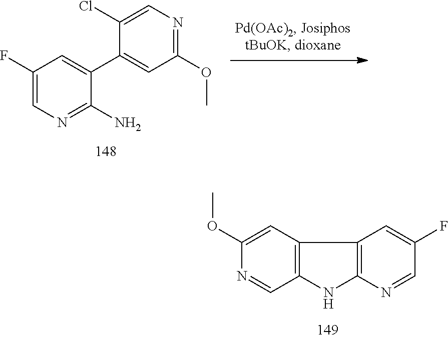

99 mg of (R)-(−)-1-[(S)-2-(dicyclohexylphosphino)ferrocenyl]ethyldi-tert-butylphosphine and 36.3 mg of palladium (II) acetate in 1 mL of anhydrous 1,4-dioxane are placed in a tube, under an argon atmosphere and stirred for 10 minutes at 40° C.

410 mg of 5′-chloro-5-fluoro-2′-methoxy[3,4′]bipyridyl-2-ylamine 148 and 725 mg of potassium tert-butoxide in 4 ml of anhydrous 1,4-dioxane are placed in a reactor under argon, the solution prepared previously is then added, and the tube is sealed and subjected to microwave irradiation for 2 hours at 120° C. The reaction mixture is filtered through a 0.45 μm sinter funnel and washed with dichloromethane, and the filtrate obtained is then concentrated under reduced pressure. The residue is taken up in ethyl acetate and water. After separation of the phases by settling, the organic phase is dried over magnesium sulfate, filtered and then concentrated under reduced pressure. After triturating in ethyl acetate followed by filtering under vacuum, 350 mg of 3-fluoro-6-methoxy-9H-pyrrolo[2,3-b:5,4-c′]dipyridine 149 are obtained in the form of a yellow solid.

UPLC-MS-DAD-ELSD (LS): Rt (min)=0.75; (M+H)(+): 218(+); (M−H)(−): 216(−).

Step 5: 3-fluoro-9H-pyrrolo[2,3-b:5,4-c′]dipyrid-6-ol 150

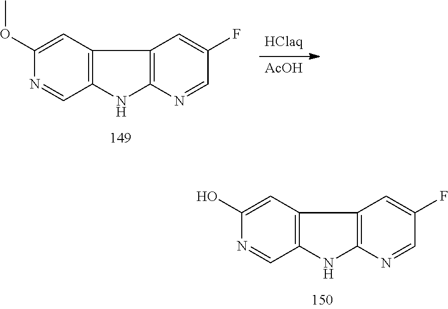

900 mg of 3-fluoro-6-methoxy-9H-pyrrolo[2,3-b:5,4-c′]dipyridine 149 in 6 ml of acetic acid and 4 ml of aqueous 37% hydrochloric acid solution are placed in a reactor, and the tube is sealed and subjected to microwave irradiation for 3 hours at 130° C. The reaction mixture is filtered under vacuum and then washed with diethyl ether to give, after drying, 1.0 g of 3-fluoro-9H-pyrrolo[2,3-b:5,4-c′]dipyrid-6-ol 150 in the form of a yellow solid.

UPLC-MS-DAD-ELSD (LS): Rt (min)=0.41; (M+H)(+): 204(+); (M−H)(−): 202(−).

Step 6: 3-fluoro-9H-pyrrolo[2,3-b:5,4-c′]dipyrid-6-yl trifluoromethanesulfonate 151

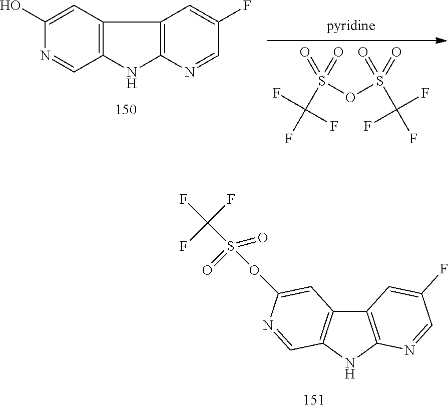

A mixture of 1 g of 3-fluoro-9H-pyrrolo[2,3-b:5,4-c′]dipyrid-6-ol 150 in 20 mL of pyridine and 5.8 ml of triflic anhydride is stirred 45 minutes at room temperature. The reaction mixture is poured into ethyl acetate and saturated aqueous bicarbonate solution. After separation of the phases by settling, the organic phase is dried over magnesium sulfate, filtered and then concentrated under reduced pressure. The residue is purified by chromatography on a column of silica, eluting with a 100/0 to 0/100 heptane/ethyl acetate mixture to give 828 mg of 3-fluoro-9H-pyrrolo[2,3-b:5,4-c′]dipyrid-6-yl trifluoromethanesulfonate 151 in the form of a beige-coloured solid.

UPLC-MS-DAD-ELSD (LS): Rt (min)=1.22; (M+H)(+): 336(+); (M−H)(−): 334(−).

Step 7: 3-fluoro-6-(5-methoxypyrid-3-yl)-9H-pyrrolo[2,3-b:5,4-c′]dipyridine 152

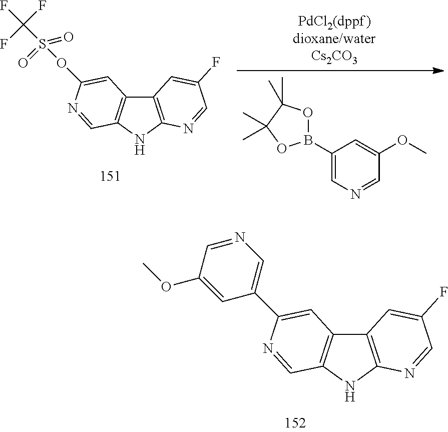

100 mg of 3-fluoro-9H-pyrrolo[2,3-b:5,4-c′]dipyrid-6-yl trifluoromethanesulfonate 151, 105 mg of 3-methoxy-5-pyridineboronic acid pinacol ester, 292 mg of caesium carbonate, 11 mg of 1,1′-bis(diphenylphosphino)ferrocenedichloropalladium(II) in 8 mL of 1,4-dioxane and 2 mL of water are placed in a reactor, and the tube is sealed and subjected to microwave irradiation for 30 minutes at 125° C. The reaction mixture is poured into water and ethyl acetate. After separation of the phases by settling, the organic phase is dried over magnesium sulfate, filtered and then concentrated under reduced pressure. The residue is purified by chromatography on a column of silica, eluting with pure ethyl acetate to give 65 mg of 3-fluoro-6-(5-methoxypyrid-3-yl)-9H-pyrrolo[2,3-b:5,4-c′]dipyridine 152 in the form of a yellow solid.

1H NMR (400 MHz, DMSO-d6) δ □ppm 3.95 (s, 3 H) 8.05 (dd, J=2.8, 1.8 Hz, 1 H) 8.31 (d, J=2.7 Hz, 1 H) 8.64-8.66 (m, 2 H) 8.93 (d, J=1.0 Hz, 1 H) 8.95 (d, J=1.7 Hz, 1 H) 9.05 (d, J=1.0 Hz, 1 H) 12.39 (br. s., 1 H).

LC-MS (7 min): Rt (min)=2.68; [M+H]⁺: m/z 295; [M−H]⁻: m/z 293.

### Example 160

### 3-fluoro-6-(4-methoxypyrid-3-yl)-9H-pyrrolo[2,3-b:5,4-c′]dipyridine 153

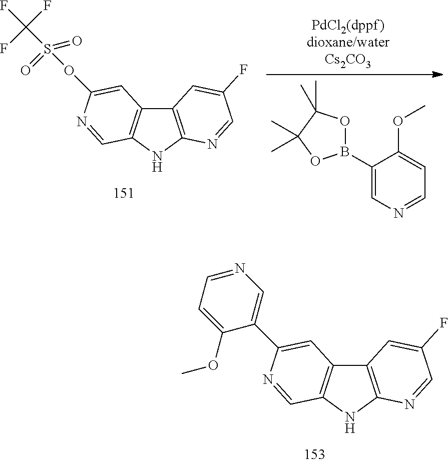

In a manner similar to that for compound 152, 10 mg of 3-fluoro-6-(4-methoxypyrid-3-yl)-9H-pyrrolo[2,3-b:5,4-c′]dipyridine 153 are obtained from 100 mg of 3-fluoro-9H-pyrrolo[2,3-b:5,4-c′]-dipyrid-6-yl trifluoromethanesulfonate 151 and 68 mg of 4-methoxy-3-pyridineboronic acid.

1H NMR (400 MHz, DMSO-d6) δ □ppm 3.97 (s, 3 H) 7.22 (d, J=5.9 Hz, 1 H) 8.47 (d, J=5.9 Hz, 1 H) 8.62-8.64 (m, 2 H) 8.73 (dd, J=8.9, 2.8 Hz, 1 H) 8.84 (s, 1 H) 9.03 (d, J=1.0 Hz, 1 H) 12.31 (br. s., 1 H).

LC-MS (7 min): Rt (min)=2.19; [M+H]⁺: m/z 295; [M−H]⁻: m/z 293.

### Example 161

### 3-fluoro-6-[5-(methylsulfanyl)pyrid-3-yl]-9H-pyrrolo[2,3-b:5,4-c′]dipyridine 154

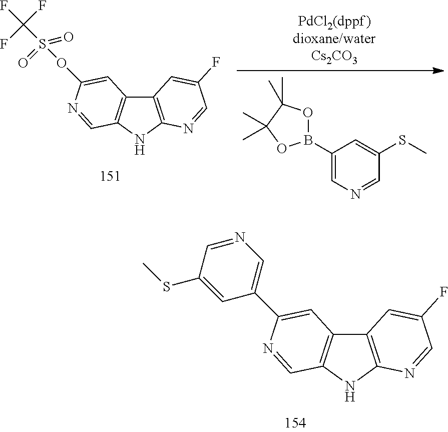

In a manner similar to that for compound 152, 50 mg of 3-fluoro-6-[5-(methylsulfanyl)pyrid-3-yl]-9H-pyrrolo[2,3-b:5,4-c′]dipyridine 154 are obtained from 100 mg of 3-fluoro-9H-pyrrolo[2,3-b:5,4-c′]-dipyrid-6-yl trifluoromethanesulfonate 151 and 141 mg of 5-(methylthio)pyridine-3-boronic acid.

1H NMR (400 MHz, DMSO-d6) δ □ppm 2.64 (s, 3 H) 8.36 (t, J=2.1 Hz, 1 H) 8.49 (d, J=2.2 Hz, 1 H) 8.62-8.67 (m, 2 H) 8.94 (d, J=1.0 Hz, 1 H) 9.05 (d, J=1.0 Hz, 1 H) 9.11 (d, J=2.0 Hz, 1 H) 12.37 (br. s., 1 H).

UPLC-SQD: Rt (min)=0.67; [M+H]⁺: m/z 311; [M−H]⁻: m/z 309.

### Example 162

### 3-fluoro-6-(thiophen-3-yl)-9H-pyrrolo[2,3-b:5,4-c′]dipyridine 155

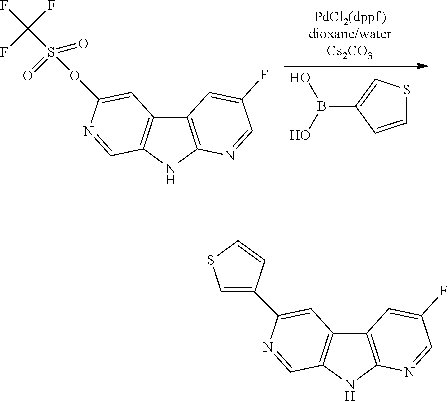

In a manner similar to that for compound 152, 89 mg of 3-fluoro-6-(thiophen-3-yl)-9H-pyrrolo[2,3-b:5,4-c′]dipyridine 155 are obtained from 150 mg of 3-fluoro-9H-pyrrolo[2,3-b:5,4-c′]dipyrid-6-yl trifluoromethanesulfonate 151 and 86 mg of thiophenyl-3-boronic acid.

1H NMR (400 MHz, DMSO-d6) δ ppm 7.67 (dd, J=3.1 and 5.1 Hz, 1 H); 7.80 (dd, J=1.3 and 5.1 Hz, 1 H); 8.05 (dd, J=1.3 and 3.1 Hz, 1 H); 8.61 (dd, J=2.8 and 7.7 Hz, 1 H); 8.62 (d, J=2.8 Hz, 1 H); 8.67 (d, J=1.2 Hz, 1 H); 8.93 (d, J=1.2 Hz, 1 H); 12.25 (broad s, 1 H)

LC-MS-DAD-ELSD: 268(−)=(M−H)(−); 270(+)=(M+H)(+) Rt (min)=2.79

### Example 163

### 3-methoxy-6-(1-methyl-1H-pyrazol-4-yl)-9H-pyrrolo[2,3-b:5,4-c′]dipyridine 161

Step 1: 5-chloro-2-methoxy-4-(trimethylstannyl)pyridine 156

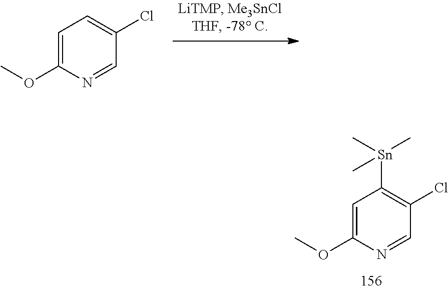

A mixture of 10 g of 5-chloro-2-methoxypyridine and 220 mL of tetrahydrofuran is cooled to −78° C., followed by gradual addition of a freshly prepared solution of 14.1 mL of 2,2,6,6-tetramethylpiperidine in 50 mL of tetrahydrofuran and 36.4 mL of 2.3N n-butyllithium in hexane. After stirring for 4 hours at −78° C., 17.3 g of trimethyltin chloride dissolved in 30 mL of tetrahydrofuran are added to the reaction mixture. The reaction mixture is stirred at room temperature for 18 hours and then treated with 200 mL of water and 200 mL of aqueous 10% ammonium chloride solution and extracted with 500 ml and then 200 mL of ethyl acetate. The combined organic phases are dried over magnesium sulfate, filtered and then concentrated to dryness under reduced pressure. The residue is purified by chromatography on a column of silica, eluting with dichloromethane, to give 17.7 g of 5-chloro-2-methoxy-4-(trimethylstannyl)pyridine 156 in the form of a colourless oil.

UPLC-MS-DAD-ELSD: Rt (min)=1.24; [M+H]⁺: m/z 308.

1H NMR (400 MHz, DMSO-d6) δ □ppm: 0.16 (t, J=29.6 Hz, 9 H) 3.62 (s, 3 H) 6.61 (t, J=20.5 Hz, 1 H) 7.90 (t, J=8.3 Hz, 1 H).

Step 2: N-(5′-chloro-2′,5-dimethoxy-3,4′-bipyrid-2-yl)-2,2-dimethylpropanamide 157

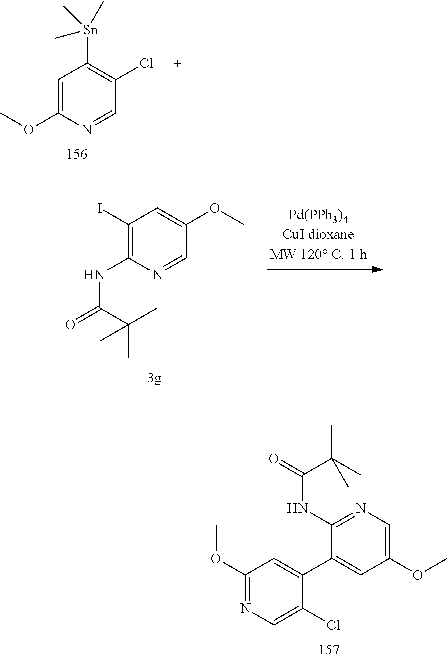

1.67 g of N-(3-iodo-5-methoxypyrid-2-yl)-2,2-dimethylpropanamide 3 g, 2.00 g of 5-chloro-2-methoxy-4-(trimethylstannyl)pyridine 156, 404 mg of tetrakis(triphenylphosphine)palladium(0) and 200 mg of copper iodide in 15 mL of 1,4-dioxane are placed in a reactor under argon and subjected to microwave irradiation for 1 hour at 120° C. A further 202 mg of tetrakis(triphenylphosphine)-palladium(0) and 100 mg of copper iodide are added and the mixture is again subjected to microwave irradiation for 1 hour at 120° C. After 60 hours at 25° C., a further 100 mg of tetrakis(triphenyl-phosphine)palladium(0), 50 mg of copper iodide and 0.50 g of stannyl derivative are added and the mixture is subjected to microwave irradiation for 1 hour at 120° C.

The reaction mixture is poured into water and ethyl acetate and the suspension obtained is then filtered through Celite. After separation of the phases by settling, the aqueous phase is extracted with ethyl acetate. The combined organic phases are dried over magnesium sulfate, filtered and then concentrated under reduced pressure. The residue is purified by chromatography on a column of silica, eluting with a 50/50 to 0/100 heptane/ethyl acetate mixture to give 1.28 g of N-(5′-chloro-2′,5-dimethoxy-3,4′-bipyrid-2-yl)-2,2-dimethylpropanamide 157 in the form of a pale yellow solid.

UPLC-MS-DAD-ELSD (LS): Rt (min)=1.11; (M+H)(+): 350(+)/352(+), presence of a chlorine atom.

Step 3: 3,6-dimethoxy-9H-pyrrolo[2,3-b:5,4-c′]dipyridine 158

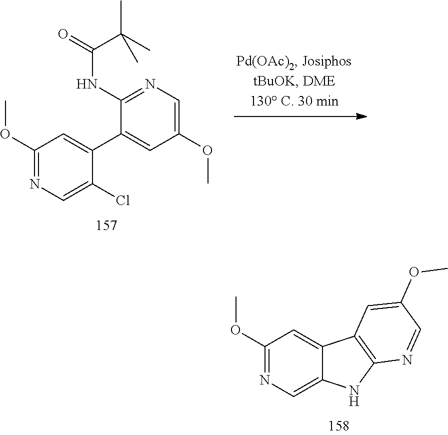

54 mg of palladium acetate and 152 mg of (R)-(−)-1-[(S)-2-(dicyclohexylphosphino)ferrocenyl]-ethyldi-tert-butylphosphine in 2 mL of 1,4-dioxane are placed in a tube under argon and stirred for 10 minutes at 40° C. The solution is then added to a suspension of 1.20 g of N-(5′-chloro-2′,5-dimethoxy-3,4′-bipyrid-2-yl)-2,2-dimethylpropanamide 157 and 770 mg of potassium tert-butoxide in 1.75 mL of 1,4-dioxane. The reaction mixture is subjected to microwave irradiation for 30 minutes at 130° C.

The reaction mixture is taken up in ethyl acetate and filtered through Celite and then washed three times with water. The organic phase is dried over magnesium sulfate, filtered and then concentrated under reduced pressure. The residue is purified by chromatography on a column of silica, eluting with a 50/50 to 0/100 heptane/ethyl acetate mixture to give 417 mg of 3,6-dimethoxy-9H-pyrrolo[2,3-b:5,4-c′]dipyridine in the form of a yellow solid 158.

UPLC-MS-DAD-ELSD (LS): Rt (min)=0.73; (M+H)(+): 230(+).

Step 4: 3-methoxy-9H-pyrrolo[2,3-b:5,4-c′]dipyrid-6-ol 159

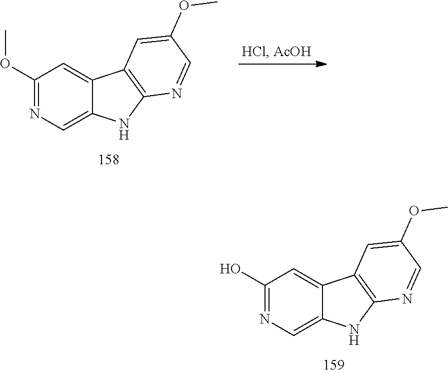

700 mg of 3,6-dimethoxy-9H-pyrrolo[2,3-b:5,4-c′]dipyridine 158 in 8.1 ml of acetic acid and 2.7 ml of concentrated hydrochloric acid are placed in a reactor and the mixture is subjected to microwave irradiation for 2.5 hours at 130° C. The reaction medium is left for 16 hours at 25° C. and then filtered to give 810 mg of 3-methoxy-9H-pyrrolo[2,3-b:5,4-c′]dipyrid-6-ol in the form of an ochre-coloured solid 159.

UPLC-MS-DAD-ELSD (LS): Rt (min)=0.46; (M+H)(+): 216(+).

Step 5: 3-methoxy-9H-pyrrolo[2,3-b:5,4-c′]dipyrid-6-yl trifluoromethanesulfonate 160

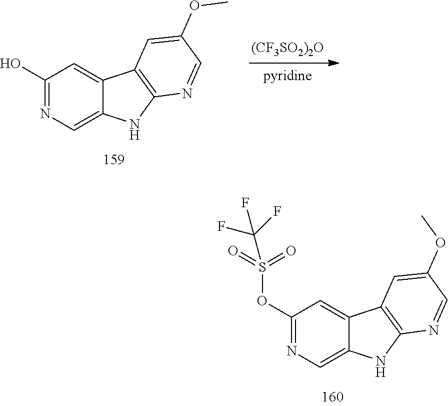

To a solution of 860 mg of 3-methoxy-9H-pyrrolo[2,3-b:5,4-c′]dipyrid-6-ol 159 in 40 mL of pyridine under argon is added 0.65 ml of trifluoromethanesulfonic anhydride. After 30 minutes at 25° C., 0.65 ml of trifluoromethanesulfonic anhydride is added and the reaction medium is stirred for 30 minutes and then concentrated. This residue is taken up in ethyl acetate and then washed three times with 5% aqueous ammonia solution. The combined aqueous phases are extracted twice with ethyl acetate and the combined organic phases are then dried over magnesium sulfate, treated with carbon black, filtered through Celite and then concentrated under reduced pressure to give 941 mg of 3-methoxy-9H-pyrrolo[2,3-b:5,4-c′]dipyrid-6-yl trifluoromethanesulfonate 160 in the form of an ochre-coloured solid.

UPLC-MS-DAD-ELSD (LS): Rt (min)=1.26; (M+H)(+): 348(+).

Step 6: 3-methoxy-6-(1-methyl-1H-pyrazol-4-yl)-9H-pyrrolo[2,3-b:5,4-c′]dipyridine 161

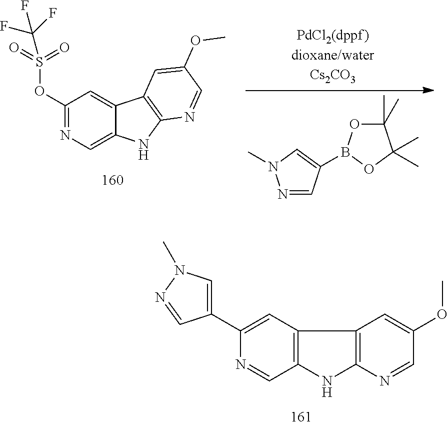

66 mg of 3-methoxy-9H-pyrrolo[2,3-b:5,4-c′]dipyrid-6-yl trifluoromethanesulfonate 160, 29 mg of 1-methyl-4-(4,4,5,5-tetramethyl-1,3,2-dioxaborolan-2-yl)-1H-pyrazole, 5 mg of 1,1′-bis(diphenyl-phosphino)ferrocenedichloropalladium(II) in 0.69 mL of 1,4-dioxane and 0.275 mL of aqueous 1.5 M caesium carbonate solution are placed in a reactor under argon and the mixture is then subjected to microwave irradiation for 30 minutes at 150° C.

The reaction mixture is taken up in ethyl acetate and filtered through Celite and then washed with water. The organic phase is dried over magnesium sulfate, treated with carbon black, filtered through Celite and then concentrated under reduced pressure to give 12 mg of 3-methoxy-6-(1-methyl-1H-pyrazol-4-yl)-9H-pyrrolo[2,3-b:5,4-c′]dipyridine 161 in the form of a brown solid.

1H NMR (400 MHz, DMSO-d6) δ □ppm 3.91 (s, 3 H); 3.93 (s, 3 H); 7.97 (s, 1 H); 8.18 (s, 1H); 8.26 (d, J=2.7 Hz, 1 H); 8.35 (d, J=2.7 Hz, 1 H); 8.40 (broad s, 1 H); 8.80 (d, J=1.0 Hz, 1 H); 11.83 (broad s, 1 H).

UPLC-SQD: Rt (min)=0.42; [M+H]⁺: m/z 280; [M−H]⁻: m/z 278.

### Example 164

### N,N-diethyl-2-[4-(3-methoxy-9H-pyrrolo[2,3-b:5,4-c′]dipyrid-6-yl)-3,5-dimethyl-1H-pyrazol-1-yl]ethanamine 162

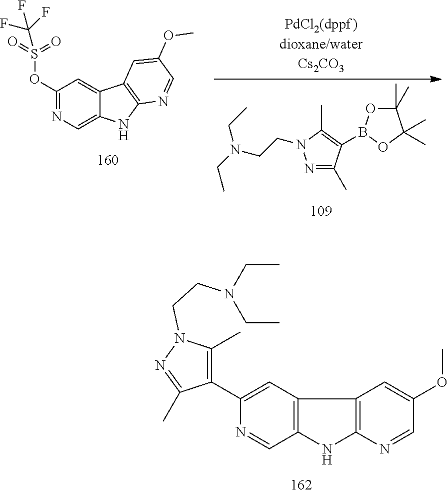

52 mg of 3-methoxy-9H-pyrrolo[2,3-b:5,4-c′]dipyrid-6-yl trifluoromethanesulfonate 160, 53 mg of ester boronic acid 109, 6.5 mg of 1,1′-bis(diphenylphosphino)ferrocenedichloropalladium(II) in 0.75 mL of 1,4-dioxane and 0.30 mL of aqueous 1.5 M caesium carbonate solution are placed in a reactor under argon and the mixture is then subjected to microwave irradiation for 30 minutes at 150° C.

The reaction mixture is treated with water and extracted with an 80/20 ethyl acetate/THF mixture. The combined organic phases are dried over magnesium sulfate, treated with carbon black, filtered through Celite and then concentrated under reduced pressure. The residue is purified by chromatography on a column of silica, eluting with an 80/20 dichloromethane/methanol mixture to give 6 mg of N,N-diethyl-2-[4-(3-methoxy-9H-pyrrolo[2,3-b:5,4-c′]dipyrid-6-yl)-3,5-dimethyl-1H-pyrazol-1-yl]ethan-amine 162.

1H NMR (400 MHz, DMSO-d6) δ □ppm 0.95 (t, J=7.1 Hz, 6 H); 2.30 (s, 3 H); 2.42 (s, 3 H); 2.50 to 2.55 (partially masked m, 4 H); 2.75 (t, J=6.7 Hz, 2 H); 3.92 (s, 3 H); 4.06 (t, J=6.7 Hz, 2 H); 8.10 (s, 1 H); 8.34 (d, J=2.9 Hz, 1 H); 8.39 (d, J=2.9 Hz, 1 H); 8.89 (s, 1 H); 11.84 (broad s, 1 H).

UPLC-SQD: Rt (min)=0.36; [M+H]⁺: m/z 393; [M−H]⁻: m/z 391.

### Example 165

### N-[2-(dimethylamino)ethyl]-2-[4-(3-methoxy-9H-pyrrolo[2,3-b:5,4-c′]dipyrid-6-yl)-1H-pyrazol-1-yl]acetamide 164

Step 1: N-[2-(dimethylamino)ethyl]-2-[4-(4,4,5,5-tetramethyl-1,3,2-dioxaborolan-2-yl)-1H-pyrazol-1-yl]acetamide 163

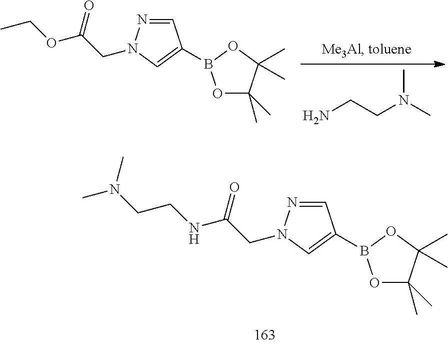

To a solution of 120 μl of N,N-dimethylethylenediamine in 3 mL of toluene under argon at 15° C. is added dropwise 0.5 mL of a 2M solution of trimethylaluminium in toluene. After 5 minutes at 20° C., a solution of 280 mg of 1-(ethoxycarbonylmethyl)-1H-pyrazole-4-boronic acid pinacol ester in 1.5 mL of toluene is added to the reaction medium. The reaction medium is stirred for 1 hour at 20° C. and then poured into 15 mL of aqueous 1M potassium sodium tartrate solution and then extracted with twice 20 mL of dichloromethane. The combined organic phases are dried over magnesium sulfate, filtered and then concentrated and dried under reduced pressure to give 125 mg of N-[2-(dimethylamino)ethyl]-2-[4-(4,4,5,5-tetramethyl-1,3,2-dioxaborolan-2-yl)-1H-pyrazol-1-yl]acetamide 163 in the form of a pale yellow oil.

LC-MS (7 min): Rt (min)=2.22; [M+H]⁺: m/z 323.

### Step 2

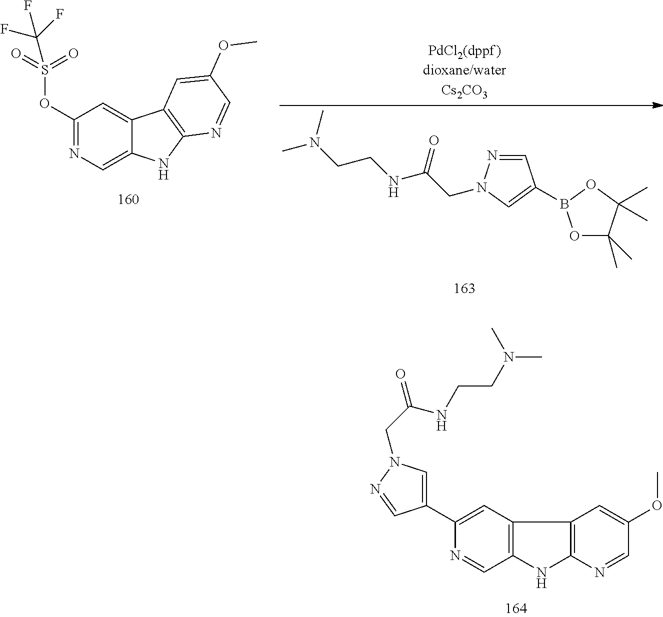

52 mg of 3-methoxy-9H-pyrrolo[2,3-b:5,4-c′]dipyrid-6-yl trifluoromethanesulfonate 160, 53 mg of N-[2-(dimethylamino)ethyl]-2-[4-(4,4,5,5-tetramethyl-1,3,2-dioxaborolan-2-yl)-1H-pyrazol-1-yl]acetamide 163, 7 mg of 1,1′-bis(diphenylphosphino)ferrocenedichloropalladium(II) in 0.75 mL of 1,4-dioxane and 0.30 mL of aqueous 1.5 M caesium carbonate solution are placed in a reactor under argon and the mixture is then subjected to microwave irradiation for 45 minutes at 150° C. The reaction mixture is concentrated and the residue obtained is then taken up in DMF, silica is added, and this mixture is concentrated under reduced pressure to give a solid deposit, which is purified by chromatography on a column of silica, eluting with an 80/20/1 dichloromethane/meth-anol/concentrated aqueous ammonia mixture to give 25 mg of N-[2-(dimethylamino)ethyl]-2-[4-(3-methoxy-9H-pyrrolo[2,3-b:5,4-c′]dipyrid-6-yl)-1H-pyrazol-1-yl]acetamide 164 in the form of a brown solid.

1H NMR (400 MHz, DMSO-d6) δ □ppm 2.56 to 2.69 (broad m, 6 H); 2.86 to 3.04 (broad m, 2 H); 3.37 to 3.45 (broad m, 2 H); 3.93 (s, 3 H); 4.90 (s, 2H); 8.02 (s, 1 H); 8.23 (s, 1 H); 8.28 (d, J=2.9 Hz, 1 H); 8.29 to 8.33 (broad m, 1 H); 8.36 (d, J=2.9 Hz, 1H); 8.44 (s, 1 H); 8.80 (s, 1 H); 9.58 to 10.29 (broad m, 1 H); 11.89 (broad s, 1 H).

UPLC-SQD: Rt (min)=0.31; [M+H]⁺: m/z 394; [M−H]⁻: m/z 392

### Example 166

### N,N-diethyl-3-[4-(3-methoxy-9H-pyrrolo[2,3-b:5,4-c′]dipyrid-6-yl)-1H-pyrazol-1-yl]-propan-1-amine 165

### Step 1

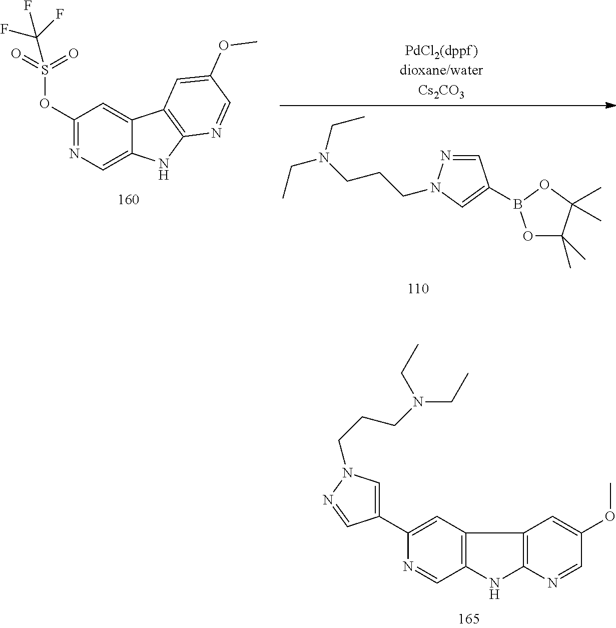

37 mg of 3-methoxy-9H-pyrrolo[2,3-b:5,4-c′]dipyrid-6-yl trifluoromethanesulfonate 160, 36 mg of N,N-diethyl-3-[4-(4,4,5,5-tetramethyl-1,3,2-dioxaborolan-2-yl)-1H-pyrazol-1-yl]propan-1-amine 110, 5 mg of 1,1′-bis(diphenylphosphino)ferrocenedichloropalladium(II) in 0.50 mL of 1,4-dioxane and 0.21 mL of aqueous 1.5 M caesium carbonate solution are placed in a reactor under argon and the mixture is then subjected to microwave irradiation for 45 minutes at 150° C. The reaction mixture is concentrated and the residue obtained is then taken up in DMF, silica is added, and this mixture is concentrated under reduced pressure to give a solid deposit, which is purified by chromatography on a column of silica, eluting with an 80/20/1 dichloromethane/methanol/concentrated aqueous ammonia mixture to give 18 mg of N,N-diethyl-3-[4-(3-methoxy-9H-pyrrolo[2,3-b:5,4-c′]dipyrid-6-yl)-1H-pyrazol-1-yl]propan-1-amine 165.

1H NMR (400 MHz, DMSO-d6) δ □ppm 0.90 to 1.13 (broad m, 6 H); 1.92 to 2.09 (broad m, 2 H); 2.35 to 3.20 (partially masked broad m, 6 H); 3.93 (s, 3 H); 4.15 to 4.25 (broad m, 2 H); 8.02 (s, 1 H); 8.24 (s, 1 H); 8.27 (d, J=2.9 Hz, 1 H); 8.35 (d, J=2.9 Hz, 1 H); 8.42 (broad s, 1 H); 8.81 (d, J=1.2 Hz, 1 H); 11.85 (broad s, 1 H).

UPLC-SQD: Rt (min)=0.36; [M+H]⁺: m/z 379.

### Example 167

### 2-methyl-2-propyl {5-[4-(3-methoxy-9H-pyrrolo[2,3-b:5,4-c′]dipyrid-6-yl)-1H-pyrazol-1-yl]pentyl}carbamate 167

Step 1: 2-methyl-2-propyl {5-[4-(4,4,5,5-tetramethyl-1,3,2-dioxaborolan-2-yl)-1H-pyrazol-1-yl]-pentyl}carbamate 166

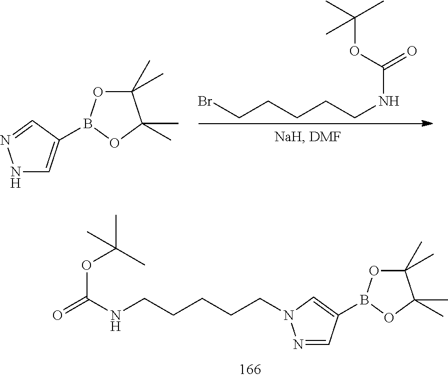

100 mg of 4-(4,4,5,5-tetramethyl-1,3,2-dioxaborolan-2-yl)-1H-pyrazole, 137 mg of N-(5-bromopentyl)-2,2-dimethylpropanamide, 671 mg of caesium carbonate and 2.0 mL of tetrahydrofuran are introduced into a microwave reactor of suitable size. The mixture is irradiated for 1 hour at 130° C. The reaction medium is concentrated and then taken up in ethyl acetate and washed twice with water. The organic phase is dried over magnesium sulfate, filtered and then concentrated under reduced pressure to give 145 mg of 2-methyl-2-propyl {5-[4-(4,4,5,5-tetramethyl-1,3,2-dioxaborolan-2-yl)-1H-pyrazol-1-yl]pentyl}carbamate 166 in the form of a colourless oil, which is used in crude form in the following step.

UPLC-SQD: Rt (min)=1.03; [M+H]⁺: m/z 380.

Step 2: 2-methyl-2-propyl {5-[4-(3-methoxy-9H-pyrrolo[2,3-b:5,4-c′]dipyrid-6-yl)-1H-pyrazol-1-yl]-pentyl}carbamate 167

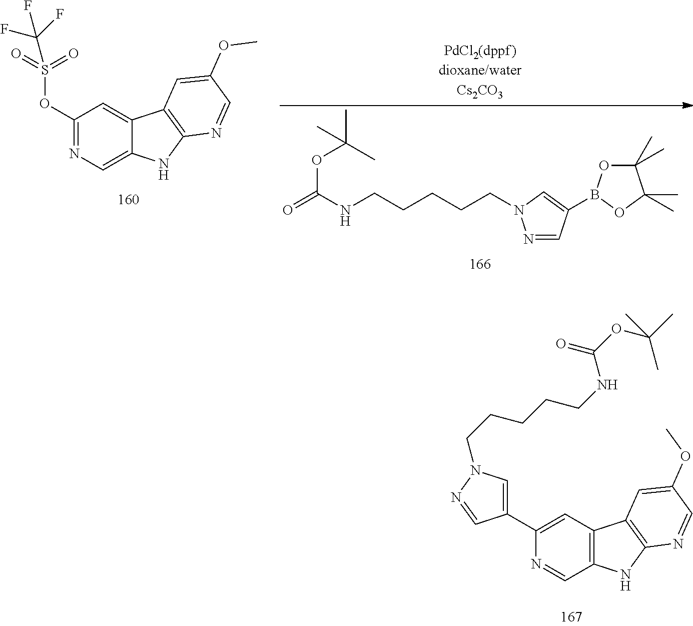

70 mg of 3-methoxy-9H-pyrrolo[2,3-b:5,4-c′]dipyrid-6-yl trifluoromethanesulfonate 160, 91 mg of 2-methyl-2-propyl {5-[4-(4,4,5,5-tetramethyl-1,3,2-dioxaborolan-2-yl)-1H-pyrazol-1-yl]pentyl}-carbamate 166, 9 mg of 1,1′-bis(diphenylphosphino)ferrocenedichloropalladium(II) in 1.0 mL of 1,4-dioxane and 0.4 mL of aqueous 1.5 M caesium carbonate solution are placed in a reactor under argon and the mixture is then subjected to microwave irradiation for 45 minutes at 150° C. The reaction mixture is concentrated and the residue obtained is then taken up in DMSO and purified by HPLC in acidic medium to give 32 mg of 2-methyl-2-propyl {5-[4-(3-methoxy-9H-pyrrolo[2,3-b:5,4-c′]-dipyrid-6-yl)-1H-pyrazol-1-yl]pentyl}carbamate 167 in the form of a salt with trifluoroacetic acid.

1H NMR (400 MHz, DMSO-d6) δ □ppm 1.19 to 1.29 (m, 2 H); 1.35 (s, 9 H); 1.37 to 1.46 (m, 2 H); 1.78 to 1.88 (m, 2 H); 2.87 to 2.93 (m, 2 H); 3.95 (s, 3 H); 4.19 (t, J=6.9 Hz, 2 H); 6.81 (t, J=5.6 Hz, 1 H); 8.05 to 8.17 (m, 1 H); 8.30 to 8.42 (m, 2 H); 8.45 to 8.58 (m, 1 H); 8.60 to 8.79 (broad m, 1 H); 8.85 to 8.95 (m, 1 H); 11.85 to 12.74 (broad m, 1 H).

### Example 168

### 5-[4-(3-methoxy-9H-pyrrolo[2,3-b:5,4-c′]dipyrid-6-yl)-1H-pyrazol-1-yl]pentan-1-amine 168

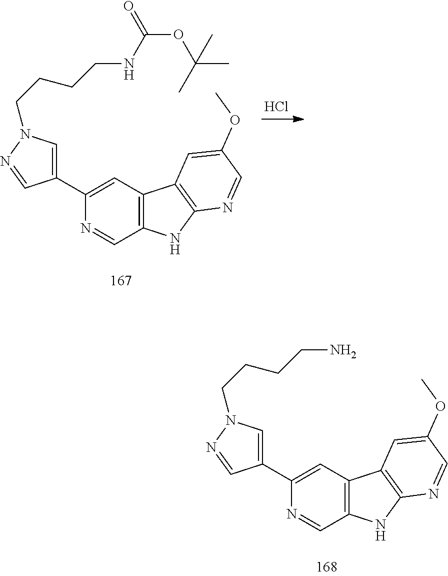

To 150 mg of product of compound 167 are added 2 mL of 4N hydrochloric acid solution in 1,4-dioxane. The reaction medium is stirred with ultrasonication at 25° C. for 1 hour and then filtered; the solid obtained is rinsed three times with ethyl acetate to give 135 mg of 5-[4-(3-methoxy-9H-pyrrolo-[2,3-b:5,4-c′]dipyrid-6-yl)-1H-pyrazol-1-yl]pentan-1-amine 168 in the form of the hydrochloride as an ochre-coloured solid.

1H NMR (400 MHz, DMSO-d6) δ □ppm 1.29 to 1.41 (m, 2 H); 1.55 to 1.68 (m, 2 H); 1.79 to 1.94 (m, 2 H); 2.72 to 2.84 (m, 2 H); 3.97 (s, 3 H); 4.25 (t, J=6.7 Hz, 2 H); 7.72 to 7.92 (broad m, 3 H); 8.26 to 8.34 (broad m, 1 H); 8.42 to 8.47 (m, 1 H); 8.56 to 8.63 (m, 1 H); 8.63 to 8.72 (broad m, 1 H); 8.89 to 8.93 (m, 1 H); 8.95 to 9.10 (broad m, 1 H); 12.41 to 13.03 (broad m, 1 H).

UPLC-SQD: Rt (min)=0.35; [M+H]⁺: m/z 351.

### Example 169

### 3-methoxy-6-{1-[2-(1-methylpiperid-2-yl)ethyl]-1H-pyrazol-4-yl}-9H-pyrrolo[2,3-b:5,4-c′]dipyridine 170

Step 1: 1-methyl-2-{2-[4-(4,4,5,5-tetramethyl-1,3,2-dioxaborolan-2-yl)-1H-pyrazol-1-yl]ethyl}-piperidine 169

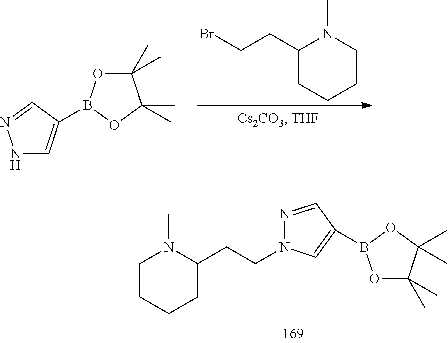

388 mg of 4-(4,4,5,5-tetramethyl-1,3,2-dioxaborolan-2-yl)-1H-pyrazole, 412 mg of 2-(2-bromoethyl)-1-methylpiperidine, 2.6 g of caesium carbonate and 8 mL of tetrahydrofuran are introduced into a microwave reactor of suitable size. The mixture is irradiated for 1 hour at 130° C. The reaction medium is diluted with ethyl acetate and washed three times with water. The organic phase is dried over magnesium sulfate, filtered and then concentrated under reduced pressure to give 482 mg of 1-methyl-2-{2-[4-(4,4,5,5-tetramethyl-1,3,2-dioxaborolan-2-yl)-1H-pyrazol-1-yl]ethyl}piperidine 169 in the form of a colourless oil, which is used in crude form in the following step.

UPLC-SQD: Rt (min)=0.51; [M+H]⁺: m/z 320.

### Step 2

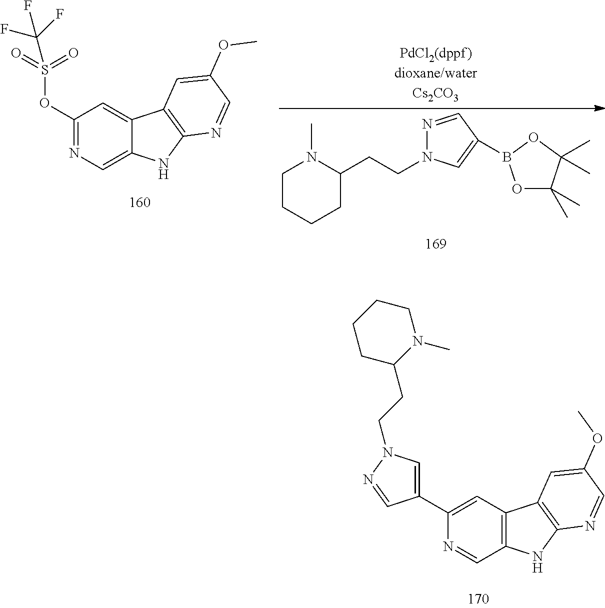

70 mg of 3-methoxy-9H-pyrrolo[2,3-b:5,4-c′]dipyrid-6-yl trifluoromethanesulfonate 160, 91 mg of 1-methyl-2-{2-[4-(4,4,5,5-tetramethyl-1,3,2-dioxaborolan-2-yl)-1H-pyrazol-1-yl]ethyl}piperidine 169, 9 mg of 1,1′-bis(diphenylphosphino)ferrocenedichloropalladium(II) in 1.0 mL of 1,4-dioxane and 0.4 mL of aqueous 1.5 M caesium carbonate solution are placed in a reactor under argon and the mixture is then subjected to microwave irradiation for 45 minutes at 150° C. The reaction mixture is concentrated and the residue obtained is then taken up in DMSO and purified by HPLC in acidic medium to give 32 mg of 3-methoxy-6-{1-[2-(1-methylpiperid-2-yl)ethyl]-1H-pyrazol-4-yl}-9H-pyrrolo[2,3-b:5,4-c′]dipyridine 170.

1H NMR (400 MHz, DMSO-d6) δ □ppm 1.04 to 3.58 (m, 14 H); 3.93 (s, 3 H); 4.24 (m, 2 H); 8.02 (s, 1 H); 8.26 (d, J=2.9 Hz, 1 H); 8.30 (s, 1 H); 8.36 (d, J=2.9 Hz, 1 H); 8.41 (broad s, 1 H); 8.81 (d, J=1.2 Hz, 1 H); 11.86 (s, 1 H).

UPLC-SQD: Rt (min)=0.36; [M+H]⁺: m/z 391; [M+2H]²⁺: m/z 196 (base peak).

### Example 170

### methyl 4-{6-[1-(prop-2-en-1-yl)-1H-pyrazol-4-yl]-9H-pyrrolo[2,3-b:5,4-c′]dipyrid-4-yl}benzoate 177

Step 1: 5′-chloro-2′,4-dimethoxy-3,4′-bipyrid-2-amine 171

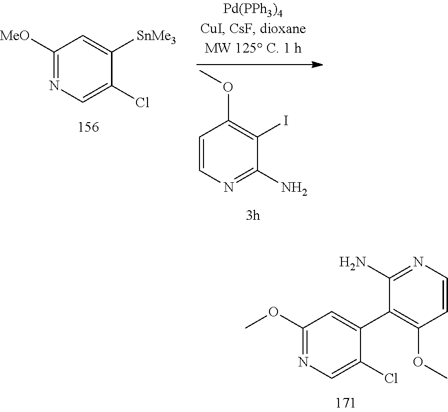

368 mg of 5-chloro-2-methoxy-4-(trimethylstannyl)pyridine 156, 250 mg of 3-iodo-4-methoxypyrid-2-ylamine 3h, 304 mg of caesium fluoride and 38 mg of copper iodide in 2 mL of dimethylformamide are placed in a tube, 116 mg of tetrakis(triphenylphosphine)palladium(0) and 2 mL of dimethylformamide are then added and the tube is sealed and subjected to microwave irradiation at 125° C. for 2 hours. The reaction mixture is filtered through Celite, rinsed with 10 mL of ethyl acetate and then washed with twice 10 mL of water. After separation of the phases by settling, the organic phase is dried over magnesium sulfate, filtered and then concentrated to dryness under reduced pressure. The residue is purified by chromatography on a column of silica, eluting with a 50/50 to 0/100 heptane/ethyl acetate mixture to give 125 mg of 5′-chloro-2′,4-dimethoxy-3,4′-bipyrid-2-amine 171 in the form of a white solid.

UPLC-MS-DAD-ELSD: Rt (min)=0.44; [M+H]⁺: m/z 266.

1H NMR (400 MHz, DMSO-d6) δ □ppm: 3.68 (s, 3 H) 3.87 (s, 3 H) 5.40 (s, 2 H) 6.42 (d, J=5.9 Hz, 1 H) 6.72 (d, J=0.5 Hz, 1 H) 7.94 (d, J=5.9 Hz, 1 H) 8.28 (d, J=0.5 Hz, 1 H).

Step 2: 4,6-dimethoxy-9H-pyrrolo[2,3-b:5,4-c′]dipyridine 172

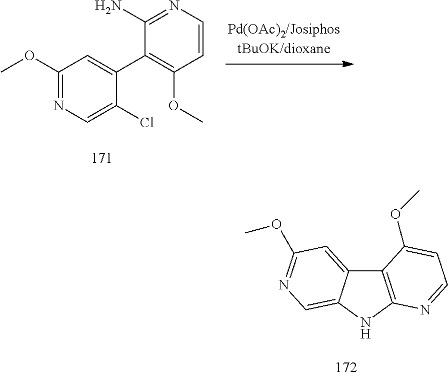

10.3 mg of (R)-(−)-1-[(S)-2-(dicyclohexylphosphino)ferrocenyl]ethyldi-tert-butylphosphine and 3.8 mg of palladium (II) acetate in 0.35 mL of anhydrous 1,4-dioxane are placed in a 2 ml tube under an argon atmosphere, and the mixture is stirred for 10 minutes at 35° C.

45 mg of 5′-chloro-2′,4-dimethoxy-3,4′-bipyrid-2-amine 171 and 38 mg of potassium tert-butoxide in 0.35 mL of anhydrous 1,4-dioxane are placed in a 2 ml reactor under argon, the solution prepared previously and 0.20 mL, of 1,4-dioxane are then added, and the tube is sealed and subjected to microwave irradiation for 1 hour at 130° C. The reaction mixture is diluted with a 90/10 dichloromethane/methanol mixture and then filtered. After concentrating under reduced pressure, the residue is purified by chromatography on a column of silica, eluting with a 98/2 to 94/6 dichloromethane/methanol mixture to give 28.5 mg of 4,6-dimethoxy-9H-pyrrolo[2,3-b:5,4-c′]-dipyridine 172 in the form of a yellow solid.

UPLC-MS-DAD-ELSD: Rt (min)=0.40; [M+H]⁺: m/z 230.

1H NMR (400 MHz, DMSO-d6) δ □ppm: 3.89 (s, 3 H) 4.09 (s, 3 H) 6.85 (d, J=5.9 Hz, 1 H) 7.30 (d, J=1.0 Hz, 1 H) 8.39-8.42 (m, 2 H) 11.70 (br. s., 1 H).

Step 3: 9H-pyrrolo[2,3-b:5,4-c′]dipyridine-4,6-diol hydrochloride 173

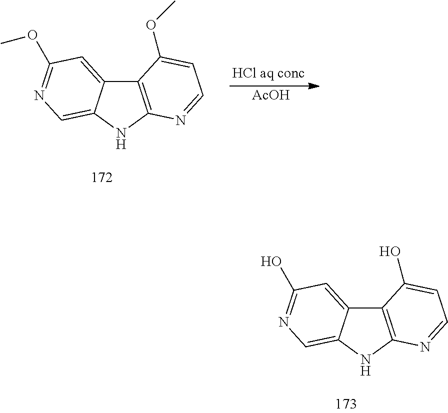

1.52 g of 4,6-dimethoxy-9H-pyrrolo[2,3-b:5,4-c′]dipyridine 172 in 22.1 ml of acetic acid and 7.3 mL of 37% hydrochloric acid solution are placed in a 20 ml reactor, and the tube is sealed and subjected to microwave irradiation for 2 hours at 140° C. After concentrating the reaction mixture, the solid obtained is slurried in twice 25 ml of diethyl ether and then dried under reduced pressure for 18 hours to give 1.72 g of 9H-pyrrolo[2,3-b:5,4-c′]dipyridine-4,6-diol hydrochloride 173 in the form of a dark beige-coloured solid.

UPLC-MS-DAD-ELSD: Rt (min)=0.14; [M+H]⁺: m/z 202; [M−H]⁻: m/z 200.

1H NMR (400 MHz, DMSO-d6) δ □ppm: 6.48 (m, 1 H) 7.62 (s, 1 H) 8.06 (d, J=7.1 Hz, 1 H) 8.34 (s, 1 H) 12.48 (br. s, 1 H)

Step 4: 9H-pyrrolo[2,3-b:5,4-c′]dipyridine-4,6-diylbis(trifluoromethanesulfonate) 174

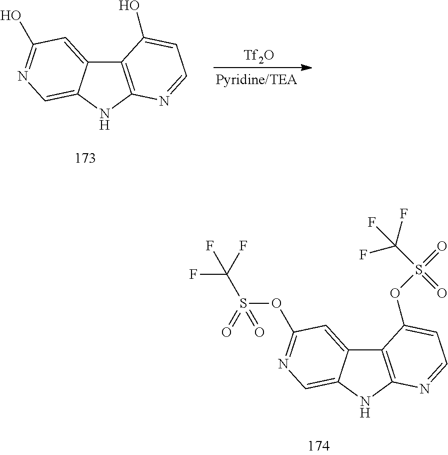

A mixture of 1.72 g of 9H-pyrrolo[2,3-b:5,4-c′]dipyridine-4,6-diol hydrochloride 173 in 35 mL of pyridine and 9.1 mL of triethylamine is cooled to 5° C., followed by addition of 2.8 ml of trifluoromethanesulfonic anhydride. The reaction mixture is stirred at 0-5° C. for 1 hour and then poured into a mixture of 200 mL of water and 50 mL of saturated aqueous sodium chloride solution and extracted with 250 mL of ethyl acetate. After separation of the phases by settling, the aqueous phase is extracted with 200 ml ethyl acetate and the organic phases are then combined and concentrated under vacuum. The residue is taken up in a mixture of 100 mL of an 80/20 dichloromethane/ethyl acetate mixture, 6.0 g of silica are added, and this mixture is concentrated under reduced pressure. The solid deposit formed is purified by chromatography on a column of silica, eluting with a 100/0 to 80/20 dichloromethane/ethyl acetate mixture to give 124 mg of 9H-pyrrolo[2,3-b:5,4-c′]dipyridine-4,6-diyl bis(trifluoromethanesulfonate) 174 in the form of a rust-coloured solid.

UPLC-MS-DAD-ELSD: Rt (min)=4.81; [M+H]³⁰: m/z 466; [M−H]⁻: m/z 464.

1H NMR (400 MHz, DMSO-d6) δ □ppm: 7.59 (d, J=5.6 Hz, 1 H) 7.96 (s, 1 H) 8.88 (d, J=0.7 Hz, 1 H) 8.89 (d, J=5.6 Hz, 1 H) 13.32 (br. s., 1 H).

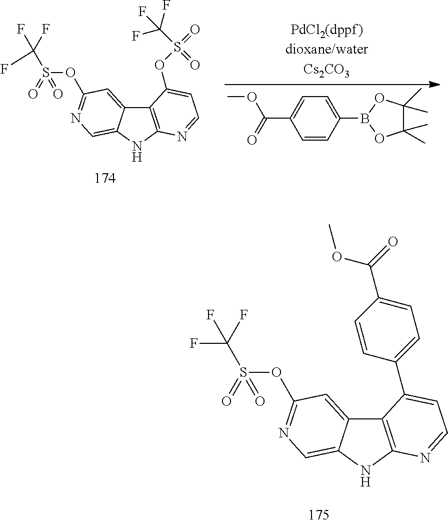

158 mg of 9H-pyrrolo[2,3-b:5,4-c′]dipyridine-4,6-diyl bis(trifluoromethanesulfonate) 174, 89 mg of boronate, 166 mg of caesium carbonate, 1.55 mL of dioxane, 25 mg of 1,1′-bis(diphenyl-phosphino)ferrocenedichloropalladium(II) and 0.15 mL of water are introduced into a microwave reactor of suitable size, under argon. The mixture is irradiated for 15 minutes at 140° C. The suspension obtained is diluted with ethyl acetate and washed with saturated aqueous ammonium chloride solution. The aqueous phase is extracted with ethyl acetate and the organic phases are then combined, dried over magnesium sulfate, filtered and concentrated under reduced pressure. The residue is purified by chromatography on a column of silica, eluting with an 80/20 to 50/50 dichloromethane/ethyl acetate mixture to give 30 mg of 175 in the form of a yellow solid.

UPLC-SQD: Rt (min)=1.08; [M+H]⁺: m/z 452; [M−H]⁻: m/z 450.

### Step 6:

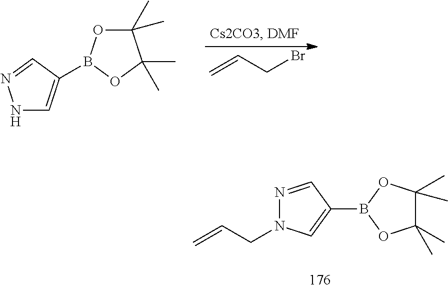

621 mg of 4-(4,4,5,5-tetramethyl-1,3,2-dioxaborolan-2-yl)-1H-pyrazole, 2.08 g of caesium carbonate, 16 mL, of dimethylformamide and 0.55 mL of allyl bromide are introduced into a microwave reactor of suitable size. The mixture is irradiated for 1 hour at 100° C. The suspension obtained is diluted with ethyl acetate and washed with aqueous sodium bicarbonate solution. The aqueous phase is extracted with ethyl acetate and the organic phases are then combined, dried over magnesium sulfate, filtered and concentrated under reduced pressure to give 292 mg (39%) of 1-(prop-2-enyl)-4-(4,4,5,5-tetramethyl-1,3,2-dioxaborolan-2-yl)-1H-pyrazole 176.

LC-MS (7 min): Rt (min)=3.68; [M+H]⁺: m/z 235.

Step 7:

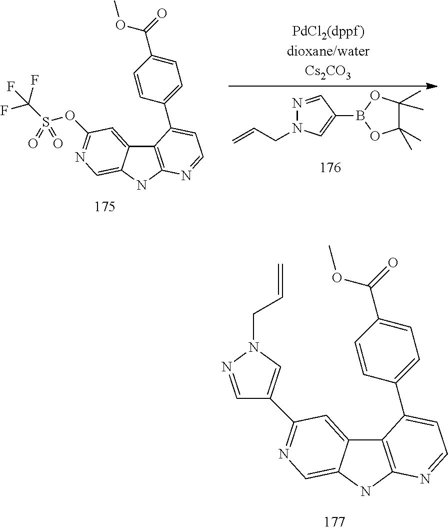

28 mg of the product of step 5 175, 22 mg of boronate 176 prepared in step 6, 30 mg of caesium carbonate, 0.45 mL of dioxane, 5 mg of 1,1′-bis(diphenylphosphino)ferrocenedichloropalladium(II) and 50 μl of water are introduced into a microwave reactor of suitable size, under argon. The mixture is irradiated for 30 minutes at 130° C. A further 4 mg of 1,1′-bis(diphenylphosphino)ferrocene-dichloropalladium(II), 15 mg of boronate 176 prepared in step 6, and 0.1 mL of dioxane are added and this mixture is irradiated for a further 1 hour at 140° C. The suspension obtained is diluted with ethyl acetate and washed with water. The aqueous phase is extracted with ethyl acetate and the organic phases are then combined, dried over magnesium sulfate, filtered and then concentrated under reduced pressure. The residue is purified by chromatography on a column of silica, eluting with a 100/0 to 96/4 dichloromethane/methanol mixture to give 8 mg of methyl 4-{6-[1-(prop-2-en-1-yl)-1H-pyrazol-4-yl]-9H-pyrrolo[2,3-b:5,4-c′]dipyrid-4-yl}benzoate 177 in the form of an ochre-coloured solid.

1H NMR (400 MHz, DMSO-d6) δ □ppm: 3.96 (s, 3 H); 4.77 (broad d, J=5.7 Hz, 2 H); 5.15 (broad d, J=16.9 Hz, 1 H); 5.21 (broad d, J=9.8 Hz, 1 H); 5.96 to 6.11 (m, 1 H); 7.28 (d, J=4.9 Hz, 1 H); 7.61 (s, 1 H); 7.67 (s, 1 H); 7.93 (d, J=8.3 Hz, 2 H); 8.00 (s, 1 H); 8.27 (d, J=8.3 Hz, 2 H); 8.65 (d, J=4.9 Hz, 1 H); 8.90 (s, 1 H); 12.34 to 12.41 (broad s, 1 H).

LC-MS (7 min): Rt (min)=3.19; [M+H]⁺: m/z 410; [M−H]⁻: m/z 408.

### Example 171

### N-{-4-[3-fluoro-6-(1-methyl-1H-pyrazol-4-yl)-9H-pyrrolo[2,3-b:5,4-c′]dipyrid-4-yl]-phenyl}methane sulfonamide 183

Step 1

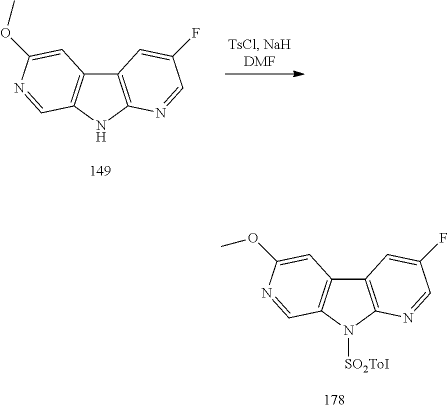

7.6 g of 3-fluoro-6-methoxy-9H-pyrrolo[2,3-b:5,4-c′]dipyridine in 180 mL of dimethylformamide and 2.38 g of 60% sodium hydride in oil are placed in a 500 ml reactor, under argon. After stirring for 3 hours at room temperature, 13.3 g of para-toluenesulfonyl chloride dissolved in 20 mL of dimethylformamide are added. The reaction medium is stirred for 3 hours at room temperature and then poured into aqueous 5% sodium hydrogen carbonate solution. The crude product is recovered by filtration and air-dried. After redissolving in dichloromethane, 24 g of silica are added and the whole is concentrated under reduced pressure. Purification by chromatography on a column of silica, eluting with a 100/0 to 95/5 dichloromethane/methanol mixture, gives 11.6 g of 3-fluoro-9-[(4-methylphenyl)-sulfonyl]-6-methoxy-9H-pyrrolo[2,3-b:5,4-c′]dipyridine.

UPLC-MS-DAD-ELSD: 372.11(+)=(M+H)(+) Rt (min)=1.35;

### Step 2

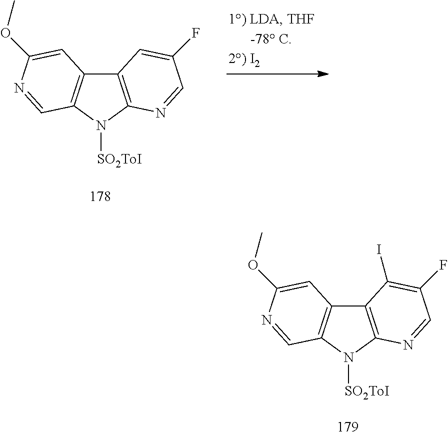

3.1 mL of diisopropylamine are placed in 40 mL of tetrahydrofuran in a dry three-necked flask under argon. After stirring and cooling to −78° C., 8.1 mL of 2.5N n-butyllithium in hexane are added dropwise. The reaction mixture is stirred for 15 minutes at −78° C., followed by addition of 5.0 g of 3-fluoro-9-[(4-methylphenyl)sulfonyl]-6-methoxy-9H-pyrrolo[2,3-b:5,4-c′]dipyridine 178 in 250 mL of tetrahydrofuran. After stirring for 2 hours at −78° C., 5.46 g of iodine in 10 mL of tetrahydrofuran are added. After stirring for 1 hour, the reaction mixture is poured into 400 ml of aqueous 10% ammonium chloride solution and 250 mL of water, and extracted twice with 400 mL of ethyl acetate. The organic phases are washed with aqueous 5% sodium thiosulfate solution, dried over sodium sulfate, filtered and concentrated to dryness under reduced pressure. 6.57 g of 3-fluoro-4-iodo-9-[(4-methylphenyl)sulfonyl]-6-methoxy-9H-pyrrolo[2,3-b:5,4-c′]dipyridine 179 are obtained.

UPLC-MS-DAD-ELSD: 498.01(+) Rt (min)=1.43;

### Step 3

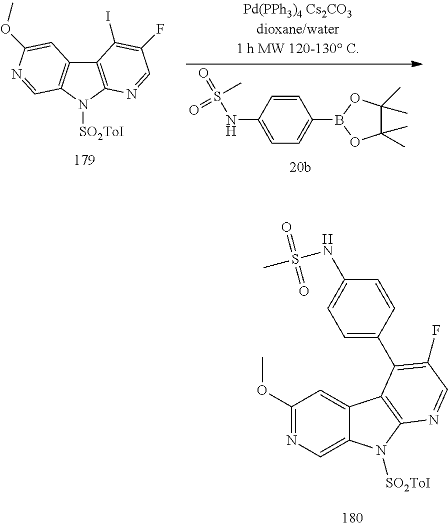

278 mg (0.84 mmol) of 3-fluoro-4-iodo-9-[(4-methylphenyl)sulfonyl]-6-methoxy-9H-pyrrolo[2,3-b:5,4-c′]dipyridine 179, 1.67 mmol of the boronate 20b, 77 mg of tetrakis(triphenylphosphine)-palladium(0), 273 mg of caesium carbonate, 5.5 mL of dioxane and 1.3 mL of water are introduced into a microwave reactor of suitable size. The mixture is irradiated for 1 hour at 120. 3 mL of methanol are added and the mixture is then poured into water (50 mL) and ethyl acetate (100 mL), the phases are separated and the aqueous phase is again extracted with 100 mL of ethyl acetate. The combined organic phases are dried over magnesium sulfate, filtered and then concentrated under reduced pressure. The residue is purified by chromatography on silica gel (60 g of silica, gradient: 100/0 to 50/50 dichloromethane/ethyl acetate) to give 250 mg (83%) of the expected compound N-[4-(3-fluoro-9-[(4-methylphenyl)sulfonyl]-6-methoxy-9H-pyrrolo[2,3-b:5,4-c′]dipyrid-4-yl]phenyl}-methanesulfonamide 180.

UPLC-MS-DAD-ELSD: 541.14(+)=(M+H)(+) Rt (min)=1.31;

Step 4

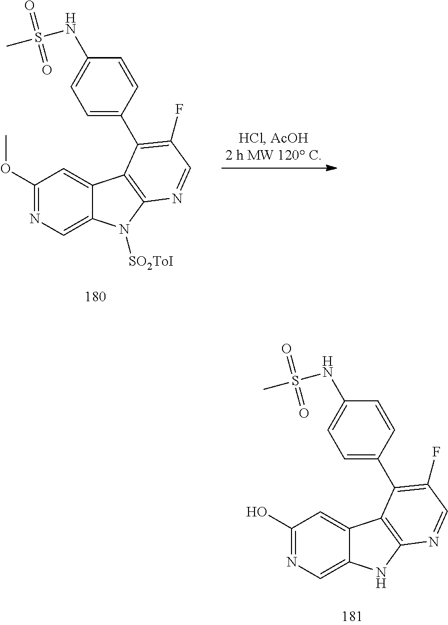

To a solution of 305 mg of N-[4-(3-fluoro-9-[(4-methylphenyl)sulfonyl]-6-methoxy-9H-pyrrolo[2,3-b:5,4-c′]dipyrid-4-yl]phenyl}methanesulfonamide 180 in 5.5 mL of acetic acid is added 0.5 mL of 37% hydrochloric acid solution. The mixture is heated by microwave for 2 hours at 120° C., and poured into a mixture of ethyl acetate and aqueous 5% potassium carbonate solution with vigorous stirring. The pH is brought to 7 by adding aqueous 5N HCl solution. After separating the phases, the organic phase is dried over MgSO₄, filtered and then concentrated under reduced pressure to give 99 mg of N-[4-(3-fluoro-6-hydroxy-9H-pyrrolo[2,3-b:5,4-c′]dipyrid-4-yl]phenyl}methanesulfonamide 181.

UPLC-MS-DAD-ELSD: 373.09(+)=(M+H)(+) Rt (min)=0.65;

### Step 5

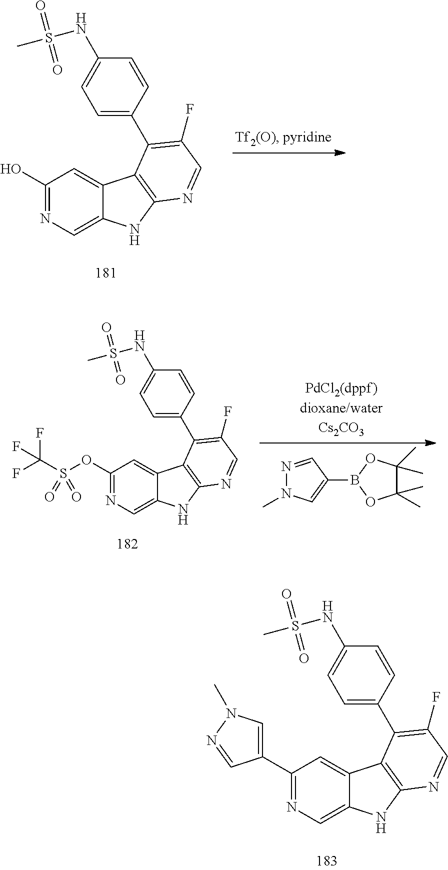

To a suspension of 98 mg of N-[4-(3-fluoro-6-hydroxy-9H-pyrrolo[2,3-b:5,4-c′]dipyrid-4-yl]phenyl}-methanesulfonamide 181 in 5 mL of pyridine is added 0.27 ml of trifluoromethanesulfonic anhydride. The reaction medium is stirred for 2 hours and then concentrated under reduced pressure. The reaction medium is poured into a mixture of ethyl acetate and aqueous 5% sodium bicarbonate solution with vigorous stirring. After the separating the phases, the organic phase is dried over MgSO₄, filtered and then concentrated under reduced pressure.

UPLC-MS-DAD-ELSD: 504=(M+H)(+) Rt (min)=1.14

The brown solid 182 is placed in a microwave tube with 20 μmol of 1,1′-bis(diphenylphosphino)-ferrocenedichloropalladium(II), 0.89 mmol of caesium carbonate, 4 mL of dioxane and 1 mL of water and 0.52 mmol of 1-methyl-4-(4,4,5,5-tetramethyl-1,3,2,dioxaborolan-2-yl)-1H-pyrazole. The mixture obtained is then irradiated for 1 hour at 120° C., 1 mL of methanol is added and the mixture is then poured into water (25 mL) and ethyl acetate (50 mL), the phases are separated and the aqueous phase is again extracted with 50 mL of ethyl acetate. The organic phases are combined and dried over magnesium sulfate, filtered and then concentrated under reduced pressure. The residue is purified by chromatography on silica gel (30 g of silica, gradient: 100/0 to 90/10 dichloromethane/methanol) to give 70 mg (62%) of the expected compound N-{-4-[3-fluoro-6-(1-methyl-1H-pyrazol-4-yl)-9H-pyrrolo[2,3-b:5,4-c′]dipyrid-4-yl]phenyl}methane sulfonamide 183.

1H NMR (400 MHz, DMSO-d6) δ ppm: 3.17 (s, 3 H); 3.85 (s, 3 H); 7.40 (d, J=0.9 Hz, 1 H); 7.51 (d, J=8.6 Hz, 2 H); 7.59 (s, 1 H); 7.69 (d, J=8.6 Hz, 2 H); 7.91 (s, 1 H); 8.67 (d, J=2.5 Hz, 1 H); 8.87 (d, J=0.9 Hz, 1 H); 9.92 to 10.24 (broad m, 1 H); 12.26 (broad s, 1 H)

UPLC-SQD: Rt (min)=0.55; MH+=437+; MH−=435

### Example 172 (187) and Example 173

### (4-methylpiperazin-1-yl)(9H-pyrrolo[2,3-b:5,4-c′]dipyrid-6-yl)-methanone 188

Step 1: 5-chloro-2-cyano-4-trimethylstannylpyridine 184

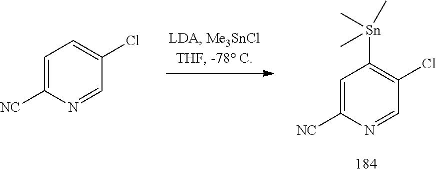

5 g of 5-chloro-2-cyanopyridine, 9.35 g of trimethyltin chloride and 200 mL of THF are placed in a dry one-necked flask under argon. The mixture is stirred and cooled to −78° C., followed by dropwise addition of 19.85 ml of a commercial solution of LDA (2N in toluene) over 45 minutes. The medium turns yellow and then brown. After stirring for 3 hours, the reaction medium is hydrolysed with ammonium chloride solution, and the aqueous phase is then extracted with ethyl acetate. The organic phase is dried over magnesium sulfate, filtered and concentrated under reduced pressure. The crude residue obtained is purified by chromatography on silica gel using a gradient of 0 to 2% ethyl acetate in heptane. The fractions containing the expected product are combined and concentrated under reduced pressure to give 3.4 g (31%) of 5-chloro-2-cyano-4-trimethylstannylpyridine 184 in the form of a white solid.

UPLC-MS-DAD-ELSD: 302.97(+)=(M+H)(+) (isotope profile corresponding to a tin derivative) Rt (min)=1.39

Step 2: 9H-pyrrolo[2,3-b:5,4-c′]dipyridine-6-carbonitrile 186

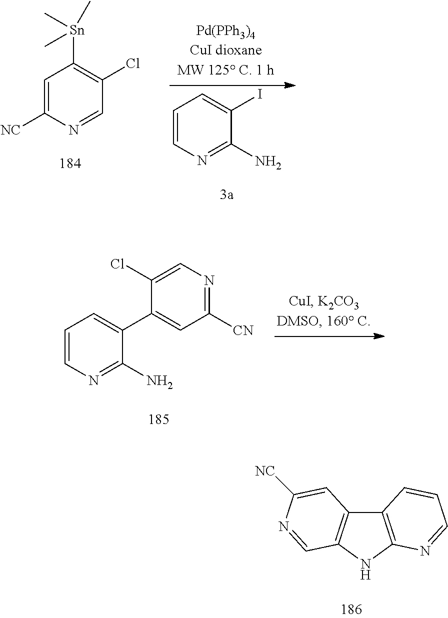

2.48 g of 2-amino-3-iodopyridine, 3.4 g of 5-chloro-2-cyano-4-trimethylstannylpyridine 184, 912 mg of tetrakis(triphenylphosphine)palladium(0), 451 mg of copper iodide and 15 mL of dioxane are introduced into a microwave reactor of suitable size. The reaction medium is irradiated for 1 hour at 120° C. and then hydrolysed with 75 ml of aqueous 10% sodium bicarbonate solution and 5 mL of water. The aqueous phase is extracted twice with 50 mL of ethyl acetate, and the combined organic phases are then dried over sodium sulfate, filtered and concentrated under reduced pressure. The crude residue is then crystallized by trituration in heptane. The product 185 recovered by filtration is dissolved in 80 mL of DMSO, then 1.32 g of copper iodide and 14.3 g of potassium carbonate are added. The mixture is heated overnight at 160° C. After cooling, the reaction medium is poured into a mixture of ethyl acetate and 28% aqueous ammonia with vigorous stirring. After stirring for 1 hour, the phases are separated and the organic phase is dried over MgSO₄, filtered and then concentrated under reduced pressure. The expected product is crystallized by trituration from dichloromethane, to give 1.1 g (50% over the two steps) of 9H-pyrrolo[2,3-b:5,4-c′]dipyridine-6-carbonitrile.

UPLC-MS-DAD-ELSD: 195(+)=(M+H)(+) Rt (min)=0.69

Step 3: 9H-pyrrolo[2,3-b:5,4-c′]dipyridine-6-carboxylic acid 187

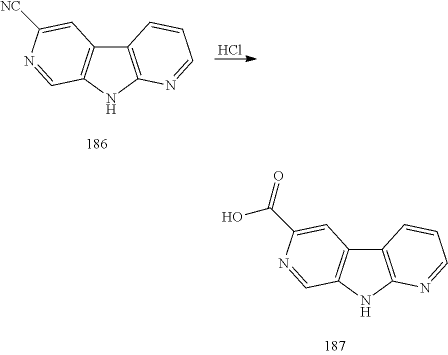

1.1 g of 9H-pyrrolo[2,3-b:5,4-c′]dipyridine-6-carbonitrile 186 are dissolved in 10 ml of aqueous 6N hydrochloric acid solution in a microwave machine tube. The mixture is heated by microwave at 140° C. for 1 hour. After cooling, the mixture is taken up in water and ethyl acetate, and the precipitate is filtered off. The pH of the filtrate is brought to 4, and the new precipitate formed is also isolated by filtration. The two solids have the same profile in UPLC-MS. 1.03 g (86%) of 9H-pyrrolo[2,3-b:5,4-c′]dipyridine-6-carboxylic acid 187 are obtained.

UPLC-SQD: Rt (min)=0.25; MH+=214+; MH−=212

1H NMR (400 MHz, DMSO-d6) δ ppm: 7.58 (dd, J=4.8 and 8.0 Hz, 1 H); 8.86 to 8.93 (m, 1 H); 9.08 to 9.17 (m, 2 H); 9.48 (broad s, 1H)

### Step 4

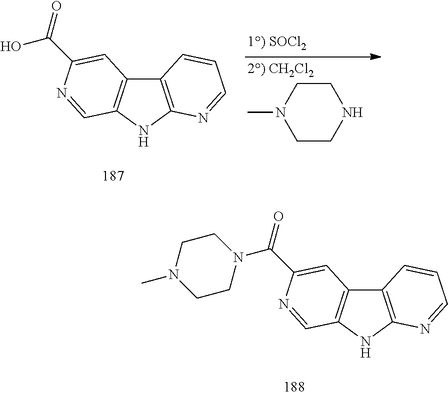

45 mg of 9H-pyrrolo[2,3-b:5,4-c′]dipyridine-6-carboxylic acid 187 and 5 mL of thionyl chloride are placed in a one-necked flask. The mixture is refluxed overnight with stirring and then concentrated under reduced pressure. The crude product is taken up in 5 mL of dichloromethane, and 215 μl of methylpiperazine are then added. After 1 hour, the reaction medium is concentrated under vacuum. The product is purified by preparative HPLC in acidic medium using a gradient of water+0.07% trifluoroacetic acid/acetonitrile+0.07% trifluoroacetic acid, to give 40 mg (50%) of (4-methylpiperazin-1-yl)(9H-pyrrolo[2,3-b:5,4-c′]dipyrid-6-yl)methanone 188.

Mass spectrometry: Spectrum acquired by chemical ionization (reactant gas: ammonia) on a Waters GCT of machine (direct introduction without LC): [M+H]+: m/z 296

1H NMR (400 MHz, DMSO-d6) δ ppm: 2.87 (s, 3 H); 3.05 to 3.67 (broad m, 6 H); 4.34 to 4.80 (broad m, 2 H); 7.37 (dd, J=4.9 and 7.9 Hz, 1 H); 8.61 (d, J=0.7 Hz, 1 H); 8.65 (dd, J=1.8 and 4.9 Hz, 1 H); 8.79 (dd, J=1.8 and 7.9 Hz, 1 H); 8.89 (d, J=0.7 Hz, 1 H); 9.62 to 10.06 (broad m, 1 H); 12.46 (s, 1 H)

The table below shows the correspondence between the compounds described in the schemes and preparations hereinabove, and, for the compounds that constitute products of formula (I), their respective example numbers from 1 to 173 in the present invention.

| Example | Compound |
|---|---|
| No. | No. |
| Example 1 | 5a |
| Example 2 | 5b |
| Example 3 | 5c |
| Example 4 | 5d |
| Example 5 | 5e |
| Example 6 | 5f |
| Example 7 | 5g |
| Example 8 | 5h |
| Example 9 | 6 |
| Example | 9a |
| 10 | |
| Example | 9b |
| 11 | |
| Example | 9c |
| 12 | |
| Example | 10 |
| 13 | |
| Example | 11 |
| 14 | |
| Example | 12 |
| 15 | |
| Example | 13 |
| 16 | |
| Example | 14 |
| 17 | |
| Example | 15 |
| 18 | |
| Example | 16 |
| 19 | |
| Example | 19 |
| 20 | |
| Example | 21a |
| 21 | |
| Example | 21b |
| 22 | |
| Example | 21c |
| 23 | |
| Example | 21d |
| 24 | |
| Example | 21e |
| 25 | |
| Example | 21f |
| 26 | |
| Example | 21g |
| 27 | |
| Example | 21h |
| 28 | |
| Example | 21i |
| 29 | |
| Example | 21j |
| 30 | |
| Example | 21k |
| 31 | |
| Example | 22 |
| 32 | |
| Example | 23 |
| 33 | |
| Example | 27a |
| 34 | |
| Example | 27b |
| 35 | |
| Example | 27c |
| 36 | |
| Example | 29 |
| 37 | |
| Example | 31 |
| 38 | |
| Example | 39a |
| 39 | |
| Example | 39b |
| 40 | |
| Example | 39c |
| 41 | |
| Example | 42 |
| 42 | |
| Example | 45 |
| 43 | |
| Example | 46 |
| 44 | |
| Example | 59 |
| 45 | |
| Example | 61 |
| 46 | |
| Example | 64 |
| 47 | |
| Example | 66 |
| 48 | |
| Example | 68 |
| 49 | |
| Example | 70 |
| 50 | |
| Example | 71a |
| 51 | |
| Example | 71b |
| 52 | |
| Example | 71c |
| 53 | |
| Example | 71d |
| 54 | |
| Example | 71e |
| 55 | |
| Example | 71f |
| 56 | |
| Example | 71g |
| 57 | |
| Example | 71h |
| 58 | |
| Example | 71i |
| 59 | |
| Example | 71j |
| 60 | |
| Example | 71k |
| 61 | |
| Example | 71l |
| 62 | |
| Example | 71m |
| 63 | |
| Example | 71n |
| 64 | |
| Example | 71o |
| 65 | |
| Example | 71p |
| 66 | |
| Example | 71q |
| 67 | |
| Example | 71r |
| 68 | |
| Example | 71s |
| 69 | |
| Example | 71t |
| 70 | |
| Example | 71u |
| 71 | |
| Example | 71v |
| 72 | |
| Example | 71w |
| 73 | |
| Example | 71x |
| 74 | |
| Example | 72a |
| 75 | |
| Example | 72b |
| 76 | |
| Example | 72c |
| 77 | |
| Example | 72d |
| 78 | |
| Example | 72e |
| 79 | |
| Example | 72f |
| 80 | |
| Example | 72g |
| 81 | |
| Example | 72h |
| 82 | |
| Example | 72i |
| 83 | |
| Example | 72j |
| 84 | |
| Example | 72k |
| 85 | |
| Example | 72l |
| 86 | |
| Example | 72m |
| 87 | |
| Example | 72n |
| 88 | |
| Example | 72o |
| 89 | |
| Example | 73 |
| 90 | |
| Example | 74 |
| 91 | |
| Example | 75 |
| 92 | |
| Example | 77 |
| 93 | |
| Example | 78 |
| 94 | |
| Example | 79 |
| 95 | |
| Example | 81 |
| 96 | |
| Example | 83 |
| 97 | |
| Example | 85 |
| 98 | |
| Example | 87 |
| 99 | |
| Example | 89 |
| 100 | |
| Example | 90 |
| 101 | |
| Example | 92 |
| 102 | |
| Example | 94 |
| 103 | |
| Example | 95 |
| 104 | |
| Example | 96 |
| 105 | |
| Example | 97 |
| 106 | |
| Example | 98 |
| 107 | |
| Example | 99 |
| 108 | |
| Example | 102 |
| 109 | |
| Example | 103 |
| 110 | |
| Example | 104 |
| 111 | |
| Example | 105 |
| 112 | |
| Example | 106 |
| 113 | |
| Example | 107 |
| 114 | |
| Example | 111a |
| 115 | |
| Example | 111b |
| 116 | |
| Example | 111c |
| 117 | |
| Example | 111d |
| 118 | |
| Example | 111e |
| 119 | |
| Example | 111f |
| 120 | |
| Example | 111g |
| 121 | |
| Example | 111h |
| 122 | |
| Example | 111i |
| 123 | |
| Example | 111j |
| 124 | |
| Example | 111k |
| 125 | |
| Example | 111l |
| 126 | |
| Example | 111m |
| 127 | |
| Example | 113 |
| 128 | |
| Example | 114 |
| 129 | |
| Example | 115 |
| 130 | |
| Example | 116 |
| 131 | |
| Example | 117 |
| 132 | |
| Example | 118 |
| 133 | |
| Example | 119 |
| 134 | |
| Example | 120 |
| 135 | |
| Example | 121 |
| 136 | |
| Example | 122 |
| 137 | |
| Example | 123 |
| 138 | |
| Example | 124 |
| 139 | |
| Example | 125 |
| 140 | |
| Example | 126 |
| 141 | |
| Example | 127 |
| 142 | |
| Example | 128 |
| 143 | |
| Example | 129 |
| 144 | |
| Example | 130 |
| 145 | |
| Example | 131 |
| 146 | |
| Example | 132 |
| 147 | |
| Example | 133 |
| 148 | |
| Example | 134 |
| 149 | |
| Example | 135 |
| 150 | |
| Example | 136 |
| 151 | |
| Example | 138 |
| 152 | |
| Example | 140 |
| 153 | |
| Example | 142 |
| 154 | |
| Example | 143 |
| 155 | |
| Example | 144 |
| 156 | |
| Example | 145 |
| 157 | |
| Example | 146 |
| 158 | |
| Example | 152 |
| 159 | |
| Example | 153 |
| 160 | |
| Example | 154 |
| 161 | |
| Example | 155 |
| 162 | |
| Example | 161 |
| 163 | |
| Example | 162 |
| 164 | |
| Example | 164 |
| 165 | |
| Example | 165 |
| 166 | |
| Example | 167 |
| 167 | |
| Example | 168 |
| 168 | |
| Example | 170 |
| 169 | |
| Example | 177 |
| 170 | |
| Example | 183 |
| 171 | |
| Example | 187 |
| 172 | |
| Example | 188 |
| 173 | |

### In Vitro Biochemical Test Procedures

The pharmacological properties of the compounds of the invention may be confirmed by a certain number of pharmacological assays. The examples of pharmacological assays that follow were performed with compounds according to the invention.

### Example 1

### TR-FRET Assay

In order to determine the inhibition of activation of Pim kinases, the compounds of the invention are tested in accordance with a routinely used in vitro TR-FRET assay (Time-Resolved Fluorescence Resonance Energy Transfer). The TR-FRET assay is based on detecting the phosphorylation of the specific residue Ser112 in the Bad protein, which was found to be a natural substrate for Pim kinases in cells. For the assay, the following reagents are used:

Pim kinase—His6-labelled recombinant full-length human Pim-1, Pim-2 or Pim-3 protein (prepared according to J. Mol. Biol. (2005) 348, 183-193);
Bad—His6-labelled recombinant full-length human Bad protein (prepared according to J. Mol. Biol. (2005) 348, 183-193);
α-His6-APC—murine monoclonal antibody conjugated to allophycocyanin SureLight™ directed against the His6 label (Perkin-Elmer, No. AD0059H, Waltham, Mass., United States);
β-P˜Bad-Eu—murine monoclonal antibody (Cell Signaling Technology #9296B, Danvers, Mass., United States) directed against phosphoBad (Ser112) (7E11) custom-labelled by Perkin-Elmer with the reagent LANCE™ Eu-W1024.

The assay is based on Perkin-Elmer's LANCE™ technology: the Eu-labelled antibody binds to phospho-Ser112 and generates a TR-FRET signal by interaction with the APC-labelled antibody directed against His6, bound to the His6 label of Bad. The TR-FRET signal is detected using a SpectraMax M5 plate reader (Molecular Devices) with the following settings: λex=340 nm, λem1=615 nm, λem2=665 nm. The ratio of the fluorescence signal at 665 nm to the fluorescence signal at 615 nm is used as the signal reading for the IC₅₀ (calculation is based on the 4-parameter logistic model). The assay is performed in a 384-well format; the liquid manipulations are performed using a Beckman 3000 liquid manipulations station. The test compounds are tested at 10 concentration points in duplicate; the highest compound concentration is typically equal to 30 μM. The ATP concentration is equal to 40 μM, which is equivalent to the apparent K_{M} value.

### Example 2

### Radiometric Filter-Binding Assay

In order to confirm their power, the compounds of the invention may be counter-selected by using a radiometric filter-binding assay. This assay measures the phosphorylation of a synthetic peptide (RSRHSSYPAGT) corresponding to amino acids 107-117 of the murine Bad protein, which includes the Ser112 phosphorylation site (Upstate No. 12-542), in the presence of ³³P-ATP as second substrate. The reaction is performed in the same format as for the TR-FRET assay described above. During the reaction, the basic substrate peptide binds to the phosphocellulose filter, and the level of phosphorylation is quantified by liquid scintillation counting. Once again, the ATP concentration is 40 μM, which is equivalent to the apparent K_{M} value.

### Example 3

### Cell Viability Assay

The representative compounds of the invention are also screened as regards their effects on cell proliferation and viability using a variety of human tumour cell lines, which are representative of various pathological indications. These cell lines include:
Models of haematological cancers:
   TF-1 (acute myelogenic leukaemia; AML M6 at the time of diagnosis);
   KG-1 (AML; erythroleukaemia evolving into AML);
   KG-1a (AML; sub-clone derived from immature KG-1);
   EOL-1 (AML; eosinophilic leukaemia);
   PL-21 (AML; M3);
   ML-2 (AML; T-NHL evolving into T-ALL evolving into AML M4);
   HL-60 (AML, M3);
   Kasumi-1 (AML);
   GDM-1 (AML);
   K-562 (CML—chronic myelogenic leukaemia; blastic crisis);
   JURL-MK1 (CML; blastic crisis);
   DND-41 (T-ALL—-cell acute lymphoblastic leukaemia);
   Jurkat (T-ALL); NALM-6 (B-ALL—B-cell ALL);
   CEM (ALL; lymphosarcoma evolving into ALL);
   Jeko-1 (B-NHL—B-cell non-Hodgkin lymphoma; mantle-cell lymphoma derived from a variant with large cells as a leukaemic transformation);
   WSU-DLCL2 (B-NHL; large B-cell diffuse lymphoma);
   RL (B-NHL; undifferentiated diffuse);
   OCI-Ly10 (B-NHL);
   DoHH-2 (B-NHL);
   RPMI-8226 (MM—multiple myeloma);
   JVM-2 (B-CLL—B-cell chronic lymphocytic leukaemia); and
   JVM-3 (B-CLL)
   MV4-11 (AML)
   MOLM13 (AML).
Solid tumour models:
   HCT-116 (bowel cancer);
   HT-29 (bowel cancer);
   HC-15 (bowel cancer);
   H460 (lung cancer; non-small-cell lung cancer);
   A375 (melanoma);
   B16F10 (melanoma);
   MDA-A1 (breast cancer);
   MDA-MB231 (breast cancer);
   MDA-MB231 adr (breast cancer);
   PANC-1 (pancreatic cancer); and
   PC-3 (prostate cancer).

In order to measure the viability, the tumour cells are incubated in a 96-well or 384-well format for 48, 72 or 96 hours, preferably 72 hours, with a compound of the invention at dilutions of a factor 3 with, in general, nine doses in total, the highest dose being equal to 10 μM or 30 μM. The cell viability is evaluated by adding CellTiter-Blue® (Promega, Madison, Wis., United States) for 4 hours and end-point readings are taken using a SpectraMax Genmini EM (Molecular Devices, Sunnyvale, Calif., United States). The CellTiter-Blue® cell viability assay measures the ability of the cells in culture to reduce resazurin to resorufin, the fluorescence signal intensity being directly proportional to the number of live cells. The EC₅₀ represents the concentration of compound that leads to a 50% reduction in the viability/proliferative expansion of the cells.

### Biochemical Results

The biochemical results are expressed according to the following classification:
Class A: IC50 between 1 nM and 100 nM
Class B: IC50 between 100 nM and 1000 nM (or 1 μM)
Class C: IC50 between 1 μM and 5 μM

### Class D: IC50>5 μM

| Example | Compound | IC50 | IC50 | IC50 | IC50 | IC50 |
|---|---|---|---|---|---|---|
| No. | No. | Pim1 | Pim2 | Pim3 | PLK1 | PI3K |
| Example 1 | 5a | A | C | B | B | |
| Example 2 | 5b | B | | | B | |
| Example 3 | 5c | B | | | | |
| Example 4 | 5d | A | C | B | | |
| Example 5 | 5e | A | D | B | D | |
| Example 6 | 5f | B | C | C | D | |
| Example 7 | 5g | A | B | A | B | |
| Example 8 | 5h | A | C | B | B | |
| Example 9 | 6 | B | C | B | | |
| Example 10 | 9a | A | C | A | B | |
| Example 11 | 9b | C | | | | |
| Example 12 | 9c | B | | | | |
| Example 13 | 10 | B | C | B | | |
| Example 14 | 11 | A | C | A | D | |
| Example 15 | 12 | A | B | A | D | |
| Example 16 | 13 | C | C | B | | |
| Example 17 | 14 | B | D | B | | |
| Example 18 | 15 | A | D | B | | |
| Example 19 | 16 | B | | | | |
| Example 20 | 19 | A | | | | |
| Example 21 | 21a | A | D | B | A | C |
| Example 22 | 21b | A | B | A | A | B |
| Example 23 | 21c | A | D | B | C | D |
| Example 24 | 21d | B | D | C | | |
| Example 25 | 21e | A | D | B | | |
| Example 26 | 21f | A | C | B | | B |
| Example 27 | 21g | A | D | B | | D |
| Example 28 | 21h | A | D | B | A | C |
| Example 29 | 21i | B | D | B | | |
| Example 30 | 21j | A | C | B | | |
| Example 31 | 21k | B | D | C | | |
| Example 32 | 22 | A | C | A | A | |
| Example 33 | 23 | A | B | B | | |
| Example 34 | 27a | A | D | C | B | |
| Example 35 | 27b | B | D | C | | |
| Example 36 | 27c | B | D | C | C | |
| Example 37 | 29 | B | D | C | | |
| Example 38 | 31 | B | D | B | | |
| Example 39 | 39a | A | C | B | C | |
| Example 40 | 39b | B | D | C | D | |
| Example 41 | 39c | C | D | D | | |
| Example 42 | 42 | C | | | | |
| Example 44 | 46 | A | A | A | A | A |
| Example 45 | 59 | A | C | A | A | |
| Example 46 | 61 | A | B | A | | |
| Example 47 | 64 | A | C | A | B | |
| Example 48 | 66 | A | B | A | B | |
| Example 49 | 68 | A | A | A | B | |
| Example 51 | 71a | A | C | B | | |
| Example 52 | 71b | B | D | B | | |
| Example 53 | 71c | A | C | B | | |
| Example 54 | 71d | A | D | B | | |
| Example 55 | 71e | A | C | B | | |
| Example 56 | 71f | C | D | B | | |
| Example 57 | 71g | C | C | B | | |
| Example 58 | 71h | C | D | C | | |
| Example 59 | 71i | A | D | B | | |
| Example 60 | 71j | A | C | B | | |
| Example 61 | 71k | A | C | A | | |
| Example 62 | 71l | A | C | A | A | |
| Example 63 | 71m | A | D | B | A | |
| Example 64 | 71n | B | D | C | | |
| Example 65 | 71o | B | D | C | | |
| Example 66 | 71p | A | B | A | A | |
| Example 67 | 71q | C | D | C | | |
| Example 68 | 71r | A | C | B | | |
| Example 69 | 71s | B | D | B | | |
| Example 70 | 71t | C | D | D | | |
| Example 71 | 71u | A | B | B | | |
| Example 72 | 71v | B | D | C | | |
| Example 73 | 71w | B | D | C | | |
| Example 74 | 71x | B | C | B | | |
| Example 75 | 72a | A | C | B | | |
| Example 76 | 72b | A | B | A | | |
| Example 77 | 72c | B | D | B | | |
| Example 78 | 72d | A | D | B | | |
| Example 79 | 72e | A | C | A | B | C |
| Example 80 | 72f | B | D | B | | |
| Example 81 | 72g | A | C | B | | |
| Example 82 | 72h | A | C | B | | |
| Example 83 | 72i | B | D | B | | |
| Example 84 | 72j | B | C | B | | |
| Example 85 | 72k | A | C | B | | |
| Example 86 | 72l | A | B | A | B | D |
| Example 87 | 72m | A | B | A | | |
| Example 88 | 72n | A | B | A | C | |
| Example 89 | 72o | A | C | B | C | |
| Example 90 | 73 | B | D | B | | |
| Example 91 | 74 | A | C | A | | |
| Example 92 | 75 | A | C | A | B | |
| Example 94 | 78 | A | B | A | A | |
| Example 95 | 79 | A | B | B | A | |
| Example 96 | 81 | A | B | A | B | |
| Example 97 | 83 | A | B | A | A | |
| Example 98 | 85 | A | A | A | B | |
| Example 99 | 87 | A | C | B | B | |
| Example 100 | 89 | A | B | A | A | |
| Example 101 | 90 | A | B | A | A | |
| Example 105 | 96 | A | D | B | | |
| Example 106 | 97 | A | D | A | | |
| Example 108 | 99 | A | D | A | | |
| Example 109 | 102 | A | D | B | | |
| Example 110 | 103 | A | C | B | C | |
| Example 111 | 104 | A | C | B | | |
| Example 112 | 105 | A | B | A | B | |
| Example 113 | 106 | A | C | A | C | |
| Example 114 | 107 | B | C | B | | |
| Example 115 | 111a | A | C | A | C | |
| Example 116 | 111b | A | D | A | C | |
| Example 117 | 111c | A | C | A | | |
| Example 118 | 111d | A | B | A | B | |
| Example 119 | 111e | A | B | A | B | |
| Example 120 | 111f | A | D | A | B | |
| Example 121 | 111g | A | C | A | C | |
| Example 122 | 111h | A | B | A | C | |
| Example 123 | 111i | A | B | A | C | |
| Example 124 | 111j | A | B | A | B | |
| Example 125 | 111k | A | | A | C | |
| Example 126 | 111l | A | D | B | C | |
| Example 127 | 111m | A | B | A | C | |
| Example 129 | 114 | B | | B | | |
| Example 130 | 115 | A | B | A | | |
| Example 131 | 116 | B | | B | | |
| Example 132 | 117 | A | B | A | | |
| Example 133 | 118 | A | | B | | |
| Example 134 | 119 | A | D | A | | |
| Example 135 | 120 | A | B | A | | |
| Example 136 | 121 | A | D | A | | |
| Example 137 | 122 | A | B | A | | |
| Example 138 | 123 | A | B | A | | |
| Example 139 | 124 | A | C | A | | |
| Example 140 | 125 | A | C | A | | |
| Example 141 | 126 | A | C | A | | |
| Example 142 | 127 | A | D | A | | |
| Example 143 | 128 | B | | B | | |
| Example 144 | 129 | A | D | A | | |
| Example 145 | 130 | A | B | A | | |
| Example 146 | 131 | A | B | A | | |
| Example 147 | 132 | A | B | A | | |
| Example 148 | 133 | A | B | A | | |
| Example 149 | 134 | A | B | A | | |
| Example 150 | 135 | A | B | A | | |
| Example 151 | 136 | A | B | A | | |
| Example 152 | 138 | A | B | A | | |
| Example 153 | 140 | A | C | A | | |
| Example 155 | 143 | A | B | A | | |
| Example 156 | 144 | A | D | B | | |
| Example 157 | 145 | A | B | A | | |
| Example 158 | 146 | A | B | A | | |
| Example 159 | 152 | C | D | C | | |
| Example 160 | 153 | B | D | C | | |
| Example 161 | 154 | B | D | B | | |
| Example 162 | 155 | D | | D | C | |
| Example 163 | 161 | A | B | A | C | |
| Example 165 | 164 | B | D | B | D | |
| Example 166 | 165 | B | D | C | | |
| Example 167 | 167 | B | C | B | D | |
| Example 168 | 168 | B | D | B | | |
| Example 169 | 170 | B | D | B | | |
| Example 170 | 177 | A | B | B | | |
| Example 171 | 183 | A | A | A | | |
| Example 172 | 187 | D | D | | D | |

### Biochemical Results of the Comparatives

| Compound | | | |
|---|---|---|---|
| No. | IC50 Pim1 | IC50 Pim2 | IC50 Pim3 |
| 51 | D | D | D |
| 56 | D | D | D |

### Cell Results

The cell proliferation results are expressed according to the following classification:

Class A: IC50 between 1 nM and 100 nM
Class B: IC50 between 100 nM and 1000 nM (or 1 μM)
Class C: IC50 between 1 μM and 5 μM

| | | EC50 | EC50 | IC50 | IC50 | EC50 | EC50 | EC50 |
|---|---|---|---|---|---|---|---|---|
| Example | Compound | EOL-1 μM | KG-1a μM | MV4-11 μM | MOLM-13 μM | HCT116 μM | B16F10 μM | H460 μM |
| No. | No. | (lymphoma) | (leukaemia) | (myeloma) | (myeloma) | (bowel) | (melanoma) | (lung) |
| 1 | 5a | C | C | C | | | | |
| 5 | 5e | B | B | B | | | | |
| 7 | 5g | B | | B | B | | | |
| 8 | 5h | B | | B | B | | | |
| 10 | 9a | B | B | B | | | | |
| 14 | 11 | B | C | B | | | | |
| 15 | 12 | B | B | B | | | | |
| 22 | 21b | A | | A | A | A | A | A |
| 33 | 23 | B | | C | B | | | |
| 39 | 39a | B | B | C | C | | | |
| 44 | 46 | A | | A | A | | | |
| 49 | 68 | A | | A | | | | |
| 86 | 72l | B | | B | | | | |
| 92 | 75 | B | | C | | | | |
| 94 | 78 | A | | A | B | | | |
| 96 | 81 | A | | B | B | | | |
| 97 | 83 | B | | B | B | | | |
| 98 | 85 | A | | B | B | | | |
| 100 | 89 | A | | B | B | | | |
| 119 | 111e | A | | B | B | | | |
| 120 | 111f | A | | A | B | | | |
| 122 | 111h | A | | A | | | | |
| 123 | 111i | A | | A | A | | | |
| 127 | 111m | A | | A | | | | |
| 128 | 113 | A | | A | A | | | |
| 130 | 115 | A | | A | A | | | |
| 163 | 161 | B | | B | | | | |

1. A compound having the general formula (I) below: 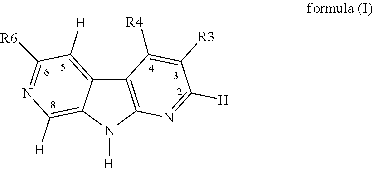
in which
R3 and R4 may be, independently of each other:
1. H;
2. halogen;
3. CF₃;
4. substituted oxy;
5. optionally substituted alkoxy;
6. optionally substituted amino;
7. substituted carbonyl;
8. optionally substituted carboxyl;
9. optionally substituted amide;
10. sulfur in different oxidation states (II or IV or VI) such as optionally substituted sulfides, sulfoxides or sulfones;
11. C₁-C₁₀ linear, branched or cyclic alkyl optionally comprising an optionally substituted heteroatom;
12. optionally substituted linear, branched or cyclic C₂-C₇ alkenyl;
13. optionally substituted linear or branched C₂-C₆ alkynyl;
14. optionally substituted aryl or heteroaryl;
15. optionally substituted heterocycloalkyl;
R6 being a heteroaryl (5- or 6-membered with 1 to 4 heteroatoms chosen from N, S and O) bonded to the azacarboline unit either via a C or via an N belonging to R6, R6 being optionally substituted; R6 also possibly representing C(O)NR1a R1b or an optionally substituted heterocycloalkyl or —C(O) optionally substituted heterocycloalkyl, such that R1a and R1b may be, independently of each other:
1. H;
2. optionally monosubstituted or disubstituted linear or branched or cyclic (C₃-C₇) C₁-C₁₀ alkyl;
3. optionally monosubstituted or disubstituted linear or branched C₂-C₆ alkenyl;
4. optionally monosubstituted or disubstituted linear or branched C₂-C₆ alkynyl;
5. optionally monosubstituted or disubstituted aryl;
6. optionally monosubstituted or disubstituted heteroaryl;
7. optionally monosubstituted or disubstituted benzyl;
8. optionally monosubstituted or disubstituted COalkyl;
9. optionally monosubstituted or disubstituted COaryl;
10. optionally monosubstituted or disubstituted COheteroaryl;
11. optionally monosubstituted or disubstituted CO2alkyl;
12. optionally monosubstituted or disubstituted CO2aryl;
13. optionally monosubstituted or disubstituted CO2heteroaryl;
14. CONH2;
15. optionally monosubstituted or disubstituted CONHalkyl;
16. optionally monosubstituted or disubstituted CONHaryl;
17. optionally monosubstituted or disubstituted CONHheteroaryl;
18. optionally monosubstituted or disubstituted CON(alkyl)2;
19. optionally monosubstituted or disubstituted CON(aryl)2;
20. optionally monosubstituted or disubstituted CON(heteroaryl)2;
the said products of formula (I) being in the form of the base or of an acid-addition salt.

2. A compound of formula (I) as defined in claim 1, in which
R3 and R4 may be, independently of each other:
1. H;
2. F;
3. Cl;
4. Br;
5. I;
6. CF₃;
7. OR2a;
8. NR1a R1b;
9. COR2a;
10. CO₂R2a;
11. CO(NR1a R1b);
12. SR2a;
13. SOR2a;
14. SO₂R2a;
15. linear or branched or cyclic (C₃-C₇) C₁-C₁₀ alkyl optionally monosubstituted or disubstituted or trisubstituted with R2a, R2b, R2c;
16. linear or branched or cyclic (C₃-C₇) C₂-C₆ alkenyl optionally monosubstituted or disubstituted or trisubstituted with R2a, R2b, R2c;
17. linear or branched C₂-C₆ alkynyl optionally monosubstituted or disubstituted or trisubstituted with R2a, R2b, R2c;
18. aryl or heteroaryl optionally monosubstituted or disubstituted or trisubstituted with R2a, R2b, R2c;
19. heterocycloalkyl optionally monosubstituted or disubstituted or trisubstituted with R2a, R2b, R2c;
R6 being a heteroaryl (5- or 6-membered with 1 to 4 heteroatoms N, S or O) bonded to the azacarboline unit either via a C or an N belonging to R6, R6 also possibly representing C(O)NR1a R1b or an optionally substituted heterocycloalkyl or —C(O) optionally substituted heterocycloalkyl; R6 being optionally monosubstituted or disubstituted or trisubstituted with R2a, R2b, R2c;
in which:
R1a and R1b may be, independently of each other:
1. H;
2. linear or branched or cyclic (C₃-C₇) C₁-C₁₀ alkyl optionally monosubstituted or disubstituted with R2a R2b;
3. linear or branched C₂-C₆ alkenyl optionally monosubstituted or disubstituted with R2a R2b;
4. linear or branched C₂-C₆ alkynyl optionally monosubstituted or disubstituted with R2a R2b;
5. aryl optionally monosubstituted or disubstituted with R2a R2b;
6. heteroaryl optionally monosubstituted or disubstituted with R2a R2b;
7. benzyl optionally monosubstituted or disubstituted with R2a R2b;
8. COalkyl optionally monosubstituted or disubstituted with R2a R2b;
9. COaryl optionally monosubstituted or disubstituted with R2a R2b;
10. COheteroaryl optionally monosubstituted or disubstituted with R2a R2b;
11. CO₂alkyl optionally monosubstituted or disubstituted with R2a R2b;
12. CO₂aryl optionally monosubstituted or disubstituted with R2a R2b;
13. CO₂heteroaryl optionally monosubstituted or disubstituted with R2a R2b;
14. CONH₂;
15. CONHalkyl optionally monosubstituted or disubstituted with R2a R2b;
16. CONHaryl optionally monosubstituted or disubstituted with R2a R2b;
17. CONHheteroaryl optionally monosubstituted or disubstituted with R2a R2b;
18. CON(alkyl)₂ optionally monosubstituted or disubstituted with R2a R2b;
19. CON(aryl)₂ optionally monosubstituted or disubstituted with R2a R2b;
20. CON(heteroaryl)₂ optionally monosubstituted or disubstituted with R2a R2b;
in which R2a, R2b and R2c are chosen, independently of each other, from:
1. F;
2. Cl;
3. Br;
4. I;
5. CF₃;
6. linear or branched C₁-C₁₀ alkyl optionally monosubstituted or polysubstituted with different R3a;
7. C₃-C₇ cycloalkyl optionally monosubstituted or polysubstituted with different R3a;
8. C₂-C₆ alkenyl optionally monosubstituted or polysubstituted with different R3a;
9. C₂-C₆ alkynyl optionally monosubstituted or polysubstituted with different R3a;
10. OH;
11. linear or branched O—(C₁-C₁₀)alkyl optionally monosubstituted or polysubstituted with different R3a;
12. O—(C₃-C₇)cycloalkyl optionally monosubstituted or polysubstituted with different R3a;
13. O-aryl optionally monosubstituted or polysubstituted with different R3a;
14. aryl optionally monosubstituted or polysubstituted with different R3a;
15. heteroaryl optionally monosubstituted or polysubstituted with different R3a;
16. heterocycloalkyl optionally monosubstituted or polysubstituted with different R3a;
17. NH₂;
18. NH—((C₁-C₁₀)alkyl or (C₃-C₇)cycloalkyl or heterocycloalkyl), each group being optionally monosubstituted or polysubstituted with different R3a;
19. N((C₁-C₁₀)alkyl or (C₃-C₇)cycloalkyl)₂, each group being optionally monosubstituted or polysubstituted with different R3a;
20. NH-(aryl or heteroaryl) optionally monosubstituted or polysubstituted with different R3a;
21. N(aryl or heteroaryl)₂, each group being optionally monosubstituted or polysubstituted with different R3a;
22. N(aryl or heteroaryl)((C₁-C₁₀)alkyl or (C₃-C₇)cycloalkyl), each group being optionally monosubstituted or polysubstituted with different R3a;
23. NHC(O)R3a;
24. N((C₁-C₁₀)alkylC(O)R3a;
25. N(R3a)C(O)R3b;
26. NHS(O₂)R3a;
27. N((C₁-C₁₀)alkylS(O₂)R3a;
28. N(R3a)S(O)₂R3b;
29. CO₂R3a;
30. SR3a;
31. SOR3a;
32. SO₂R3a;
in which R3a and R3b are chosen from:
1. halogen;
2. CF₃;
3. linear or branched C₁-C₁₀ alkyl;
4. C₃-C₇ cycloalkyl;
5. C₂-C₆ alkenyl;
6. C₂-C₆ alkynyl;
7. C₁-C₁₀ alkylhydroxy;
8. C₁-C₁₀ alkoxy;
9. C₁-C₁₀ alkylamino;
10. OH;
11. linear, branched or cyclic (C₃-C₇) O—(C₁-C₁₀)alkyl;
12. O-aryl;
13. aryl;
14. heteroaryl;
15. heterocycloalkyl;
16. NH₂;
17. NH—((C₁-C₁₀)alkyl or (C₃-C₇)cycloalkyl);
18. N((C₁-C₁₀)alkyl or (C₃-C₇)cycloalkyl)₂;
19. NH-(aryl or heteroaryl);
20. N(aryl or heteroaryl)₂;
21. N(aryl or heteroaryl)((C₁-C₁₀)alkyl or (C₃-C₇)cycloalkyl);
22. NHC(O)—((C₁-C₁₀)alkyl or (C₃-C₇)cycloalkyl or heterocycloalkyl);
23. NHC(O)-(aryl or heteroaryl);
24. NHS(O)₂((C₁-C₁₀)alkyl or (C₃-C₇)cycloalkyl or heterocycloalkyl);
25. NHS(O)₂-(aryl or heteroaryl);
26. CO (linear or branched C₁-C₁₀ alkyl);
27. CO(C₁-C₁₀ alkylamino);
28. CO₂ (linear or branched C₁-C₁₀ alkyl);
29. C(O)NH (linear or branched C₁-C₁₀ alkyl);
30. C(O)N (linear or branched C₁-C₁₀ alkyl)₂;
31. S (linear or branched C₁-C₁₀ alkyl);
32. SO (linear or branched C₁-C₁₀ alkyl);
33. SO₂ (linear or branched C₁-C₁₀ alkyl)
34. C(O)(heterocycloalkyl);
the said products of formula (I) being in the form of the base or of an acid-addition salt.

3. A compound according to claim 1, characterized in that:
R3 and R4 may be, independently of each other:
1. H;
2. F;
3. Cl;
4. Br;
5. I;
6. CF₃;
7. OR2a;
8. NR1aR1b;
9. COR2a;
10. CO₂R2a;
11. CO(NR1aR1b);
12. SR2a;
13. SOR2a;
14. SO₂R2a;
15. linear or branched or cyclic (C₃-C₇) C₁-C₁₀ alkyl optionally monosubstituted or disubstituted or trisubstituted with R2a, R2b, R2c;
16. linear or branched or cyclic (C₃-C₇) C₂-C₆ alkenyl optionally monosubstituted or disubstituted or trisubstituted with R2a, R2b, R2c;
17. linear or branched C₂-C₆ alkynyl optionally monosubstituted or disubstituted or trisubstituted with R2a, R2b, R2c;
18. aryl or heteroaryl optionally monosubstituted or disubstituted or trisubstituted with R2a, R2b, R2c;
19. heterocycloalkyl optionally monosubstituted or disubstituted or trisubstituted with R2a, R2b, R2c;
R6 being a heteroaryl (5- or 6-membered with 1 to 4 heteroatoms N, S or O) bonded to the azacarboline unit either via a C or an N belonging to R6, R6 being optionally monosubstituted or disubstituted or trisubstituted with R2a, R2b, R2c;
in which:
R1a and R1b may be, independently of each other:
1. H;
2. linear or branched or cyclic (C₃-C₇) C₁-C₁₀ alkyl optionally monosubstituted or disubstituted with R2a R2b;
3. linear or branched C₂-C₆ alkenyl optionally monosubstituted or disubstituted with R2a R2b;
4. linear or branched C₂-C₆ alkynyl optionally monosubstituted or disubstituted with R2a R2b;
5. aryl optionally monosubstituted or disubstituted with R2a R2b;
6. heteroaryl optionally monosubstituted or disubstituted with R2a R2b;
7. benzyl optionally monosubstituted or disubstituted with R2a R2b;
8. COalkyl optionally monosubstituted or disubstituted with R2a R2b;
9. COaryl optionally monosubstituted or disubstituted with R2a R2b;
10. COheteroaryl optionally monosubstituted or disubstituted with R2a R2b;
11. CO₂alkyl optionally monosubstituted or disubstituted with R2a R2b;
12. CO₂aryl optionally monosubstituted or disubstituted with R2a R2b;
13. CO₂heteroaryl optionally monosubstituted or disubstituted with R2a R2b;
14. CONH₂;
15. CONHalkyl optionally monosubstituted or disubstituted with R2a R2b;
16. CONHaryl optionally monosubstituted or disubstituted with R2a R2b;
17. CONHheteroaryl optionally monosubstituted or disubstituted with R2a R2b;
18. CON(alkyl)₂ optionally monosubstituted or disubstituted with R2a R2b;
19. CON(aryl)₂ optionally monosubstituted or disubstituted with R2a R2b;
20. CON(heteroaryl)₂ optionally monosubstituted or disubstituted with R2a R2b;
in which R2a, R2b and R2c are chosen, independently of each other, from:
1. F;
2. Cl;
3. Br;
4. I;
5, CF₃;
6. linear or branched C₁-C₁₀ alkyl optionally monosubstituted or polysubstituted with different R3a;
7. C₃-C₇ cycloalkyl optionally monosubstituted or polysubstituted with different R3a;
8. C₂-C₆ alkenyl optionally monosubstituted or polysubstituted with different R3a;
9. C₂-C₆ alkynyl optionally monosubstituted or polysubstituted with different R3a;
10. OH;
11. linear or branched O—(C₁-C₁₀)alkyl optionally monosubstituted or polysubstituted with different R3a;
12. O—(C₃-C₇)cycloalkyl optionally monosubstituted or polysubstituted with different R3a;
13. O-aryl optionally monosubstituted or polysubstituted with different R3a;
14. aryl optionally monosubstituted or polysubstituted with different R3a;
15. heteroaryl optionally monosubstituted or polysubstituted with different R3a;
16. heterocycloalkyl optionally monosubstituted or polysubstituted with different R3a;
17. NH₂;
18. NH—((C₁-C₁₀)alkyl or (C₃-C₇)cycloalkyl or heterocycloalkyl), each group being optionally monosubstituted or polysubstituted with different R3a;
19. N((C₁-C₁₀)alkyl or (C₃-C₇)cycloalkyl)₂, each group being optionally monosubstituted or polysubstituted with different R3a;
20. NH-(aryl or heteroaryl) optionally monosubstituted or polysubstituted with different R3a;
21. N(aryl or heteroaryl)₂, each group being optionally monosubstituted or polysubstituted with different R3a;
22. N(aryl or heteroaryl)((C₁-C₁₀)alkyl or (C₃-C₇)cycloalkyl), each group being optionally monosubstituted or polysubstituted with different R3a;
23. NHC(O)R3a;
24. N((C₁-C₁₀)alkylC(O)R3a;
25. NHC(O)—((C₁-C₁₀)alkyl or (C₃-C₇)cycloalkyl or heterocycloalkyl), each group being optionally monosubstituted or polysubstituted with different R3a;
26. NC(O)((C₁-C₁₀)alkyl or (C₃-C₇)cycloalkyl or heterocycloalkyl)₂, each group being optionally monosubstituted or polysubstituted with different R3a;
27. NHC(O)-(aryl or heteroaryl) optionally monosubstituted or polysubstituted with different R3a;
28. NC(O)(aryl or heteroaryl)₂, each group being optionally monosubstituted or polysubstituted with different R3a;
29. NC(O)(aryl or heteroaryl)((C₁-C₁₀)alkyl or (C₃-C₇)cycloalkyl or heterocycloalkyl), each group being optionally monosubstituted or polysubstituted with different R3a;
30. NHS(O₂)R3a;
31. N((C₁-C₁₀)alkylS(O₂)R3a;
32. NHS(O₂)—((C₁-C₁₀)alkyl or (C₃-C₇)cycloalkyl or heterocycloalkyl), each group being optionally monosubstituted or polysubstituted with different R3a;
33. NS(O₂)((C₁-C₁₀)alkyl or (C₃-C₇)cycloalkyl or heterocycloalkyl)₂, each group being optionally monosubstituted or polysubstituted with different R3a;
34. NHS(O₂)-(aryl or heteroaryl) optionally monosubstituted or polysubstituted with different R3a;
35. NS(O₂)(aryl or heteroaryl)₂, each group being optionally monosubstituted or polysubstituted with different R3a;
36. NS(O₂)(aryl or heteroaryl)((C₁-C₁₀)alkyl or (C₃-C₇)cycloalkyl or heterocycloalkyl), each group being optionally monosubstituted or polysubstituted with different R3a; COR3a;
37. CO₂R3a;
38. SR3a;
39. SOR3a;
40. SO₂R3a;
in which R3a is chosen from:
1. halogen;
2. CF₃;
3. linear or branched C₁-C₁₀ alkyl;
4. C₃-C₇cycloalkyl;
5. C₂-C₆ alkenyl;
6. C₂-C₆ alkynyl;
7. OH;
8. linear, branched or cyclic (C₃-C₇) O—(C₁-C₁₀)alkyl;
9. O-aryl;
10. aryl;
11. heteroaryl;
12. heterocycloalkyl;
13. NH₂;
14. NH((C₁-C₁₀)alkyl or (C₃-C₇)cycloalkyl);
15. N((C₁-C₁₀)alkyl or (C₃-C₇)cycloalkyl)₂;
16. NH-(aryl or heteroaryl);
17. N(aryl or heteroaryl)₂;
18. N(aryl or heteroaryl)((C₁-C₁₀)alkyl or (C₃-C₇)cycloalkyl);
19. NHC(O)—((C₁-C₁₀)alkyl or (C₃-C₇)cycloalkyl or heterocycloalkyl);
20. NC(O)((C₁-C₁₀)alkyl or (C₃-C₇)cycloalkyl or heterocycloalkyl)₂;
21. NHC(O)-(aryl or heteroaryl);
22. NC(O)(aryl or heteroaryl)₂;
23. NC(O)(aryl or heteroaryl)((C₁-C₁₀)alkyl or (C₃-C₇)cycloalkyl or heterocycloalkyl);
24. NHS(O₂)—((C₁-C₁₀)alkyl or (C₃-C₇)cycloalkyl or heterocycloalkyl);
25. NS(O₂)((C₁-C₁₀)alkyl or (C₃-C₇)cycloalkyl or heterocycloalkyl)₂;
26. NHS(O₂)-(aryl or heteroaryl);
27. NS(O₂)(aryl or heteroaryl)₂;
28. NS(O₂)(aryl or heteroaryl)((C₁-C₁₀)alkyl or (C₃-C₇)cycloalkyl or heterocycloalkyl);
29. CO (linear or branched C₁-C₁₀ alkyl);
30. CO₂ (linear or branched C₁-C₁₀ alkyl);
31. C(O)NH (linear or branched C₁-C₁₀ alkyl);
32. C(O)N (linear or branched C₁-C₁₀ alkyl)₂;
33. S (linear or branched C₁-C₁₀ alkyl);
34. SO (linear or branched C₁-C₁₀ alkyl);
35. SO₂ (linear or branched C₁-C₁₀ alkyl).

4. A compound according to claim 1, chosen from:
N-{4-[3-fluoro-6-(pyrid-3-yl)-9H-pyrrolo[2,3-b:5,4-c′]dipyrid-4-yl]phenyl}methanesulfon-amide;
N-{4-[3-methoxy-6-(pyrid-3-yl)-9H-pyrrolo[2,3-b:5,4-c′]dipyrid-4-yl]phenyl}methanesulfon-amide;
4-(3,5-dimethoxyphenyl)-3-fluoro-6-(pyrid-3-yl)-9H-pyrrolo[2,3-b:5,4-c′]dipyridine;
4-cyclopropyl-3-fluoro-6-(pyrid-3-yl)-9H-pyrrolo[2,3-b:5,4-c′]dipyridine;
3-methoxy-6-(pyrid-3-yl)-9H-pyrrolo[2,3-b:5,4-c′]dipyridine;
N-cyclopropyl-4-[3-fluoro-6-(pyrid-3-yl)-9H-pyrrolo[2,3-b:5,4-c′]dipyrid-4-yl]benzenesulfonamide;
3-hydroxy-2,2-dimethylpropyl 6-(pyrid-3-yl)-9H-pyrrolo[2,3-b:5,4-c′]dipyridine-3-carboxylate;
4-methoxy-6-(pyrid-3-yl)-9H-pyrrolo[2,3-b:5,4-c′]dipyridine;
3-[3-fluoro-6-(pyrid-3-yl)-9H-pyrrolo[2,3-b:5,4-c′]dipyrid-4-yl]phenol;
4-[(E)-2-cyclopropylethenyl]-3-fluoro-6-(pyrid-3-yl)-9H-pyrrolo[2,3-b:5,4-c′]dipyridine;
4-(3,5-difluorophenyl)-3-fluoro-6-(pyrid-3-yl)-9H-pyrrolo[2,3-b:5,4-c′]dipyridine;
2-methylpropan-2-yl 6-(pyrid-3-yl)-9H-pyrrolo[2,3-b:5,4-c′]dipyridine-3-carboxylate;
3-fluoro-4-iodo-6-(pyrid-3-yl)-9H-pyrrolo[2,3-b:5,4-c′]dipyridine;
4-[3-fluoro-6-(pyrid-3-yl)-9H-pyrrolo[2,3-b:5,4-c′]dipyrid-4-yl]butane-1,2-diol;
[3-fluoro-6-(pyrid-3-yl)-9H-pyrrolo[2,3-b:5,4-c′]dipyrid-4-yl](phenyl)methanone;
3-[3-fluoro-6-(pyrid-3-yl)-9H-pyrrolo[2,3-b:5,4-c′]dipyrid-4-yl]benzenesulfonamide;
3-(morpholin-4-yl)-6-(pyrid-3-yl)-9H-pyrrolo[2,3-b:5,4-c′]dipyridine;
6-(1-methyl-1 H-pyrazol-4-yl)-9H-pyrrolo[2,3-b:5,4-c′]dipyridine;
3-fluoro-4-(morpholin-4-yl)-6-(pyrid-3-yl)-9H-pyrrolo[2,3-b:5,4-c′]dipyridine;
2-methylpropyl 6-(pyrid-3-yl)-9H-pyrrolo[2,3-b:5,4-c′]dipyridine-3-carboxylate;
N-methyl-N-propyl-6-(pyrid-3-yl)-9H-pyrrolo[2,3-b:5,4-c′]dipyrid-3-amine;
ethyl 6-(pyrid-3-yl)-9H-pyrrolo[2,3-b:5,4-c′]dipyridine-3-carboxylate;
6-(pyrid-3-yl)-9H-pyrrolo[2,3-b:5,4-c′]dipyridine;
3-fluoro-4-methyl-6-(pyrid-3-yl)-9H-pyrrolo[2,3-b:5,4-c′]dipyridine;
3-fluoro-6-(pyrid-3-yl)-9H-pyrrolo[2,3-b:5,4-c′]dipyridine;
4-chloro-3-fluoro-6-(pyrid-3-yl)-9H-pyrrolo[2,3-b:5,4-c′]dipyridine;
3-fluoro-4-[(E)-2-phenylethenyl]-6-(pyrid-3-yl)-9H-pyrrolo[2,3-b:5,4-c′]dipyridine;
3-chloro-6-(pyrid-3-yl)-9H-pyrrolo[2,3-b:5,4-c′]dipyridine;
3-bromo-6-(pyrid-3-yl)-9H-pyrrolo[2,3-b:5,4-c′]dipyridine;
ethyl (2E)-3-[3-fluoro-6-(pyrid-3-yl)-9H-pyrrolo[2,3-b:5,4-c′]dipyrid-4-yl]prop-2-enoate;
3-fluoro-4-[3-(morpholin-4-yl)phenyl]-6-(pyrid-3-yl)-9H-pyrrolo[2,3-b:5,4-c′]dipyridine;
6-(pyrid-3-yl)-9H-pyrrolo[2,3-b:5,4-c′]dipyridine-3-carboxylic acid;
[6-(pyrid-3-yl)-9H-pyrrolo[2,3-b:5,4-c′]dipyrid-3-yl]methanol;
methyl 6-(pyrid-3-yl)-9H-pyrrolo[2,3-b:5,4-c′]dipyridine-3-carboxylate;
N-methyl-N-propyl-6-(pyrid-3-yl)-9H-pyrrolo[2,3-b:5,4-c′]dipyridine-3-carboxamide;
3-fluoro-N-methyl-N-phenyl-6-(pyrid-3-yl)-9H-pyrrolo[2,3-b:5,4-c′]dipyridine-4-carboxamide;
4-{methyl[6-(pyrid-3-yl)-9H-pyrrolo[2,3-b:5,4-c′]dipyrid-3-yl]amino}-1-(pyrrolidin-1-yl)butan-1-one;
6-(furan-3-yl)-9H-pyrrolo[2,3-b:5,4-c′]dipyridine;
[3-fluoro-6-(pyrid-3-yl)-9H-pyrrolo[2,3-b:5,4-c′]dipyrid-4-yl](morpholin-4-yl)methanone;
6-(5-fluoropyrid-3-yl)-9H-pyrrolo[2,3-b:5,4-c′]dipyridine;
2-[6-(pyrid-3-yl)-9H-pyrrolo[2,3-b:5,4-c′]dipyrid-3-yl]propan-2-ol;
6-(6-fluoropyrid-3-yl)-9H-pyrrolo[2,3-b:5,4-c′]dipyridine;
N,N-diethyl-6-(pyrid-3-yl)-9H-pyrrolo[2,3-b:5,4-c′]dipyrid-3-amine;
3-(pyrid-3-yl)-9H-pyrrolo[2,3-b:5,4-c′]dipyridine;
3-methoxy-6-(pyrid-3-yl)-9H-pyrrolo[2,3-c: 5,4-c′]dipyridine;
1-chloro-N-{4-[3-fluoro-6-(pyrid-3-yl)-9H-pyrrolo[2,3-b:5,4-c′]dipyrid-4-yl]phenyl}methane-sulfonamide;
3-(4-methylpiperazin-1-yl)-6-(pyrid-3-yl)-9H-pyrrolo[2,3-b:5,4-c′]dipyridine;
N-{4-[3-fluoro-6-(pyrid-3-yl)-9H-pyrrolo[2,3-b:5,4-c′]dipyrid-4-yl]phenyl}cyclopropanesulfonamide;
N-{4-[3-fluoro-6-(pyrid-3-yl)-9H-pyrrolo[2,3-b:5,4-c′]dipyrid-4-yl]-2-methoxyphenyl}-methanesulfonamide;
N-{4-[3-fluoro-6-(1-methyl-1 H-pyrazol-4-yl)-9H-pyrrolo[2,3-b:5,4-c′]dipyrid-4-yl]phenyl}-methanesulfonamide;
3-fluoro-6-(5-methoxypyrid-3-yl)-9H-pyrrolo[2,3-b:5,4-c′]dipyridine;
3-fluoro-6-(4-methoxypyrid-3-yl)-9H-pyrrolo[2,3-b:5,4-c′]dipyridine;
6-(1-benzyl-1 H-pyrazol-4-yl)-3-fluoro-9H-pyrrolo[2,3-b:5,4-c′]dipyridine;
3-fluoro-6-(1-methyl-1 H-pyrazol-4-yl)-9H-pyrrolo[2,3-b:5,4-c′]dipyridine;
3-fluoro-6-[1-(2-methylpropyl)-1 H-pyrazol-4-yl]-9H-pyrrolo[2,3-b:5,4-c′]dipyridine;
3-fluoro-6-[5-(methylsulfanyl)pyrid-3-yl]-9H-pyrrolo[2,3-b:5,4-c′]dipyridine;
4-[3-fluoro-6-(pyrid-3-yl)-9H-pyrrolo[2,3-b:5,4-c′]dipyrid-4-yl]-2-methylbut-3-yn-2-ol;
4-[3-fluoro-6-(pyrid-3-yl)-9H-pyrrolo[2,3-b:5,4-c′]dipyrid-4-yl]-2-methylbut-3-yn-2-amine;
N-{4-[3-fluoro-6-(pyrid-3-yl)-9H-pyrrolo[2,3-b:5,4-c′]dipyrid-4-yl]-2-methylbut-3-yn-2-yl}-methanesulfonamide;
3-fluoro-4-[3-methyl-3-(piperazin-1-yl)but-1-yn-1-yl]-6-(pyrid-3-yl)-9H-pyrrolo[2,3-b:5,4-c′]-dipyridine;
4-[3-methoxy-6-(pyrid-3-yl)-9H-pyrrolo[2,3-b:5,4-c′]dipyrid-4-yl]-2-methylbut-3-yn-2-ol;
4-[3-methoxy-6-(pyrid-3-yl)-9H-pyrrolo[2,3-b:5,4-c′]dipyrid-4-yl]-2-methylbut-3-yn-2-amine;
N-{4-[3-methoxy-6-(pyrid-3-yl)-9H-pyrrolo[2,3-b:5,4-c′]dipyrid-4-yl]-2-methylbut-3-yn-2-yl}-methanesulfonamide;
3-methoxy-4-[3-methyl-3-(piperazin-1-yl)but-1-yn-1-yl]-6-(pyrid-3-yl)-9H-pyrrolo[2,3-b:5,4-c′]dipyridine;
3-fluoro-4-[4-(4-methylpiperazin-1-yl)piperid-1-yl]-6-(pyrid-3-yl)-9H-pyrrolo[2,3-b:5,4-c′]-dipyridine;
2-(4-{1-[3-fluoro-6-(pyrid-3-yl)-9H-pyrrolo[2,3-b:5,4-c′]dipyrid-4-yl]piperid-4-yl}piperazin-1-yl)ethanol;
3-fluoro-4-[4-(morpholin-4-yl)piperid-1-yl]-6-(pyrid-3-yl)-9H-pyrrolo[2,3-b:5,4-c′]dipyridine;
3-fluoro-4-[4-(propan-2-yl)piperazin-1-yl]-6-(pyrid-3-yl)-9H-pyrrolo[2,3-b:5,4-c′]dipyridine;
4-(4-cyclopropylpiperazin-1-yl)-3-fluoro-6-(pyrid-3-yl)-9H-pyrrolo[2,3-b:5,4-c′]dipyridine;
4-(4-ethylpiperazin-1-yl)-3-fluoro-6-(pyrid-3-yl)-9H-pyrrolo[2,3-b:5,4-c′]dipyridine;
3-fluoro-4-[4-(1-methylpiperid-4-yl)piperazin-1-yl]-6-(pyrid-3-yl)-9H-pyrrolo[2,3-b:5,4-c′]-dipyridine;
3-methoxy-4-[4-(4-methylpiperazin-1-yl)piperid-1-yl]-6-(pyrid-3-yl)-9H-pyrrolo[2,3-b:5,4-c′]-dipyridine:
2-(4-{1-[3-methoxy-6-(pyrid-3-yl)-9H-pyrrolo[2,3-b:5,4-c′]dipyrid-4-yl]piperid-4-yl}piperazin-1-yl)ethanol;
3-methoxy-4-[4-(morpholin-4-yl)piperid-1-yl]-6-(pyrid-3-yl)-9H-pyrrolo[2,3-b:5,4-c′]-dipyridine:
3-methoxy-4-[4-(1-methylpiperid-4-yl)piperazin-1-yl]-6-(pyrid-3-yl)-9H-pyrrolo[2,3-b:5,4-c′]dipyridine;
3-methoxy-4-[4-(propan-2-yl)piperazin-1-yl]-6-(pyrid-3-yl)-9H-pyrrolo[2,3-b:5,4-c′]dipyridine;
4-(4-cyclopropylpiperazin-1-yl)-3-methoxy-6-(pyrid-3-yl)-9H-pyrrolo[2,3-b:5,4-c′]dipyridine:
4-(4-ethylpiperazin-1-yl)-3-methoxy-6-(pyrid-3-yl)-9H-pyrrolo[2,3-b:5,4-c′]dipyridine;
3-methoxy-4-[4-(methylsulfonyl)piperazin-1-yl]-6-(pyrid-3-yl)-9H-pyrrolo[2,3-b:5,4-c′]-dipyridine;
3-fluoro-4-[4-(methylsulfonyl)piperazin-1-yl]-6-(pyrid-3-yl)-9H-pyrrolo[2,3-b:5,4-c′]-dipyridine:
3-{4-[3-fluoro-6-(pyrid-3-yl)-9H-pyrrolo[2,3-b:5,4-c′]dipyrid-4-yl]phenyl}propanoic acid;
3-fluoro-4-(6-methoxypyrid-3-yl)-6-(pyrid-3-yl)-9H-pyrrolo[2,3-b:5,4-c′]dipyridine;
N-{3-[3-fluoro-6-(pyrid-3-yl)-9H-pyrrolo[2,3-b:5,4-c′]dipyrid-4-yl]phenyl}methanesulfon-amide;
3-fluoro-4-(4-methylthiophen-2-yl)-6-(pyrid-3-yl)-9H-pyrrolo[2,3-b:5,4-c′]dipyridine;
3-fluoro-4-(1 H-indol-6-yl)-6-(pyrid-3-yl)-9H-pyrrolo[2,3-b:5,4-c′]dipyridine;
{2-[3-fluoro-6-(pyrid-3-yl)-9H-pyrrolo[2,3-b:5,4-c′]dipyrid-4-yl]phenyl}methanol;
3-fluoro-4-(4-methylthiophen-3-yl)-6-(pyrid-3-yl)-9H-pyrrolo[2,3-b:5,4-c′]dipyridine;
3-[3-fluoro-6-(pyrid-3-yl)-9H-pyrrolo[2,3-b:5,4-c′]dipyrid-4-yl]-N,N-dimethylaniline;
3-fluoro-4-(5-methylfuran-2-yl)-6-(pyrid-3-yl)-9H-pyrrolo[2,3-b:5,4-c′]dipyridine;
3-fluoro-4-(1-methyl-1 H-indol-5-yl)-6-(pyrid-3-yl)-9H-pyrrolo[2,3-b:5,4-c′]dipyridine;
3-fluoro-4-(1-methyl-1 H-pyrazol-4-yl)-6-(pyrid-3-yl)-9H-pyrrolo[2,3-b:5,4-c′]dipyridine;
N-{4-[3-fluoro-6-(pyrid-3-yl)-9H-pyrrolo[2,3-b:5,4-c′]dipyrid-4-yl]benzyl}acetamide;
N-{3-[3-fluoro-6-(pyrid-3-yl)-9H-pyrrolo[2,3-b:5,4-c′]dipyrid-4-yl]benzyl}methanesulfon-amide;
3-fluoro-4-(2-methoxyphenyl)-6-(pyrid-3-yl)-9H-pyrrolo[2,3-b:5,4-c′]dipyridine;
4-(2-ethoxypyrid-3-yl)-3-fluoro-6-(pyrid-3-yl)-9H-pyrrolo[2,3-b:5,4-c′]dipyridine;
4-({3-[3-fluoro-6-(pyrid-3-yl)-9H-pyrrolo[2,3-b:5,4-c′]dipyrid-4-yl]phenyl}amino)-4-oxobutanoic acid;
N-{-4-[3-fluoro-6-(pyrid-3-yl)-9H-pyrrolo[2,3-b:5,4-c′]dipyrid-4-yl]benzyl}methanesulfonamide;
{-4-[3-fluoro-6-(pyrid-3-yl)-9H-pyrrolo[2,3-b:5,4-c′]dipyrid-4-yl]phenyl}(morpholin-4-yl)methanone;
3-fluoro-4-(1-methyl-1 H-pyrazol-5-yl)-6-(pyrid-3-yl)-9H-pyrrolo[2,3-b:5,4-c′]dipyridine;
1-{2-[3-fluoro-6-(pyrid-3-yl)-9H-pyrrolo[2,3-b:5,4-c′]dipyrid-4-yl]phenyl}-N,N-dimethyl-methanamine;
2-[3-fluoro-6-(pyrid-3-yl)-9,4-pyrrolo[2,3-b:5,4-c′]dipyrid-4-yl]benzonitrile;
1-chloro-N-{4-[3-fluoro-6-(pyrid-3-yl)-9H-pyrrolo[2,3-b:5,4-c′]dipyrid-4-yl]phenyl}methane-sulfonamide;
3-(4-methylpiperazin-1-yl)-6-(pyrid-3-yl)-9H-pyrrolo[2,3-b:5,4-c′]dipyridine;
N-{4-[3-fluoro-6-(pyrid-3-yl)-9H-pyrrolo[2,3-b:5,4-c′]dipyrid-4-yl]phenyl}cyclopropanesulfonamide;
N-{4-[3-fluoro-6-(pyrid-3-yl)-9H-pyrrolo[2,3-b:5,4-c′]dipyrid-4-yl]-2-methoxyphenyl}-methanesulfonamide;
N-{4-[3-fluoro-6-(1-methyl-1 H-pyrazol-4-yl)-9H-pyrrolo[2,3-b:5,4-c′]dipyrid-4-yl]phenyl}-methanesulfonamide;
3-fluoro-6-(5-methoxypyrid-3-yl)-9H-pyrrolo[2,3-b:5,4-c′]dipyridine;
3-fluoro-6-(4-methoxypyrid-3-yl)-9H-pyrrolo[2,3-b:5,4-c′]dipyridine;
6-(1-benzyl-1H-pyrazol-4-yl)-3-fluoro-9H-pyrrolo[2,3-b:5,4-c′]dipyridine;
3-fluoro-6-(1-methyl-1 H-pyrazol-4-yl)-9H-pyrrolo[2,3-b:5,4-c′]dipyridine;
3-fluoro-6-[1-(2-methylpropyl)-1 H-pyrazol-4-yl]-9H-pyrrolo[2,3-b:5,4-c′]dipyridine;
3-fluoro-6-[5-(methylsulfanyl)pyrid-3-yl]-9H-pyrrolo[2,3-b:5,4-c′]dipyridine;
3-fluoro-6-{1-[2-(morpholin-4-yl)ethyl]-1H-pyrazol-4-yl}-9H-pyrrolo[2,3-b:5,4-c′]dipyridine;
3-fluoro-4-[4-(propan-2-yl)piperazin-1-yl]-6-(pyrid-3-yl)-9H-pyrrolo[2,3-b:5,4-c′]dipyridine;
3-fluoro-4-(piperid-1-yl)-6-(pyrid-3-yl)-9H-pyrrolo[2,3-b:5,4-c′]dipyridine;
4-[3-fluoro-6-(pyrid-3-yl)-9H-pyrrolo[2,3-b:5,4-c′]dipyrid-4-yl]-2-methylbut-3-yn-2-amine;
4-[3-fluoro-6-(pyrid-3-yl)-9H-pyrrolo[2,3-b:5,4-c′]dipyrid-4-yl]-2-methylbut-3-yn-2-ol;
4-[3-(4-ethylpiperazin-1-yl)-3-methylbut-1-yn-1-yl]-3-fluoro-6-(pyrid-3-yl)-9H-pyrrolo[2,3-b:5,4-c′]dipyridine;
3-{4-[3-fluoro-6-(pyrid-3-yl)-9H-pyrrolo[2,3-b:5,4-c′]dipyrid-4-yl]phenyl}propanoic acid;
3-fluoro-4-(6-methoxypyrid-3-yl)-6-(pyrid-3-yl)-9H-pyrrolo[2,3-b:5,4-c′]dipyridine;
N-{3-[3-fluoro-6-(pyrid-3-yl)-9H-pyrrolo[2,3-b:5,4-c′]dipyrid-4-yl]phenyl}methanesulfon-amide;
3-fluoro-4-(4-methylthiophen-2-yl)-6-(pyrid-3-yl)-9H-pyrrolo[2,3-b:5,4-c′]dipyridine;
3-fluoro-4-(1 H-indol-6-yl)-6-(pyrid-3-yl)-9H-pyrrolo[2,3-b:5,4-c′]dipyridine;
{2-[3-fluoro-6-(pyrid-3-yl)-9H-pyrrolo[2,3-b:5,4-c′]dipyrid-4-yl]phenyl}methanol;
3-fluoro-4-(4-methylthiophen-3-yl)-6-(pyrid-3-yl)-9H-pyrrolo[2,3-b:5,4-c′]dipyridine;
3-[3-fluoro-6-(pyrid-3-yl)-9H-pyrrolo[2,3-b:5,4-c′]dipyrid-4-yl]-N,N-dimethylaniline;
3-fluoro-4-(1-methyl-1H-indol-5-yl)-6-(pyrid-3-yl)-9H-pyrrolo[2,3-b:5,4-c′]dipyridine;
3-fluoro-4-(1-methyl-1H-pyrazol-4-yl)-6-(pyrid-3-yl)-9H-pyrrolo[2,3-b:5,4-c′]dipyridine;
N-{4-[3-fluoro-6-(pyrid-3-yl)-9H-pyrrolo[2,3-b:5,4-c′]dipyrid-4-yl]benzyl}acetamide;
N-{3-[3-fluoro-6-(pyrid-3-yl)-9H-pyrrolo[2,3-b:5,4-c′]dipyrid-4-yl]benzyl}methanesulfonamide;
3-fluoro-4-(2-methoxyphenyl)-6-(pyrid-3-yl)-9H-pyrrolo[2,3-b:5,4-c′]dipyridine;
4-(2-ethoxypyrid-3-yl)-3-fluoro-6-(pyrid-3-yl)-9H-pyrrolo[2,3-b:5,4-c′]dipyridine;
4-({3-[3-fluoro-6-(pyrid-3-yl)-9H-pyrrolo[2,3-b:5,4-c′]dipyrid-4-yl]phenyl}amino)-4-oxobutanoic acid;
N-{4-[3-fluoro-6-(pyrid-3-yl)-9H-pyrrolo[2,3-b:5,4-c′]dipyrid-4-yl]benzyl}methanesulfon-amide;
3-fluoro-4-(1-methyl-1 H-pyrazol-5-yl)-6-(pyrid-3-yl)-9H-pyrrolo[2,3-b:5,4-c′]dipyridine;
N-{4-[3-fluoro-6-(pyrid-3-yl)-9H-pyrrolo[2,3-b:5,4-c′]dipyrid-4-yl]phenyl}-2-methylpropanamide;
3-fluoro-4,6-di(pyrid-3-yl)-9H-pyrrolo[2,3-b:5,4-c′]dipyridine;
N-{2-[3-fluoro-6-(pyrid-3-yl)-9H-pyrrolo[2,3-b:5,4-c′]dipyrid-4-yl]phenyl}methanesulfon-amide;
3-fluoro-4-(1 H-pyrazol-4-yl)-6-(pyrid-3-yl)-9H-pyrrolo[2,3-b:5,4-c′]dipyridine;
3-fluoro-4-[3-(methylsulfonyl)phenyl]-6-(pyrid-3-yl)-9H-pyrrolo[2,3-b:5,4-c′]dipyridine;
3-fluoro-4-(2-methoxypyrimidin-5-yl)-6-(pyrid-3-yl)-9H-pyrrolo[2,3-b:5,4-c′]dipyridine;
5-[3-fluoro-6-(pyrid-3-yl)-9H-pyrrolo[2,3-b:5,4-c′]dipyrid-4-yl]pyrid-2-amine;
3-fluoro-4-[4-(1-methylpiperid-4-yl)piperazin-1-yl]-6-(pyrid-3-yl)-9H-pyrrolo[2,3-b:5,4-c′]-dipyridine;
3-fluoro-4-[4-(morpholin-4-yl)piperid-1-yl]-6-(pyrid-3-yl)-9H-pyrrolo[2,3-b:5,4-c′]dipyridine;
N,N-diethyl-2-{4-[3-fluoro-6-(pyrid-3-yl)-9H-pyrrolo[2,3-b:5,4-c′]dipyrid-4-yl]piperazin-1-yl}-ethanamine;
3-fluoro-4-(4-methyl-1,4-diazepan-1-yl)-6-(pyrid-3-yl)-9H-pyrrolo[2,3-b:5,4-c′]dipyridine;
2-{4-[3-fluoro-6-(pyrid-3-yl)-9H-pyrrolo[2,3-b:5,4-c′]dipyrid-4-yl]piperazin-1-yl}ethanol;
3-fluoro-4-[4-(4-methylpiperazin-1-yl)piperid-1-yl]-6-(pyrid-3-yl)-9H-pyrrolo[2,3-b:5,4-c′]-dipyridine;
N-{4-[3-fluoro-6-(pyrid-3-yl)-9H-pyrrolo[2,3-b:5,4-c′]dipyrid-4-yl]phenyl}-N-methylmethane-sulfonamide;
3-(piperazin-1-yl)-6-(pyrid-3-yl)-9H-pyrrolo[2,3-b:5,4-c′]dipyridine;
6-(pyrid-3-yl)-9H-pyrrolo[2,3-b:5,4-c′]dipyrid-3-amine;
4-(1.4′-bipiperid-1′-yl)-3-fluoro-6-(pyrid-3-yl)-9H-pyrrolo[2,3-b:5,4-c′]dipyridine;
1-[3-fluoro-6-(pyrid-3-yl)-9H-pyrrolo[2,3-b:5,4-c′]dipyrid-4-yl]-N,N-dimethylpiperid-4-amine;
3-fluoro-6-(pyrid-3-yl)-4-[4-(pyrrolidin-1-yl)piperid-1-yl]-9H-pyrrolo[2,3-b:5,4-c′]dipyridine;
3-fluoro-4-{4-[3-(piperid-1-yl)propyl]piperazin-1-yl}-6-(pyrid-3-yl)-9H-pyrrolo[2,3-b:5,4-c′]-dipyridine;
3-fluoro-4-{4-[3-(morpholin-4-yl)propyl]piperazin-1-yl}-6-(pyrid-3-yl)-9H-pyrrolo[2,3-b:5,4-c′]dipyridine;
3-{-4-[3-fluoro-6-(pyrid-3-yl)-9H-pyrrolo[2,3-b:5,4-c′]dipyrid-4-yl]piperazin-1-yl}-N,N-dipropylpropan-1-amine;
3-ethoxy-6-(pyrid-3-yl)-9H-pyrrolo[2,3-b:5,4-c′]dipyridine;
3-iodo-6-(pyrid-3-yl)-9H-pyrrolo[2,3-b:5,4-c′]dipyridine;
3-{1-[2-(morpholin-4-yl)ethyl]-1 H-pyrazol-4-yl}-6-(pyrid-3-yl)-9H-pyrrolo[2,3-b:5,4-c′]-dipyridine;
3-(1-methyl-1H-pyrazol-3-yl)-6-(pyrid-3-yl)-9H-pyrrolo[2,3-b:5,4-c′]dipyridine;
N,N-diethyl-3-{4-[3-fluoro-6-(pyrid-3-yl)-9H-pyrrolo[2,3-b:5,4-c′]dipyrid-4-yl]piperazin-1-yl}-propan-1-amine;
N,N-diethyl-2-{4-[6-(pyrid-3-yl)-9H-pyrrolo[2,3-b:5,4-c′]dipyrid-3-yl]-1 H-pyrazol-1-yl}ethan-amine;
3-fluoro-4-methoxy-6-(pyrid-3-yl)-9H-pyrrolo[2,3-b:5,4-c′]dipyridine;
3-[1-(2-methylpropyl)-1 H-pyrazol-4-yl]-6-(pyrid-3-yl)-9H-pyrrolo[2,3-b:5,4-c′]dipyridine;
3-[4-(morpholin-4-yl)phenyl]-6-(pyrid-3-yl)-9H-pyrrolo[2,3-b:5,4-c′]dipyridine;
N-propyl-6-(pyrid-3-yl)-9H-pyrrolo[2,3-b:5,4-c′]dipyrid-3-amine;
3-{4-[4-(propan-2-yl)piperazin-1-yl]phenyl}-6-(pyrid-3-yl)-9H-pyrrolo[2,3-b:5,4-c′]dipyridine;
6-(pyrid-3-yl)-3-(2,2,2-trifluoroethoxy)-9H-pyrrolo[2,3-b:5,4-c′]dipyridine;
3-fluoro-9H-pyrrolo[2,3-b:5,4-c′]dipyridine-6-carbonitrile;
3-(2-methoxyethoxy)-6-(pyrid-3-yl)-9H-pyrrolo[2,3-b:5,4-c′]dipyridine;
3-{1-[3-(4-methylpiperazin-1-yl)propyl]-1 H-pyrazol-4-yl}-6-(pyrid-3-yl)-9H-pyrrolo[2,3-b:5,4-c′]dipyridine;
{3-[6-(pyrid-3-yl)-9H-pyrrolo[2,3-b:5,4-c′]dipyrid-3-yl]phenyl}methanol;
N,N-diethyl-3-[6-(pyrid-3-yl)-9H-pyrrolo[2,3-b:5,4-c′]dipyrid-3-yl]benzamide;
3-(3,5-dimethyl-1 H-pyrazol-4-yl)-6-(pyrid-3-yl)-9H-pyrrolo[2,3-b:5,4-c′]dipyridine;
2-{3,5-dimethyl-4-[6-(pyrid-3-yl)-9H-pyrrolo[2,3-b:5,4-c′]dipyrid-3-yl]-1 H-pyrazol-1-yl}-N,N-diethylethanamine;
3-methoxy-6-(1-methyl-1 H-pyrazol-4-yl)-9H-pyrrolo[2,3-b:5,4-c′]dipyridine;
methyl 4-{6-[1-(prop-2-en-1-yl)-1 H-pyrazol-4-yl]-9H-pyrrolo[2,3-b:5,4-c′]dipyrid-4-yl}benzoate;
N,N-diethyl-2-[4-(3-methoxy-9H-pyrrolo[2,3-b:5,4-c′]dipyrid-6-yl)-3,5-dimethyl-1 H-pyrazol-1-yl]ethanamine;
N-[2-(dimethylamino)ethyl]-2-[4-(3-methoxy-9H-pyrrolo[2,3-b:5,4-c′]dipyrid-6-yl)-1 H-pyrazol-1-yl]acetamide;
3-(1 H-pyrazol-4-yl)-6-(pyrid-3-yl)-9H-pyrrolo[2,3-b:5,4-c′]dipyridine;
N,N-diethyl-3-{4-[6-(pyrid-3-yl)-9H-pyrrolo[2,3-b:5,4-c′]dipyrid-3-yl]-1 H-pyrazol-1-yl}-propan-1-amine;
N,N-diethyl-3-[4-(3-methoxy-9H-pyrrolo[2,3-b:5,4-c′]dipyrid-6-yl)-1 H-pyrazol-1-yl]propan-1-amine;
9H-pyrrolo[2,3-b:5,4-c′]dipyridine-6-carboxylic acid;
N-[3-(dimethylamino)propyl]-N-{4-[3-fluoro-6-(pyrid-3-yl)-9H-pyrrolo[2,3-b:5,4-c′]dipyrid-4-yl]phenyl}methanesulfonamide;
(4-methylpiperazin-1-yl)(9H-pyrrolo[2,3-b:5,4-c′]dipyrid-6-yl)methanone;
5-[4-(3-methoxy-9H-pyrrolo[2,3-b:5,4-c′]dipyrid-6-yl)-1 H-pyrazol-1-yl]pentan-1-amine;
2-methyl-2-propyl {5-[4-(3-methoxy-9H-pyrrolo[2,3-b:5,4-c′]dipyrid-6-yl)-1 H-pyrazol-1-yl]-pentyl}carbamate;
3-methoxy-6-{1-[2-(1-methylpiperid-2-yl)ethyl]-1 H-pyrazol-4-yl}-9H-pyrrolo[2,3-b:5,4-c′]dipyridine;
3-{4-[3-fluoro-6-(pyrid-3-yl)-9H-pyrrolo[2,3-b:5,4-c′]dipyrid-4-yl]phenoxy}-N,N-dimethylpropan-1-amine;
4-[3-fluoro-6-(pyrid-3-yl)-9H-pyrrolo[2,3-b:5,4-c′]dipyrid-4-yl]phenol;
2-{4-[3-fluoro-6-(pyrid-3-yl)-9H-pyrrolo[2,3-b:5,4-c′]dipyrid-4-yl]phenoxy}-N,N-dimethyl-ethanamine;
3-{1-[(1-ethylpyrrolidin-2-yl)methyl]-1H-pyrazol-4-yl}-6-(pyrid-3-yl)-9H-pyrrolo[2,3-b:5,4-c′]dipyridine;
3-fluoro-6-(pyrid-3-yl)-4-{4-[2-(pyrrolidin-1-yl)ethoxy]phenyl}-9H-pyrrolo[2,3-b:5,4-c′]-dipyridine;
3-fluoro-6-(thiophen-3-yl)-9H-pyrrolo[2,3-b:5,4-c′]dipyridine;
2-methyl-2-propyl 4-{4-[6-(pyrid-3-yl)-9H-pyrrolo[2,3-b:5,4-c′]dipyrid-3-yl]-phenyl}piperazine-1-carboxylate;
3-{4-[3-fluoro-6-(pyrid-3-yl)-9H-pyrrolo[2,3-b:5,4-c′]dipyrid-4-yl]phenoxy}-N,N,2-trimethyl-propan-1-amine;
3-fluoro-4-{4-[2-(morpholin-4-yl)ethoxy]phenyl}-6-(pyrid-3-yl)-9H-pyrrolo[2,3-b:5,4-c′]-dipyridine;
N,N-diethyl-2-{4-[3-fluoro-6-(pyrid-3-yl)-9H-pyrrolo[2,3-b:5,4-c′]dipyrid-4-yl]phenoxy}ethan-amine;
N-[2-(dimethylamino)ethyl]-5-[3-fluoro-6-(pyrid-3-yl)-9H-pyrrolo[2,3-b:5/4-c′]dipyrid-4-yl]-pyridine-2-carboxamide;
1-{4-[3-fluoro-6-(pyrid-3-yl)-9H-pyrrolo[2,3-b:5,4-c′]dipyrid-4-yl]phenoxy}-3-(morpholin-4-yl)propan-2-ol;
N-ethyl-3-{4-[3-fluoro-6-(pyrid-3-yl)-9H-pyrrolo[2,3-b:5,4-c′]dipyrid-4-yl]phenoxy}propan-1-amine;
4-[6-(pyrid-3-yl)-9H-pyrrolo[2,3-b:5,4-c′]dipyrid-3-yl]phenol;
3-[4-(piperazin-1-yl)phenyl]-6-(pyrid-3-yl)-9H-pyrrolo[2,3-b:5,4-c′]dipyridine;
3-fluoro-6-(isoquinolin-4-yl)-9H-pyrrolo[2,3-b:5,4-c′]dipyridine;
N,N-dimethyl-3-{4-[6-(pyrid-3-yl)-9H-pyrrolo[2,3-b:5,4-c′]dipyrid-3-yl]phenoxy}propan-1-amine;
3-{4-[3-(piperid-1-yl)propoxy]phenyl}-6-(pyrid-3-yl)-9H-pyrrolo[2,3-b:5,4-c′]dipyridine;
3-{4-[2-(morpholin-4-yl)ethoxy]phenyl}-6-(pyrid-3-yl)-9H-pyrrolo[2,3-b:5,4-c′]dipyridine;
3-{4-[3-(morpholin-4-yl)propoxy]phenyl}-6-(pyrid-3-yl)-9H-pyrrolo[2,3-b:5,4-c′]dipyridine;
3-{4-[2-(1H-imidazol-1-yl)ethoxy]phenyl}-6-(pyrid-3-yl)-9H-pyrrolo[2,3-b:5,4-c′]dipyridine;
3-(4-{3-[4-(methylsulfonyl)piperazin-1-yl]propoxy}phenyl)-6-(pyrid-3-yl)-9H-pyrrolo[2,3-b:5,4-c′]dipyridine;
N,N-diethyl-2-{3-[6-(pyrid-3-yl)-9H-pyrrolo[2,3-b:5,4-c′]dipyrid-3-yl]phenoxy}ethanamine;
2-methyl-2-propyl 4-{3-[6-(pyrid-3-yl)-9H-pyrrolo dipyrid-3-yl]-phenyl}piperazine-1-carboxylate;
N,N,4-triethyl-5-[6-(pyrid-3-yl)-9H-pyrrolo[2,3-b:5,4-c′]dipyrid-3-yl]pyrid-2-amine;
3-[3-(piperazin-1-yl)phenyl]-6-(pyrid-3-yl)-9H-pyrrolo[2,3-b:5,4-c′]dipyridine hydrochloride;
N,N-diethyl-2-({4-[3-fluoro-6-(pyrid-3-yl)-9H-pyrrolo[2,3-b:5,4-c′]dipyrid-4-yl]-2-methylbut-3-yn-2-yl}oxy)ethanamine;
4-[3-fluoro-6-(pyrid-3-yl)-9H-pyrrolo[2,3-b:5,4-c′]dipyrid-4-yl]-N-(prop-2-en-1-yl)aniline;
N-(2-methylpropan-2-yl)-5-(9H-pyrrolo[2,3-b:5,4-c′]dipyrid-6-yl)pyridine-3-carboxamide;
5-(3-fluoro-9H-pyrrolo[2,3-b:5,4-c′]dipyrid-6-yl)-N-(2-methylpropan-2-yl)pyridine-3 carboxamide;
3-fluoro-6-(1 H-pyrazol-4-yl)-9H-pyrrolo[2,3-b:5,4-c′]dipyridine;
(2E)-N-[4-(dimethylamino)butyl]-3-[3-fluoro-6-(pyrid-3-yl)-9H-pyrrolo[2,3-b:5,4-c′]dipyrid-4-yl]prop-2-enamide;
6-chloro-3-fluoro-9H-pyrrolo[2,3-b:5,4-c′]dipyridine;
3-{4-[6-(pyrid-3-yl)-9H-pyrrolo[2,3-b:5,4-c′]dipyrid-3-yl]phenoxy}propan-1-amine;
3-{1-[3-(4-methylpiperazin-1-yl)propyl]-1 H-pyrazol-4-yl}-6-(1-methyl-1 H-pyrazol-4-yl)-9H-pyrrolo[2,3-b:5,4-c′]dipyridine;
3-[3-(4-methylpiperazin-1-yl)phenyl]-6-(1-methyl-1 H-pyrazol-4-yl)-9H-pyrrolo[2,3-b:5,4-c′]-dipyridine;
N,N-diethyl-2-{4-[6-(1-methyl-1 H-pyrazol-4-yl)-9H-pyrrolo[2,3-b:5,4-c′]dipyrid-3-yl]-1 H-pyrazol-1-yl}ethanamine;
6-(1-methyl-1 H-pyrazol-4-yl)-3-{4-[3-(morpholin-4-yl)propoxy]phenyl}-9H-pyrrolo[2,3-b:5,4-c′]dipyridine;
N,N-diethyl-2-{3-[6-(1-methyl-1 H-pyrazol-4-yl)-9H-pyrrolo[2,3-b:5,4-c′]dipyrid-3-yl]-phenoxy}ethanamine;
3-fluoro-6-(1-methyl-1H-pyrazol-4-yl)-4-{4-[3-(piperid-1-yl)propyl]piperazin-1-yl}-9H-pyrrolo[2,3-b:5,4-c′]dipyridine;
4-[3-(4-ethylpiperazin-1-yl)-3-methylbut-1-yn-1-yl]-3-fluoro-6-(1-methyl-1 H-pyrazol-4-yl)-9H-pyrrolo[2,3-b:5,4-c′]dipyridine;
N-[3-(dimethylamino)propyl]-N-{4-[3-fluoro-6-(1-methyl-1 H-pyrazol-4-yl)-9H-pyrrolo[2,3-b:5,4-c′]dipyrid-4-yl]phenyl}methanesulfonamide;
N-ethyl-3-{4-[3-fluoro-6-(1-methyl-1 H-pyrazol-4-yl)-9H-pyrrolo[2,3-b:5,4-c′]dipyrid-4-yl]phenoxy}propan-1-amine;
N,N-diethyl-2-{4-[3-fluoro-6-(1-methyl-1 H-pyrazol-4-yl)-9H-pyrrolo[2,3-b:5,4-c′]dipyrid-4-yl]phenoxy}ethanamine;
3-{4-[3-fluoro-6-(1-methyl-1 H-pyrazol-4-yl)-9H-pyrrolo[2,3-b:5,4-c′]dipyrid-4-yl]phenoxy}-N,N,2-trimethylpropan-1-amine;
1-{4-[3-fluoro-6-(1-methyl-1 H-pyrazol-4-yl)-9H-pyrrolo[2,3-b:5,4-c′]dipyrid-4-yl]phenoxy}-3-(piperid-1-yl)propan-2-ol;
1-{4-[3-(2-methoxyethoxy)-6-(1-methyl-1H-pyrazol-4-yl)-9H-pyrrolo[2,3-b:5,4-c′]dipyrid-4-yl]phenoxy}-3-(piperid-1-yl)propan-2-ol;
3-(2-methoxyethoxy)-6-(1-methyl-1H-pyrazol-4-yl)-4-{4-[3-(piperid-1-yl)Propyl]piperazin-1-yl}-9H-pyrrolo[2,3-b:5,4-c′]dipyridine;
4-[3-(4-ethylpiperazin-1-yl)-3-methylbut-1-yn-1-yl]-3-(2-methoxyethoxy)-6-(1-methyl-1 H-pyrazol-4-yl)-9H-pyrrolo[2,3-b:5,4-c′]dipyridine;
N-[3-(dimethylamino)propyl]-N-{4-[3-(2-methoxyethoxy)-6-(1-methyl-1 H-pyrazol-4-yl)-9H-pyrrolo[2,3-b:5,4-c′]dipyrid-4-yl]phenyl}methanesulfonamide;
N-ethyl-3-{4-[3-(2-methoxyethoxy)-6-(1-methyl-1H-pyrazol-4-yl)-9H-pyrrolo[2,3-b:5,4-c′]dipyrid-4-yl]phenoxy}propan-1-amine;
3-{4-[3-(2-methoxyethoxy)-6-(1-methyl-1 H-pyrazol-4-yl)-9H-pyrrolo[2,3-b:5,4-c′]dipyrid-4-yl]phenoxy}-N,N,2-trimethylpropan-1-amine;
N,N-diethyl-2-{4-[3-(2-methoxyethoxy)-6-(1-methyl-1H-pyrazol-4-yl)-9H-pyrrolo[2,3-b:5,4-c′]dipyrid-4-yl]phenoxy}ethanamine;
1-{4-[3-(2-methoxyethoxy)-6-(1-methyl-1H-pyrazol-4-yl)-9H-pyrrolo[2,3-b:5,4-c′]dipyrid-4-yl]phenoxy}-3-(piperid-1-yl)propan-2-ol;
3-amino-1-{4-[3-fluoro-6-(pyrid-3-yl)-9H-pyrrolo[2,3-b:5,4-c′]dipyrid-4-yl]phenyl}pyrrolidine-2,5-dione;
4-({[3-fluoro-6-(pyrid-3-yl)-9H-pyrrolo[2,3-b:5,4-c′]dipyrid-4-yl]oxy}methyl)-N,N-dimethylaniline;
3-fluoro-6-(pyrid-3-yl)-9H-pyrrolo[2,3-b:5,4-c′]dipyrid-4-yl[4-(dimethylamino)phenyl]carbamate;
3-fluoro-6-(pyrid-3-yl)-9H-pyrrolo[2,3-b:5,4-c′]dipyrid-4-yl[3-(dimethylamino)propyl]carbamate;
3-[(3-fluoro-9H-pyrrolo[2,3-b:5,4-c′]dipyrid-6-yl)carbonyl]-1.5.5-trimethylimidazolidine-2.4-dione;
3-[(3-fluoro-9H-pyrrolo[2,3-b:5,4-c′]dipyrid-6-yl)carbonyl]-1-methylimidazolidine-2.4-dione;
3-[(3-fluoro-9H-pyrrolo[2,3-b:5,4-c′]dipyrid-6-yl)carbonyl]-5,5-dimethyl-1-(propan-2-yl)imid-azolidine-2.4-dione;
1-[(3-fluoro-9H-pyrrolo[2,3-b:5,4-c′]dipyrid-6-yl)carbonyl]-4.4-dimethyl-3-(propan-2-yl)imid-azolidin-2-one;
1-[(3-fluoro-9H-pyrrolo[2,3-b:5,4-c′]dipyrid-6-yl)carbonyl]-3.4.4-trimethylimidazolidin-2-one;
1-[(3-fluoro-9H-pyrrolo[2,3-b:5,4-c′]dipyrid-6-yl)carbonyl]-3-methylimidazolidin-2-one;
3-fluoro-6-(1-methyl-1 H-imidazol-5-yl)-9H-pyrrolo[2,3-b:5,4-c′]dipyridine;
3-fluoro-6-{1-methyl-5-[3-methyl-3-(4-methylpiperazin-1-yl)but-1-yn-1-yl]-1H-pyrazol-4-yl}-9H-pyrrolo[2,3-b:5,4-c′]dipyridine;
6-(5-chloro-1-methyl-1H-pyrazol-4-yl)-3-fluoro-9H-pyrrolo[2,3-b:5,4-c′]dipyridine;
6-(5-bromo-1-methyl-1H-pyrazol-4-yl)-3-fluoro-9H-pyrrolo[2,3-b:5,4-c′]dipyridine;
N-{4-[3-(dimethylamino)propoxy]benzyl}-6-(pyrid-3-yl)-9H-pyrrolo[2,3-b:5,4-c′]dipyrid-3-amine;
N-{4-[2-(dimethylamino)ethoxy]benzyl}-6-(pyrid-3-yl)-9H-pyrrolo[2,3-b:5,4-c′]dipyrid-3-amine;
6-(pyrid-3-yl)-N-{[2-(pyrid-4-yl)cyclopropyl]methyl}-9H-pyrrolo[2,3-b:5,4-c′]dipyrid-3-amine;
N-[3-fluoro-4-(piperazin-1-yl)benzyl]-6-(pyrid-3-yl)-9H-pyrrolo[2,3-b:5,4-c′]dipyrid-3-amine;
6-(pyrid-3-yl)-N-{[1-(pyrid-3-ylmethyl)-1 H-pyrrol-2-yl]methyl}-9H-pyrrolo[2,3-b:5,4-c′]-dipyrid-3-amine;
N-{4-[(dimethylamino)methyl]benzyl}-6-(pyrid-3-yl)-9H-pyrrolo[2,3-b:5,4-c′]dipyrid-3-amine;
4-methyl-N1-[6-(pyrid-3-yl)-9H-pyrrolo[2,3-b:5,4-c′]dipyrid-3-yl]pentane-1,4-diamine;
N-(4-methyl-4-nitropentyl)-6-(pyrid-3-yl)-9H-pyrrolo[2,3-b; 5,4-c′]dipyrid-3-amine;
N,N-dimethyl-N′[6-(pyrid-3-yl)-9H-pyrrolo[2,3-b:5,4-c′]dipyrid-3-yl]butane-1,4-diamine;
piperazin-1-yl[4-({[6-(pyrid-3-yl)-9H-pyrrolo[2,3-b:5,4-c′]dipyrid-3-yl]amino}methyl)phenyl]-methanone;
N-[4-(aminomethyl)benzyl]-6-(pyrid-3-yl)-9H-pyrrolo[2,3-b:5,4-c′]dipyrid-3-amine;
2-methyl-2-propyl[4-({[6-(pyrid-3-yl)-9H-pyrrolo[2,3-b:5,4-c′]dipyrid-3-yl]amino}methyl)-benzyl]carbamate;
2-methyl-2-propyl 4-{[4-({[6-(pyrid-3-yl)-9H-pyrrolo[2,3-b:5,4-c′]dipyrid-3-yl]amino}methyl)-phenyl]carbonyl}piperazine-1-carboxylate;
N-[4-(dimethylamino)benzyl]-6-(pyrid-3-yl)-9H-pyrrolo[2,3-b:5,4-c′]dipyrid-3-amine;
N-{4-[(4-methyl-1,4-diazepan-1-yl)methyl]benzyl}-6-(pyrid-3-yl)-9H-pyrrolo[2,3-b:5,4-c′]-dipyrid-3-amine;
4-(4-methyl-1,4-diazepan-1-yl)-N-[6-(pyrid-3-yl)-9H-pyrrolo[2,3-b:5,4-c′]dipyrid-3-yl]benzamide;
N-[4-(4-methyl-1,4-diazepan-1-yl)benzyl]-6-(pyrid-3-yl)-9H-pyrrolo[2,3-b:5,4-c′]dipyrid-3-amine;
3-(4-methyl-1,4-diazepan-1-yl)-N-[6-(pyrid-3-yl)-9H-pyrrolo[2,3-b:5,4-c′]dipyrid-3-yl]propanamide;
3-[(4-methyl-1,4-diazepan-1-yl)methyl]-N-[6-(pyrid-3-yl)-9H-pyrrolo[2,3-b:5,4-c′]dipyrid-3-yl]benzamide;
N-{3-[(4-methyl-1,4-diazepan-1-yl)methyl]benzyl}-6-(pyrid-3-yl)-9H-pyrrolo[2,3-b:5,4-c′]dipyrid-3-amine;
N-[2-(4-methyl-1,4-diazepan-1-yl)ethyl]-6-(pyrid-3-yl)-9H-pyrrolo[2,3-b:5,4-c′]dipyrid-3-amine;
6-(pyrid-3-yl)-9H-pyrrolo[2,3-b:5,4-c′]dipyridine-3-carbonitrile;
6-(3,5-dimethyl-1 H-pyrazol-4-yl)-3-(pyrid-3-yl)-9H-3-carboline;
2-{3-[6-(pyrid-3-yl)-9H-pyrrolo[2,3-b:5,4-c′]dipyrid-3-yl]phenoxy}ethanamine;
3-(4-{[6-(pyrid-3-yl)-9H-pyrrolo[2,3-b:5,4-c′]dipyrid-3-yl]oxy}phenyl)propan-1-ol;
N,N-dimethyl-2-(4-{[6-(pyrid-3-yl)-9H-pyrrolo[2,3-b:5,4-c′]dipyrid-3-yl]oxy}phenyl)ethan-amine;
2-(4-{[6-(pyrid-3-yl)-9H-pyrrolo[2,3-b:5,4-c′]dipyrid-3-yl]oxy}phenyl)acetamide;
N-methyl-2-(4-{[6-(pyrid-3-yl)-9H-pyrrolo[2,3-b:5,4-c′]dipyrid-3-yl]oxy}phenyl)acetamide;
N-cyclopropyl-2-(4-{[6-(pyrid-3-yl)-9H-pyrrolo[2,3-b:5,4-c′]dipyrid-3-yl]oxy}phenyl)acetamide;
N-(propan-2-yl)-1-(4-{[6-(pyrid-3-yl)-9H-pyrrolo[2,3-b:5,4-c′]dipyrid-3-yl]oxy}phenyl)propan-2-amine;
6-(pyrid-3-yl)-3-{4-[2-(pyrrolidin-1-yl)propyl]phenoxy}-9H-pyrrolo[2,3-b:5,4-c′]dipyridine;
N,N-diethyl-3-(4-{[6-(pyrid-3-yl)-9H-pyrrolo[2,3-b:5,4-c′]dipyrid-3-yl]oxy}phenyl)propan-1-amine;
N,N-diethyl-2-{[6-(pyrid-3-yl)-9H-pyrrolo[2,3-b:5,4-c′]dipyrid-3-yl]oxy}ethanamine.

5. A compound according to claim 1, chosen from:
N-{4-[3-fluoro-6-(pyrid-3-yl)-9H-pyrrolo[2,3-b:5,4-c′]dipyrid-4-yl]phenyl}methanesulfon-amide;
N-{-4-[3-methoxy-6-(pyrid-3-yl)-9H-pyrrolo[2,3-b:5,4-c′]dipyrid-4-yl]phenyl}methanesulfon-amide;
4-(3,5-dimethoxyphenyl)-3-fluoro-6-(pyrid-3-yl)-9H-pyrrolo[2,3-b:5,4-c′]dipyridine;
4-cyclopropyl-3-fluoro-6-(pyrid-3-yl)-9H-pyrrolo[2,3-b:5,4-c′]dipyridine;
3-methoxy-6-(pyrid-3-yl)-9H-pyrrolo[2,3-b:5,4-c′]dipyridine;
N-cyclopropyl-4-[3-fluoro-6-(pyrid-3-yl)-9H-pyrrolo[2,3-b:5,4-c′]dipyrid-4-yl]benzenesulfonamide;
3-hydroxy-2,2-dimethylpropyl 6-(pyrid-3-yl)-9H-pyrrolo[2,3-b:5,4-c′]dipyridine-3-carboxylate;
4-methoxy-6-(pyrid-3-yl)-9H-pyrrolo[2,3-b:5,4-c′]dipyridine;
3-[3-fluoro-6-(pyrid-3-yl)-9H-pyrrolo[2,3-b:5,4-c′]dipyrid-4-yl]phenol;
4-[(E)-2-cyclopropylethenyl]-3-fluoro-6-(pyrid-3-yl)-9H-pyrrolo[2,3-b:5,4-c′]dipyridine;
4-(3,5-difluorophenyl)-3-fluoro-6-(pyrid-3-yl)-9H-pyrrolo[2,3-b:5,4-c′]dipyridine;
2-methylpropan-2-yl 6-(pyrid-3-yl)-9H-pyrrolo[2,3-b:5,4-c′]dipyridine-3-carboxylate;
3-fluoro-4-iodo-6-(pyrid-3-yl)-9H-pyrrolo[2,3-b:5,4-c′]dipyridine;
4-[3-fluoro-6-(pyrid-3-yl)-9H-pyrrolo[2,3-b:5,4-c′]dipyrid-4-yl]butane-1,2-diol;
[3-fluoro-6-(pyrid-3-yl)-9H-pyrrolo[2,3-b:5,4-c′]dipyrid-4-yl](phenyl)methanone;
3-[3-fluoro-6-(pyrid-3-yl)-9H-pyrrolo[2,3-b:5,4-c′]dipyrid-4-yl]benzenesulfonamide;
3-(morpholin-4-yl)-6-(pyrid-3-yl)-9H-pyrrolo[2,3-b:5,4-c′]dipyridine;
6-(1-methyl-1 H-pyrazol-4-yl)-9H-pyrrolo[2,3-b:5,4-c′]dipyridine;
3-fluoro-4-(morpholin-4-yl)-6-(pyrid-3-yl)-9H-pyrrolo[2,3-b:5,4-c′]dipyridine;
2-methylpropyl 6-(pyrid-3-yl)-9H-pyrrolo[2,3-b:5,4-c′]dipyridine-3-carboxylate;
N-methyl-N-propyl-6-(pyrid-3-yl)-9H-pyrrolo[2,3-b:5,4-c′]dipyrid-3-amine;
ethyl 6-(pyrid-3-yl)-9H-pyrrolo[2,3-b:5,4-c′]dipyridine-3-carboxylate;
6-(pyrid-3-yl)-9H-pyrrolo[2,3-b:5,4-c′]dipyridine;
3-fluoro-4-methyl-6-(pyrid-3-yl)-9H-pyrrolo[2,3-b:5,4-c′]dipyridine;
3-fluoro-6-(pyrid-3-yl)-9H-pyrrolo[2,3-b:5,4-c′]dipyridine;
4-chloro-3-fluoro-6-(pyrid-3-yl)-9H-pyrrolo[2,3-b:5,4-c′]dipyridine;
3-fluoro-4-[(E)-2-phenylethenyl]-6-(pyrid-3-yl)-9H-pyrrolo[2,3-b:5,4-c′]dipyridine;
3-chloro-6-(pyrid-3-yl)-9H-pyrrolo[2,3-b:5,4-c′]dipyridine;
3-bromo-6-(pyrid-3-yl)-9H-pyrrolo[2,3-b:5,4-c′]dipyridine;
ethyl (2E)-3-[3-fluoro-6-(pyrid-3-yl)-9H-pyrrolo[2,3-b:5,4-c′]dipyrid-4-yl]prop-2-enoate;
3-fluoro-4-[3-(morpholin-4-yl)phenyl]-6-(pyrid-3-yl)-9H-pyrrolo[2,3-b:5,4-c′]dipyridine;
6-(pyrid-3-yl)-9H-pyrrolo[2,3-b:5,4-c′]dipyridine-3-carboxylic acid;
[6-(pyrid-3-yl)-9H-pyrrolo[2,3-b:5,4-c′]dipyrid-3-yl]methanol;
methyl 6-(pyrid-3-yl)-9,4-pyrrolo[2,3-b:5,4-c′]dipyridine-3-carboxylate;
N-methyl-N-propyl-6-(pyrid-3-yl)-9H-pyrrolo[2,3-b:5,4-c′]dipyridine-3-carboxamide;
3-fluoro-N-methyl-N-phenyl-6-(pyrid-3-yl)-9H-pyrrolo[2,3-b:5,4-c′]dipyridine-4-carboxamide;
4-{methyl[6-(pyrid-3-yl)-9H-pyrrolo[2,3-b:5,4-c′]dipyrid-3-yl]amino}-1-(pyrrolidin-1-yl)butan-1-one;
6-(furan-3-yl)-9H-pyrrolo[2,3-b:5,4-c′]dipyridine;
[3-fluoro-6-(pyrid-3-yl)-9H-pyrrolo[2,3-b:5,4-c′]dipyrid-4-yl](morpholin-4-yl)methanone;
6-(5-fluoropyrid-3-yl)-9H-pyrrolo[2,3-b:5,4-c′]dipyridine;
2-[6-(pyrid-3-yl)-9H-pyrrolo[2,3-b:5,4-c′]dipyrid-3-yl]propan-2-ol;
6-(6-fluoropyrid-3-yl)-9H-pyrrolo[2,3-b:5,4-c′]dipyridine;
N,N-diethyl-6-(pyrid-3-yl)-9H-pyrrolo[2,3-b:5,4-c′]dipyrid-3-amine;
3-(pyrid-3-yl)-9H-pyrrolo[2,3-b:5,4-c′]dipyridine;
3-methoxy-6-(pyrid-3-yl)-9H-pyrrolo[2,3-b:5,4-c′]dipyridine;
1-chloro-N-{4-[3-fluoro-6-(pyrid-3-yl)-9H-pyrrolo[2,3-b:5,4-c′]dipyrid-4-yl]phenyl}methane-sulfonamide;
3-(4-methylpiperazin-1-yl)-6-(pyrid-3-yl)-9H-pyrrolo[2,3-b:5,4-c′]dipyridine;
N-{4-[3-fluoro-6-(pyrid-3-yl)-9H-pyrrolo[2,3-b:5,4-c′]dipyrid-4-yl]phenyl}cyclopropanesulfonamide;
N-{4-[3-fluoro-6-(pyrid-3-yl)-9H-pyrrolo[2,3-b:5,4-c′]dipyrid-4-yl]-2-methoxyphenyl}-methanesulfonamide;
N-{4-[3-fluoro-6-(1-methyl-1H-pyrazol-4-yl)-9H-pyrrolo[2,3-b:5,4-c′]dipyrid-4-yl]phenyl}-methanesulfonamide;
3-fluoro-6-(5-methoxypyrid-3-yl)-9H-pyrrolo[2,3-b:5,4-c′]dipyridine;
3-fluoro-6-(4-methoxypyrid-3-yl)-9H-pyrrolo[2,3-b:5,4-c′]dipyridine;
6-(1-benzyl-1H-pyrazol-4-yl)-3-fluoro-9H-pyrrolo[2,3-b:5,4-c′]dipyridine;
3-fluoro-6-(1-methyl-1 H-pyrazol-4-yl)-9H-pyrrolo[2,3-b:5,4-c′]dipyridine;
3-fluoro-6-[1-(2-methylpropyl)-1H-pyrazol-4-yl]-9H-pyrrolo[2,3-b:5,4-c′]dipyridine;
3-fluoro-6-[5-(methylsulfanyl)pyrid-3-yl]-9H-pyrrolo[2,3-b:5,4-c′]dipyridine;
4-[3-fluoro-6-(pyrid-3-yl)-9H-pyrrolo[2,3-b:5,4-c′]dipyrid-4-yl]-2-methylbut-3-yn-2-ol;
4-[3-fluoro-6-(pyrid-3-yl)-9H-pyrrolo[2,3-b:5,4-c′]dipyrid-4-yl]-2-methylbut-3-yn-2-amine;
N-{4-[3-fluoro-6-(pyrid-3-yl)-9H-pyrrolo[2,3-b:5,4-c′]dipyrid-4-yl]-2-methylbut-3-yn-2-yl}-methanesulfonamide;
3-fluoro-4-[3-methyl-3-(piperazin-1-yl)but-1-yn-1-yl]-6-(pyrid-3-yl)-9H-pyrrolo[2,3-b:5,4-c′]-dipyridine;
4-[3-methoxy-6-(pyrid-3-yl)-9H-pyrrolo[2,3-b:5,4-c′]dipyrid-4-yl]-2-methylbut-3-YD-2-ol;
4-[3-methoxy-6-(pyrid-3-yl)-9H-pyrrolo[2,3-b:5,4-c′]dipyrid-4-yl]-2-methylbut-3-yn-2-amine;
N-{4-[3-methoxy-6-(pyrid-3-yl)-9H-pyrrolo[2,3-b:5,4-c′]dipyrid-4-yl]-2-methylbut-3-yn-2-yl}-methanesulfonamide;
3-methoxy-4-[3-methyl-3-(piperazin-1-yl)but-1-yn-1-yl]-6-(pyrid-3-yl)-9H-pyrrolo[2,3-b:5,4-c′]dipyridine;
3-fluoro-4-[4-(4-methylpiperazin-1-yl)piperid-1-yl]-6-(pyrid-3-yl)-9H-pyrrolo[2,3-b:5,4-c′]-dipyridine;
2-(4-{1-[3-fluoro-6-(pyrid-3-yl)-9H-pyrrolo[2,3-b:5,4-c′]dipyrid-4-yl]piperid-4-yl}piperazin-1-yl)ethanol;
3-fluoro-4-[4-(morpholin-4-yl)piperid-1-yl]-6-(pyrid-3-yl)-9H-pyrrolo[2,3-b:5,4-c′]dipyridine;
3-fluoro-4-[4-(propan-2-yl)piperazin-1-yl]-6-(pyrid-3-yl)-9H-pyrrolo[2,3-b:5,4-c′]dipyridine;
4-(4-cyclopropylpiperazin-1-yl)-3-fluoro-6-(pyrid-3-yl)-9H-pyrrolo[2,3-b:5,4-c′]dipyridine;
4-(4-ethylpiperazin-1-yl)-3-fluoro-6-(pyrid-3-yl)-9H-pyrrolo[2,3-b:5,4-c′]dipyridine;
3-fluoro-4-[4-(1-methylpiperid-4-yl)piperazin-1-yl]-6-(pyrid-3-yl)-9H-pyrrolo[2,3-b:5,4-c′]-dipyridine;
3-methoxy-4-[4-(4-methylpiperazin-4-yl)piperid-1-yl]-6-(pyrid-3-yl)-9H-pyrrolo[2,3-b:5,4-c′]dipyridine;
2-(4-{1-[3-methoxy-6-(pyrid-3-yl)-9H-pyrrolo[2,3-b:5,4-c′]dipyrid-4-yl]piperid-4-yl}piperazin-1-yl)ethanol;
3-methoxy-4-[4-(morpholin-4-yl)piperid-1-yl]-6-(pyrid-3-yl)-9H-pyrrolo[2,3-b:5,4-c′]-dipyridine;
3-methoxy-4-[4-(1-methylpiperid-4-yl)piperazin-1-yl]-6-(pyrid-3-yl)-9H-pyrrolo[2,3-b:5,4-c′]-dipyridine;
3-methoxy-4-[4-(propan-2-yl)piperazin-1-yl]-6-(pyrid-3-yl)-9H-pyrrolo[2,3-b:5,4-c′]dipyridine;
4-(4-cyclopropylpiperazin-1-yl)-3-methoxy-6-(pyrid-3-yl)-9H-pyrrolo[2,3-b:5,4-c′]dipyridine;
4-(4-ethylpiperazin-1-yl)-3-methoxy-6-(pyrid-3-yl)-9H-pyrrolo[2,3-b:5,4-c′]dipyridine;
3-methoxy-4-[4-(methylsulfonyl)piperazin-1-yl]-6-(pyrid-3-yl)-9H-pyrrolo[2,3-b:5,4-c′]-dipyridine;
3-fluoro-4-[4-(methylsulfonyl)piperazin-1-yl]-6-(pyrid-3-yl)-9H-pyrrolo[2,3-b:5,4-c′]-dipyridine;
3-{4-[3-fluoro-6-(pyrid-3-yl)-9H-pyrrolo[2,3-b:5,4-c′]dipyrid-4-yl]phenyl}propanoic acid;
3-fluoro-4-(6-methoxypyrid-3-yl)-6-(pyrid-3-yl)-9H-pyrrolo[2,3-b:5,4-c′]dipyridine;
N-{3-[3-fluoro-6-(pyrid-3-yl)-9H-pyrrolo[2,3-b:5,4-c′]dipyrid-4-yl]phenyl}methanesulfon-amide;
3-fluoro-4-(4-methylthiophen-2-yl)-6-(pyrid-3-yl)-9H-pyrrolo[2,3-b:5,4-c′]dipyridine;
3-fluoro-4-(1H-indol-6-yl)-6-(pyrid-3-yl)-9H-pyrrolo[2,3-b:5,4-c′]dipyridine;
{2-[3-fluoro-6-(pyrid-3-yl)-9H-pyrrolo[2,3-b:5,4-c′]dipyrid-4-yl]phenyl}methanol;
3-fluoro-4-(4-methylthiophen-3-yl)-6-(pyrid-3-yl)-9H-pyrrolo[2,3-b:5,4-c′]dipyridine;
3-[3-fluoro-6-(pyrid-3-yl)-9H-pyrrolo[2,3-b:5,4-c′]dipyrid-4-yl]-N,N-dimethylaniline;
3-fluoro-4-(5-methylfuran-2-yl)-6-(pyrid-3-yl)-9H-pyrrolo[2,3-b:5,4-c′]dipyridine;
3-fluoro-4-(1-methyl-1 H-indol-5-yl)-6-(pyrid-3-yl)-9H-pyrrolo[2,3-b:5,4-c′]dipyridine;
3-fluoro-4-(1-methyl-1 H-pyrazol-4-yl)-6-(pyrid-3-yl)-9H-pyrrolo[2,3-b:5,4-c′]dipyridine;
N-{4-[3-fluoro-6-(pyrid-3-yl)-9H-pyrrolo[2,3-b:5,4-c′]dipyrid-4-yl]benzyl}acetamide;
N-[3-{3-fluoro-6-(pyrid-3-yl)-9H-pyrrolo[2,3-b:5,4-c′]dipyrid-4-yl]benzyl}methanesulfon-amide;
3-fluoro-4-(2-methoxyphenyl)-6-(pyrid-3-yl)-9,4-pyrrolo[2,3-b:5,4-c′]dipyridine;
4-(2-ethoxypyrid-3-yl)-3-fluoro-6-(pyrid-3-yl)-9H-pyrrolo[2,3-b:5,4-c′]dipyridine;
4-({3-[3-fluoro-6-(pyrid-3-yl)-9H-pyrrolo[2,3-b:5,4-c′]dipyrid-4-yl]phenyl}amino)-4-oxobutanoic acid;
N-{4-[3-fluoro-6-(pyrid-3-yl)-9H-pyrrolo[2,3-b:5,4-c′]dipyrid-4-yl]benzyl}methanesulfonamide;
{4-[3-fluoro-6-(pyrid-3-yl)-9H-pyrrolo[2,3-b:5,4-c′]dipyrid-4-yl]phenyl}(morpholin-4-yl)methanone;
3-fluoro-4-(1-methyl-1 H-pyrazol-5-yl)-6-(pyrid-3-yl)-9H-pyrrolo[2,3-b:5,4-c′]dipyridine;
1-{2-[3-fluoro-6-(pyrid-3-yl)-9H-pyrrolo[2,3-b:5,4-c′]dipyrid-4-yl]phenyl}-N,N-dimethyl-methanamine;
2-[3-fluoro-6-(pyrid-3-yl)-9H-pyrrolo[2,3-b:5,4-c′]dipyrid-4-yl]benzonitrile.
1-chloro-N-{4-[3-fluoro-6-(pyrid-3-yl)-9H-pyrrolo[2,3-b:5,4-c′]dipyrid-4-yl]phenyl}methane-sulfonamide;
3-(4-methylpiperazin-1-yl)-6-(pyrid-3-yl)-9H-pyrrolo[2,3-b:5,4-c′]dipyridine;
N-{4-[3-fluoro-6-(pyrid-3-yl)-9H-pyrrolo[2,3-b:5,4-c′]dipyrid-4-yl]phenyl}cyclopropanesulfonamide;
N-{4-[3-fluoro-6-(pyrid-3-yl)-9H-pyrrolo[2,3-b:5,4-c′]dipyrid-4-yl]-2-methoxyphenyl}-methanesulfonamide;
N-{4-[3-fluoro-6-(1-methyl-1H-pyrazol-4-yl)-9H-pyrrolo[2,3-b:5,4-c′]dipyrid-4-yl]phenyl}methanesulfonamide;
3-fluoro-6-(5-methoxypyrid-3-yl)-9H-pyrrolo[2,3-b:5,4-c′]dipyridine;
3-fluoro-6-(4-methoxypyrid-3-yl)-9H-pyrrolo[2,3-b:5,4-c′]dipyridine;
6-(1-benzyl-1 H-pyrazol-4-yl)-3-fluoro-9H-pyrrolo[2,3-b:5,4-c′]dipyridine;
3-fluoro-6-(1-methyl-1H-pyrazol-4-yl)-9H-pyrrolo[2,3-b:5,4-c′]dipyridine;
3-fluoro-6-[1-(2-methylpropyl)-1H-pyrazol-4-yl]-9H-pyrrolo[2,3-b:5,4-c′]dipyridine;
3-fluoro-6-[5-(methylsulfanyl)pyrid-3-yl]-9H-pyrrolo[2,3-b:5,4-c′]dipyridine;
3-fluoro-6-{1-[2-(morpholin-4-yl)ethyl]-1 H-pyrazol-4-yl}-9H-pyrrolo[2,3-b:5,4-c′]dipyridine;
3-fluoro-4-[4-(propan-2-yl)piperazin-1-yl]-6-(pyrid-3-yl)-9H-pyrrolo[2,3-b:5,4-c′]dipyridine;
3-fluoro-4-(piperid-1-yl)-6-(pyrid-3-yl)-9H-pyrrolo[2,3-b:5,4-c′]dipyridine;
4-[3-fluoro-6-(pyrid-3-yl)-9H-pyrrolo[2,3-b:5,4-c′]dipyrid-4-yl]-2-methylbut-3-yn-2-amine;
4-[3-fluoro-6-(pyrid-3-yl)-9H-pyrrolo[2,3-b:5,4-c′]dipyrid-4-yl]-2-methylbut-3-yn-2-ol;
4-[3-(4-ethylpiperazin-1-yl)-3-methylbut-1-yn-1-yl]-3-fluoro-6-(pyrid-3-yl)-9H-pyrrolo[2,3-b:5,4-c′]dipyridine;
3-{4-[3-fluoro-6-(pyrid-3-yl)-9H-pyrrolo[2,3-b:5,4-c′]dipyrid-4-yl]phenyl}propanoic acid;
3-fluoro-4-(6-methoxypyrid-3-yl)-6-(pyrid-3-yl)-9H-pyrrolo[2,3-b:5,4-c′]dipyridine;
N-{3-[3-fluoro-6-(pyrid-3-yl)-9H-pyrrolo[2,3-b:5,4-c′]dipyrid-4-yl]phenyl}methanesulfonamide;
3-fluoro-4-(4-methylthiophen-2-yl)-6-(pyrid-3-yl)-9H-pyrrolo[2,3-b:5,4-c′]dipyridine;
3-fluoro-4-(1H-indol-6-yl)-6-(pyrid-3-yl)-9H-pyrrolo[2,3-b:5,4-c′]dipyridine;
{2-[3-fluoro-6-(pyrid-3-yl)-9H-pyrrolo[2,3-b:5,4-c′]dipyrid-4-yl]phenyl}methanol;
3-fluoro-4-(4-methylthiophen-3-yl)-6-(pyrid-3-yl)-9H-pyrrolo[2,3-b:5,4-c′]dipyridine;
3-[3-fluoro-6-(pyrid-3-yl)-9H-pyrrolo[2,3-b:5,4-c′]dipyrid-4-yl]-N,N-dimethylaniline;
3-fluoro-4-(1-methyl-1H-indol-5-yl)-6-(pyrid-3-yl)-9H-pyrrolo[2,3-b:5,4-c′]dipyridine;
3-fluoro-4-(1-methyl-1 H-pyrazol-4-yl)-6-(pyrid-3-yl)-9H-pyrrolo[2,3-b:5,4-c′]dipyridine;
N-{4-[3-fluoro-6-(pyrid-3-yl)-9H-pyrrolo[2,3-b:5,4-c′]dipyrid-4-yl]benzyl}acetamide;
N-{3-[3-fluoro-6-(pyrid-3-yl)-9H-pyrrolo[2,3-b:5,4-c′]dipyrid-4-yl]benzyl}methanesulfonamide;
3-fluoro-4-(2-methoxyphenyl)-6-(pyrid-3-yl)-9H-pyrrolo[2,3-b:5,4-c′]dipyridine;
4-(2-ethoxypyrid-3-yl)-3-fluoro-6-(pyrid-3-yl)-9H-pyrrolo[2,3-b:5,4-c′]dipyridine;
4-({3-[3-fluoro-6-(pyrid-3-yl)-9H-pyrrolo[2,3-b:5,4-c′]dipyrid-4-yl]phenyl}amino)-4-oxobutanoic acid;
N-{4-[3-fluoro-6-(pyrid-3-yl)-9H-pyrrolo[2,3-b:5,4-c′]dipyrid-4-yl]benzyl}methanesulfonamide;
3-fluoro-4-(1-methyl-1 H-pyrazol-5-yl)-6-(pyrid-3-yl)-9H-pyrrolo[2,3-b:5,4-c′]dipyridine;
N-{4-[3-fluoro-6-(pyrid-3-yl)-9H-pyrrolo[2,3-b:5,4-C′]dipyrid-4-yl]phenyl}-2-methylpropanamide;
3-fluoro-4,6-di(pyrid-3-yl)-9H-pyrrolo[2,3-b:5,4-c′]dipyridine;
N-{2-[3-fluoro-6-(pyrid-3-yl)-9H-pyrrolo[2,3-b:5,4-c′]dipyrid-4-yl]phenyl}methanesulfon-amide;
3-fluoro-4-(1 H-pyrazol-4-yl)-6-(pyrid-3-yl)-9H-pyrrolo[2,3-b:5,4-c′]dipyridine;
3-fluoro-4-[3-(methyl sulfonyl)phenyl]-6-(pyrid-3-yl)-9H-pyrrolo[2,3-b:5,4-c′]dipyridine;
3-fluoro-4-(2-methoxypyrimidin-5-yl)-6-(pyrid-3-yl)-9H-pyrrolo[2,3-b:5,4-c′]dipyridine;
5-[3-fluoro-6-(pyrid-3-yl)-9H-pyrrolo[2,3-b:5,4-c′]dipyrid-4-yl]pyrid-2-amine;
3-fluoro-4-[4-(1-methylpiperid-4-yl)piperazin-1-yl]-6-(pyrid-3-yl)-9H-pyrrolo[2,3-b:5,4-c′]-dipyridine;
3-fluoro-4-[4-(morpholin-4-yl)piperid-1-yl]-6-(pyrid-3-yl)-9H-pyrrolo[2,3-b:5,4-c′]dipyridine;
N,N-diethyl-2-{4-[3-fluoro-6-(pyrid-3-yl)-9H-pyrrolo[2,3-b:5,4-c′]dipyrid-4-yl]piperazin-1-yl}ethanamine;
3-fluoro-4-(4-methyl-1,4-diazepan-1-yl)-6-(pyrid-3-yl)-9H-pyrrolo[2,3-b:5,4-c′]dipyridine;
2-{4-[3-fluoro-6-(pyrid-3-yl)-9H-pyrrolo[2,3-b:5,4-c′]dipyrid-4-yl]piperazin-1-yl}ethanol;
3-fluoro-4-[4-(4-methylpiperazin-1-yl)piperid-1-yl]-6-(pyrid-3-yl)-9H-pyrrolo[2,3-b:5,4-c′]dipyridine;
N-{4-[3-fluoro-6-(pyrid-3-yl)-9H-pyrrolo[2,3-b:5,4-c′]dipyrid-4-yl]phenyl}-N-methylmethane-sulfonamide;
3-(piperazin-1-yl)-6-(pyrid-3-yl)-9H-pyrrolo[2,3-b:5,4-c′]dipyridine;
6-(pyrid-3-yl)-9H-pyrrolo[2,3-b:5,4-c′]dipyrid-3-amine;
4-(1,4′-bipiperid-1′-yl)-3-fluoro-6-(pyrid-3-yl)-9H-pyrrolo[2,3-b:5,4-c′]dipyridine;
1-[3-fluoro-6-(pyrid-3-yl)-9H-pyrrolo[2,3-b:5,4-c′]dipyrid-4-yl]-N,N-dimethylpiperid-4-amine;
3-fluoro-6-(pyrid-3-yl)-4-[4-(pyrrolidin-1-yl)piperid-1-yl]-9H-pyrrolo[2,3-b:5,4-c′]dipyridine;
3-fluoro-4-{4-[3-(piperid-1-yl)propyl]piperazin-1-yl}-6-(pyrid-3-yl)-9H-pyrrolo[2,3-b:5,4-c′]-dipyridine;
3-fluoro-4-{4-[3-(morpholin-4-yl)propyl]piperazin-1-yl}-6-(pyrid-3-yl)-9H-pyrrolo[2,3-b:5,4-c′]dipyridine;
3-{-4-[3-fluoro-6-(pyrid-3-yl)-9H-pyrrolo[2,3-b:5,4-c′]dipyrid-4-yl]piperazin-1-yl}-N,N-dipropylpropan-1-amine;
3-ethoxy-6-(pyrid-3-yl)-9H-pyrrolo[2,3-b:5,4-c′]dipyridine;
3-iodo-6-(pyrid-3-yl)-9H-pyrrolo[2,3-b:5,4-c′]dipyridine;
3-{1-[2-(morpholin-4-yl)ethyl]-1H-pyrazol-4-yl}-6-(pyrid-3-yl)-9H-pyrrolo[2,3-b:5,4-c′]-dipyridine;
3-(1-methyl-1H-pyrazol-3-yl)-6-(pyrid-3-yl)-9H-pyrrolo[2,3-b:5,4-c′]dipyridine;
N,N-diethyl-3-{4-[3-fluoro-6-(pyrid-3-yl)-9H-pyrrolo[2,3-b:5,4-c′]dipyrid-4-yl]piperazin-1-yl}-propan-1-amine;
N,N-diethyl-2-{4-[6-(pyrid-3-yl)-9H-pyrrolo[2,3-b:5,4-c′]dipyrid-3-yl]-1H-pyrazol-1-yl}ethan-amine;
3-fluoro-4-methoxy-6-(pyrid-3-yl)-9H-pyrrolo[2,3-b:5,4-c′]dipyridine;
3-[1-(2-methylpropyl)-1H-pyrazol-4-yl]-6-(pyrid-3-yl)-9H-pyrrolo[2,3-b:5,4-c′]dipyridine;
3-[4-(morpholin-4-yl)phenyl]-6-(pyrid-3-yl)-9H-pyrrolo[2,3-b:5,4-c′]dipyridine;
N-propyl-6-(pyrid-3-yl)-9H-pyrrolo[2,3-b:5,4-c′]dipyrid-3-amine;
3-{4-[4-(propan-2-yl)piperazin-1-yl]phenyl}-6-(pyrid-3-yl)-9H-pyrrolo[2,3-b:5,4-c′]dipyridine;
6-(pyrid-3-yl)-3-(2,2,2-trifluoroethoxy)-9H-pyrrolo[2,3-b:5,4-c′]dipyridine;
3-fluoro-9H-pyrrolo[2,3-b:5,4-c′]dipyridine-6-carbonitrile;
3-(2-methoxyethoxy)-6-(pyrid-3-yl)-9H-pyrrolo[2,3-b:5,4-c′]dipyridine;
3-{1-[3-(4-methylpiperazin-1-yl)propyl]-1H-pyrazol-4-yl}-6-(pyrid-3-yl)-9H-pyrrolo[2,3-b:5,4-c′]dipyridine;
{3-[6-(pyrid-3-yl)-9H-pyrrolo[2,3-b:5,4-c′]dipyrid-3-yl]phenyl}methanol;
N,N-diethyl-3-[6-(pyrid-3-yl)-9H-pyrrolo[2,3-b:5,4-c′]dipyrid-3-yl]benzamide;
3-(3,5-dimethyl-1H-pyrazol-4-yl)-6-(pyrid-3-yl)-9H-pyrrolo[2,3-b:5,4-c′]dipyridine;
2-{3,5-dimethyl-4-[6-(pyrid-3-yl)-9H-pyrrolo[2,3-b:5,4-c′]dipyrid-3-yl]-1 H-pyrazol-1-yl}-N,N-diethylethanamine;
3-methoxy-6-(1-methyl-1 H-pyrazol-4-yl)-9H-pyrrolo[2,3-b:5,4-c′]dipyridine;
methyl 4-{6-[1-(prop-2-en-1-yl)-1H-pyrazol-4-yl]-9H-pyrrolo[2,3-b:5,4-c′]dipyrid-4-yl}benzoate;
N,N-diethyl-2-[4-(3-methoxy-9H-pyrrolo[2,3-b:5,4-c′]dipyrid-6-yl)-3,5-dimethyl-1 H-pyrazol-1-yl]ethanamine;
N-[2-(dimethylamino)ethyl]-2-[4-(3-methoxy-9H-pyrrolo[2,3-b:5,4-c′]dipyrid-6-yl)-1 H-pyrazol-1-yl]acetamide;
3-(1 H-pyrazol-4-yl)-6-(pyrid-3-yl)-9H-pyrrolo[2,3-b:5,4-c′]dipyridine;
N,N-diethyl-3-{4-[6-(pyrid-3-yl)-9H-pyrrolo[2,3-b:5,4-c′]dipyrid-3-yl]-1 H-pyrazol-1-yl}-propan-1-amine;
N,N-diethyl-3-[4-(3-methoxy-9H-pyrrolo[2,3-b; 5,4-c′]dipyrid-6-yl)-1 H-pyrazol-1-yl]propan-1-amine;
9H-pyrrolo[2,3-b:5,4-c′]dipyridine-6-carboxylic acid;
N-[3-(dimethylamino)propyl]-N-{4-[3-fluoro-6-(pyrid-3-yl)-9H-pyrrolo[2,3-C′]dipyrid-4-yl]phenyl}methanesulfonamide;
(4-methylpiperazin-1-yl)(9H-pyrrolo[2,3-b:5,4-c′]dipyrid-6-yl)methanone;
5-[4-(3-methoxy-9H-pyrrolo[2,3-b:5,4-c′]dipyrid-6-yl)-1 H-pyrazol-1-yl]pentan-1-amine;
2-methyl-2-propyl {5-[4-(3-methoxy-9H-pyrrolo[2,3-b:5,4-c′]dipyrid-6-yl)-1 H-pyrazol-1-yl]pentyl}carbamate;
3-methoxy-6-{1-[2-(1-methylpiperid-2-yl)ethyl]-1 H-pyrazol-4-yl}-9H-pyrrolo[2,3-b:5,4-c′]-dipyridine;
3-{-4-[3-fluoro-6-(pyrid-3-yl)-9H-pyrrolo[2,3-b:5,4-c′]dipyrid-4-yl]phenoxy}-N,N-dimethylpropan-1-amine;
4-[3-fluoro-6-(pyrid-3-yl)-9H-pyrrolo[2,3-b:5,4-c′]dipyrid-4-yl]phenol;
2-{4-[3-fluoro-6-(pyrid-3-yl)-9H-pyrrolo[2,3-b:5,4-c′]dipyrid-4-yl]phenoxy}-N,N-dimethyl-ethanamine;
3-{1-[4(1-ethylpyrrolidin-2-yl)methyl]-1 H-pyrazol-4-yl}-6-(pyrid-3-yl)-9H-pyrrolo[2,3-b:5,4-c′]dipyridine;
3-fluoro-6-(pyrid-3-yl)-4-{4-[2-(pyrrolidin-1-yl)ethoxy]phenyl}-9H-pyrrolo[2,3-b:5,4-c′]-dipyridine;
3-fluoro-6-(thiophen-3-yl)-9H-pyrrolo[2,3-b:5,4-c′]dipyridine;
2-methyl-2-propyl 4-{4-[6-(pyrid-3-yl)-9H-pyrrolo[2,3-b:5,4-c′]dipyrid-3-yl]-phenyl}piperazine-1-carboxylate;
3-{4-[3-fluoro-6-(pyrid-3-yl)-9H-pyrrolo[2,3-b:5,4-c′]dipyrid-4-yl]phenoxy}-N,N,2-trimethyl-propan-1-amine;
3-fluoro-4-{4-[2-(morpholin-4-yl)ethoxy]phenyl}-6-(pyrid-3-yl)-9H-pyrrolo[2,3-b:5,4-c′]-dipyridine;
N,N-diethyl-2-{4-[3-fluoro-6-(pyrid-3-yl)-9H-pyrrolo[2,3-b:5,4-c′]dipyrid-4-yl]phenoxy}ethan-amine;
N-[2-(dimethylamino)ethyl]-5-[3-fluoro-6-(pyrid-3-yl)-9H-pyrrolo[2,3-b:5,4-c′]dipyrid-4-yl]-pyridine-2-carboxamide;
1-{4-[3-fluoro-6-(pyrid-3-yl)-9H-pyrrolo[2,3-b:5,4-c′]dipyrid-4-yl]phenoxy}-3-morpholin-4-yl)propan-2-ol;
N-ethyl-3-{-4-[3-fluoro-6-(pyrid-3-yl)-9H-pyrrolo[2,3-b:5,4-c′]dipyrid-4-yl]phenoxy}propan-1-amine;
4-[6-(pyrid-3-yl)-9H-pyrrolo[2,3-b:5,4-c′]dipyrid-3-yl]phenol;
3-[4-(piperazin-1-yl)phenyl]-6-(pyrid-3-yl)-9H-pyrrolo[2,3-b:5,4-c′]dipyridine;
3-fluoro-6-(isoquinolin-4-yl)-9H-pyrrolo[2,3-b; 5,4-c′]dipyridine;
N,N-dimethyl-3-{4-[6-(pyrid-3-yl)-9H-pyrrolo[2,3-b; 5,4-c′]dipyrid-3-yl]phenoxy}propan-1-amine;
3-{-4-[3-(piperid-1-yl)propoxy]phenyl}-6-(pyrid-3-yl)-9H-pyrrolo[2,3-b:5,4-c′]dipyridine;
3-{4-[2-(morpholin-4-yl)ethoxy]phenyl}-6-(pyrid-3-yl)-9H-pyrrolo[2,3-b:5,4-c′]dipyridine;
3-{4-[3-(morpholin-4-yl)propoxy]phenyl}-6-(pyrid-3-yl)-9H-pyrrolo[2,3-b:5,4-c′]dipyridine;
3-{4-[2-(1H-imidazol-1-yl)ethoxy]phenyl}-6-(pyrid-3-yl)-9H-pyrrolo[2,3-b:5,4-c′]dipyridine;
3-(4-{3-[4-(methyl sulfonyl)piperazin-1-yl]propoxy}phenyl)-6-(pyrid-3-yl)-9H-pyrrolo[2,3-b:5,4-c′]dipyridine;
N,N-diethyl-2-{3-[6-(pyrid-3-yl)-9H-pyrrolo[2,3-b:5,4-c′]dipyrid-3-yl]phenoxy}ethanamine;
2-methyl-2-propyl 4-{3-[6-(pyrid-3-yl)-9H-pyrrolo[2,3-b:5,4-c′]dipyrid-3-yl]-phenyl}piperazine-1-carboxylate;
N,N,4-triethyl-5-[6-(pyrid-3-yl)-9H-pyrrolo[2,3-b:5,4-c′]dipyrid-3-yl]pyrid-2-amine;
3-[3-(piperazin-1-yl)phenyl]-6-(pyrid-3-yl)-9H-pyrrolo[2,3-b:5,4-c′]dipyridine hydrochloride;
N,N-diethyl-2-({4-[3-fluoro-6-(pyrid-3-yl)-9H-pyrrolo[2,3-b:5,4-c′]dipyrid-4-yl]-2-methylbut-3-yn-2-yl}oxy)ethanamine;
4-[3-fluoro-6-(pyrid-3-yl)-9H-pyrrolo[2,3-b:5,4-c′]dipyrid-4-yl]-N-(prop-2-en-1-yl)aniline;
N-(2-methylpropan-2-yl)-5-(9H-pyrrolo[2,3-b:5,4-c′]dipyrid-6-yl)pyridine-3-carboxamide;
5-(3-fluoro-9H-pyrrolo[2,3-b:5,4-c′]dipyrid-6-yl)-N-(2-methylpropan-2-yl)pyridine-3-carboxamide;
3-fluoro-6-(1H-pyrazol-4-yl)-9H-pyrrolo[2,3-b:5,4-c′]dipyridine;
(2E)-N-[4-(dimethylamino)butyl]-3-[3-fluoro-6-(pyrid-3-yl)-9H-pyrrolo[2,3-b:5,4-c′]dipyrid-4-yl]prop-2-enamide;
6-chloro-3-fluoro-9H-pyrrolo[2,3-b:5,4-c′]dipyridine;
3-{4-[6-(pyrid-3-yl)-9H-pyrrolo[2,3-b:5,4-c′]dipyrid-3-yl]phenoxy}propan-1-amine.

6. A pharmaceutical composition comprising the compound of claim 1, or an addition salt of compound with a pharmaceutically acceptable acid.

7. The pharmaceutical composition of claim 6 further comprising at least one pharmaceutically compatible excipient.

8. A method for treating cancer in a patient in need thereof comprising administering to said patient a therapeutically effective amount of the pharmaceutical composition of claim 6.

9. A method of treating diseases that are sensitive to Pim kinase deregulation in a patient in need thereof comprising administering to said patient a therapeutically effective amount of the pharmaceutical composition of claim 6.

10. (canceled)

11. (canceled)

12. The compound of claim 1 wherein said compound is a kinase inhibitor.

13. The compound of claim 1 wherein said compound is a Pim kinase inhibitors inhibitor.

14. A process for synthesizing the compound of claim 1 having the following steps in Scheme 1 below: 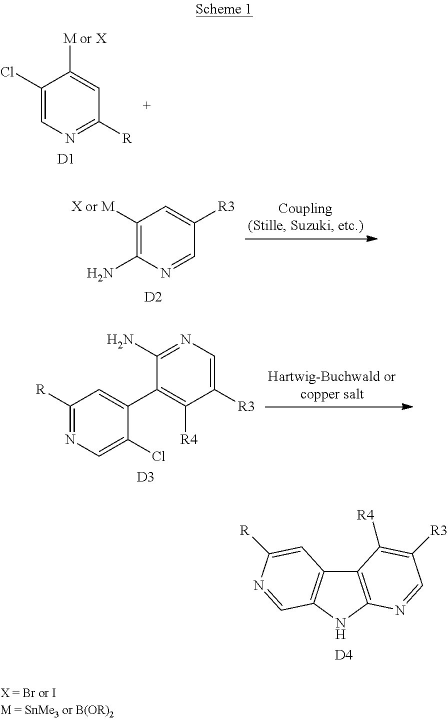
in which the substituents R3 and R4 have the meanings given hereinabove or hereinbelow, and R represents either the values of R6 as defined above, or the following values: OH, OCH₃, OS(O)₂CF₃, Cl, SCH₃, CN.

15. A compound having formula D3: 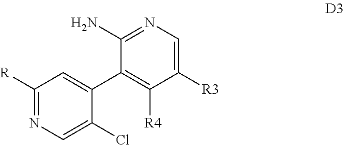
in which the substituents R3 and R4 have the meanings indicated in any one of claims 1 to 5, and R represents the values of R6 as defined in any one of claims 1 to 5 and the following values: OH, OCH₃, OS(O)₂CF₃, Cl, SCH₃, CN.

16. A compound having formula D3: 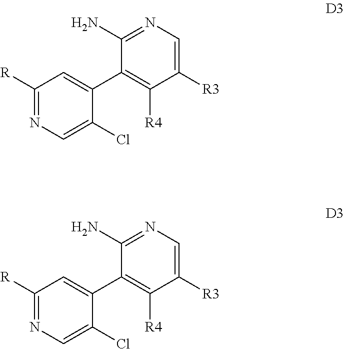
in which the substituent R3 represents a fluorine atom or a methoxy radical, and the substituent R4 represents a hydrogen atom, R being chosen from the values of R6 as defined in any one of claims 1 to 5 and the following values: OH, OCH₃, OS(O)₂CF₃, Cl, SCH₃, CN.

17. A compound having formula D4: 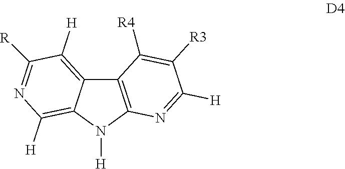
in which the substituents R3 and R4 have the meanings indicated in any one of claims 1 to 5 and R represents the following values: OH, OCH₃, OS(O)₂CF₃, Cl, SCH₃, CN.
